(19) United States
(12) Patent Application Publication
(10) Pub. No.: US 2017/0216252 A1
(43) Pub. Date: Aug. 3, 2017

(54) ANTI-BACTERIAL PYRUVATE KINASE MODULATOR COMPOUNDS, COMPOSITIONS, USES AND METHODS

(71) Applicants: SIMON FRASER UNIVERSITY, Burnaby (CA); CENTRE FOR DRUG RESEARCH AND DEVELOPMENT, Vancouver (CA)

(72) Inventors: Robert N. YOUNG, Vancouver (CA); Nag Sharwan KUMAR, Coquitlam (CA); Christophe LABRIERE, La Feuillee (FR); Jon Paul SELVAN, Thely (IN); James Brian JAQUITH, Cobourg (CA); Edith Mary DULLAGHAN, Vancouver (CA)

(21) Appl. No.: 15/325,602

(22) PCT Filed: Jul. 10, 2015
(86) PCT No.: PCT/CA2015/000434

§ 371 Date: Jan. 11, 2017

Related U.S. Application Data
(60) Provisional application No. 62/023,751, filed on Jul. 11, 2014.

(51) Int. Cl. A61K 31/404; A61K 31/5377; A61K 31/496; A61K 31/428; A61K 31/501; A61K 31/497; A61K 31/454; A61K 31/4184; A61K 31/4155; A61K 31/4439; A61K 31/4709; A61K 31/4178; A61K 31/422; A61K 31/437; A61K 31/427; A61K 31/506; A61K 45/06

(52) U.S. Cl. CPC A61K 31/404; A61K 45/06; A61K 31/5377; A61K 31/496; A61K 31/428; A61K 31/501; A61K 31/497; A61K 31/454; A61K 31/4184; A61K 31/4155; A61K 31/4439; A61K 31/4709; A61K 31/4178; A61K 31/422; A61K 31/437; A61K 31/427; A61K 31/506

(57) ABSTRACT

Compounds of general formula I that are capable of inhibiting bacterial pyruvate kinase and/or bacterial growth. The compounds may find use as antibacterial agents in therapeutic and/or non-therapeutic contexts.

### FIELD OF THE INVENTION

This invention relates generally to the field of anti-bacterial compounds. In particular, to compounds and compositions for, and methods of, treating bacterial infections, including those where the bacteria have developed resistance to other antibiotics.

### CROSS REFERENCE TO RELATED APPLICATION

This application claims the benefit of priority from U.S. Provisional Application Ser. No. 62/023,751, filed 11 Jul. 2014.

### BACKGROUND OF THE INVENTION

Infectious diseases caused by bacterial and eukaryotic pathogens continue to be a threat to human health. In particular, many bacteria are developing antibiotic resistance and the effectiveness of the available antimicrobial drugs against bacteria such as methicillin-resistant Staphylococcus aureus (MRSA) is diminishing at a rapid pace. The hospital-acquired ESKAPE pathogens (Enterococcus faecium, Staphylococcus aureus, Klebsiella pneumoniae, Acinetobacter baumannii, Pseudomonas aeruginosa and Enterobacter spp.) have also been recognised as serious heath threats.

Pyruvate kinase (PK) is known to be a significant protein and is responsible for catalyzing the final step of glycolysis, which involves the transfer of the phosphoryl group of phosphoenolpyruvate (PEP) to ADP to produce pyruvate and ATP (Suzuki K, et al., 2008, J Biochem, 144(3):305-312). PKs exist as homotetramers of identical subunits of ˜50-60 KDa depending on species, each consisting of three to four domains: A, B, C, and N-terminal domains. The N-terminal helical domain is absent in prokaryotic PKs and can be removed from human erythrocyte PK with no effect on its stability or activity (Valentini et al., 2002, J. Biol. Chem., 277:23807-23814). While there are four mammalian PK isozymes, M1, M2, L (liver), and R (red blood cell), with different primary structures, kinetic properties, and tissue distributions to satisfy the metabolic requirements of various tissues, most bacteria and lower eukaryotes have only one PK isoenzyme. Only a few bacterial species, specifically E. coli and Salmonella typhimurium, have two isoenzymes.

Inhibitors of bacterial PKs identified by structural modelling and in silico library screening have been described (Zoraghi et al., 2011, Antimicrob. Agents Chemother., 55:2042-2053; International Patent Application No. PCT/CA2011/001175 (WO 2012/051708)). A class of MRSA PK inhibitors derived from a naturally occurring marine alkaloid has also been described (Kumar et al., 2014, Bioog. Med. Chem., 22:1708-1725).

Several indole- or benzimidazole-containing compounds have been described as having anti-mycobacterial activity (Matyk et al., 2005, Il Farmaco, 60:399-408), anti-microbial activity (International Patent Application No. PCT/US2003/027963 (WO 2005/033065), or broad spectrum anti-bacterial activity (U.S. Pat. No. 8,691,859).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates generally to antibacterial pyruvate kinase modulator compounds, compositions, uses and methods. In one aspect, the invention relates to a method of treating a subject known to have or suspected of having a bacterial infection, the method comprising administering to the subject an effective amount of a compound of general formula I:

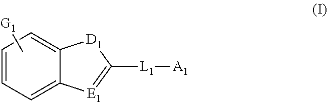

or a salt thereof, wherein:
L₁ is —CH₂CH₂—, —CHCH—, —CC—, —C(O)NH—, —NHC(O)—, —C(O)—, —N═CH—, —CH₂R₅—, —NHCH₂—,

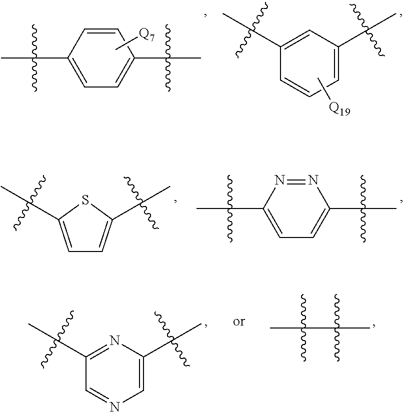

wherein R₅ is CH₂CH₂, NHCH₂, NH, SCH₂, O, or S, and wherein each Q₇ and Q₁₉ are independently H, NO₂, or OMe;
A₁ is

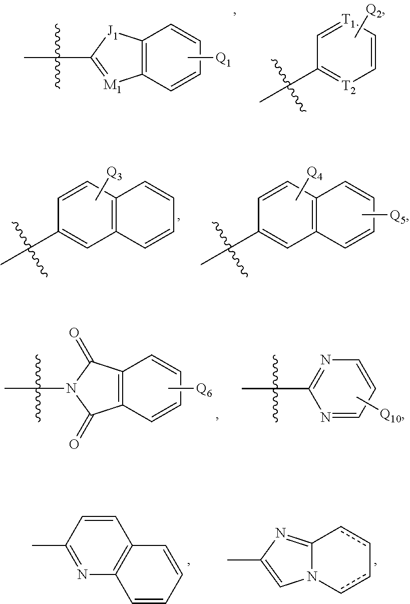

wherein [symbol] represents a single or double bond, or a 5-membered heteroaryl optionally substituted with Q₁₁ and containing 1 or 2 heteroatoms each selected from N, O and S;
each G₁ is independently H, Br, F, Cl, I, OR₁, SR₁, SO₂R₁, C(O)R₁, C(O)OR₁, unsubstituted phenyl, substituted phenyl, unsubstituted alkyl, substituted alkyl, unsubstituted allyl, or substituted allyl, wherein the alkyl or allyl is 1-6 carbons in length, wherein the substitutions to the phenyl, alkyl, or allyl are optionally Br, F, Cl, I, OH, OMe, or N₃, and wherein R₁ is H or Me;
D₁ is S, O, N—H, N—CH₃, N—CH₂—OCH₃, N—CH₂—CH₃, N—CH₂—COOH, N—CH₂—CH₂OH, CH₂, CH—CH₃, CH—CH₂—OCH₃, CH—CH₂—CH₃, CH—CH₂—COOH, CH—CH₂—CH₂OH, N—R₂, or CH—R₂, wherein R₂ is

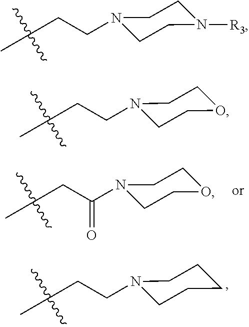

wherein R₃ is H, unsubstituted alkyl, or substituted alkyl, wherein the alkyl is 1-6 carbons in length, and the alkyl is optionally substituted with Br, F, Cl, I, OH, OMe, or N₃;
E₁ is N, C—H, C—CH₃, C—C(O)OR₄, C—C(O)R₆₃, C—Cl, C—Br, C—F, or C—COR₄, wherein R₄ is H or Me, and R₆₃ is CH₃, CH₂CH₃, CH₂CH₂CH₃, CH(CH₃)₂ or CF₃, and wherein if 1)₁ is CH₂, CH—CH₃, CH—CH₂—OCH₃, CH—CH₂—CH₃, CH—CH₂—COOH, CH—CH₂—CH₂OH, or CH—R₂, E₁ is N;
each Q₁ is independently H, Br, F, Cl, I,

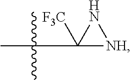 [SMILES: *C1(C(F)(F)F)NN1]

OR₆, SR₆, SO₂R₆, C(O)R₆, C(O)OR₆, N₃, unsubstituted phenyl, substituted phenyl, unsubstituted alkyl, substituted alkyl, unsubstituted allyl, or substituted allyl, wherein the alkyl or allyl is 1-6 carbons in length, wherein R₆ is H or Me, and wherein the substituted phenyl, alkyl, or allyl is optionally substituted with Q₈;
each Q₂ is independently H, Br, F, Cl, I,

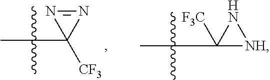 [SMILES: *C1(C(F)(F)F)N=N1 ; *C1(C(F)(F)F)NN1]

N₃, OR₇, SR₇, SO₂R₇, C(O)R₇, NO₂,

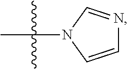 [SMILES: *n1ccnc1]

unsubstituted phenyl, substituted phenyl, unsubstituted alkyl, substituted alkyl, unsubstituted allyl, or substituted allyl, wherein the alkyl or allyl is 1-6 carbons in length, wherein R₇ is H or Me, and wherein the substituted phenyl, alkyl, or allyl is optionally substituted with Q₉;
each Q₃ is independently H, Br, F, Cl, I, or OR₈, wherein R₈ is H or Me;
each Q₄ is independently H, Br, F, Cl, I, or OR₉, wherein R₉ is H or Me;
each Q₅ is independently H, Br, F, Cl, I, or OR₁₀, wherein R₁₀ is H or Me;
each Q₆ is independently H, Br, F, Cl, I, or OR₁₁, wherein R₁₁ is H or Me;
each Q₈ is independently Br, F, Cl, I, Me, or OR₁₂, wherein R₁₂ is H or Me;
each Q₉ is independently Br, F, Cl, I, Me, or OR₁₃, wherein R₁₃ is H or Me;
each Q₁₀ is independently H, Br, F, Cl or I;
each Q₁₁ is independently H, Me, unsubstituted phenyl or substituted phenyl, wherein the substituted phenyl is optionally substituted with Q₈;
J₁ is S, O, N—H, N—CH₃, N—CH₂—OCH₃, N—CH₂—COOH, N—CH₂—CH₂OH, CH—CH₃, N—R₁₄, or CH—R₁₄, wherein R₁₄ is

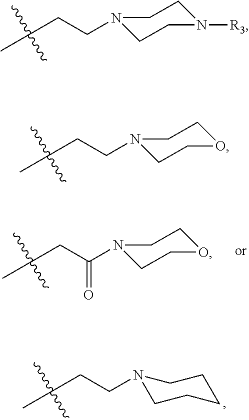

wherein R₃ is H, unsubstituted alkyl, or substituted alkyl, wherein the alkyl is 1-6 carbons in length, and the alkyl is optionally substituted with Br, F, Cl, I, OH, OMe, or N₃;
M₁ is N, C—H, C—CH₃, C—C(O)OR₄, C—C(O)R₆₃, or C—CH(CH₃)₂, wherein R₄ is H or Me, and R₆₃ is CH₃, CH₂CH₃, CH₂CH₂CH₃, CH(CH₃)₂ or CF₃;
T₁ is N or C—H; and
T₂ is N or C—H,
and wherein
(A) when A₁ is

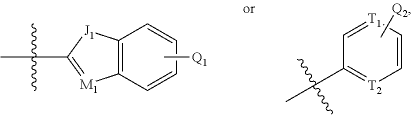

and T₁ and T₂ are each C—H, then at least one of G₁, Q₁ or Q₂ is Br, F, Cl or I; and
(B) the compound is not one of the following:

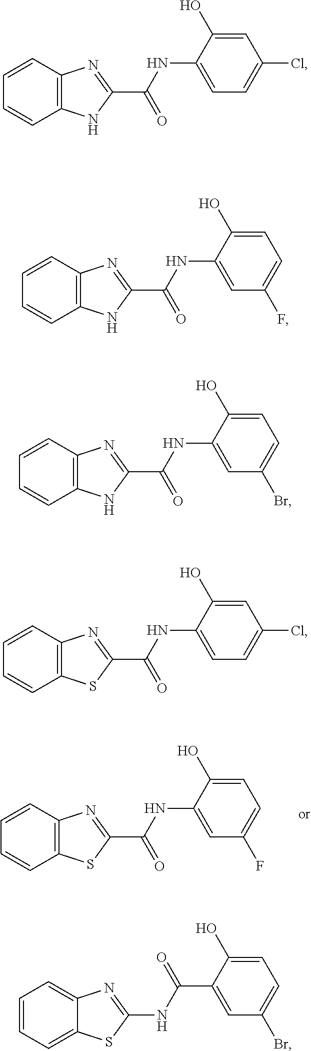 [SMILES: O=C(Nc1ccc(Cl)cc1O)c1nc2ccccc2[nH]1 ; O=C(Nc1cc(F)ccc1O)c1nc2ccccc2[nH]1 ; O=C(Nc1cc(Br)ccc1O)c1nc2ccccc2[nH]1 ; O=C(Nc1ccc(Cl)cc1O)c1nc2ccccc2s1 ; O=C(Nc1cc(F)ccc1O)c1nc2ccccc2s1 ; O=C(Nc1nc2ccccc2s1)c1cc(Br)ccc1O]

wherein the compound, or salt thereof, has anti-bacterial activity.

One aspect of the invention relates to a method of treating a subject known to have or suspected of having a bacterial infection, the method comprising administering to the subject an effective amount of a compound selected from the compounds shown in Table B and Table C, or a salt thereof, wherein the compound or salt thereof has anti-bacterial activity.

One aspect of the invention relates to a method of inhibiting a pyruvate kinase (PK) from a bacterial strain comprising contacting the pyruvate kinase with an effective amount of a compound of general formula I, as defined above, or a salt thereof, wherein the compound or salt thereof has bacterial PK inhibitory activity.

One aspect of the invention relates to a method of inhibiting a pyruvate kinase (PK) from a bacterial strain comprising contacting the pyruvate kinase with an effective amount of a compound selected from the compounds shown in Table B and Table C, or a salt thereof, wherein the compound or salt thereof has bacterial PK inhibitory activity.

One aspect of the invention relates to a method of inhibiting growth of at least one bacterial strain comprising contacting bacteria of the bacterial strain with an effective amount of a compound of general formula I, as defined above, or a salt thereof, wherein the compound or salt thereof has anti-bacterial activity.

One aspect of the invention relates to a method of inhibiting growth of at least one bacterial strain comprising contacting bacteria of the bacterial strain with an effective amount of a compound from the compounds shown in Table B and Table C, or a salt thereof, wherein the compound or salt thereof has anti-bacterial activity.

One aspect of the invention relates to a method of inhibiting growth of at least one bacterial strain in a substrate or on a surface comprising contacting the substrate or surface with an effective amount of a compound of general formula I, as defined above, or a salt thereof, wherein the compound or salt thereof has anti-bacterial activity.

One aspect of the invention relates to a method of inhibiting growth of at least one bacterial strain in a substrate or on a surface comprising contacting the substrate or surface with an effective amount of a compound selected from the compounds shown in Table B and Table C, or a salt thereof, wherein the compound or salt thereof has anti-bacterial activity.

One aspect of the invention relates to a pharmaceutical composition comprising a compound of general formula I, as defined above, or a salt thereof, and a pharmaceutically acceptable carrier.

One aspect of the invention relates to a pharmaceutical composition comprising a compound selected from the compounds shown in Table B and Table C, or a salt thereof, and a pharmaceutically acceptable carrier.

One aspect of the invention relates to a compound of general formula I:

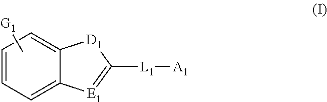

or a salt thereof, wherein:
L₁ is —CH₂CH₂—, —CHCH—, —CC—, —C(O)NH—, —NHC(O)—, —C(O)—, —N═CH—, —CH₂R₅—, —NHCH₂—,

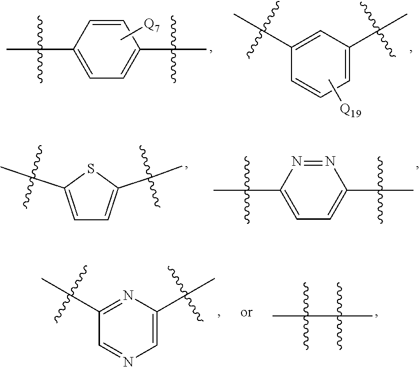

wherein R₅ is CH₂CH₂, NHCH₂, NH, SCH₂, O, or S, and wherein each Q₇ and Q₁₉ are independently H, NO₂, or OMe;
A₁ is

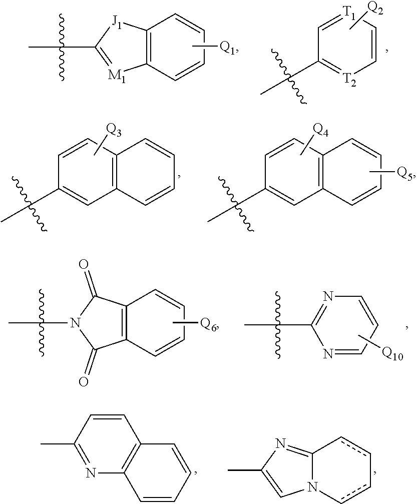

wherein [symbol] represents a single or double bond, or a 5-membered heteroaryl optionally substituted with Q₁₁ and containing 1 or 2 heteroatoms each selected from N, O and S;
each G₁ is independently H, Br, F, Cl, I, OR₁, SR₁, SO₂R₁, C(O)R₁, C(O)OR₁, unsubstituted phenyl, substituted phenyl, unsubstituted alkyl, substituted alkyl, unsubstituted allyl, or substituted allyl, wherein the alkyl or allyl is 1-6 carbons in length, wherein the substitutions to the phenyl, alkyl, or allyl are optionally Br, F, Cl, I, OH, OMe, or N₃, and wherein R₁ is H or Me;
D₁ is S, O, N—H, N—CH₃, N—CH₂—OCH₃, N—CH₂—CH₃, N—CH₂—COOH, N—CH₂—CH₂OH, CH₂, CH—CH₃, CH—CH₂—OCH₃, CH—CH₂—CH₃, CH—CH₂—COOH, CH—CH₂—CH₂OH, N—R₂, or CH—R₂, wherein R₂ is

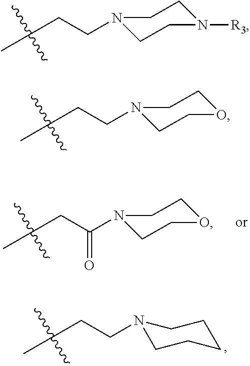

wherein R₃ is H, unsubstituted alkyl, or substituted alkyl, wherein the alkyl is 1-6 carbons in length, and the alkyl is optionally substituted with Br, F, Cl, I, OH, OMe, or N₃;
E₁ is N, C—H, C—CH₃, C—C(O)OR₄, C—C(O)R₆₃, C—Cl, C—Br, C—F, or C—COR₄, wherein R₄ is H or Me, and R₆₃ is CH₃, CH₂CH₃, CH₂CH₂CH₃, CH(CH₃)₂ or CF₃, and wherein if D₁ is CH₂, CH—CH₃, CH—CH₂—OCH₃, CH—CH₂—CH₃, CH—CH₂—COOH, CH—CH₂—CH₂OH, or CH—R₂, E₁ is N;
each Q₁ is independently H, Br, F, Cl, I,

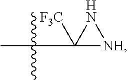 [SMILES: *C1(C(F)(F)F)NN1]

OR₆, SR₆, SO₂R₆, C(O)R₆, C(O)OR₆, N₃, unsubstituted phenyl, substituted phenyl, unsubstituted alkyl, substituted alkyl, unsubstituted allyl, or substituted allyl, wherein the alkyl or allyl is 1-6 carbons in length, wherein R₆ is H or Me, and wherein the substituted phenyl, alkyl, or allyl is optionally substituted with Q₈;
each Q₂ is independently H, Br, F, Cl, I,

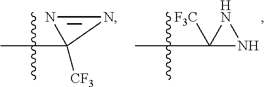 [SMILES: *C1(C(F)(F)F)N=N1 ; *C1(C(F)(F)F)NN1]

N₃, OR₇, SR₇, SO₂R₇, C(O)R₇, NO₂,

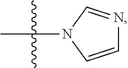 [SMILES: *n1ccnc1]

unsubstituted phenyl, substituted phenyl, unsubstituted alkyl, substituted alkyl, unsubstituted allyl, or substituted allyl, wherein the alkyl or allyl is 1-6 carbons in length, wherein R₇ is H or Me, and wherein the substituted phenyl, alkyl, or allyl is optionally substituted with Q₉;
each Q₃ is independently H, Br, F, Cl, I, or OR₈, wherein R₈ is H or Me;
each Q₄ is independently H, Br, F, Cl, I, or OR₉, wherein R₉ is H or Me;
each Q₅ is independently H, Br, F, Cl, I, or OR₁₀, wherein R₁₀ is H or Me;
each Q₆ is independently H, Br, F, Cl, I, or OR₁₁, wherein R₁₁ is H or Me;
each Q₈ is independently Br, F, Cl, I, Me, or OR₁₂, wherein R₁₂ is H or Me;
each Q₉ is independently Br, F, Cl, I, Me, or OR₁₃, wherein R₁₃ is H or Me;
each Q₁₀ is independently H, Br, F, Cl or I;
each Q₁₁ is independently H, Me, unsubstituted phenyl or substituted phenyl, wherein the substituted phenyl is optionally substituted with Q₈;
J₁ is S, O, N—H, N—CH₃, N—CH₂—OCH₃, N—CH₂—COOH, N—CH₂—CH₂OH, CH—CH₃, N—R₁₄, or CH—R₁₄, wherein R₁₄ is

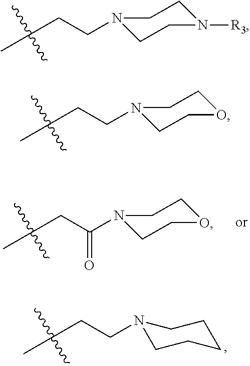

wherein R₃ is H, unsubstituted alkyl, or substituted alkyl, wherein the alkyl is 1-6 carbons in length, and the alkyl is optionally substituted with Br, F, Cl, I, OH, OMe, or N₃;
M₁ is N, C—H, C—CH₃, C—C(O)OR₄, C—C(O)R₆₃, or C—CH(CH₃)₂, wherein R₄ is H or Me, and R₆₃ is CH₃, CH₂CH₃, CH₂CH₂CH₃, CH(CH₃)₂ or CF₃;
T₁ is N or C—H; and
T₂ is N or C—H,
and wherein:
(A) when A₁ is

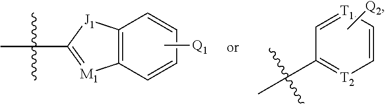

and T₁ and T₂ are each C—H, then at least one of G₁, Q₁ or Q₂ is Br, F, Cl or I; and
(B) the compound is not one of the compounds shown in Table A or

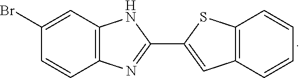 [SMILES: Brc1ccc2nc(-c3cc4ccccc4s3)[nH]c2c1]

as well as pharmaceutical compositions comprising the compound, or salt thereof, and a pharmaceutically acceptable carrier.

One aspect of the invention relates to a compound of general formula I, as defined above, or a salt thereof, for use to treat a subject known to have or suspected of having a bacterial infection, wherein the compound or salt thereof has anti-bacterial activity. One aspect of the invention relates to a compound selected from the compounds shown in Table B and Table C, or a salt thereof, for use to treat a subject known to have or suspected of having a bacterial infection, wherein the compound or salt thereof has anti-bacterial activity.

One aspect of the invention relates to a use of a compound of general formula I, as defined above, or a salt thereof, to treat a subject known to have or suspected of having a bacterial infection, wherein the compound or salt thereof has anti-bacterial activity. One aspect of the invention relates to a use of a compound selected from the compounds shown in Table B and Table C, or a salt thereof, to treat a subject known to have or suspected of having a bacterial infection, wherein the compound or salt thereof has anti-bacterial activity.

One aspect of the invention relates to a use of a compound of general formula I, as defined above, or a salt thereof, in the manufacture of a medicament for treating a subject known to have or suspected of having a bacterial infection, wherein the compound or salt thereof has anti-bacterial activity. One aspect of the invention relates to a use of a compound selected from the compounds shown in Table B and Table C, or a salt thereof, in the manufacture of a medicament for treat a subject known to have or suspected of having a bacterial infection, wherein the compound or salt thereof has anti-bacterial activity.

One aspect of the invention relates to a compound of general formula I, as defined above, or a salt thereof, for use to inhibit a pyruvate kinase (PK) from a bacterial strain, wherein the compound or salt thereof has bacterial PK inhibitory activity. One aspect of the invention relates to a compound selected from the compounds shown in Table B and Table C, or a salt thereof, for use to inhibit a pyruvate kinase (PK) from a bacterial strain, wherein the compound or salt thereof has bacterial PK inhibitory activity.

One aspect of the invention relates to a use of a compound of general formula I, as defined above, or a salt thereof, to inhibit a pyruvate kinase (PK) from a bacterial strain, wherein the compound or salt thereof has bacterial PK inhibitory activity. One aspect of the invention relates to a use of a compound selected from the compounds shown in Table B and Table C, or a salt thereof, to inhibit a pyruvate kinase (PK) from a bacterial strain, wherein the compound or salt thereof has bacterial PK inhibitory activity.

One aspect of the invention relates to a use of a compound of general formula I, as defined above, or a salt thereof, in the manufacture of a medicament for inhibiting a pyruvate kinase (PK) from a bacterial strain, wherein the compound or salt thereof has bacterial PK inhibitory activity. One aspect of the invention relates to a use of a compound selected from the compounds shown in Table B and Table C, or a salt thereof, in the manufacture of a medicament for inhibiting a pyruvate kinase (PK) from a bacterial strain, wherein the compound or salt thereof has bacterial PK inhibitory activity.

In one aspect, the invention relates to a compound of general formula I, as defined above, or a salt thereof, for use to inhibit growth of at least one bacterial strain, wherein the compound or salt thereof has anti-bacterial activity. In one aspect, the invention relates to a compound selected from the compounds shown in Table B and Table C, or a salt thereof, for use to inhibit growth of at least one bacterial strain, wherein the compound or salt thereof has anti-bacterial activity.

In one aspect, the invention relates to a use of a compound of general formula I, as defined above, or a salt thereof, to inhibit growth of at least one bacterial strain, wherein the compound or salt thereof has anti-bacterial activity. In one aspect, the invention relates to a use of a compound from the compounds shown in Table B and Table C, or a salt thereof, to inhibit growth of at least one bacterial strain, wherein the compound or salt thereof has anti-bacterial activity.

In one aspect, the invention relates to a use of a compound of general formula I, as defined above, or a salt thereof, in the manufacture of a medicament for inhibiting growth of at least one bacterial strain, wherein the compound or salt thereof has anti-bacterial activity. In one aspect, the invention relates to a use of a compound selected from the compounds shown in Table B and Table C, or a salt thereof, in the manufacture of a medicament for inhibiting growth of at least one bacterial strain, wherein the compound or salt thereof has anti-bacterial activity.

### BRIEF DESCRIPTION OF THE DRAWINGS

These and other features of the invention will become more apparent in the following detailed description in which reference is made to the appended drawings.

FIG. 1 depicts the MRSA PK inhibitors 1-4.

FIG. 2 depicts a general synthesis pathway (Scheme 1) for bis-indoles 10 and 12.

FIG. 3 depicts a general synthesis pathway (Scheme 2) for bis-indoles 14, 15, 17 and 20.

FIG. 4 depicts a general synthesis pathway (Scheme 3) for compounds containing a mono-indole coupled with various heterocycles.

FIG. 5 depicts a general synthesis pathway (Scheme 4) for bis-indole compounds containing an acetylene linker.

FIG. 6 depicts a general synthesis pathway (Scheme 5) for the bis-indoles 27 and 28.

FIG. 7 depicts a general synthesis pathway (Scheme 6) for bis-indoles with an aryl linker.

FIG. 8 depicts a general synthesis pathway (Scheme 7) for compounds 36-39.

FIG. 9 depicts a general synthesis pathway (Scheme 23) for compounds 143 and 145.

FIG. 10 depicts a general synthesis pathway (Scheme 24) for compounds 157-159.

FIG. 11 presents the results of an assessment of induction of resistance by compounds 167 and 178 in MRSA and shows that after 30 passages in 0.5×MIC MRSA MW2 (USA400) did not develop resistance to either compound.

FIG. 12 presents the results of an in vivo efficacy study in neutropenic mouse MSSA thigh infection model of exemplary compound 178.

### DETAILED DESCRIPTION OF THE INVENTION

The invention relates generally to compounds of general formula I as shown below that are capable of inhibiting bacterial pyruvate kinase and/or bacterial growth. The compounds may find use as antibacterial agents in therapeutic and/or non-therapeutic contexts.

### Definitions

Unless defined otherwise, all technical and scientific terms used herein have the same meaning as commonly understood by one of ordinary skill in the art to which this invention belongs.

Administration of a compounds as disclosed herein “in combination with” one or more further therapeutic agents is intended to include simultaneous (concurrent) administration and consecutive administration. Consecutive administration is intended to encompass various orders of administration of the therapeutic agent(s) and the disclosed compound(s) to a subject with administration of the therapeutic agent(s) and the compound(s) being separated by a defined time period that may be short (for example in the order of minutes) or extended (for example in the order of days or weeks).

The term “inhibit” and grammatical variations thereof, as used herein, means to reduce, halt or hold in check, and thus inhibition may be complete or partial and may be of short or long term duration.

The term “effective amount,” as used herein, means the amount of a compound or composition that will produce a desired biological response in a subject or system. For example, an “effective amount” of an antibacterial agent may be defined as the amount of the antibacterial agent that inhibits the growth of a selected bacterial strain.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any disclosed method, use or composition, and vice versa. Furthermore, compounds, compositions and kits of the invention can be used to achieve the disclosed methods and uses.

### Compounds

One aspect of the invention relates to compounds of the general Formula I:

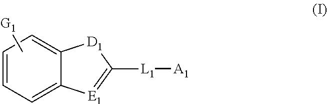

and salts thereof, wherein:
L₁ is —CH₂CH₂—, —CHCH—, —CC—, —C(O)NH—, —NHC(O)—, —C(O)—, —N═CH—, —CH₂R₅—, —NHCH₂—,

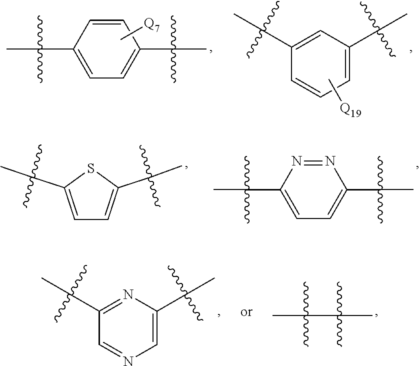

wherein R₅ is CH₂CH₂, NHCH₂, NH, SCH₂, O, or S, and wherein each Q₇ and Q₁₉ are independently H, NO₂, or OMe;
A₁ is

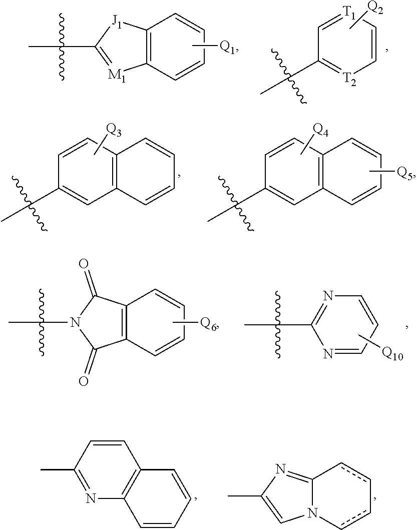

wherein [symbol] represents a single or double bond, or a 5-membered heteroaryl optionally substituted with Q₁₁ and containing 1 or 2 heteroatoms each selected from N, O and S;
each G₁ is independently H, Br, F, Cl, I, OR₁, SR₁, SO₂R₁, C(O)R₁, C(O)OR₁,

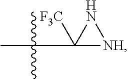 [SMILES: *C1(C(F)(F)F)NN1]

N₃, unsubstituted phenyl, substituted phenyl, unsubstituted alkyl, substituted alkyl, unsubstituted allyl, or substituted allyl, wherein the alkyl or allyl is 1-6 carbons in length, wherein the substitutions to the phenyl, alkyl, or allyl are optionally Br, F, Cl, I, OH, OMe, Me, or N₃, and wherein R₁ is H or Me;
D₁ is S, O, N—H, N—CH₃, N—CH₂—OCH₃, N—CH₂—CH₃, N—CH₂—COOH, N—CH₂—CH₂OH, CH₂, CH—CH₃, CH—CH₂—OCH₃, CH—CH₂—CH₃, CH—CH₂—COOH, CH—CH₂—CH₂OH, N—R₂, or CH—R₂, wherein R₂ is

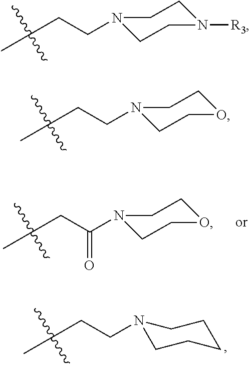

wherein R₃ is H, unsubstituted alkyl, or substituted alkyl, wherein the alkyl is 1-6 carbons in length, and the alkyl is optionally substituted with Br, F, Cl, I, OH, OMe, or N₃;
E₁ is N, C—H, C—CH₃, C—C(O)OR₄, C—C(O)R₆₃, C—Cl, C—Br, C—F, or C—COR₄, wherein R₄ is H or Me, and R₆₃ is CH₃, CH₂CH₃, CH₂CH₂CH₃, CH(CH₃)₂ or CF₃, and wherein if D₁ is CH₂, CH—CH₃, CH—CH₂—OCH₃, CH—CH₂—CH₃, CH—CH₂—COOH, CH—CH₂—CH₂OH, or CH—R₂, E₁ is N;

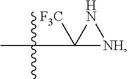 [SMILES: *C1(C(F)(F)F)NN1]

each Q₁ is independently H, Br, F, Cl, I, OR₆, SR₆, SO₂R₆, C(O)R₆, C(O)OR₆, N₃, unsubstituted phenyl, substituted phenyl, unsubstituted alkyl, substituted alkyl, unsubstituted allyl, or substituted allyl, wherein the alkyl or allyl is 1-6 carbons in length, wherein R₆ is H or Me, and wherein the substituted phenyl, alkyl, or allyl is optionally substituted with Q₈;

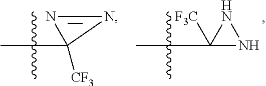 [SMILES: *C1(C(F)(F)F)N=N1 ; *C1(C(F)(F)F)NN1]

each Q₂ is independently H, Br, F, Cl, I, N₃, OR₇, SR₇, SO₂R₇, C(O)R₇, NO₂,

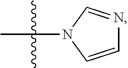 [SMILES: *n1ccnc1]

unsubstituted phenyl, substituted phenyl, unsubstituted alkyl, substituted alkyl, unsubstituted allyl, or substituted allyl, wherein the alkyl or allyl is 1-6 carbons in length, wherein R₇ is H or Me, and wherein the substituted phenyl, alkyl, or allyl is optionally substituted with Q₉;
each Q₃ is independently H, Br, F, Cl, I, or OR₈, wherein R₈ is H or Me;
each Q₄ is independently H, Br, F, Cl, I, or OR₉, wherein R₉ is H or Me;
each Q₅ is independently H, Br, F, Cl, I, or OR₁₀, wherein Rₘ is H or Me;
each Q₆ is independently H, Br, F, Cl, I, or OR₁₁, wherein R₁₁ is H or Me;
each Q₈ is independently Br, F, Cl, I, Me, or OR₁₂, wherein R₁₂ is H or Me;
each Q₉ is independently Br, F, Cl, I, Me, or OR₁₃, wherein R₁₃ is H or Me;
each Q₁₀ is independently H, Br, F, Cl or I;
each Q₁₁ is independently H, Me, unsubstituted phenyl or substituted phenyl, wherein the substituted phenyl is optionally substituted with Q₈;
J₁ is S, O, N—H, N—CH₃, N—CH₂—OCH₃, N—CH₂—COOH, N—CH₂—CH₂OH, CH—CH₃, N—R₁₄, or CH—R₁₄, wherein R₁₄ is

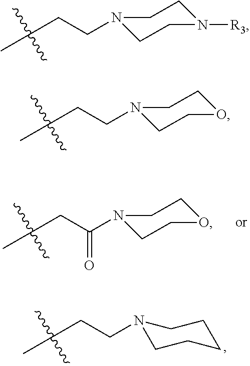

wherein R₃ is H, unsubstituted alkyl, or substituted alkyl, wherein the alkyl is 1-6 carbons in length, and the alkyl is optionally substituted with Br, F, Cl, I, OH, OMe, or N₃;
M₁ is N, C—H, C—CH₃, C—C(O)OR₄, C—C(O)R₆₃, or C—CH(CH₃)₂, wherein R₄ is H or Me, and R₆₃ is CH₃, CH₂CH₃, CH₂CH₂CH₃, CH(CH₃)₂ or CF₃;
T₁ is N or C—H; and
T₂ is N or C—H.

Certain embodiments of the invention relate to compounds of general formula I, wherein:
(A) when A₁ is

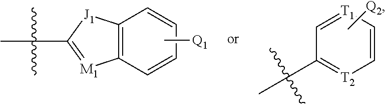

and T₁ and T₂ are each C—H, then at least one of G₁, Q₁ or Q₂ is Br, F, Cl or I; and
(B) the compound is not one of the following:

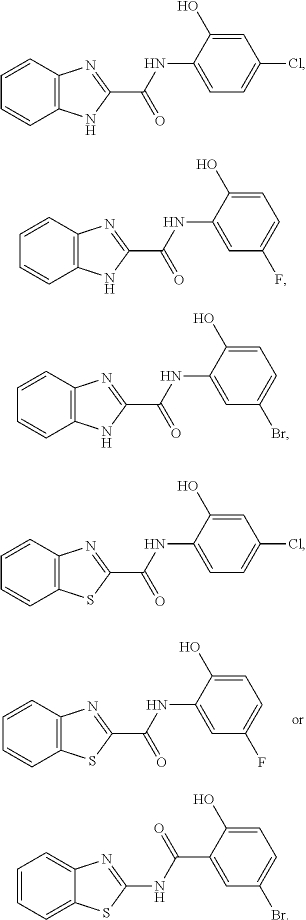 [SMILES: O=C(Nc1ccc(Cl)cc1O)c1nc2ccccc2[nH]1 ; O=C(Nc1cc(F)ccc1O)c1nc2ccccc2[nH]1 ; O=C(Nc1cc(Br)ccc1O)c1nc2ccccc2[nH]1 ; O=C(Nc1ccc(Cl)cc1O)c1nc2ccccc2s1 ; O=C(Nc1cc(F)ccc1O)c1nc2ccccc2s1 ; O=C(Nc1nc2ccccc2s1)c1cc(Br)ccc1O]

Certain embodiments of the invention relate to compounds of general formula I, wherein:
(A) when A₁ is

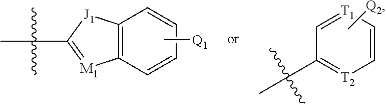

and T₁ and T₂ are each C—H, then at least one of G₁, Q₁ or Q₂ is Br, F, Cl or I; and
(B) the compound is not one of the compounds shown in Table A or

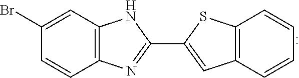 [SMILES: Brc1ccc2nc(-c3cc4ccccc4s3)[nH]c2c1]

TABLE A

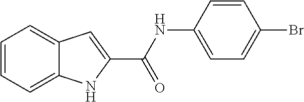 [SMILES: O=C(Nc1ccc(Br)cc1)c1cc2ccccc2[nH]1]

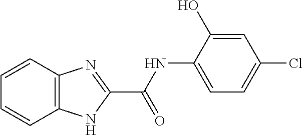 [SMILES: O=C(Nc1ccc(Cl)cc1O)c1nc2ccccc2[nH]1]

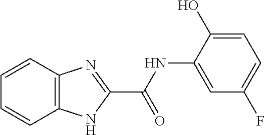 [SMILES: O=C(Nc1cc(F)ccc1O)c1nc2ccccc2[nH]1]

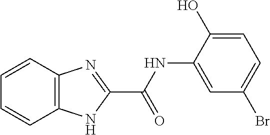 [SMILES: O=C(Nc1cc(Br)ccc1O)c1nc2ccccc2[nH]1]

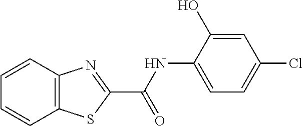 [SMILES: O=C(Nc1ccc(Cl)cc1O)c1nc2ccccc2s1]

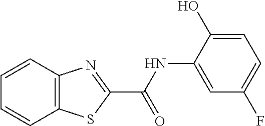 [SMILES: O=C(Nc1cc(F)ccc1O)c1nc2ccccc2s1]

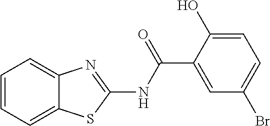 [SMILES: O=C(Nc1nc2ccccc2s1)c1cc(Br)ccc1O]

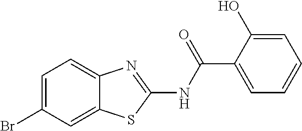 [SMILES: O=C(Nc1nc2ccc(Br)cc2s1)c1ccccc1O]

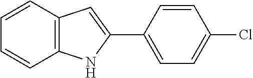 [SMILES: Clc1ccc(-c2cc3ccccc3[nH]2)cc1]

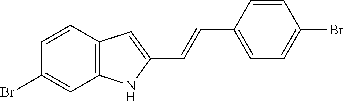 [SMILES: Brc1ccc(/C=C/c2cc3ccc(Br)cc3[nH]2)cc1]

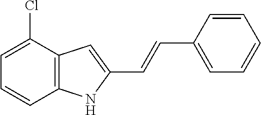 [SMILES: Clc1cccc2[nH]c(/C=C/c3ccccc3)cc12]

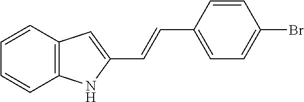 [SMILES: Brc1ccc(/C=C/c2cc3ccccc3[nH]2)cc1]

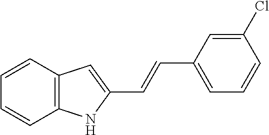 [SMILES: Clc1cccc(/C=C/c2cc3ccccc3[nH]2)c1]

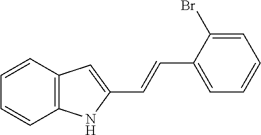 [SMILES: Brc1ccccc1/C=C/c1cc2ccccc2[nH]1]

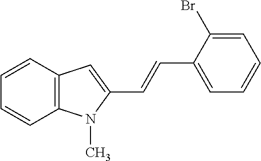 [SMILES: Cn1c(/C=C/c2ccccc2Br)cc2ccccc21]

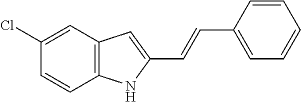 [SMILES: Clc1ccc2[nH]c(/C=C/c3ccccc3)cc2c1]

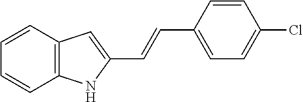 [SMILES: Clc1ccc(/C=C/c2cc3ccccc3[nH]2)cc1]

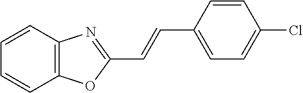 [SMILES: Clc1ccc(/C=C/c2nc3ccccc3o2)cc1]

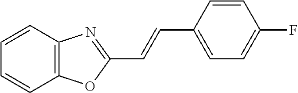 [SMILES: Fc1ccc(/C=C/c2nc3ccccc3o2)cc1]

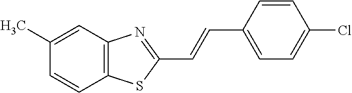 [SMILES: Cc1ccc2sc(/C=C/c3ccc(Cl)cc3)nc2c1]

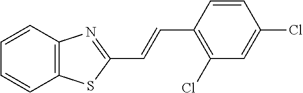 [SMILES: Clc1ccc(/C=C/c2nc3ccccc3s2)c(Cl)c1]

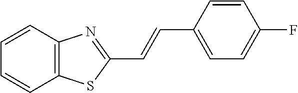 [SMILES: Fc1ccc(/C=C/c2nc3ccccc3s2)cc1]

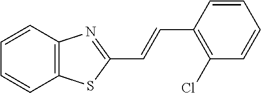 [SMILES: Clc1ccccc1/C=C/c1nc2ccccc2s1]

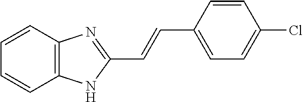 [SMILES: Clc1ccc(/C=C/c2nc3ccccc3[nH]2)cc1]

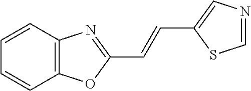 [SMILES: C(=C\c1cncs1)/c1nc2ccccc2o1]

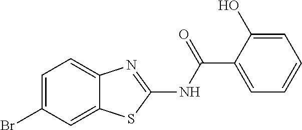 [SMILES: O=C(Nc1nc2ccc(Br)cc2s1)c1ccccc1O]

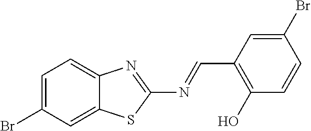 [SMILES: Oc1ccc(Br)cc1/C=N/c1nc2ccc(Br)cc2s1]

Certain embodiments of the invention relate to compounds of general formula I, wherein the compound is not one of the compounds shown in Table B (below).

In certain embodiments, in compounds of general formula I or salts thereof, when
(i) E₁ is C—CH₃, C—C(O)CH(CH₃)₂, C—C(O)OH, C—C(O)CH₃, C—Cl, C—Br, C—F, or C—COMe,
(ii) A₁ is

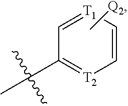

(iii) T₁ and T₂ are each C—H, and
(iv) L₁ is

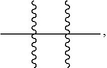 [SMILES: **]

then D₁ is not N—CH₃ or N—CH₂—CH₃.

In certain embodiments, in compounds of general formula I or salts thereof:
L₁ is —CH₂CH₂—, —CHCH—, —CC—, —C(O)NH—, —NHC(O)—, —C(O)—, —N═CH—, —CH₂R₅—, —NHCH₂—,

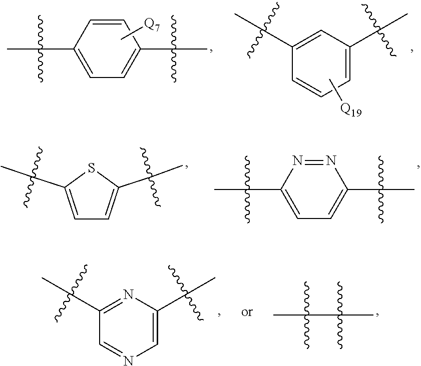

wherein R₅ is NHCH₂, NH, SCH₂, or S, and wherein each Q₇ and Q₁₉ are independently H, NO₂, or OMe;
A₁ is

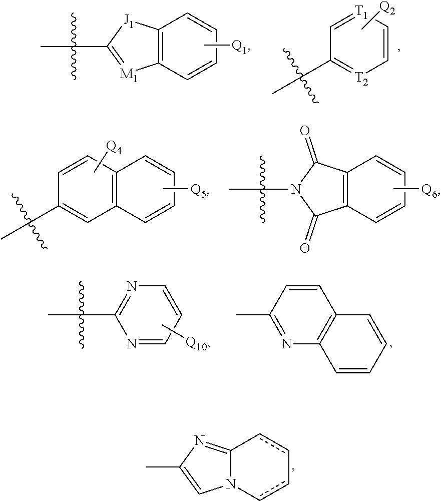

wherein [symbol] represents a single or double bond, or a 5-membered heteroaryl optionally substituted with Q₁₁ and containing 1 or 2 heteroatoms each selected from N, O and S;

each G₁ is independently H, Br, F, Cl, OR₁, C(O)R₁, C(O)OR₁, unsubstituted phenyl, substituted phenyl, unsubstituted alkyl, substituted alkyl, wherein the alkyl is 1-3 carbons in length, wherein the substitutions to the phenyl or alkyl are optionally Br, F, Cl, I, OH, OMe, or N₃, and wherein R₁ is H or Me;
D₁ is S, N—H, N—CH₃, N—CH₂—OCH₃, N—CH₂—CH₃, N—CH₂—COOH, N—CH₂—CH₂OH, N—R₂, wherein R₂ is

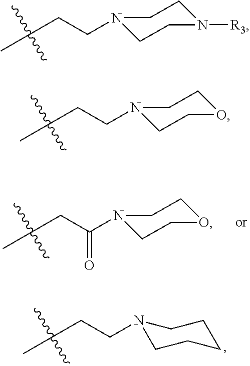

wherein R₃ is H, unsubstituted alkyl, or substituted alkyl, wherein the alkyl is 1-6 carbons in length, and the alkyl is optionally substituted with Br, F, Cl, I, OH, OMe, or N₃;
E₁ is N, C—H, C—CH₃, C—C(O)OR₄, C—C(O)R₆₃, C—Cl, or C—OR₄, wherein R₄ is H or Me, and R₆₃ is CH₃, CH₂CH₃, CH₂CH₂CH₃, CH(CH₃)₂ or CF₃;
each Q₁ is independently; H, Br, F, Cl,

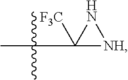 [SMILES: *C1(C(F)(F)F)NN1]

OR₆, C(O)R₆, C(O)OR₆, N₃, unsubstituted phenyl, substituted phenyl, unsubstituted alkyl, or substituted alkyl, wherein the alkyl is 1-3 carbons in length, wherein R₆ is H or Me, and wherein the substituted phenyl or substituted alkyl is optionally substituted with Q₈;
each Q₂ is independently H, Br, F, Cl,

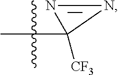 [SMILES: *C1(C(F)(F)F)N=N1]

N₃, OR₇, C(O)R₇, NO₂,

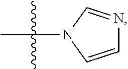 [SMILES: *n1ccnc1]

unsubstituted phenyl, substituted phenyl, unsubstituted alkyl, or substituted alkyl, wherein the alkyl is 1-3 carbons in length, wherein R₇ is H or Me, and wherein the substituted phenyl or substituted alkyl is optionally substituted with Q₉;
each Q₄ is independently H, Br, F, Cl, or OR₉, wherein R₉ is H or Me;
each Q₅ is independently H, Br, F, Cl, or OR₁₀, wherein R₁₀ is H or Me;
each Q₆ is independently H, Br, F, Cl, or OR₁₁, wherein R₁₁ is H or Me;
each Q₈ is independently Br, F, Cl, Me, or OR₁₂, wherein R₁₂ is H or Me;
each Q₉ is independently Br, F, Cl, Me, or OR₁₃, wherein R₁₃ is H or Me;
each Q₁₀ is independently H, Br, F or Cl;
each Q₁₁ is independently H, Me, unsubstituted phenyl or substituted phenyl, wherein the substituted phenyl is optionally substituted with Q₈;
J₁ is S, O, N—H, N—CH₃, N—CH₂—OCH₃, N—CH₂—COOH, N—CH₂—CH₂OH, or N—R₁₄, wherein R₁₄ is

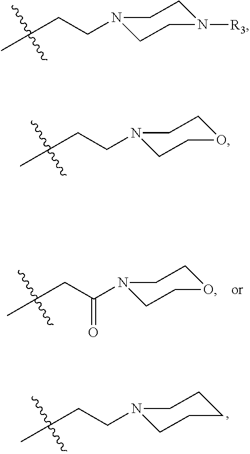

wherein R₃ is H or Me;
M₁ is N, C—H, C—CH₃, C—C(O)OR₄, or C—C(O)R₆₃, wherein R₄ is H or Me, and R₆₃ is CH₃, CH₂CH₃, CH₂CH₂CH₃, CH(CH₃)₂ or CF₃;
T₁ is N or C—H; and
T₂ is N or C—H.

In certain embodiments, compounds of general formula I include compounds of formula 2:

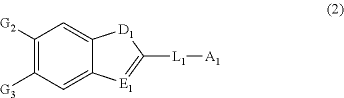

and salts thereof, wherein:
G₂ is H, Br, F, Cl, I, OR₁, SR₁, SO₂R₁, C(O)R₁, OMe, unsubstituted phenyl, optionally substituted phenyl, unsubstituted or optionally substituted alkyl, unsubstituted or optionally substituted allyl, wherein the alkyl or allyl is 1-6 carbons in length, wherein the substitutions are optionally Br, F, Cl, I, OH, OMe, or N₃, and wherein R₁ is H or Me;
G₃ is H, Br, F, Cl, I, OR₁, SR₁, SO₂R₁, C(O)R₁, OMe, unsubstituted phenyl, substituted phenyl, unsubstituted or optionally substituted alkyl, unsubstituted or optionally substituted allyl, wherein the alkyl or allyl is 1-6 carbons in length, wherein the substitutions are optionally Br, F, Cl, I, OH, OMe, or N₃, and wherein R₁ is H or Me; and
D₁, E₁, L₁ and A₁ are as described above for general formula I.

In certain embodiments, in compounds of formula 2, G₂ is Br, F, Cl or I. In some embodiments, in compounds of formula 2, G₂ is Br, F or Cl. In some embodiments, in compounds of formula 2, G₂ is Br.

In certain embodiments, in compounds of general formula I, or salts thereof:
each G₁ is independently H, Br, F, Cl, OMe, C(O)R₁, or C(O)OR₁, wherein R₁ is H or Me, and
each Q₁ is independently; H, Br, F, Cl,

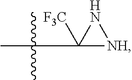 [SMILES: *C1(C(F)(F)F)NN1]

OMe, C(O)R₆, C(O)OR₆, N₃, unsubstituted phenyl, substituted phenyl, unsubstituted alkyl, or substituted alkyl, wherein the alkyl is 1-3 carbons in length, wherein R₆ is H or Me, and wherein the substituted phenyl or substituted alkyl is optionally substituted with Q₈.

In certain embodiments, in compounds of general formula I or formula 2, or salts thereof:
D₁ is S, O, N—H, N—CH₃, N—CH₂—OCH₃, N—CH₂—CH₃, N—CH₂—COOH, N—CH₂—CH₂OH, or N—R₂, wherein R₂ is

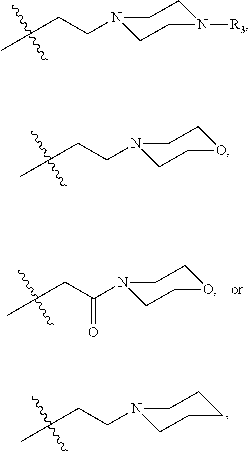

wherein R₃ is H, unsubstituted alkyl, or substituted alkyl, wherein the alkyl is 1-6 carbons in length, and the alkyl is optionally substituted with Br, F, Cl, I, OH, OMe, or N₃,
and either:
A₁ is

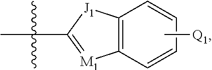

and E₁ is C—CH₃, C—C(O)OR₄, C—C(O)R₆₃, or C—OR₄, wherein R₄ is H or Me, and R₆₃ is CH₃, CH₂CH₃, CH₂CH₂CH₃, CH(CH₃)₂ or CF₃,
or
A₁ is

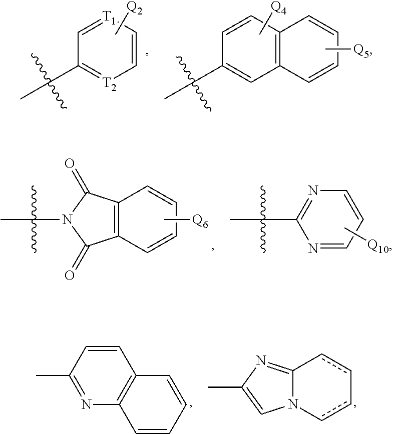

wherein [symbol] represents a single or double bond, or a 5-membered heteroaryl optionally substituted with Q₁₁ and containing 1 or 2 heteroatoms each selected from N, O and S, and E₁ is N, C—H, C—CH₃, C—C(O)OR₄, C—C(O)R₆₃, C—Cl, or C—OR₄, wherein R₄ is H or Me, and R₆₃ is CH₃, CH₂CH₃, CH₂CH₂CH₃, CH(CH₃)₂ or CF₃.

In certain embodiments, in compounds of general formula I or formula 2, or salts thereof:
A₁ is

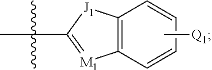

D₁ is S, O, N—H, N—CH₃, N—CH₂—OCH₃, N—CH₂—CH₃, N—CH₂—COOH, N—CH₂—CH₂OH, N—R₂, wherein R₂ is

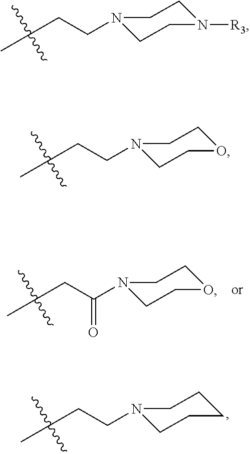

wherein R₃ is H, unsubstituted alkyl, or substituted alkyl, wherein the alkyl is 1-6 carbons in length, and the alkyl is optionally substituted with Br, F, Cl, I, OH, OMe, or N₃, and
E₁ is C—CH₃, C—C(O)OR₄, C—C(O)R₆₃, or C—OR₄, wherein R₄ is H or Me, and R₆₃ is CH₃, CH₂CH₃, CH₂CH₂CH₃, CH(CH₃)₂ or CF₃.

In certain embodiments, in compounds of general formula I or formula 2, or salts thereof:
A₁ is

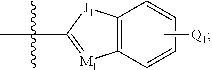

D₁ is S, O, N—H, N—CH₃, N—CH₂—OCH₃, N—CH₂—CH₃, N—CH₂—COOH, N—CH₂—CH₂OH, and
E₁ is C—CH₃, C—C(O)OR₄, or C—C(O)R₆₃, wherein R₄ is H or Me, and R₆₃ is CH₃, CH₂CH₃, CH₂CH₂CH₃, CH(CH₃)₂ or CF₃.

In certain embodiments, in compounds of general formula I, or salts thereof:
A₁ is

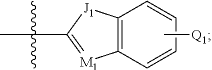

D₁ is S, O, N—H, N—CH₃, N—CH₂—OCH₃, N—CH₂—CH₃, N—CH₂—COOH, N—CH₂—CH₂OH, N—R₂, wherein R₂ is

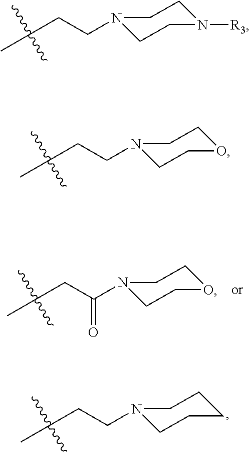

wherein R₃ is H, unsubstituted alkyl, or substituted alkyl, wherein the alkyl is 1-6 carbons in length, and the alkyl is optionally substituted with Br, F, Cl, I, OH, OMe, or N₃;
E₁ is C—CH₃, C—C(O)OR₄, C—C(O)R₆₃, or C—OR₄, wherein R₄ is H or Me, and R₆₃ is CH₃, CH₂CH₃, CH₂CH₂CH₃, CH(CH₃)₂ or CF₃;
each G₁ is independently H, Br, F, Cl, OMe, C(O)R₁, C(O)OR₁, unsubstituted phenyl, substituted phenyl, unsubstituted alkyl or substituted alkyl, wherein the alkyl is 1-3 carbons in length, wherein the substitutions to the phenyl or alkyl are optionally Br, F, Cl, I, OH, OMe, or N₃, and wherein R₁ is H or Me;
each Q₁ is independently; H, Br, F, Cl,

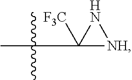 [SMILES: *C1(C(F)(F)F)NN1]

OMe, C(O)R₆, C(O)OR₆, N₃, unsubstituted phenyl, substituted phenyl, unsubstituted alkyl, or substituted alkyl, wherein the alkyl is 1-3 carbons in length, wherein R₆ is H or Me, and wherein the substituted phenyl or substituted alkyl is optionally substituted with Q₈.

In certain embodiments, in compounds of general formula I, or salts thereof:
A₁ is

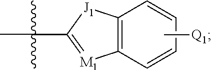

D₁ is S, O, N—H, N—CH₃, N—CH₂—OCH₃, N—CH₂—CH₃, N—CH₂—COOH, N—CH₂—CH₂OH;
E₁ is C—CH₃, C—C(O)OR₄, or C—C(O)R₆₃, wherein R₄ is H or Me, and R₆₃ is CH₃, CH₂CH₃, CH₂CH₂CH₃, CH(CH₃)₂ or CF₃;
each G₁ is independently H, Br, F, Cl, OMe, C(O)R₁, or C(O)OR₁, wherein R₁ is H or Me, and
each Q₁ is independently; H, Br, F, Cl,

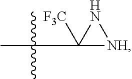 [SMILES: *C1(C(F)(F)F)NN1]

OMe, C(O)R₆, C(O)OR₆, N₃, unsubstituted phenyl, substituted phenyl, unsubstituted alkyl, or substituted alkyl, wherein the alkyl is 1-3 carbons in length, wherein R₆ is H or Me, and wherein the substituted phenyl or substituted alkyl is optionally substituted with Q₈.

In certain embodiments, in compounds of general formula I or formula 2, or salts thereof:
A₁ is

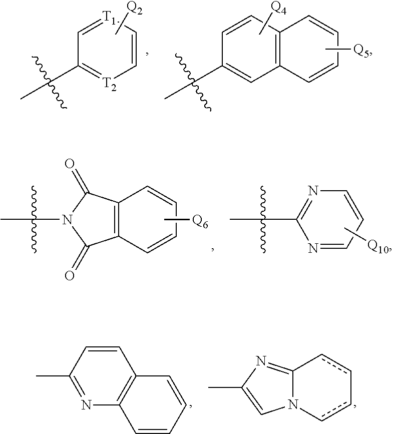

wherein [symbol] represents a single or double bond, or a 5-membered heteroaryl optionally substituted with Q₁₁ and containing 1 or 2 heteroatoms each selected from N, O and S;
D₁ is S, O, N—H, N—CH₃, N—CH₂—OCH₃, N—CH₂—CH₃, N—CH₂—COOH, N—CH₂—CH₂OH, or N—R₂, wherein R₂ is

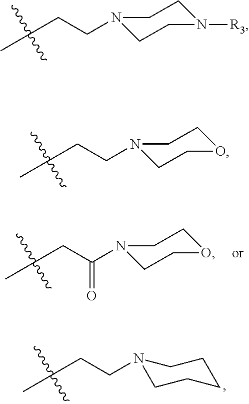

wherein R₃ is H, unsubstituted alkyl, or substituted alkyl, wherein the alkyl is 1-6 carbons in length, and the alkyl is optionally substituted with Br, F, Cl, I, OH, OMe, or N₃, and
E₁ is N, C—H, C—CH₃, C—C(O)OR₄, C—C(O)R₆₃, C—Cl, or C—OR₄, wherein R₄ is H or Me, and R₆₃ is CH₃, CH₂CH₃, CH₂CH₂CH₃, CH(CH₃)₂ or CF₃.

In certain embodiments, in compounds of general formula I or formula 2, or salts thereof:
A₁ is

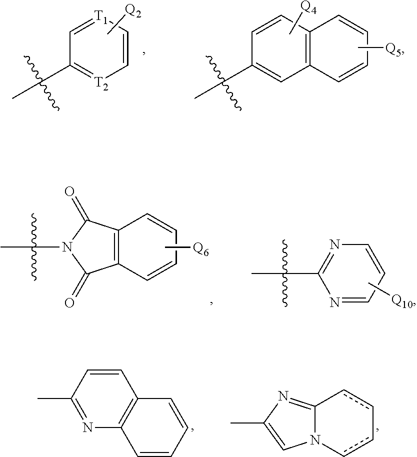

wherein [symbol] represents a single or double bond, or a 5-membered heteroaryl selected from:

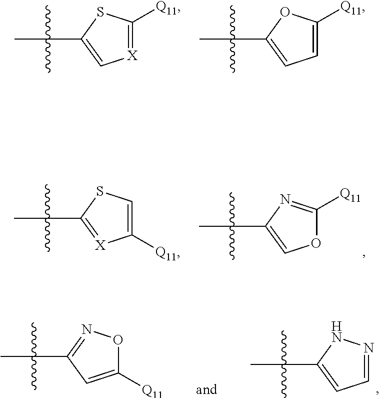

wherein X is N or CH;
D₁ is S, O, N—H, N—CH₃, N—CH₂—OCH₃, N—CH₂—CH₃, N—CH₂—COOH, N—CH₂—CH₂OH, or N—R₂, wherein R₂ is

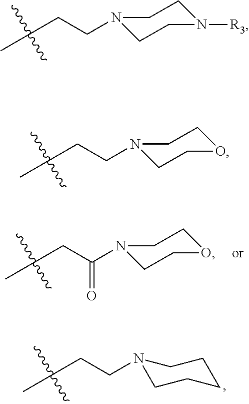

wherein R₃ is H, unsubstituted alkyl, or substituted alkyl, wherein the alkyl is 1-6 carbons in length, and the alkyl is optionally substituted with Br, F, Cl, I, OH, OMe, or N₃, and
E₁ is N, C—H, C—CH₃, C—C(O)OR₄, C—C(O)R₆₃, C—Cl, or C—OR₄, wherein R₄ is H or Me, and R₆₃ is CH₃, CH₂CH₃, CH₂CH₂CH₃, CH(CH₃)₂ or CF₃.

In certain embodiments, in compounds of general formula I:
A₁ is

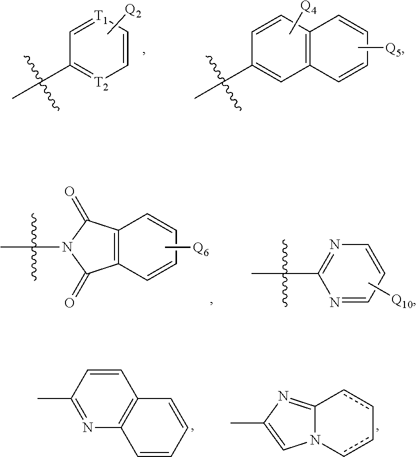

wherein [symbol] represents a single or double bond, or a 5-membered heteroaryl selected from:

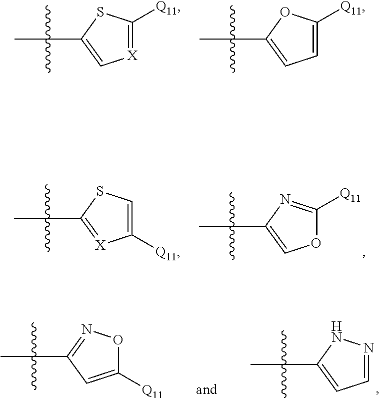

wherein X is N or CH;
D₁ is S, O, N—H, N—CH₃, N—CH₂—OCH₃, N—CH₂—CH₃, N—CH₂—COOH, N—CH₂—CH₂OH, or N—R₂, wherein R₂ is

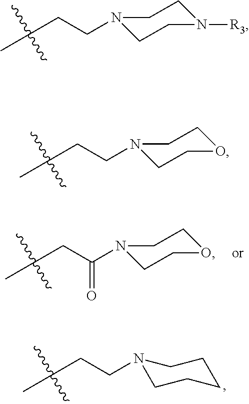

wherein R₃ is H, unsubstituted alkyl, or substituted alkyl, wherein the alkyl is 1-6 carbons in length, and the alkyl is optionally substituted with Br, F, Cl, I, OH, OMe, or N₃;
E₁ is N, C—H, C—CH₃, C—C(O)OR₄, C—C(O)R₆₃, C—Cl, or C—OR₄, wherein R₄ is H or Me, and R₆₃ is CH₃, CH₂CH₃, CH₂CH₂CH₃, CH(CH₃)₂ or CF₃;
each G₁ is independently H, Br, F, Cl, OMe, C(O)R₁, C(O)OR₁, unsubstituted phenyl, substituted phenyl, unsubstituted alkyl or substituted alkyl, wherein the alkyl is 1-3 carbons in length, wherein the substitutions to the phenyl or alkyl are optionally Br, F, Cl, I, OH, OMe, or N₃, and wherein R₁ is H or Me;
each Q₂ is independently H, Br, F, Cl,

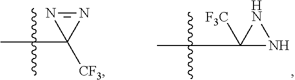 [SMILES: *C1(C(F)(F)F)N=N1 ; *C1(C(F)(F)F)NN1]

N₃, OMe, C(O)R₇, NO₂,

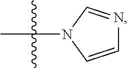 [SMILES: *n1ccnc1]

unsubstituted phenyl, substituted phenyl, unsubstituted alkyl, or substituted alkyl, wherein the alkyl is 1-3 carbons in length, wherein R₇ is H or Me, and wherein the substituted phenyl or substituted alkyl is optionally substituted with Q₉;
each Q₄ is independently H, Br, F, Cl, or OMe;
each Q₅ is independently H, Br, F, Cl, or OMe;
each Q₆ is independently H, Br, F, Cl, or OMe;
each Q₁₀ is independently H, Br, F or Cl;
each Q₁₁ is independently H, Me, unsubstituted phenyl or substituted phenyl, wherein the substituted phenyl is optionally substituted with Q₈.

In certain embodiments, in compounds of general formula I, or salts thereof, each G₁ is independently H, Br, F, Cl, OR₁, C(O)R₁, or C(O)OR₁, wherein R₁ is H or Me.

In certain embodiments, in compounds of general formula I, or salts thereof, each G₁ is independently H, Br, F, Cl, OMe, C(O)R₁, or C(O)OR₁, wherein R₁ is H or Me.

In certain embodiments, in compounds of general formula I or formula 2, or salts thereof,
D₁ is S, O, N—H, N—CH₃, N—CH₂—OCH₃, N—CH₂—CH₃, N—CH₂—COOH, N—CH₂—CH₂OH, and
E₁ is C—CH₃, C—C(O)OR₄, or C—C(O)R₆₃, wherein R₄ is H or Me, and R₆₃ is CH₃, CH₂CH₃, CH₂CH₂CH₃, CH(CH₃)₂ or CF₃.

In certain embodiments, in compounds of general formula I or formula 2, or salts thereof, D₁ is S, N—H, or N—CH₃.

In certain embodiments, L₁ may be —CH₂CH₂—, —CHCH—, —CC—, —C(O)NH—, —NHC(O)—, —C(O)—, —N═CH—, —CH₂R₅—

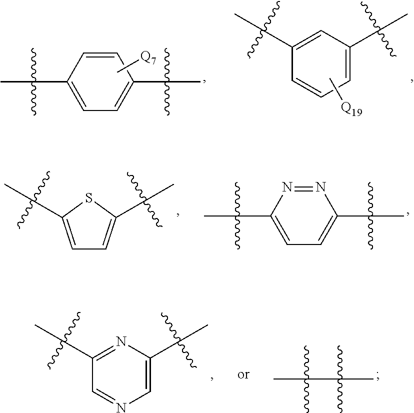

for example, L₁ may be —C(O)NH—, —NHC(O)—, or —C(O)—; or L₁ may be —C(O)NH— or —NHC(O)—; or L₁ may be —CH₂CH₂—, —CHCH—, or —CC—; or L₁ may be —CH₂CH₂—, —CHCH—, —CC—, —C(O)NH—, or —NHC(O)—; or L₁ may be —CH₂CH₂—, —CHCH—, —CC—, —C(O)NH—, —NHC(O)—, or —C(O)—; or L₁ may be —CH₂CH₂—, —CHCH—, —CC—, —C(O)NH—, —NHC(O)—, —C(O)—, —N═CH—, or —CH₂R₅—; or L₁ may be

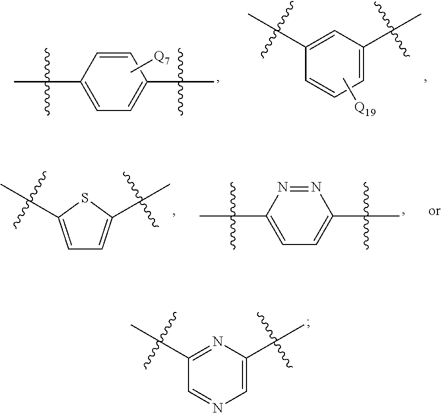

### or L₁ may be

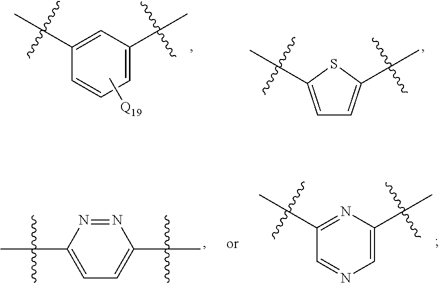

### or L₁ may be

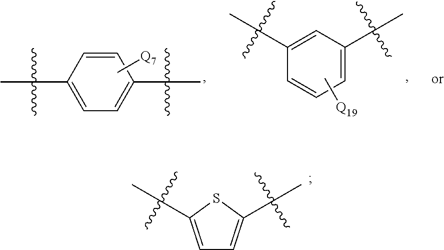

### or L₁ may be

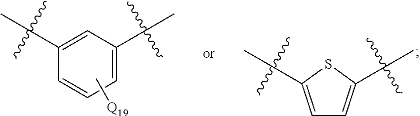

### or L₁ may be

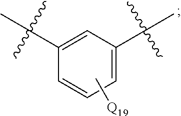

### or L₁ may be

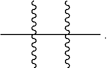 [SMILES: **]

In certain embodiments, R₅ may be CH₂CH₂, 0 or S, for example, R₅ may be CH₂CH₂ or O; or R₅ may be CH₂CH₂ or S; or R₅ may be O or S. In some embodiments, R₅ may be CH₂CH₂. In some embodiments, R₅ may be O. In some embodiments, R₅ may be S.

In certain embodiments, each Q₇ and Q₁₉ may independently be H, Br, Cl, F, I, NO₂, or OMe; for example, each Q₇ and Q₁₉ may independently be H, Br, NO₂, or OMe; or each Q₇ and Q₁₉ may independently be H, NO₂, or OMe; or each Q₇ and Q₁₉ may independently be H, Br, Cl, or F; or each Q₇ and Q₁₉ may independently be H or Br; or each Q₇ and Q₁₉ may independently be H or NO₂; or each Q₇ and Q₁₉ may independently be H or OMe. In some embodiments, each Q₇ and Q₁₉ may be H.

In certain embodiments, A₁ may be

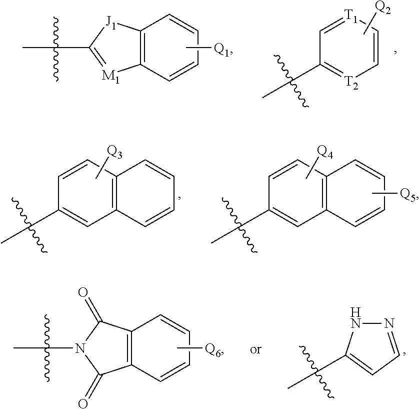

for example, A₁ may be

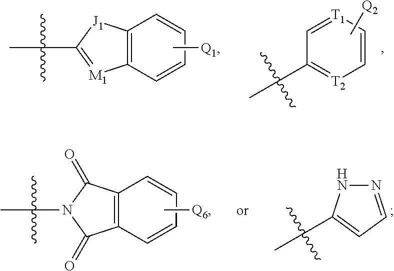

### or A₁ may be

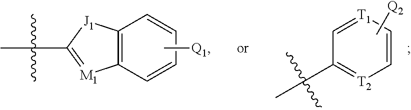

### or A₁ may be

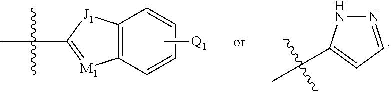

In some embodiments, A₁ may be

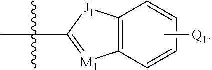

In some embodiments, A₁ may be

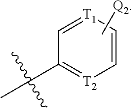

In some embodiments, A₁ may be

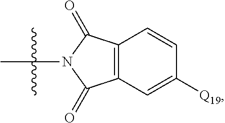

wherein Q₁₉ may be Br, Cl, I, F, or H. In some embodiments, A₁ may be

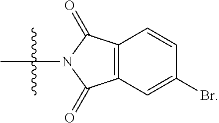 [SMILES: *N1C(=O)c2ccc(Br)cc2C1=O]

In certain embodiments, each G₁ may be independently H, Br, F, Cl, I, OR₁, SO₂R₁, C(O)R₁, unsubstituted phenyl, substituted phenyl, unsubstituted alkyl, substituted alkyl, unsubstituted allyl or substituted allyl, for example, each G₁ may be independently H, Br, F, Cl, I, OR₁, SR₁, SO₂R₁, C(O)R₁, unsubstituted phenyl, substituted phenyl, unsubstituted alkyl or substituted alkyl; or each G₁ may be H, Br, F, Cl, OR₁, C(O)R₁ or C(O)OR₁; or each G₁ may be H, Br, F, Cl, OMe, C(O)R₁ or C(O)OR₁; or each G₁ may be independently Br, F, Cl, OR₁, unsubstituted phenyl, or substituted phenyl; or each G₁ may be independently H, Br, F, Cl, unsubstituted phenyl, or substituted phenyl; or each G₁ may be independently H, Br, unsubstituted phenyl, or substituted phenyl; or each G₁ may be independently H, Br, F, or Cl; or each G₁ may be independently H, Br, or CI; or each G₁ may be independently H or Br; or each G₁ may be H. In some embodiments, R₁ in G₁ may be Me. In some embodiments, R₁ in G₁ may be H.

In certain embodiments, the alkyl or allyl comprised by G₁ may be 1-6 carbons in length, for example, 1-5 carbons in length, 1-4 carbons in length or 1-3 carbons in length. In some embodiments, the substitutions to the phenyl, alkyl, or allyl in G₁ may be one or more of Br, F, Cl, I, OH, OMe, or N₃, for example, the substitutions to the phenyl, alkyl, or allyl may be one or more of Br, F, Cl, OH, OMe, or N₃; or the substitutions to the phenyl, alkyl, or allyl may be one or both of Br or OH; or the substitutions to the phenyl, alkyl or allyl may be Br or OH.

In certain embodiments, G₂ may be H, Br, F, Cl, I, OR₁, SR₁, SO₂R₁, C(O)R₁, OMe, unsubstituted phenyl, substituted phenyl, unsubstituted alkyl, substituted alkyl, unsubstituted allyl, or substituted allyl, for example, G₂ may be H, Br, F, Cl, I, OR₁, OMe, unsubstituted phenyl, substituted phenyl, unsubstituted alkyl, substituted alkyl, unsubstituted allyl, or substituted allyl; or G₂ may be H, Br, F, Cl, I, OMe, unsubstituted phenyl, substituted phenyl, unsubstituted alkyl, substituted alkyl, unsubstituted allyl, or substituted allyl; or G₂ may be H, Br, F, Cl, I, OMe, unsubstituted phenyl, or substituted phenyl; or G₂ may be H, Br, F, Cl, I, or OMe; or G₂ may be H, Br, F, Cl, or OMe; or G₂ may be H, Br, F, or Cl; or G₂ may be H or Br. In some embodiments, R₁ in G₂ may be Me. In some embodiments, R₁ in G₂ may be H.

In certain embodiments, the alkyl or allyl comprised by G₂ may be 1-6 carbons in length, for example, 1-5 carbons in length, 1-4 carbons in length or 1-3 carbons in length. In some embodiments, the substitutions on the phenyl, alkyl or allyl of G₂ may be Br, F, Cl, I, OH, OMe, or N₃, for example, the substitutions may be Br, F, Cl, OH, or OMe.

In certain embodiments, G₃ may be H, Br, F, Cl, I, OR₁, SR₁, SO₂R₁, C(O)R₁, OMe, unsubstituted phenyl, substituted phenyl, unsubstituted alkyl, substituted alkyl, unsubstituted allyl, or substituted allyl, for example, G₃ may be H, Br, F, Cl, I, OR₁, OMe, unsubstituted phenyl, substituted phenyl, unsubstituted alkyl, substituted alkyl, unsubstituted allyl, or substituted allyl; or G₃ may be H, Br, F, Cl, I, OR₁, OMe, unsubstituted phenyl, substituted phenyl, unsubstituted alkyl, substituted alkyl, unsubstituted allyl, or substituted allyl; or G₃ may be H, Br, F, Cl, I, OMe, unsubstituted phenyl, or substituted phenyl; or G₃ may be H, Br, F, Cl, I, or OMe; or G₃ may be H, Br, F, Cl, or OMe; or G₃ may be H, Br, F, or Cl; or G₃ may be H or Br. In some embodiments, R₁ in G₃ may be Me. In some embodiments, R₁ in G₃ may be H.

In certain embodiments, the alkyl or allyl in G₃ may be 1-6 carbons in length, for example, 1-5 carbons in length, 1-4 carbons in length or 1-3 carbons in length. In some embodiments, the substitutions on the phenyl, alkyl or allyl of G₃ may be Br, F, Cl, I, OH, OMe, or N₃, for example, the substitutions may be Br, F, Cl, OH, or OMe.

In certain embodiments, D₁ may be S, O, N—H, N—CH₃, N—CH₂—OCH₃, N—CH₂—CH₃, N—CH₂—COOH, N—CH₂—CH₂OH, CH₂, CH—CH₃, CH—CH₂—OCH₃, CH—CH₂—CH₃, CH—CH₂—COOH, CH—CH₂—CH₂OH, N—R₂, or CH—R₂, for example, D₁ may be S, O, N—H, N—CH₃, N—CH₂—OCH₃, N—CH₂—CH₃, N—CH₂—COOH, N—CH₂—CH₂OH, CH₂, CH—CH₃, CH—CH₂—OCH₃, CH—CH₂—CH₃, CH—CH₂—COOH, or CH—CH₂—CH₂OH; or D₁ may be S, O, N—H, N—CH₃, CH₂, or CH—CH₃; or D₁ may be N—H, N—CH₃, CH₂, or CH—CH₃; or D₁ may be S, N—H or N—CH₃; or D₁ may be S, O, N—H, or CH₂.

In some embodiments, R₂ may be

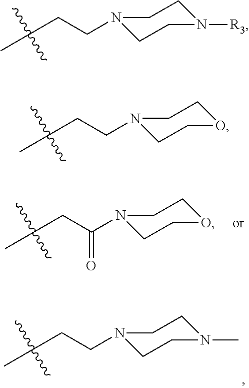

for example, R₂ may be

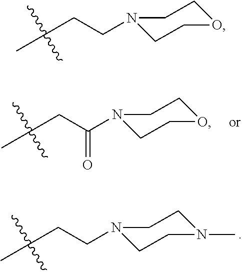

In some embodiments, R₃ may be H, unsubstituted alkyl, or substituted alkyl, in which the alkyl may be 1-6 carbons in length, for example, 1-5 carbons in length, 1-4 carbons in length or 1-3 carbons in length. In some embodiments, the alkyl in R₃ may optionally be substituted with Br, F, Cl, I, OH, OMe, or N₃, for example, the alkyl may optionally be substituted with Br, F, Cl, OH, OMe, or N₃; or with Br, F, Cl, OH, or OMe; or with Br, F, Cl, or OH; or with Br or OH.

In certain embodiments, E₁ may be N, C—H, C—CH₃, C—C(O)CH(CH₃)₂, C—C(O)OCH₃, C—C(O)CH₃, C—Cl, C—Br, C—F, or C—COR₄, for example, E₁ may be N, C—H, C—CH₃, C—Cl, C—Br, C—F, C—C(O)CH(CH₃)₂, C—C(O)OCH₃, or C—C(O)CH₃; or E₁ may be N, C—H, or C—CH₃; or E₁ may be N, or C—H; or E₁ may be C—Cl, C—F, or C—Br; or E₁ may be C—C(O)CH(CH₃)₂, C—C(O)OCH₃, or C—C(O)CH₃. In some embodiments, E₁ may be N, C—H, C—CH₃, C—C(O)OR₄, C—C(O)R₆₃, C—Cl or C—COR₄, for example, E₁ may be N, C—H, C—CH₃, C—C(O)OR₄, C—C(O)R₆₃ or C—Cl. In some embodiments, R₄ may be H. In some embodiments, R₄ may be Me.

In certain embodiments, each Q₁ may be independently H, Br, F, Cl, I,

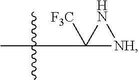 [SMILES: *C1(C(F)(F)F)NN1]

OR₆, SR₆, SO₂R₆, C(O)R₆, unsubstituted phenyl, substituted phenyl, unsubstituted alkyl, substituted alkyl, unsubstituted allyl, or substituted allyl, for example, each Q₁ may be independently H, Br, F, Cl, I,

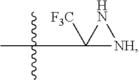 [SMILES: *C1(C(F)(F)F)NN1]

OR₆, unsubstituted phenyl, substituted phenyl, unsubstituted alkyl, substituted alkyl, unsubstituted allyl, or substituted allyl; or each Q₁ may be independently H, Br, F, Cl, I,

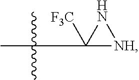 [SMILES: *C1(C(F)(F)F)NN1]

OR₆, C(O)R₆, unsubstituted phenyl, substituted phenyl, unsubstituted alkyl, or substituted alkyl; or each Q₁ may be independently H, Br, F, Cl, I, or

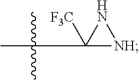 [SMILES: *C1(C(F)(F)F)NN1]

or each Q₁ may be independently H, Br, F, or Cl; or each Q₁ may be independently H or Br; or each Q₁ may be independently H, Br, or F; or each Q₁ may be independently H, Br, or

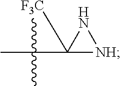 [SMILES: *C1(C(F)(F)F)NN1]

or each Q₁ may be independently H, Br, or Cl. In some embodiments, each Q₁ may be independently H, Br, F, Cl,

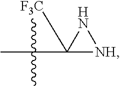 [SMILES: *C1(C(F)(F)F)NN1]

OR₆, C(O)R₆, C(O)OR₆, unsubstituted phenyl, substituted phenyl, unsubstituted alkyl or substituted alkyl, for example, each Q₁ may be independently H, Br, F, Cl, OMe, C(O)R₆, C(O)OR₆, unsubstituted phenyl, substituted phenyl, unsubstituted alkyl or substituted alkyl. In some embodiments, R₆ may be H. In some embodiments, R₆ may be Me. The alkyl or allyl comprised by Q₁ may be 1-6 carbons in length, for example, 1-5 carbons in length, 1-4 carbons in length or 1-3 carbons in length.

In certain embodiments, each Q₂ may be independently H, Br, F, Cl, I,

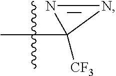 [SMILES: *C1(C(F)(F)F)N=N1]

N₃, OR₇, SR₇, SO₂R₇, C(O)R₇, unsubstituted phenyl, substituted phenyl, unsubstituted alkyl, substituted alkyl, unsubstituted allyl, or substituted allyl, for example, each Q₂ may be independently H, Br, F, Cl, I,

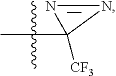 [SMILES: *C1(C(F)(F)F)N=N1]

N₃, OR₇, unsubstituted phenyl, substituted phenyl, unsubstituted alkyl, substituted alkyl, unsubstituted allyl, or substituted allyl; or each Q₂ may be independently H, Br, F, Cl, I,

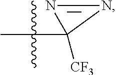 [SMILES: *C1(C(F)(F)F)N=N1]

N₃, unsubstituted phenyl, substituted phenyl, unsubstituted alkyl, substituted alkyl, unsubstituted allyl, or substituted allyl; or each Q₂ may be independently H, Br, F, Cl, OR₇,

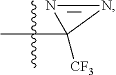 [SMILES: *C1(C(F)(F)F)N=N1]

N₃, unsubstituted phenyl, substituted phenyl, unsubstituted alkyl, or substituted alkyl; or each Q₂ may be independently H, Br, F, Cl, I,

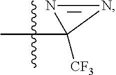 [SMILES: *C1(C(F)(F)F)N=N1]

N₃, OR₇, unsubstituted phenyl, or substituted phenyl; or each Q₂ may be independently H, Br, F, Cl,

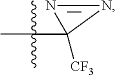 [SMILES: *C1(C(F)(F)F)N=N1]

N₃, unsubstituted phenyl, or substituted phenyl; or each Q₂ may be independently H, Br, F, Cl, N₃, or

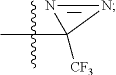 [SMILES: *C1(C(F)(F)F)N=N1]

or each Q₂ may be independently H, Br, F, or Cl; or each Q₂ may be independently H, Br, or OH; or each Q₂ may be independently H or Br; or each Q₂ may be N₃. The alkyl or allyl comprised by Q₃ may be 1-6 carbons in length, for example, 1-5 carbons in length, 1-4 carbons in length or 1-3 carbons in length. In some embodiments, R₇ may be H. In some embodiments, R₇ may be Me.

In certain embodiments, each Q₃ may be independently H, Br, F, Cl, I, or OR₈, for example, each Q₃ may be independently H, Br, F, Cl, or OR₈; or each Q₃ may be independently H, Br, F, or Cl; or each Q may be independently H, Br, or OR₈; or each Q may be independently H or Br. In some embodiments, R₈ may be H. In some embodiments, R₈ may be Me.

In certain embodiments, each Q may be independently H, Br, F, Cl, I, or OR₉, for example, each Q₄ may be independently H, Br, F, Cl, or OR₉; or each Q₄ may be independently H, Br, F, Cl, or I; or each Q₄ may be independently H, Br, F, or Cl; or each Q may be independently H, Br, or OR₉; or each Q₄ may be independently H or Br. In some embodiments, R₉ may be H. In some embodiments, R₉ may be Me.

In certain embodiments, each Q₅ may be independently H, Br, F, Cl, I, or OR₁₀, for example, each Q₅ may be independently H, Br, F, Cl, or OR₁₀; or each Q₅ may be independently H, Br, F, or OR₁₀; or each Q₅ may be independently H, Br, or OR₁₀; or each Q₅ may each independently be H, Br, Cl, or OR₁₀; or each Q₅ may be independently H or Br; or each Q₅ may be independently H or OR₁₀. In some embodiments, R₁₀ may be H. In some embodiments, R₁₀ may be Me.

In certain embodiments, each Q₆ may be independently H, Br, F, Cl, I, or OR₁₁, for example, each Q₆ may be independently H, Br, F, Cl, or OR₁₁; or each Q₆ may be independently H, Br, F, or OR₁₁; or each Q₆ may be independently H, Br, or OR₁₁; or each Q₆ may be independently H, Br, Cl, or OR₁₁; or each Q₆ may be independently H or Br; or each Q₆ may be independently H or OR₁₁. In some embodiments, R₁₁ may be H. In some embodiments, R₁₁ may be Me.

In certain embodiments, each Q₈ may be independently Br, F, Cl, I, Me, or OR₁₂, for example, each Q₈ may be independently Br, F, Cl, Me, or OR₁₂; or each Q₈ may be independently Br, Me, or OR₁₂; or each Q₈ may be independently Br, F, Cl, or Me; or each Q₈ may be independently Br, Me, or OR₁₂; or each Q₈ may be independently Br or Me. In some embodiments, R₁₂ may be H. In some embodiments, R₁₂ may be Me.

In certain embodiments, each Q₉ may be independently Br, F, Cl, I, Me, or OR₁₃, for example, each Q₉ may be independently Br, F, Cl, Me, or OR₁₃; or each Q₉ may be independently Br, F, Me, or OR₁₃; or each Q₉ may be independently Br, Cl, Me, or OR₁₃; or each Q₉ may be independently Br, Me, or OR₁₃; or each Q₉ may be independently Br or Me. In some embodiments, R₁₃ may be H. In some embodiments, R₁₃ may be Me.

In certain embodiments, each Q₁₀ may be independently H, Br, F or CI, for example, each Q₁₀ may be independently H or Cl.

In certain embodiments, each Q₁₁ may be independently H, Me or unsubstituted phenyl.

In certain embodiments, J₁ may be S, O, N—H, N—CH₃, CH—CH₃, N—R₁₄, or CH—R₁₄, for example, J₁ may be S, O, N—H, N—CH₃, or CH—CH₃; or J₁ may be S, O, N—H, N—CH₃, CH—CH₃, or N—R₁₄; or J₁ may be S, O, N—H, N—CH₃, CH—CH₃, or CH—R₁₄; or J₁ may be N—H, N—CH₃, or CH—CH₃. In some embodiments, J₁ may be N—H. In some embodiments, J₁ may be CH—CH₃. In some embodiments, J₁ may be N—CH₃. In some embodiments, J₁ may be S or O, for example, J₁ may be 0; or J₁ may be S. In some embodiments. R₁₄ may be

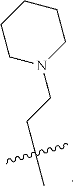 [SMILES: *CCN1CCCCC1]

In some embodiments, R₁₄ may be

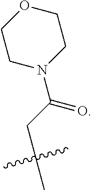 [SMILES: *CC(=O)N1CCOCC1]

In some embodiments, R₁₄ may be

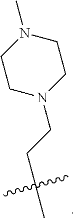 [SMILES: *CCN1CCN(C)CC1]

In certain embodiments, M₁ may be N, C—H, C—CH₃, C—C(O)CH₃, C—C(O)OCH₃, or C—CH(CH₃)₂, for example, M₁ may be N, C—H, C—CH₃, C—C(O)CH₃, or C—C(O)OCH₃; or M₁ may be N, C—H, C—CH₃, C—C(O)CH₃, or C—CH(CH₃)₂; or M₁ may be N, C—H, C—CH₃, C—C(O)OCH₃, or C—CH(CH₃)₂; or M₁ may be N, C—H, or C—CH₃; or M₁ may be C—C(O)CH₃, C—C(O)OCH₃, or C—CH(CH₃)₂. In some embodiments, M₁ may be N. In some embodiments, M₁ may be C—H. In some embodiments, M₁ may be C—CH₃. In some embodiments, M₁ may be C—C(O)CH₃. In some embodiments, M₁ may be C—C(O)OCH₃. In some embodiments, M₁ may be C—CH(CH₃)₂.

In certain embodiments, T₁ and T₂ may each independently be N or C—H. In some embodiments, T₁ may be N and T₂ may be N. In some embodiments, T₁ may be N and T₂ may be C—H. In some embodiments, T₁ may be C—H and T₂ may be N. In some embodiments, T₁ may be C—H and T₂ may be C—H. In some embodiments, at least one of T₁ and T₂ is N.

Combinations of any of the foregoing embodiments for compounds of general formula I are also contemplated and each combination forms a separate embodiment for the purposes of the present disclosure. Likewise, embodiments recited with respect to compounds of general formula I are also contemplated as embodiments of the invention with respect to compounds of formula 2.

In certain embodiments, compounds of general formula I include compounds of general formula II and general formula III, and salts thereof:

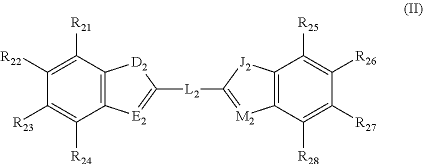

wherein:
L₂ is —CH₂CH₂—, —CHCH—, —CC—, —C(O)NH—, —NHC(O)—, —C(O)—, —N═CH—, —CH₂R₃₅—, —NHCH₂—,

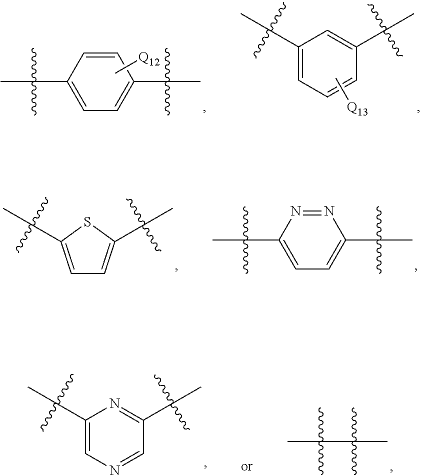

wherein R₃₅ is CH₂CH₂, NHCH₂, NH, SCH₂, S or O, and wherein each Q₁₂ and Q₁₃ are independently H, NO₂, or OMe;
D₂ is S, O, N—H, N—CH₃, N—CH₂—OCH₃, N—CH₂—CH₃, N—CH₂—COOH, N—CH₂—CH₂OH, or N—R₂₉, wherein R₂₉ is

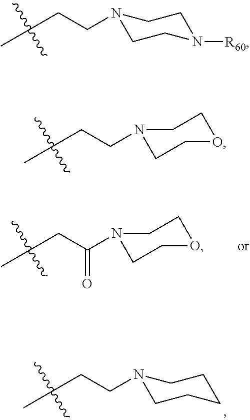

wherein R₆₀ is H, unsubstituted alkyl, or substituted alkyl, wherein the alkyl is 1-6 carbons in length, and the alkyl is optionally substituted with Br, F, Cl, I, OH, OMe, or N₃;
E₂ is C—CH₃, C—C(O)R₅₇, or C—C(O)OR₃₆, wherein R₃₆ is H or Me, and R₅₇ is CH₃, CH₂CH₃, CH₂CH₂CH₃, CH(CH₃)₂ or CF₃;
J₂ is S, O, N—H, N—CH₃, N—CH₂—OCH₃, N—CH₂—COOH, N—CH₂—CH₂OH, or N—R₃₈, wherein R₃₈ is

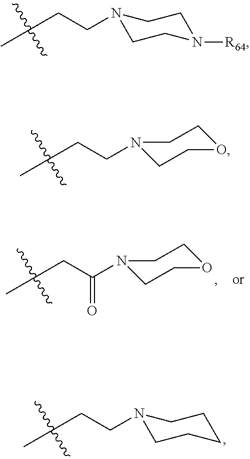

wherein R₆₄ is H, unsubstituted alkyl, or substituted alkyl, wherein the alkyl is 1-6 carbons in length, and the alkyl is optionally substituted with Br, F, Cl, I, OH, OMe, or N₃;
M₂ is N, C—H, C—CH₃, C—C(O)R₅₇, or C—C(O)OR₃₆, wherein R₃₆ is H or Me, and R₅₇ is CH₃, CH₂CH₃, CH₂CH₂CH₃, CH(CH₃)₂ or CF₃;
each of R₂₁, R₂₂, R₂₃, R₂₄, R₂₅, R₂₆, R₂₇ and R₂₈ is independently H, Br, F, Cl, I,

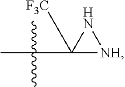 [SMILES: *C1(C(F)(F)F)NN1]

OR₂₉, C(O)R₂₉, C(O)OR₂₉, N₃, unsubstituted phenyl, substituted phenyl, unsubstituted alkyl, or substituted alkyl, wherein the alkyl is 1-6 carbons in length, wherein R₂₉ is H or Me, and wherein the substituted phenyl or substituted alkyl is optionally substituted with Q₁₄, and
each Q₁₄ is independently Br, F, Cl, I, Me, or OR₃₇, wherein R₃₇ is H or Me;

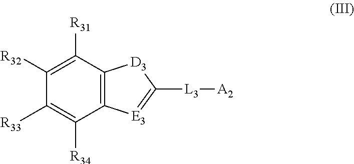

wherein:
L₃ is —CH₂CH₂—, —CHCH—, —CC—, —C(O)NH—, —NHC(O)—, —C(O)—, —N═CH—, —CH₂R₃₉—, —NHCH₂—,

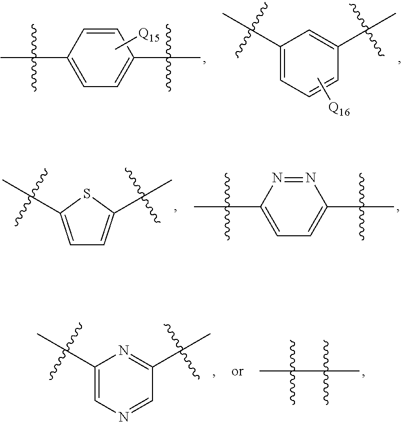

wherein R₃₉ is CH₂CH₂, NHCH₂, NH, SCH₂, S or O, and wherein each Q₁₅ and Q₁₆ are independently H, NO₂, or OMe;
A₂ is

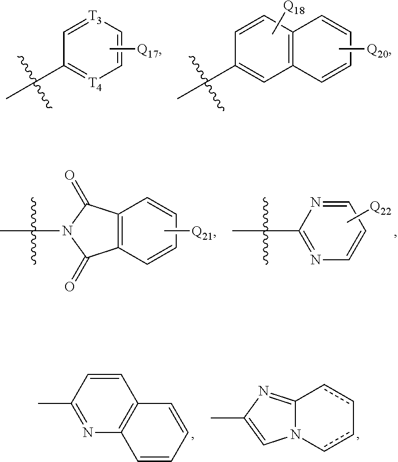

wherein [symbol] represents a single or double bond, or a 5-membered heteroaryl optionally substituted with Q₂₃ and containing 1 or 2 heteroatoms each selected from N, O and S;
D₃ is S, O, N—H, N—CH₃, N—CH₂—OCH₃, N—CH₂—CH₃, N—CH₂—COOH, N—CH₂—CH₂OH, or N—R₆₁, wherein R₆₁ is

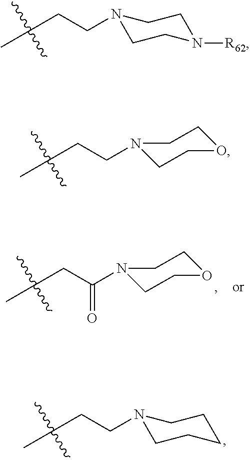

wherein R₆₂ is H, unsubstituted alkyl, or substituted alkyl, wherein the alkyl is 1-6 carbons in length, and the alkyl is optionally substituted with Br, F, Cl, I, OH, OMe, or N₃;
E₃ is N, C—H, C—Cl, C—CH₃, C—C(O)R₅₉, or C—C(O)OR₄₀, wherein R₄₀ is H or Me, and R₅₉ is CH₃, CH₂CH₃, CH₂CH₂CH₃, CH(CH₃)₂ or CF₃;
each of R₃₁, R₃₂, R₃₃ and R₃₄ is independently H, Br, F, Cl, I,

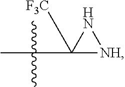 [SMILES: *C1(C(F)(F)F)NN1]

OR₄₁, C(O)R₄₁, C(O)OR₄₁, N₃, unsubstituted phenyl, substituted phenyl, unsubstituted alkyl, or substituted alkyl, wherein the alkyl is 1-6 carbons in length, wherein R₄₁ is H or Me, and wherein the substituted phenyl or substituted alkyl is optionally substituted with Q₂₄;
each Q₁₇ is independently H, Br, F, Cl, I,

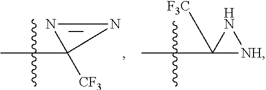 [SMILES: *C1(C(F)(F)F)N=N1 ; *C1(C(F)(F)F)NN1]

N₃, OR₄₂, C(O)R₄₂, NO₂,

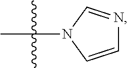 [SMILES: *n1ccnc1]

unsubstituted phenyl, substituted phenyl, unsubstituted alkyl, or substituted alkyl, wherein the alkyl is 1-3 carbons in length, wherein R₄₂ is H or Me, and wherein the substituted phenyl or substituted alkyl is optionally substituted with Q₂4;
each Q₁₈ is independently H, Br, F, Cl, I, or OR₄₃, wherein R₄₃ is H or Me;
each Q₂₀ is independently H, Br, F, Cl, I, or OR₄₄, wherein R₄₄ is H or Me;
each Q₂₁ is independently H, Br, F, Cl, I, or OR₄₅, wherein R₄₅ is H or Me;
each Q₂₂ is independently H, Br, F, Cl or I;
each Q₂₃ is independently H, Me, unsubstituted phenyl, or substituted phenyl, wherein the substituted phenyl is optionally substituted with Q₂₄;
each Q₂₄ is independently Br, F, Cl, I, Me, or OR₄₆, wherein R₄₆ is H or Me;
T₃ is N or C—H; and
T₄ is N or C—H.

Certain embodiments of the invention relate to compounds of general formula II, or a salt thereof, wherein at least one of R₂₁, R₂₂, R₂₃, R₂₄, R₂₅, R₂₆, R₂₇ and R₂₈ is Br, F, Cl or I.

In certain embodiments, in compounds of general formula II, or salts thereof:
L₂ is —CH₂CH₂—, —CHCH—, —CC—, —C(O)NH—, —NHC(O)—, —C(O)—, —N═CH—, —CH₂R₃₅—, —NHCH₂—,

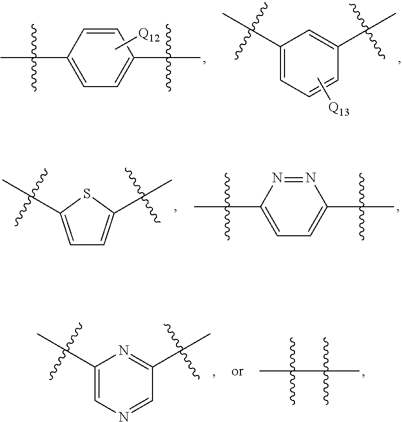

wherein R₃₅ is NHCH₂, NH, SCH₂, or S, and wherein each Q₁₂ and Q₁₃ are independently H, NO₂, or OMe;
D₂ is S, N—H, N—CH₃, N—CH₂—OCH₃, N—CH₂—CH₃, N—CH₂—COOH, N—CH₂—CH₂OH, or N—R₂₉, wherein R₂₉ is

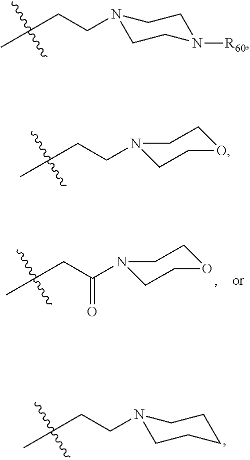

wherein R₆₀ is H, unsubstituted alkyl, or substituted alkyl, wherein the alkyl is 1-6 carbons in length, and the alkyl is optionally substituted with Br, F, Cl, I, OH, OMe, or N₃;
E₂ is C—CH₃, C—C(O)CH(CH₃)₂, C—C(O)CH₃, C—C(O)CF₃, or C—C(O)OR₃₆, wherein R₃₆ is H or Me;
each of R₂₁, R₂₂, R₂₃, R₂₄, R₂₅, R₂₆, R₂₇ and R₂₈ is independently H, Br, F, Cl,

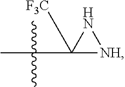 [SMILES: *C1(C(F)(F)F)NN1]

OR₂₉, C(O)R₂₉, C(O)OR₂₉, N₃, unsubstituted phenyl, substituted phenyl, unsubstituted alkyl, or substituted alkyl, wherein the alkyl is 1-3 carbons in length, wherein R₂₉ is H or Me, and wherein the substituted phenyl or substituted alkyl is optionally substituted with Q₁₄;
each Q₁₄ is independently Br, F, Cl, Me, or OR₃₇, wherein R₃₇ is H or Me;
J₂ is S, O, N—H, N—CH₃, N—CH₂—OCH₃, N—CH₂—COOH, N—CH₂—CH₂OH, or N—R₃₈, wherein R₃₈ is

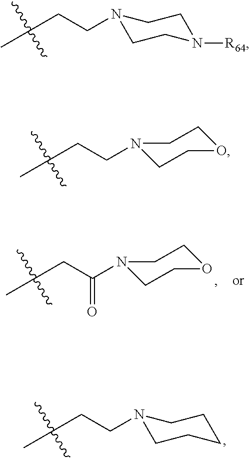

wherein R₆₄ is H, unsubstituted alkyl, or substituted alkyl, wherein the alkyl is 1-6 carbons in length, and the alkyl is optionally substituted with Br, F, Cl, I, OH, OMe, or N₃; and
M₂ is N, C—H, C—C(O)CH₃, C—C(O)CF₃, or C—C(O)OR₃₆, wherein R₃₆ is H or Me.

In certain embodiments, in compounds of general formula II, or salts thereof, L₂ is —CH₂CH₂—, —CHCH—, —CC—, —C(O)NH—, —NHC(O)—, —C(O)—, —N═CH—, —CH₂R₃₅—, —NHCH₂—, or

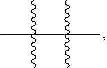

wherein R₃₅ is NHCH₂, NH, SCH₂, or S.

In certain embodiments, in compounds of general formula II, or salts thereof, L₂ is —CH₂CH₂—, —CHCH—, —CC—, —C(O)NH—, —NHC(O)— or

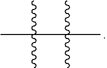

In certain embodiments, in compounds of general formula II, or salts thereof, L₂ is —CH₂CH₂—, —CHCH—, —CC— or

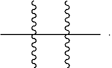

In certain embodiments, in compounds of general formula II, or salts thereof, each of R₂₁, R₂₂, R₂₃ and R₂₄ is independently H, Br, F, Cl, OR₂₉, C(O)R₂₉, or C(O)OR₂₉, wherein R₂₉ is H or Me.

In certain embodiments, in compounds of general formula II, or salts thereof, each of R₂₁, R₂₂, R₂₃ and R₂₄ is independently H, Br, F, Cl, OMe, C(O)R₂₉, or C(O)OR₂₉, wherein R₂₉ is H or Me.

In certain embodiments, in compounds of general formula II, or salts thereof, each of R₂₅, R₂₆, R₂₇ and R₂₈ is independently H, Br, F, Cl, OR₂₉, C(O)R₂₉, or C(O)OR₂₉, wherein R₂₉ is H or Me.

In certain embodiments, in compounds of general formula II, or salts thereof, each of R₂₅, R₂₆, R₂₇ and R₂₈ is independently H, Br, F, Cl, OMe, C(O)R₂₉, or C(O)OR₂₉, wherein R₂₉ is H or Me.

In certain embodiments, in compounds of general formula II, or salts thereof, at least one of R₂₁, R₂₂, R₂₃ and R₂₄ is Br, F, Cl or I.

In certain embodiments, in compounds of general formula II, or salts thereof, at least one of R₂₁, R₂₂, R₂₃ and R₂₄ is Br.

In certain embodiments, in compounds of general formula II, or salts thereof, R₂₂ is Br.

In certain embodiments, in compounds of general formula II, or salts thereof, D₂ is N—H, N—CH₃, N—CH₂—OCH₃, N—CH₂—CH₃, N—CH₂—COOH, N—CH₂—CH₂OH, or N—R₂₉.

In certain embodiments, in compounds of general formula II, or salts thereof, D₂ is S, N—H, or N—CH₃;

In certain embodiments, in compounds of general formula II, or salts thereof, D₂ is N—H or N—CH₃.

In certain embodiments, in compounds of general formula II, or salts thereof:
D₂ is N—H, N—CH₃, N—CH₂—OCH₃, N—CH₂—CH₃, N—CH₂—COOH, N—CH₂—CH₂OH, or N—R₂₉, and
R₂₂ is Br.

In certain embodiments, in compounds of general formula II, or salts thereof, E₂ is C—C(O)R₅₇ or C—C(O)OR₃₆.

In certain embodiments, in compounds of general formula II, or salts thereof, E₂ is C—C(O)CF₃, C—C(O)OMe or C—C(O)OH.

In certain embodiments, in compounds of general formula II, or salts thereof:
D₂ is N—H or N—CH₃, and
E₂ is C—C(O)R₅₇ or C—C(O)OR₃₆.

Combinations of any of the foregoing embodiments for compounds of general formula II are also contemplated and each combination forms a separate embodiment for the purposes of the present disclosure.

Certain embodiments of the invention relate to compounds of general formula III, or salts thereof, wherein:
(A) when A₂ is

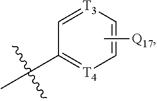

and T₃ and T₄ are each C—H, then at least one of R₃₁, R₃₂, R₃₃, R₃₄ or Q₁₇ is Br, F, Cl or I; and
(B) the compound is not one of the following:

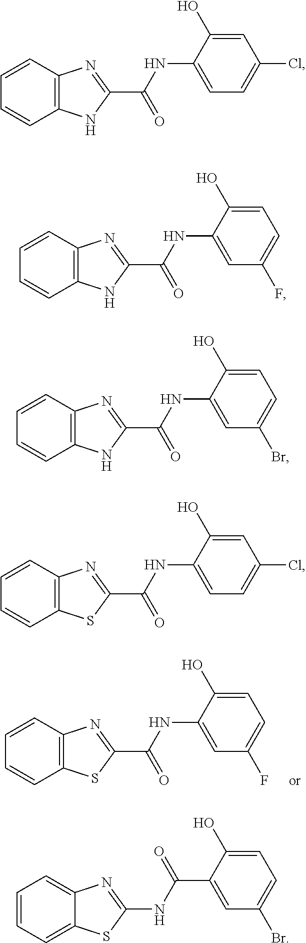 [SMILES: O=C(Nc1ccc(Cl)cc1O)c1nc2ccccc2[nH]1 ; O=C(Nc1cc(F)ccc1O)c1nc2ccccc2[nH]1 ; O=C(Nc1cc(Br)ccc1O)c1nc2ccccc2[nH]1 ; O=C(Nc1ccc(Cl)cc1O)c1nc2ccccc2s1 ; O=C(Nc1cc(F)ccc1O)c1nc2ccccc2s1 ; O=C(Nc1nc2ccccc2s1)c1cc(Br)ccc1O]

Certain embodiments of the invention relate to compounds of general formula III, or salts thereof, wherein:
(A) when A₂ is

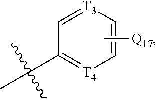

and T₃ and T₄ are each C—H, then at least one of R₃₁, R₃₂, R₃₃, R₃₄ or Q₁₇ is Br, F, Cl or I; and
(B) the compound is not one of the compounds shown in Table A.

In certain embodiments, in compounds of general formula III, or salts thereof:
L₃ is —CH₂CH₂—, —CHCH—, —CC—, —C(O)NH—, —NHC(O)—, —C(O)—, —N—CH—, —CH₂R₃₉—, —NHCH₂—,

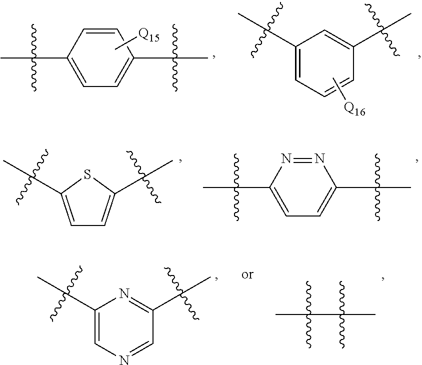

wherein R₃₉ is NHCH₂, NH, SCH₂, or S, and wherein each Q₁₅ and Q₁₆ are independently H, NO₂, or OMe;
A₂ is

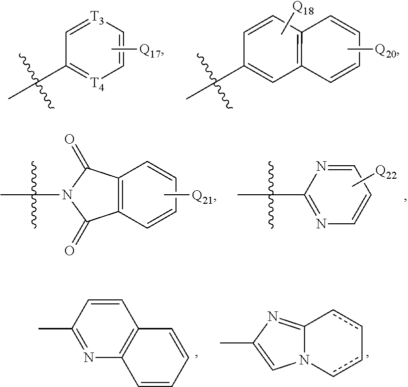

wherein [symbol] represents a single or double bond, or a 5-membered heteroaryl optionally substituted with Q₂₃ and containing 1 or 2 heteroatoms each selected from N, O and S;
D₃ is S, N—H, N—CH₃, N—CH₂—OCH₃, N—CH₂—CH₃, N—CH₂—COOH, N—CH₂—CH₂OH, or N—R₆₁, wherein R₆₁ is

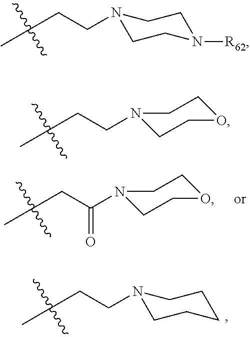

wherein R₆₂ is H, unsubstituted alkyl, or substituted alkyl, wherein the alkyl is 1-6 carbons in length, and the alkyl is optionally substituted with Br, F, Cl, I, OH, OMe, or N₃;
E₃ is N, C—H, C—Cl, C—CH₃, C—C(O)CH(CH₃)₂, C—C(O)CH₃, C—C(O)CF₃, or C—C(O)OR₄₀, wherein R₄₀ is H or Me;
each of R₃₁, R₃₂, R₃₃ and R₃₄ is independently H, Br, F, Cl,

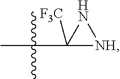 [SMILES: *C1(C(F)(F)F)NN1]

OR₄₁, C(O)R₄₁, C(O)OR₄₁, N₃, unsubstituted phenyl, substituted phenyl, unsubstituted alkyl, or substituted alkyl, wherein the alkyl is 1-3 carbons in length, wherein R₄₁ is H or Me, and wherein the substituted phenyl or substituted alkyl is optionally substituted with Q₂₄;
each Q₁₇ is independently H, Br, F, Cl,

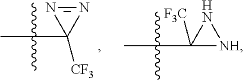 [SMILES: *C1(C(F)(F)F)N=N1 ; *C1(C(F)(F)F)NN1]

N₃, OR₄₂, C(O)R₄₂, NO₂,

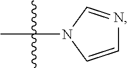 [SMILES: *n1ccnc1]

unsubstituted phenyl, substituted phenyl, unsubstituted alkyl, or substituted alkyl, wherein the alkyl is 1-3 carbons in length, wherein R₄₂ is H or Me, and wherein the substituted phenyl or substituted alkyl is optionally substituted with Q₂₄;
each Q₁₈ is independently H, Br, F, Cl, or OR₄₃, wherein R₄₃ is H or Me;
each Q₂₀ is independently H, Br, F, Cl, or OR₄₄, wherein R₄₄ is H or Me;
each Q₂₁ is independently H, Br, F, Cl, or OR₄₅, wherein R₄₅ is H or Me;
each Q₂₂ is independently H, Br, F or Cl;
each Q₂₃ is independently H, Me, unsubstituted phenyl, or substituted phenyl, wherein the substituted phenyl is optionally substituted with Q₂₄;
each Q₂₄ is independently Br, F, Cl, Me, or OR₄₆, wherein R₄₆ is H or Me;
T₃ is N or C—H; and
T₄ is N or C—H.

In certain embodiments, in compounds of general formula III, or salts thereof, L₃ is —CH₂CH₂—, —CHCH—, —CC—, —C(O)NH—, —NHC(O)—, —C(O)—, —N═CH—, —CH₂R₃₉—, —NHCH₂—, or

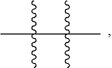

wherein R₃₉ is NHCH₂, NH, SCH₂, or S.

In certain embodiments, in compounds of general formula III, or salts thereof, L₃ is —CH₂CH₂—, —CHCH—, —CC—, —C(O)NH—, —NHC(O)—, CH₂R₃₉—, —NHCH₂—, or

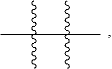

wherein R₃₉ is NHCH₂, NH, SCH₂, or S.

In certain embodiments, in compounds of general formula III, or salts thereof, L₃ is —CH₂CH₂—, —CHCH—, —CC—, —C(O)NH— or —NHC(O)—.

In certain embodiments, in compounds of general formula III, or salts thereof, each of R₃₁, R₃₂, R₃₃ and R₃₄ is independently H, Br, F, Cl, OR₄₁, C(O)R₄₁, or C(O)OR₄₁, wherein R₄₁ is H or Me.

In certain embodiments, in compounds of general formula III, or salts thereof, each of R₃₁, R₃₂, R₃₃ and R₃₄ is independently H, Br, F, Cl, OMe, C(O)R₄₁, or C(O)OR₄₁, wherein R₄₁ is H or Me.

In certain embodiments, in compounds of general formula III, or salts thereof, at least one of R₃₁, R₃₂, R₃₃ and R₃₄ is Br, F, Cl, or I.

In certain embodiments, in compounds of general formula III, or salts thereof, at least one of R₃₁, R₃₂, R₃₃ and R₃₄ is Br.

In certain embodiments, in compounds of general formula III, or salts thereof, R₃₂ is Br.

In certain embodiments, in compounds of general formula III, or salts thereof:
A₂ is

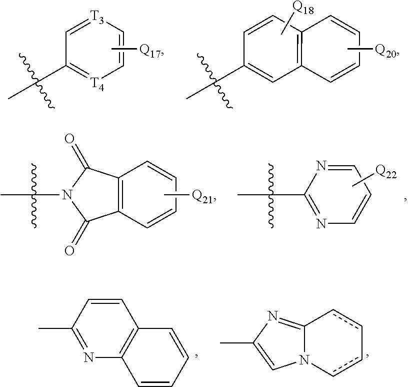

wherein [symbol] represents a single or double bond, or a 5-membered heteroaryl selected from:

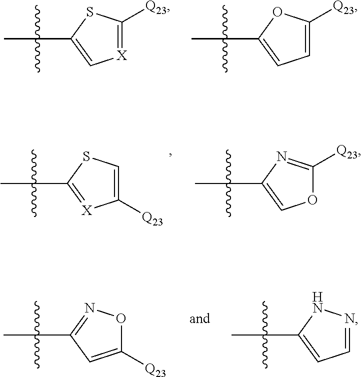

wherein X is N or CH.

In certain embodiments, in compounds of general formula III, or salts thereof:
A₂ is

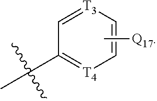

In certain embodiments, in compounds of general formula III, or salts thereof:
A₂ is

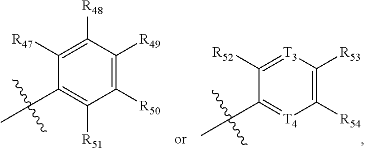

wherein:
R₄₇, R₄₈, R₄₉, R₅₀ and R₅₁ are each independently H, Br, F, Cl, I,

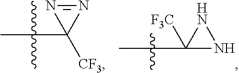 [SMILES: *C1(C(F)(F)F)N=N1 ; *C1(C(F)(F)F)NN1]

N₃, OR₅₅, C(O)R₅₅, NO₂,

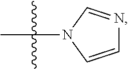 [SMILES: *n1ccnc1]

unsubstituted phenyl, substituted phenyl, unsubstituted alkyl, or substituted alkyl, wherein the alkyl is 1-3 carbons in length, wherein R₅₅ is H or Me, and wherein the substituted phenyl or substituted alkyl is optionally substituted with one or more of Br, F, Cl, I, Me, OMe and OH,
T₃ and T₄ are each independently N or C—H, provided that at least one of T₃ and T₄ is N, and
R₅₂, R₅₃ and R₅₄ are each independently H, Br, F, Cl, I, OR₅₆, unsubstituted phenyl, substituted phenyl, unsubstituted alkyl or substituted alkyl, wherein the alkyl is 1-3 carbons in length, wherein R₅₆ is H or Me, and wherein the substituted phenyl or substituted alkyl is optionally substituted with one or more of Br, F, Cl, I, Me, OMe and OH.

In certain embodiments, in compounds of general formula III, or salts thereof:
A₂ is

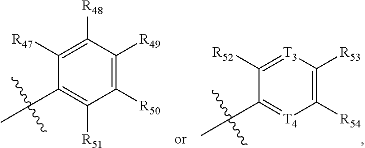

wherein:
R₄₇, R₄₈, R₄₉, R₅₀ and R₅₁ are each independently H, Br, F, Cl, I,

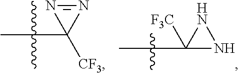 [SMILES: *C1(C(F)(F)F)N=N1 ; *C1(C(F)(F)F)NN1]

N₃, OMe, C(O)R₅₅, NO₂,

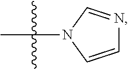 [SMILES: *n1ccnc1]

unsubstituted phenyl, substituted phenyl, unsubstituted alkyl, or substituted alkyl, wherein the alkyl is 1-3 carbons in length, wherein R₅₅ is H or Me, and wherein the substituted phenyl or substituted alkyl is optionally substituted with one or more of Br, F, Cl, I, Me, OMe and OH,
T₃ and T₄ are each independently N or C—H, provided that at least one of T₃ and T₄ is N, and
R₅₂, R₅₃ and R₅₄ are each independently H, Br, F, Cl, I, OMe, unsubstituted phenyl, substituted phenyl, unsubstituted alkyl or substituted alkyl, wherein the alkyl is 1-3 carbons in length, wherein the substituted phenyl or substituted alkyl is optionally substituted with one or more of Br, F, Cl, I, Me, OMe and OH.

In certain embodiments, in compounds of general formula III, or salts thereof, D₃ is S, N—H, or N—CH₃.

In certain embodiments, in compounds of general formula III, or salts thereof, D₃ is N—H, N—CH₃, N—CH₂—OCH₃, N—CH₂—CH₃, N—CH₂—COOH, N—CH₂—CH₂OH, or N—R₆₁.

In certain embodiments, in compounds of general formula III, or salts thereof, D₃ is N—H or N—CH₃.

In certain embodiments, in compounds of general formula III, or salts thereof:
D₃ is N—H, N—CH₃, N—CH₂—OCH₃, N—CH₂—CH₃, N—CH₂—COOH, N—CH₂—CH₂OH, or N—R₆₁, and
R₃₂ is Br.

In certain embodiments, in compounds of general formula III, or salts thereof, E₃ is C—C(O)R₅₉ or C—C(O)OR₄₀.

In certain embodiments, in compounds of general formula III, or salts thereof, E₃ is C—C(O)CF₃, C—C(O)OMe or C—C(O)OH.

In certain embodiments, in compounds of general formula III, or salts thereof:
D₃ is N—H or N—CH₃, and
E₃ is C—C(O)R₅₉ or C—C(O)OR₄₀.

Combinations of any of the foregoing embodiments for compounds of general formula III are also contemplated and each combination forms a separate embodiment for the purposes of the present disclosure.

In certain embodiments, compounds of general formula I, or salts thereof, comprise compounds of formulae 3, 4, 5, 6A, 6B, 7A, 7B and 7C, or salts thereof:

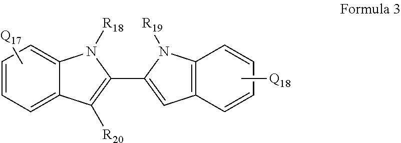

wherein:
R₁₈ is H or CH₃;
R₁₉ is H, CH₃, CH₂OCH₃, CH₂COOH, CH₂CH₂OH,

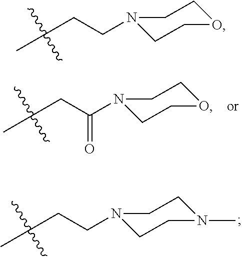

R₂₀ is C(O)Me, C(O)CF₃, C(O)OH or C(O)OMe;
each Q₁₇ is independently Br or H, and
each Q₁₈ is independently H, Cl, F, Br, OMe, substituted phenyl or unsubstituted phenyl.

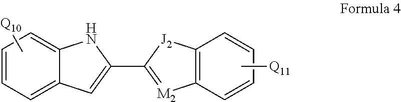

wherein:
each Q₁₀ and each Q₁₁ is independently Br or H;
J₂ is S or O, and
M₂ is N or CH.

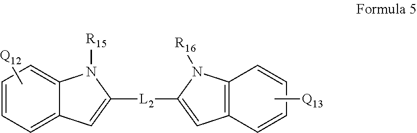

wherein:
each Q₁₂ and each Q₁₃ is independently Br or H;
L₂ is —CH₂CH₂—, —CHCH—, —CC—,

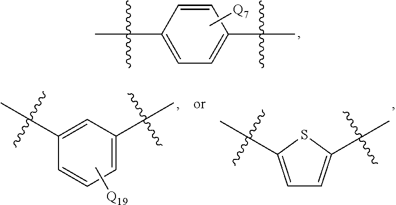

wherein each Q₇ and each Q₁₉ is independently H, NO₂, or OMe, and
each R₁₅ and each R₁₆ is independently H or CH₃.

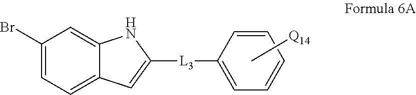

wherein:
each Q₁₄ is independently Br, Cl, or H, and
L₃ is —CH₂CH₂— or —CHCH—.

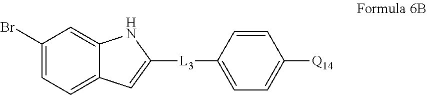

wherein:
Q₁₄ is Br, Cl, or H, and
L₃ is —CH₂CH₂— or —CHCH—.

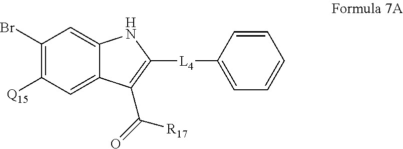

wherein:
Q₁₅ is H or Br;
R₁₇ is OH, CH₃, CH(CH₃)₂, CF₃, or OCH₃, and
L₄ is —CH₂CH₂— or —CHCH—.

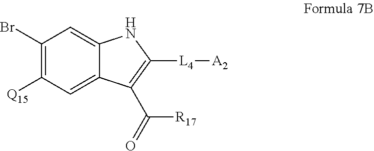

wherein:
Q₁₅ is H or Br;
R₁₇ is OH, CH₃, CH(CH₃)₂, CF₃, or OCH₃;
L₄ is —CH₂CH₂—, —CHCH—, or

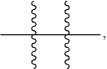 [SMILES: **]

and
A₂ is

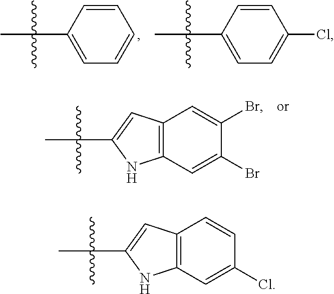

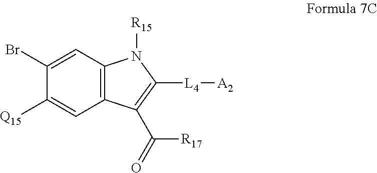

wherein:
Q₁₅ is H or Br;
R₁₅ is H or CH₃;
R₁₇ is OH, CH₃, CH(CH₃)₂, CF₃, or OCH₃;
L₄ is —CH₂CH₂—, —CHCH—, —C(O)NH—, —NHC(O)— or

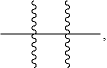 [SMILES: **]

and
A₂ is

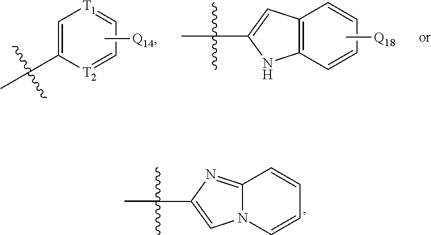

wherein each Q₁₄ is independently H, Cl, F, Br or OMe, each Q₁₈ is independently H, Cl, F, Br, OMe, substituted phenyl or unsubstituted phenyl, and T₁ and T₂ are each independently C—H or N.

Certain embodiments of the invention relate to the compounds shown in Table B and Table C, or salts thereof:

TABLE B

| | |
|---|---|
| 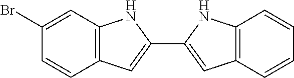 [SMILES: Brc1ccc2cc(-c3cc4ccccc4[nH]3)[nH]c2c1] | 10a |
| 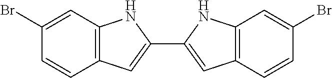 [SMILES: Brc1ccc2cc(-c3cc4ccc(Br)cc4[nH]3)[nH]c2c1] | 10b |
| 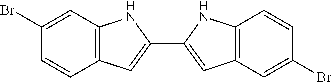 [SMILES: Brc1ccc2[nH]c(-c3cc4ccc(Br)cc4[nH]3)cc2c1] | 10c |
| 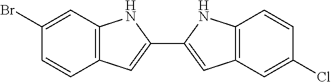 [SMILES: Clc1ccc2[nH]c(-c3cc4ccc(Br)cc4[nH]3)cc2c1] | 10d |
| 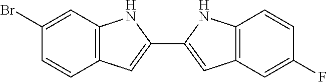 [SMILES: Fc1ccc2[nH]c(-c3cc4ccc(Br)cc4[nH]3)cc2c1] | 10e |
| 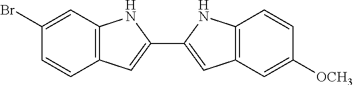 [SMILES: COc1ccc2[nH]c(-c3cc4ccc(Br)cc4[nH]3)cc2c1] | 10f |
| 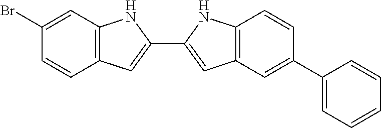 [SMILES: Brc1ccc2cc(-c3cc4cc(-c5ccccc5)ccc4[nH]3)[nH]c2c1] | 10g |
| 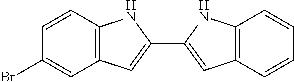 [SMILES: Brc1ccc2[nH]c(-c3cc4ccccc4[nH]3)cc2c1] | 10h |
| 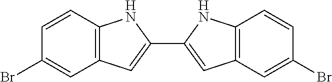 [SMILES: Brc1ccc2[nH]c(-c3cc4cc(Br)ccc4[nH]3)cc2c1] | 10i |
| 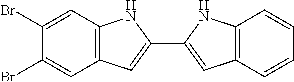 [SMILES: Brc1cc2cc(-c3cc4ccccc4[nH]3)[nH]c2cc1Br] | 10j |
| 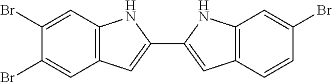 [SMILES: Brc1ccc2cc(-c3cc4cc(Br)c(Br)cc4[nH]3)[nH]c2c1] | 10k |
| 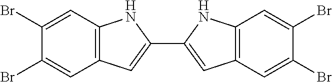 [SMILES: Brc1cc2cc(-c3cc4cc(Br)c(Br)cc4[nH]3)[nH]c2cc1Br] | 10l |
| 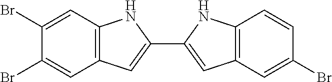 [SMILES: Brc1ccc2[nH]c(-c3cc4cc(Br)c(Br)cc4[nH]3)cc2c1] | 10m |
| 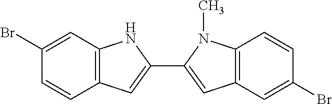 [SMILES: Cn1c(-c2cc3ccc(Br)cc3[nH]2)cc2cc(Br)ccc21] | 12a |
| 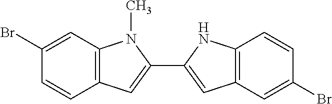 [SMILES: Cn1c(-c2cc3cc(Br)ccc3[nH]2)cc2ccc(Br)cc21] | 12b |
| 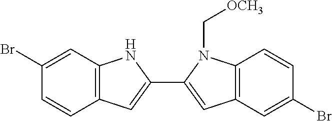 [SMILES: COCn1c(-c2cc3ccc(Br)cc3[nH]2)cc2cc(Br)ccc21] | 12c |
| 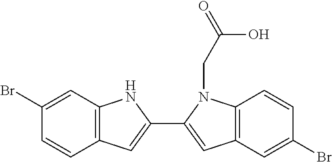 [SMILES: O=C(O)Cn1c(-c2cc3ccc(Br)cc3[nH]2)cc2cc(Br)ccc21] | 14 |
| 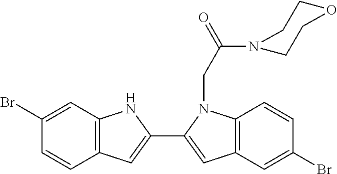 [SMILES: O=C(Cn1c(-c2cc3ccc(Br)cc3[nH]2)cc2cc(Br)ccc21)N1CCOCC1] | 15 |
| 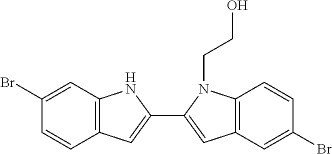 [SMILES: OCCn1c(-c2cc3ccc(Br)cc3[nH]2)cc2cc(Br)ccc21] | 17 |
| 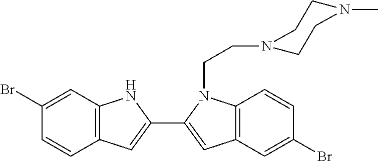 [SMILES: CN1CCN(CCn2c(-c3cc4ccc(Br)cc4[nH]3)cc3cc(Br)ccc32)CC1] | 20a |
| 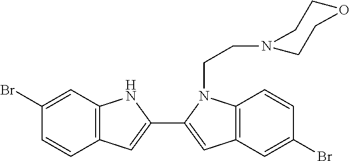 [SMILES: Brc1ccc2c(c1)cc(-c1cc3ccc(Br)cc3[nH]1)n2CCN1CCOCC1] | 20b |
| 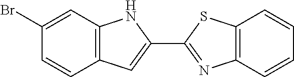 [SMILES: Brc1ccc2cc(-c3nc4ccccc4s3)[nH]c2c1] | 22a |
| 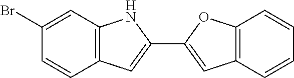 [SMILES: Brc1ccc2cc(-c3cc4ccccc4o3)[nH]c2c1] | 22b |
| 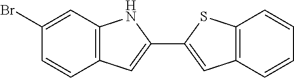 [SMILES: Brc1ccc2cc(-c3cc4ccccc4s3)[nH]c2c1] | 22c |
| 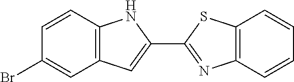 [SMILES: Brc1ccc2[nH]c(-c3nc4ccccc4s3)cc2c1] | 22d |
| 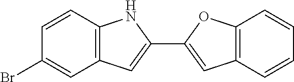 [SMILES: Brc1ccc2[nH]c(-c3cc4ccccc4o3)cc2c1] | 22e |
| 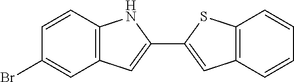 [SMILES: Brc1ccc2[nH]c(-c3cc4ccccc4s3)cc2c1] | 22f |
| 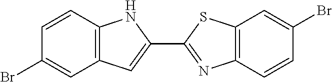 [SMILES: Brc1ccc2[nH]c(-c3nc4ccc(Br)cc4s3)cc2c1] | 22g |
| 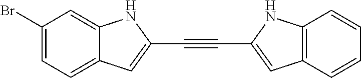 [SMILES: Brc1ccc2cc(C#Cc3cc4ccccc4[nH]3)[nH]c2c1] | 25a |
| 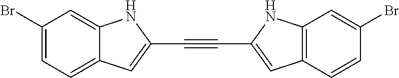 [SMILES: Brc1ccc2cc(C#Cc3cc4ccc(Br)cc4[nH]3)[nH]c2c1] | 25b |
| 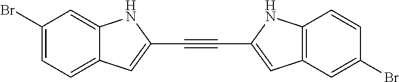 [SMILES: Brc1ccc2[nH]c(C#Cc3cc4ccc(Br)cc4[nH]3)cc2c1] | 25c |
| 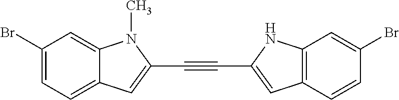 [SMILES: Cn1c(C#Cc2cc3ccc(Br)cc3[nH]2)cc2ccc(Br)cc21] | 26a |
| 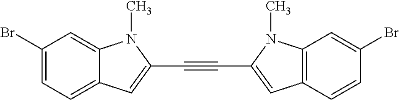 [SMILES: Cn1c(C#Cc2cc3ccc(Br)cc3n2C)cc2ccc(Br)cc21] | 26b |
| 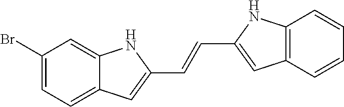 [SMILES: Brc1ccc2cc(/C=C/c3cc4ccccc4[nH]3)[nH]c2c1] | 27a |
| 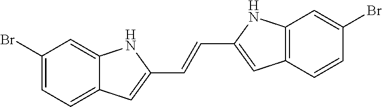 [SMILES: Brc1ccc2cc(/C=C/c3cc4ccc(Br)cc4[nH]3)[nH]c2c1] | 27b |
| 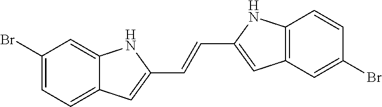 [SMILES: Brc1ccc2[nH]c(/C=C/c3cc4ccc(Br)cc4[nH]3)cc2c1] | 27c |
| 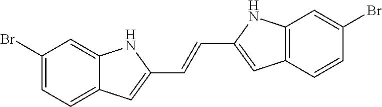 [SMILES: Brc1ccc2cc(/C=C/c3cc4ccc(Br)cc4[nH]3)[nH]c2c1] | 28a |
| 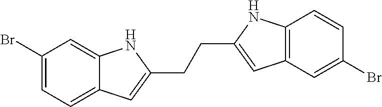 [SMILES: Brc1ccc2[nH]c(CCc3cc4ccc(Br)cc4[nH]3)cc2c1] | 28b |
| 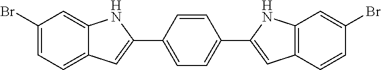 [SMILES: Brc1ccc2cc(-c3ccc(-c4cc5ccc(Br)cc5[nH]4)cc3)[nH]c2c1] | 33a |
| 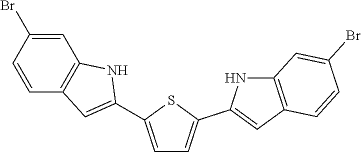 [SMILES: Brc1ccc2cc(-c3ccc(-c4cc5ccc(Br)cc5[nH]4)s3)[nH]c2c1] | 33b |
| 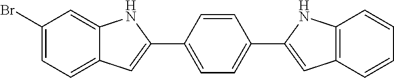 [SMILES: Brc1ccc2cc(-c3ccc(-c4cc5ccccc5[nH]4)cc3)[nH]c2c1] | 33c |
| 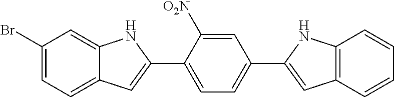 [SMILES: O=[N+]([O-])c1cc(-c2cc3ccccc3[nH]2)ccc1-c1cc2ccc(Br)cc2[nH]1] | 33d |
| 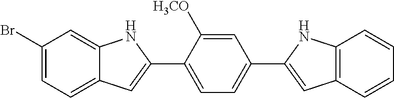 [SMILES: COc1cc(-c2cc3ccccc3[nH]2)ccc1-c1cc2ccc(Br)cc2[nH]1] | 33e |
| 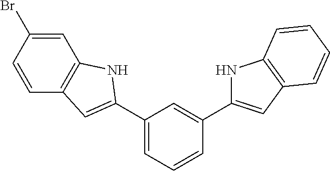 [SMILES: Brc1ccc2cc(-c3cccc(-c4cc5ccccc5[nH]4)c3)[nH]c2c1] | 33f |

TABLE C

| 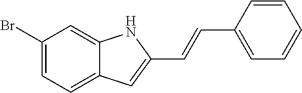 [SMILES: Brc1ccc2cc(/C=C/c3ccccc3)[nH]c2c1] | 36a |
|---|---|
| 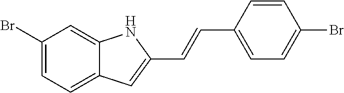 [SMILES: Brc1ccc(/C=C/c2cc3ccc(Br)cc3[nH]2)cc1] | 36b |
| 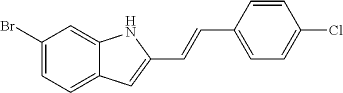 [SMILES: Clc1ccc(/C=C/c2cc3ccc(Br)cc3[nH]2)cc1] | 36c |
| 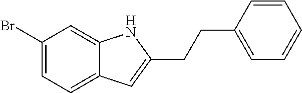 [SMILES: Brc1ccc2cc(CCc3ccccc3)[nH]c2c1] | 37a |
| 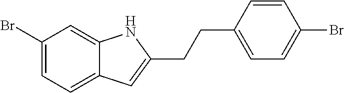 [SMILES: Brc1ccc(CCc2cc3ccc(Br)cc3[nH]2)cc1] | 37b |
| 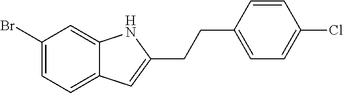 [SMILES: Clc1ccc(CCc2cc3ccc(Br)cc3[nH]2)cc1] | 37c |
| 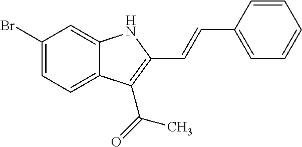 [SMILES: CC(=O)c1c(/C=C/c2ccccc2)[nH]c2cc(Br)ccc12] | 38a |
| 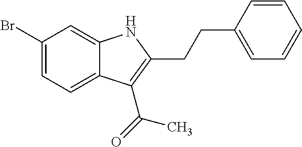 [SMILES: CC(=O)c1c(CCc2ccccc2)[nH]c2cc(Br)ccc12] | 39a |
| 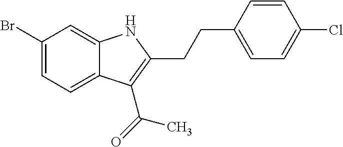 [SMILES: CC(=O)c1c(CCc2ccc(Cl)cc2)[nH]c2cc(Br)ccc12] | 39b |
| 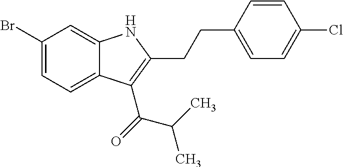 [SMILES: CC(C)C(=O)c1c(CCc2ccc(Cl)cc2)[nH]c2cc(Br)ccc12] | 39c |
| 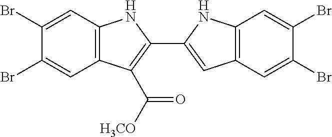 [SMILES: COC(=O)c1c(-c2cc3cc(Br)c(Br)cc3[nH]2)[nH]c2cc(Br)c(Br)cc12] | 39d |
| 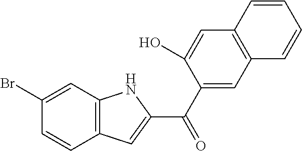 [SMILES: O=C(c1cc2ccc(Br)cc2[nH]1)c1cc2ccccc2cc1O] | 42 |
| 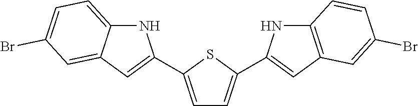 [SMILES: Brc1ccc2[nH]c(-c3ccc(-c4cc5cc(Br)ccc5[nH]4)s3)cc2c1] | 43 |
| 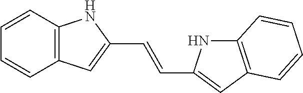 [SMILES: C(=C\c1cc2ccccc2[nH]1)/c1cc2ccccc2[nH]1] | 44 |
| 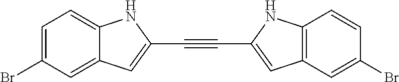 [SMILES: Brc1ccc2[nH]c(C#Cc3cc4cc(Br)ccc4[nH]3)cc2c1] | 45 |
| 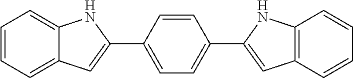 [SMILES: c1ccc2[nH]c(-c3ccc(-c4cc5ccccc5[nH]4)cc3)cc2c1] | 46 |
| 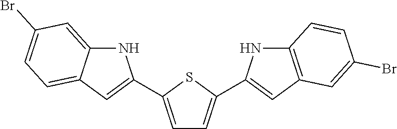 [SMILES: Brc1ccc2[nH]c(-c3ccc(-c4cc5ccc(Br)cc5[nH]4)s3)cc2c1] | 47 |
| 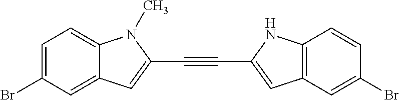 [SMILES: Cn1c(C#Cc2cc3cc(Br)ccc3[nH]2)cc2cc(Br)ccc21] | 48 |
| 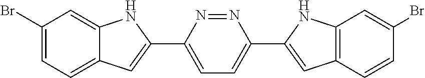 [SMILES: Brc1ccc2cc(-c3ccc(-c4cc5ccc(Br)cc5[nH]4)nn3)[nH]c2c1] | 49 |
| 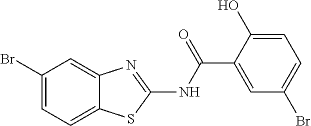 [SMILES: O=C(Nc1nc2cc(Br)ccc2s1)c1cc(Br)ccc1O] | 50 |
| 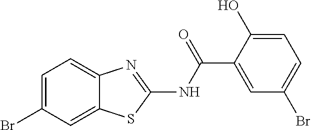 [SMILES: O=C(Nc1nc2ccc(Br)cc2s1)c1cc(Br)ccc1O] | 51 |
| 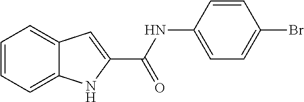 [SMILES: O=C(Nc1ccc(Br)cc1)c1cc2ccccc2[nH]1] | 52 |
| 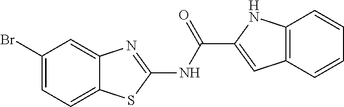 [SMILES: O=C(Nc1nc2cc(Br)ccc2s1)c1cc2ccccc2[nH]1] | 53 |
| 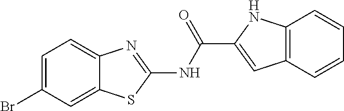 [SMILES: O=C(Nc1nc2ccc(Br)cc2s1)c1cc2ccccc2[nH]1] | 54 |
| 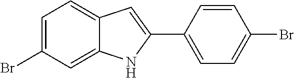 [SMILES: Brc1ccc(-c2cc3ccc(Br)cc3[nH]2)cc1] | 55 |
| 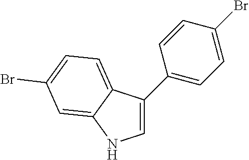 [SMILES: Brc1ccc(-c2c[nH]c3cc(Br)ccc23)cc1] | 57 |
| 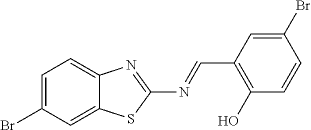 [SMILES: Oc1ccc(Br)cc1/C=N/c1nc2ccc(Br)cc2s1] | 58 |
| 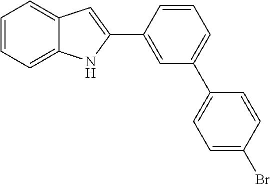 [SMILES: Brc1ccc(-c2cccc(-c3cc4ccccc4[nH]3)c2)cc1] | 59 |
| 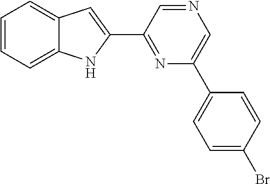 [SMILES: Brc1ccc(-c2cncc(-c3cc4ccccc4[nH]3)n2)cc1] | 60 |
| 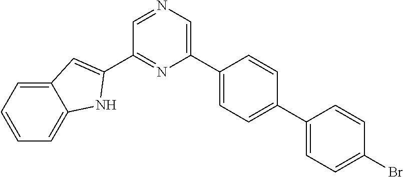 [SMILES: Brc1ccc(-c2ccc(-c3cncc(-c4cc5ccccc5[nH]4)n3)cc2)cc1] | 61 |
| 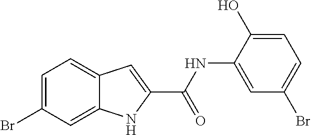 [SMILES: O=C(Nc1cc(Br)ccc1O)c1cc2ccc(Br)cc2[nH]1] | 62 |
| 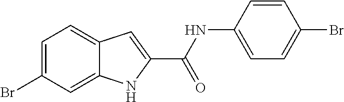 [SMILES: O=C(Nc1ccc(Br)cc1)c1cc2ccc(Br)cc2[nH]1] | 63 |
| 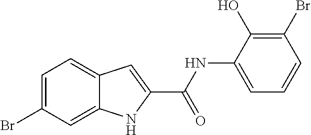 [SMILES: O=C(Nc1cccc(Br)c1O)c1cc2ccc(Br)cc2[nH]1] | 64 |
| 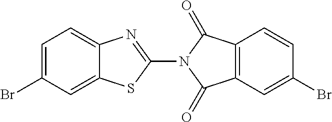 [SMILES: O=C1c2ccc(Br)cc2C(=O)N1c1nc2ccc(Br)cc2s1] | 65 |
| 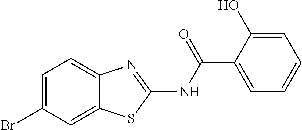 [SMILES: O=C(Nc1nc2ccc(Br)cc2s1)c1ccccc1O] | 66 |
| 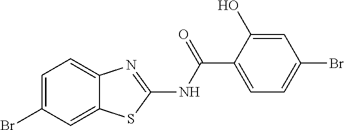 [SMILES: O=C(Nc1nc2ccc(Br)cc2s1)c1ccc(Br)cc1O] | 67 |
| 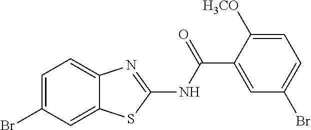 [SMILES: COc1ccc(Br)cc1C(=O)Nc1nc2ccc(Br)cc2s1] | 68 |
| 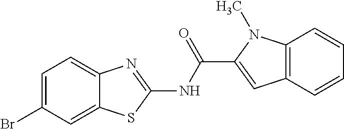 [SMILES: Cn1c(C(=O)Nc2nc3ccc(Br)cc3s2)cc2ccccc21] | 69 |
| 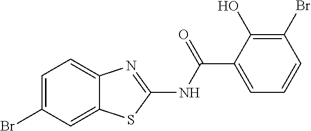 [SMILES: O=C(Nc1nc2ccc(Br)cc2s1)c1cccc(Br)c1O] | 70 |
| 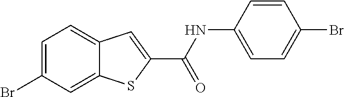 [SMILES: O=C(Nc1ccc(Br)cc1)c1cc2ccc(Br)cc2s1] | 71 |
| 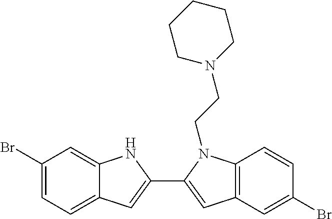 [SMILES: Brc1ccc2c(c1)cc(-c1cc3ccc(Br)cc3[nH]1)n2CCN1CCCCC1] | 72 |
| 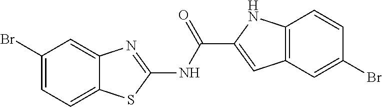 [SMILES: O=C(Nc1nc2cc(Br)ccc2s1)c1cc2cc(Br)ccc2[nH]1] | 77 |
| 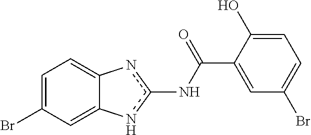 | 78 |
| 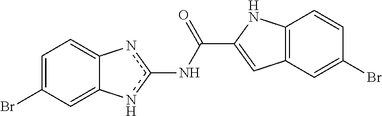 | 79 |
| 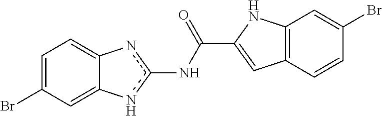 | 80 |
| 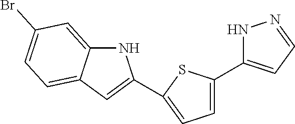 [SMILES: Brc1ccc2cc(-c3ccc(-c4ccn[nH]4)s3)[nH]c2c1] | 81 |
| 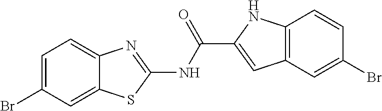 [SMILES: O=C(Nc1nc2ccc(Br)cc2s1)c1cc2cc(Br)ccc2[nH]1] | 82 |
| 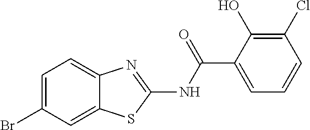 [SMILES: O=C(Nc1nc2ccc(Br)cc2s1)c1cccc(Cl)c1O] | 83 |
| 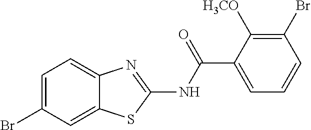 [SMILES: COc1c(Br)cccc1C(=O)Nc1nc2ccc(Br)cc2s1] | 84 |
| 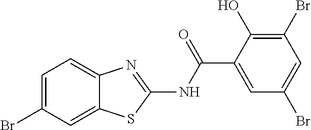 [SMILES: O=C(Nc1nc2ccc(Br)cc2s1)c1cc(Br)cc(Br)c1O] | 85 |
| 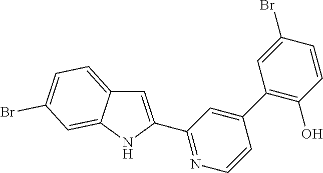 [SMILES: Oc1ccc(Br)cc1-c1ccnc(-c2cc3ccc(Br)cc3[nH]2)c1] | 86 |
| 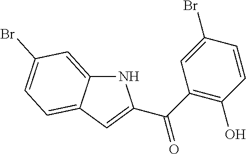 [SMILES: O=C(c1cc2ccc(Br)cc2[nH]1)c1cc(Br)ccc1O] | 87 |
| 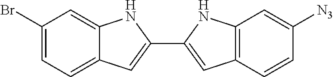 [SMILES: [N-]=[N+]=Nc1ccc2cc(-c3cc4ccc(Br)cc4[nH]3)[nH]c2c1] | 88 |
| 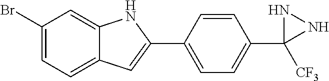 [SMILES: FC(F)(F)C1(c2ccc(-c3cc4ccc(Br)cc4[nH]3)cc2)NN1] | 89 |
| 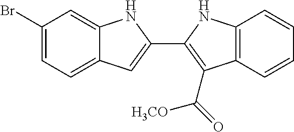 [SMILES: COC(=O)c1c(-c2cc3ccc(Br)cc3[nH]2)[nH]c2ccccc12] | 90 |
| 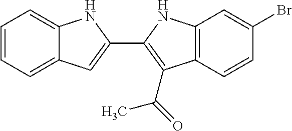 [SMILES: CC(=O)c1c(-c2cc3ccccc3[nH]2)[nH]c2cc(Br)ccc12] | 91 |
| 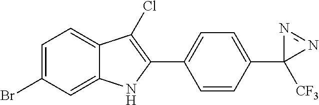 [SMILES: FC(F)(F)C1(c2ccc(-c3[nH]c4cc(Br)ccc4c3Cl)cc2)N=N1] | 92 |
| 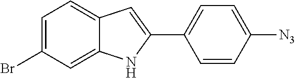 [SMILES: [N-]=[N+]=Nc1ccc(-c2cc3ccc(Br)cc3[nH]2)cc1] | 93 |
| 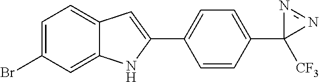 [SMILES: FC(F)(F)C1(c2ccc(-c3cc4ccc(Br)cc4[nH]3)cc2)N=N1] | 94 |
| 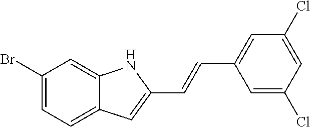 [SMILES: Clc1cc(Cl)cc(/C=C/c2cc3ccc(Br)cc3[nH]2)c1] | 95 |
| 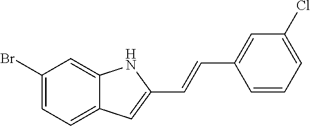 [SMILES: Clc1cccc(/C=C/c2cc3ccc(Br)cc3[nH]2)c1] | 96 |
| 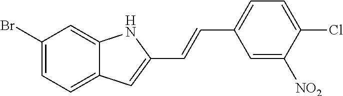 [SMILES: O=[N+]([O-])c1cc(/C=C/c2cc3ccc(Br)cc3[nH]2)ccc1Cl] | 97 |
| 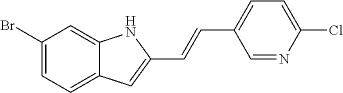 [SMILES: Clc1ccc(/C=C/c2cc3ccc(Br)cc3[nH]2)cn1] | 98 |
| 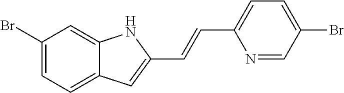 [SMILES: Brc1ccc(/C=C/c2cc3ccc(Br)cc3[nH]2)nc1] | 99 |
| 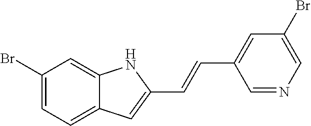 [SMILES: Brc1cncc(/C=C/c2cc3ccc(Br)cc3[nH]2)c1] | 100 |
| 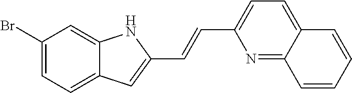 [SMILES: Brc1ccc2cc(/C=C/c3ccc4ccccc4n3)[nH]c2c1] | 101 |
| 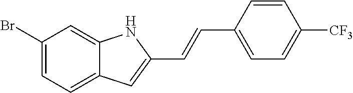 [SMILES: FC(F)(F)c1ccc(/C=C/c2cc3ccc(Br)cc3[nH]2)cc1] | 102 |
| 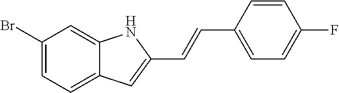 [SMILES: Fc1ccc(/C=C/c2cc3ccc(Br)cc3[nH]2)cc1] | 103 |
| 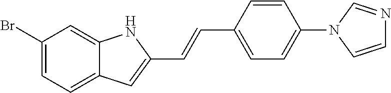 [SMILES: Brc1ccc2cc(/C=C/c3ccc(-n4ccnc4)cc3)[nH]c2c1] | 104 |
| 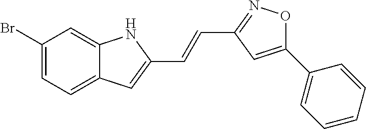 [SMILES: Brc1ccc2cc(/C=C/c3cc(-c4ccccc4)on3)[nH]c2c1] | 105 |
| 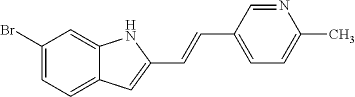 [SMILES: Cc1ccc(/C=C/c2cc3ccc(Br)cc3[nH]2)cn1] | 106 |
| 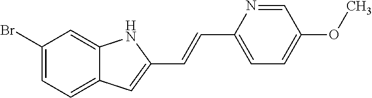 [SMILES: COc1ccc(/C=C/c2cc3ccc(Br)cc3[nH]2)nc1] | 107 |
| 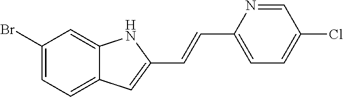 [SMILES: Clc1ccc(/C=C/c2cc3ccc(Br)cc3[nH]2)nc1] | 108 |
| 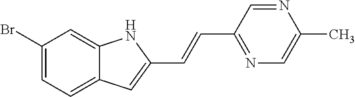 [SMILES: Cc1cnc(/C=C/c2cc3ccc(Br)cc3[nH]2)cn1] | 109 |
| 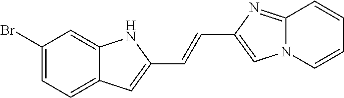 [SMILES: Brc1ccc2cc(/C=C/c3cn4ccccc4n3)[nH]c2c1] | 110 |
| 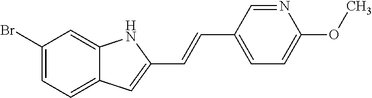 [SMILES: COc1ccc(/C=C/c2cc3ccc(Br)cc3[nH]2)cn1] | 111 |
| 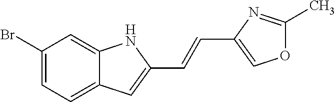 [SMILES: Cc1nc(/C=C/c2cc3ccc(Br)cc3[nH]2)co1] | 112 |
| 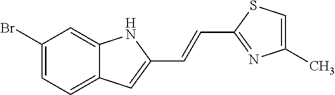 [SMILES: Cc1csc(/C=C/c2cc3ccc(Br)cc3[nH]2)n1] | 113 |
| 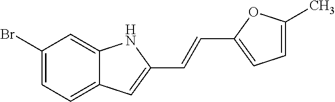 [SMILES: Cc1ccc(/C=C/c2cc3ccc(Br)cc3[nH]2)o1] | 114 |
| 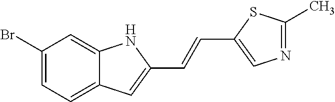 [SMILES: Cc1ncc(/C=C/c2cc3ccc(Br)cc3[nH]2)s1] | 115 |
| 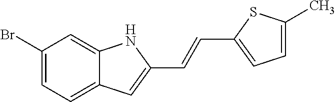 [SMILES: Cc1ccc(/C=C/c2cc3ccc(Br)cc3[nH]2)s1] | 116 |
| 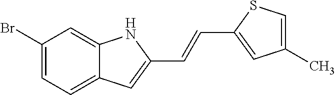 [SMILES: Cc1csc(/C=C/c2cc3ccc(Br)cc3[nH]2)c1] | 117 |
| 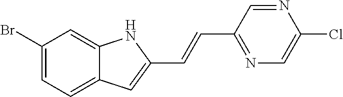 [SMILES: Clc1cnc(/C=C/c2cc3ccc(Br)cc3[nH]2)cn1] | 118 |
| 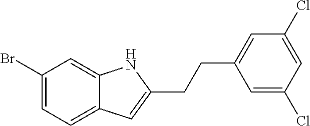 [SMILES: Clc1cc(Cl)cc(CCc2cc3ccc(Br)cc3[nH]2)c1] | 119 |
| 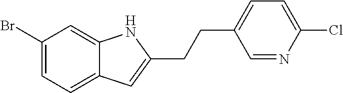 [SMILES: Clc1ccc(CCc2cc3ccc(Br)cc3[nH]2)cn1] | 120 |
| 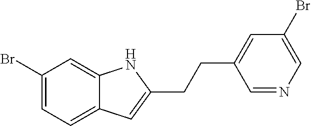 [SMILES: Brc1cncc(CCc2cc3ccc(Br)cc3[nH]2)c1] | 121 |
| 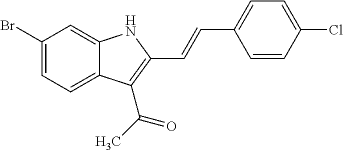 [SMILES: CC(=O)c1c(/C=C/c2ccc(Cl)cc2)[nH]c2cc(Br)ccc12] | 122 |
| 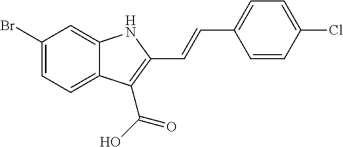 [SMILES: O=C(O)c1c(/C=C/c2ccc(Cl)cc2)[nH]c2cc(Br)ccc12] | 123 |
| 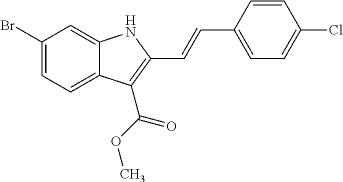 [SMILES: COC(=O)c1c(/C=C/c2ccc(Cl)cc2)[nH]c2cc(Br)ccc12] | 124 |
| 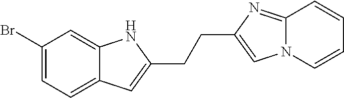 [SMILES: Brc1ccc2cc(CCc3cn4ccccc4n3)[nH]c2c1] | 125 |
| 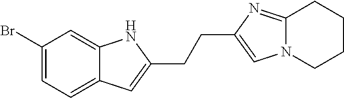 [SMILES: Brc1ccc2cc(CCc3cn4c(n3)CCCC4)[nH]c2c1] | 126 |
| 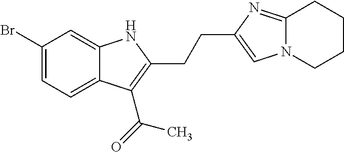 [SMILES: CC(=O)c1c(CCc2cn3c(n2)CCCC3)[nH]c2cc(Br)ccc12] | 127 |
| 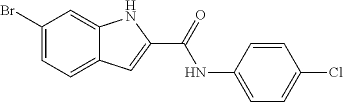 [SMILES: O=C(Nc1ccc(Cl)cc1)c1cc2ccc(Br)cc2[nH]1] | 128 |
| 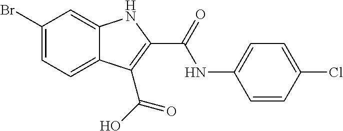 [SMILES: O=C(Nc1ccc(Cl)cc1)c1[nH]c2cc(Br)ccc2c1C(=O)O] | 129 |
| 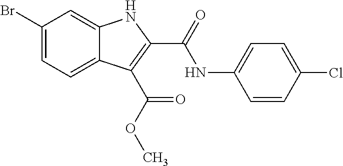 [SMILES: COC(=O)c1c(C(=O)Nc2ccc(Cl)cc2)[nH]c2cc(Br)ccc12] | 130 |
| 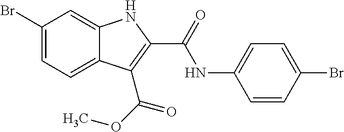 [SMILES: COC(=O)c1c(C(=O)Nc2ccc(Br)cc2)[nH]c2cc(Br)ccc12] | 131 |
| 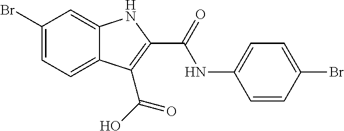 [SMILES: O=C(Nc1ccc(Br)cc1)c1[nH]c2cc(Br)ccc2c1C(=O)O] | 132 |
| 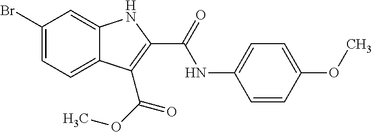 [SMILES: COC(=O)c1c(C(=O)Nc2ccc(OC)cc2)[nH]c2cc(Br)ccc12] | 133 |
| 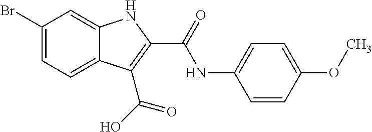 [SMILES: COc1ccc(NC(=O)c2[nH]c3cc(Br)ccc3c2C(=O)O)cc1] | 134 |
| 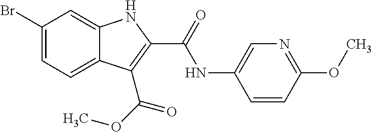 [SMILES: COC(=O)c1c(C(=O)Nc2ccc(OC)nc2)[nH]c2cc(Br)ccc12] | 135 |
| 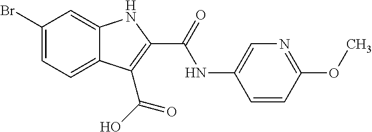 [SMILES: COc1ccc(NC(=O)c2[nH]c3cc(Br)ccc3c2C(=O)O)cn1] | 136 |
| 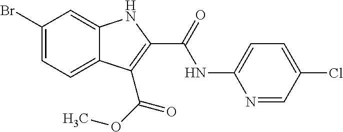 [SMILES: COC(=O)c1c(C(=O)Nc2ccc(Cl)cn2)[nH]c2cc(Br)ccc12] | 137 |
| 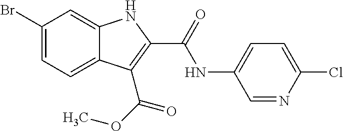 [SMILES: COC(=O)c1c(C(=O)Nc2ccc(Cl)nc2)[nH]c2cc(Br)ccc12] | 138 |
| 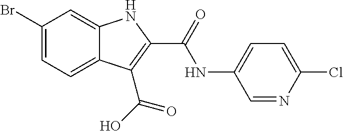 [SMILES: O=C(Nc1ccc(Cl)nc1)c1[nH]c2cc(Br)ccc2c1C(=O)O] | 139 |
| 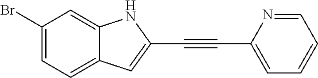 [SMILES: Brc1ccc2cc(C#Cc3ccccn3)[nH]c2c1] | 140 |
| 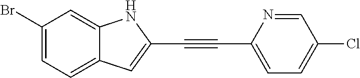 [SMILES: Clc1ccc(C#Cc2cc3ccc(Br)cc3[nH]2)nc1] | 141 |
| 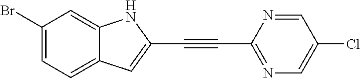 [SMILES: Clc1cnc(C#Cc2cc3ccc(Br)cc3[nH]2)nc1] | 142 |
| 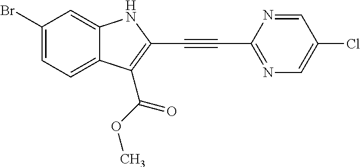 [SMILES: COC(=O)c1c(C#Cc2ncc(Cl)cn2)[nH]c2cc(Br)ccc12] | 143 |
| 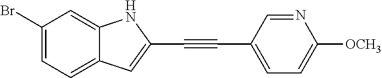 [SMILES: COc1ccc(C#Cc2cc3ccc(Br)cc3[nH]2)cn1] | 144 |
| 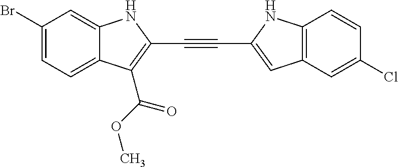 [SMILES: COC(=O)c1c(C#Cc2cc3cc(Cl)ccc3[nH]2)[nH]c2cc(Br)ccc12] | 145 |
| 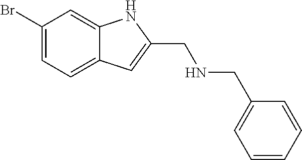 [SMILES: Brc1ccc2cc(CNCc3ccccc3)[nH]c2c1] | 146 |
| 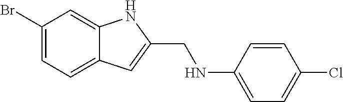 [SMILES: Clc1ccc(NCc2cc3ccc(Br)cc3[nH]2)cc1] | 147 |
| 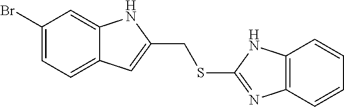 [SMILES: Brc1ccc2cc(CSc3nc4ccccc4[nH]3)[nH]c2c1] | 148 |
| 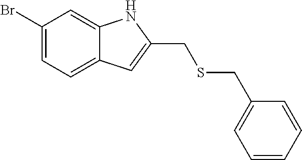 [SMILES: Brc1ccc2cc(CSCc3ccccc3)[nH]c2c1] | 149 |
| 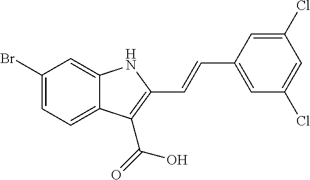 [SMILES: O=C(O)c1c(/C=C/c2cc(Cl)cc(Cl)c2)[nH]c2cc(Br)ccc12] | 150 |
| 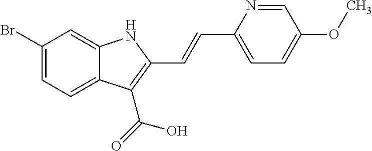 [SMILES: COc1ccc(/C=C/c2[nH]c3cc(Br)ccc3c2C(=O)O)nc1] | 151 |
| 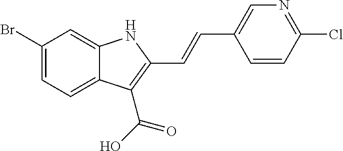 [SMILES: O=C(O)c1c(/C=C/c2ccc(Cl)nc2)[nH]c2cc(Br)ccc12] | 152 |
| 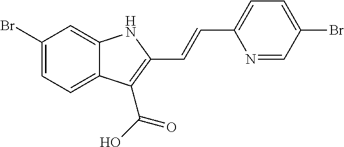 [SMILES: O=C(O)c1c(/C=C/c2ccc(Br)cn2)[nH]c2cc(Br)ccc12] | 153 |
| 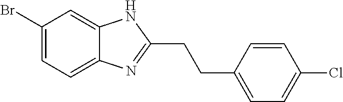 [SMILES: Clc1ccc(CCc2nc3ccc(Br)cc3[nH]2)cc1] | 154 |
| 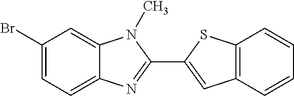 [SMILES: Cn1c(-c2cc3ccccc3s2)nc2ccc(Br)cc21] | 155 |
| 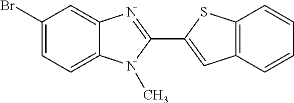 [SMILES: Cn1c(-c2cc3ccccc3s2)nc2cc(Br)ccc21] | 156 |
| 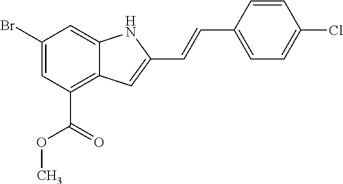 [SMILES: COC(=O)c1cc(Br)cc2[nH]c(/C=C/c3ccc(Cl)cc3)cc12] | 157 |
| 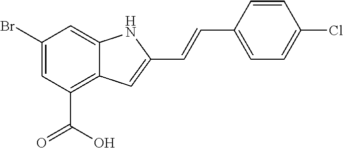 [SMILES: O=C(O)c1cc(Br)cc2[nH]c(/C=C/c3ccc(Cl)cc3)cc12] | 158 |
| 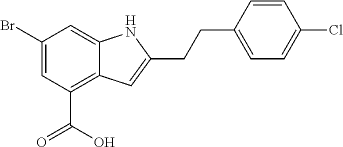 [SMILES: O=C(O)c1cc(Br)cc2[nH]c(CCc3ccc(Cl)cc3)cc12] | 159 |
| 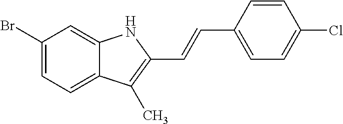 [SMILES: Cc1c(/C=C/c2ccc(Cl)cc2)[nH]c2cc(Br)ccc12] | 160 |
| 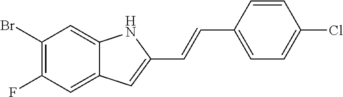 [SMILES: Fc1cc2cc(/C=C/c3ccc(Cl)cc3)[nH]c2cc1Br] | 161 |
| 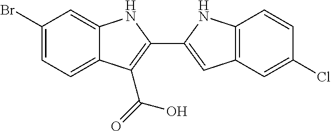 [SMILES: O=C(O)c1c(-c2cc3cc(Cl)ccc3[nH]2)[nH]c2cc(Br)ccc12] | 162 |
| 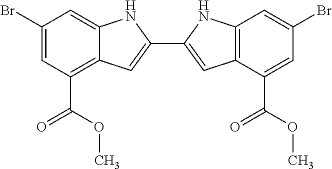 [SMILES: COC(=O)c1cc(Br)cc2[nH]c(-c3cc4c(C(=O)OC)cc(Br)cc4[nH]3)cc12] | 163 |
| 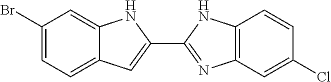 [SMILES: Clc1ccc2[nH]c(-c3cc4ccc(Br)cc4[nH]3)nc2c1] | 164 |
| 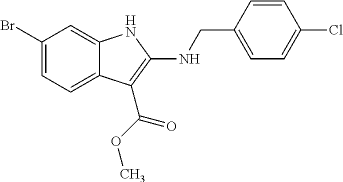 [SMILES: COC(=O)c1c(NCc2ccc(Cl)cc2)[nH]c2cc(Br)ccc12] | 165 |
| 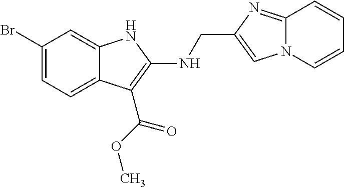 [SMILES: COC(=O)c1c(NCc2cn3ccccc3n2)[nH]c2cc(Br)ccc12] | 166 |
| 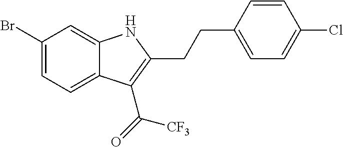 [SMILES: O=C(c1c(CCc2ccc(Cl)cc2)[nH]c2cc(Br)ccc12)C(F)(F)F] | 167 |
| 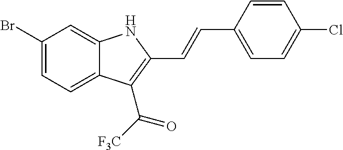 [SMILES: O=C(c1c(/C=C/c2ccc(Cl)cc2)[nH]c2cc(Br)ccc12)C(F)(F)F] | 168 |
| 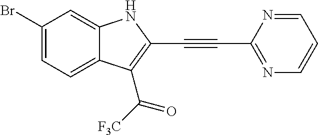 [SMILES: O=C(c1c(C#Cc2ncccn2)[nH]c2cc(Br)ccc12)C(F)(F)F] | 169 |
| 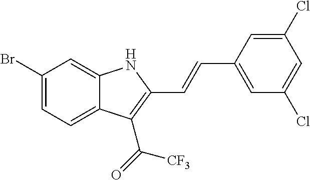 [SMILES: O=C(c1c(/C=C/c2cc(Cl)cc(Cl)c2)[nH]c2cc(Br)ccc12)C(F)(F)F] | 170 |
| 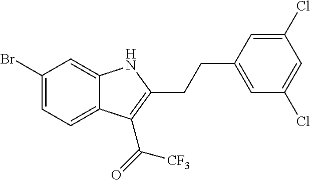 [SMILES: O=C(c1c(CCc2cc(Cl)cc(Cl)c2)[nH]c2cc(Br)ccc12)C(F)(F)F] | 171 |
| 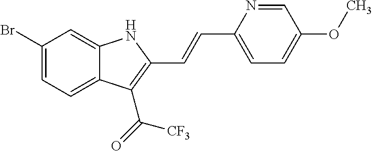 [SMILES: COc1ccc(/C=C/c2[nH]c3cc(Br)ccc3c2C(=O)C(F)(F)F)nc1] | 172 |
| 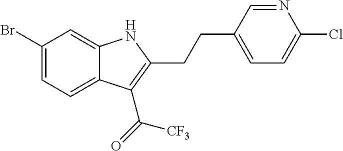 [SMILES: O=C(c1c(CCc2ccc(Cl)nc2)[nH]c2cc(Br)ccc12)C(F)(F)F] | 173 |
| 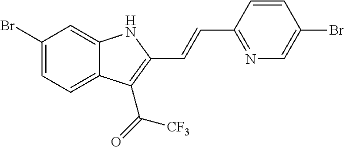 [SMILES: O=C(c1c(/C=C/c2ccc(Br)cn2)[nH]c2cc(Br)ccc12)C(F)(F)F] | 174 |
| 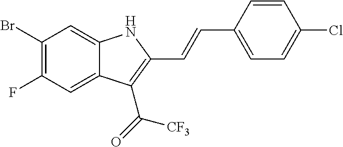 [SMILES: O=C(c1c(/C=C/c2ccc(Cl)cc2)[nH]c2cc(Br)c(F)cc12)C(F)(F)F] | 175 |
| 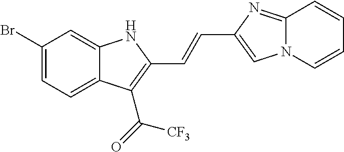 [SMILES: O=C(c1c(/C=C/c2cn3ccccc3n2)[nH]c2cc(Br)ccc12)C(F)(F)F] | 176 |
| 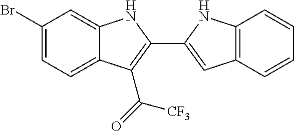 [SMILES: O=C(c1c(-c2cc3ccccc3[nH]2)[nH]c2cc(Br)ccc12)C(F)(F)F] | 177 |
| 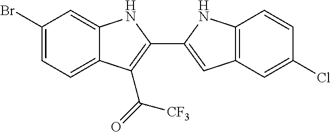 [SMILES: O=C(c1c(-c2cc3cc(Cl)ccc3[nH]2)[nH]c2cc(Br)ccc12)C(F)(F)F] | 178 |
| 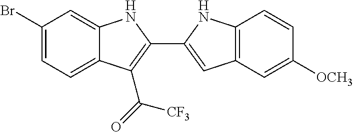 [SMILES: COc1ccc2[nH]c(-c3[nH]c4cc(Br)ccc4c3C(=O)C(F)(F)F)cc2c1] | 179 |

Certain embodiments of the invention relate to compounds selected from the following compounds, or salts thereof: 10a, 10b, 10c, 10d, 10e, 10f, 10g, 10h, 10i, 10j, 10k, 10l, 10m, 12a, 12b, 12c, 14, 15, 17, 20a, 20b, 22a, 22b, 22c, 22d, 22e, 22f, 22g, 25a, 25b, 25c, 26a, 26b, 27a, 27b, 27c, 28a, 28b, 33a, 33b, 33c, 33d, 33e, 33f, 36a, 36b, 36c, 37a, 37b, 37c, 38a, 39a, 39b, 39c, 39d, 42, 43, 45, 47, 48, 49, 50, 51, 52, 53, 54, 55, 57, 58, 59, 60, 62, 63, 64, 65, 66, 67, 68, 69, 70, 71, 72, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178 and 179, the structures of which are shown above.

Certain embodiments of the invention relate to compounds selected from the following compounds, or salts thereof: 10a, 10b, 10c, 10d, 10e, 10f, 10g, 10h, 10i, 10j, 10k, 10l, 10m, 12a, 12b, 12c, 14, 15, 17, 20a, 20b, 22a, 22b, 22c, 22d, 22e, 22f, 22g, 25a, 25b, 25c, 26a, 26b, 27a, 27b, 27c, 28a, 28b, 33a, 33b, 33c, 33d, 33e, 33f, 36a, 36c, 37a, 37b, 37c, 38a, 39a, 39b, 39c, 39d, 42, 43, 45, 47, 48, 49, 50, 51, 53, 54, 55, 57, 59, 60, 62, 63, 64, 65, 67, 68, 69, 70, 71, 72, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178 and 179, the structures of which are shown above.

Certain embodiments of the invention relate to compounds selected from the compounds shown in Table C, or salts thereof.

Certain embodiments of the invention relate to compounds of general formula II or general formula III selected from: 36a, 36b, 36c, 37a, 37b, 37c, 38a, 39a, 39b, 39c, 39d, 42, 50, 51, 52, 55, 56, 58, 59, 60, 62, 63, 64, 65, 66, 67, 68, 70, 71, 78, 81, 83, 84, 85, 86, 87, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 149, 150, 151, 152, 153, 154, 156, 157, 158, 159, 160, 161, 162, 165, 166, 167, 168, 169, 170, 171, 172, 173, 174, 175, 176, 177, 178 and 179, or salts thereof.

Certain embodiments of the invention relate to compounds of general formula II or general formula III selected from the following compounds, or salts thereof: 36a, 36b, 36c, 37a, 37b, 37c, 38a, 39a, 39b, 39c, 39d, 42, 50, 51, 52, 55, 58, 59, 60, 62, 63, 64, 65, 66, 67, 68, 70, 71, 78, 81, 83, 84, 85, 86, 87, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 149, 150, 151, 152, 153, 154, 157, 158, 159, 160, 161, 162, 165, 166, 167, 168, 169, 170, 171, 172, 173, 174, 175, 176, 177, 178 and 179, the structures of which are shown above.

Certain embodiments of the invention relate to compounds of general formula II or general formula III selected from the following compounds, or salts thereof: 36c, 37a, 37b, 37c, 38a, 39a, 39b, 39c, 39d, 42, 50, 51, 55, 59, 60, 62, 63, 64, 65, 67, 68, 70, 71, 78, 81, 83, 84, 85, 86, 87, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 149, 150, 151, 152, 153, 154, 157, 158, 159, 160, 161, 162, 165, 166, 167, 168, 169, 170, 171, 172, 173, 174, 175, 176, 177, 178 and 179, the structures of which are shown above.

It is to be understood that reference to compounds of general formula I throughout the remainder of this disclosure, includes in various embodiments, compounds of general formulae II, III, 2, 3, 4, 5, 6A, 6B, 7A, 7B and 7C to the same extent as if embodiments reciting each of these formulae individually were specifically recited.

In certain embodiments, compounds of general formula I may possess a sufficiently acidic group, a sufficiently basic group, or both functional groups, and accordingly react with a number of organic and inorganic bases, or organic and inorganic acids, to form pharmaceutically acceptable salts. The term “pharmaceutically acceptable salt” as used herein, refers to a salt of a compound that is substantially non-toxic to living organisms. Typical pharmaceutically acceptable salts include those salts prepared by reaction of a compound with a pharmaceutically acceptable mineral or organic acid or an organic or inorganic base. Such salts are known as acid addition and base addition salts.

Acids commonly employed to form acid addition salts are inorganic acids such as hydrochloric acid, hydrobromic acid, hydroiodic acid, sulphuric acid, phosphoric acid, and the like, and organic acids such as p-toluenesulphonic acid, methanesulphonic acid, oxalic acid, p-bromophenylsulphonic acid, carbonic acid, succinic acid, citric acid, benzoic acid, acetic acid, and the like. Examples of such pharmaceutically acceptable salts are the sulphate, pyrosulphate, bisulphate, sulphite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulphonate, phenylacetate, phenylpropionate, phenylbutyrate, citrate, lactate, gamma-hydroxybutyrate, glycolate, tartrate, methanesulphonate, propanesulphonate, naphthalene-1-sulfonate, napththalene-2-sulfonate, mandelate and the like. Commonly used pharmaceutically acceptable acid addition salts include those formed with mineral acids such as hydrochloric acid and hydrobromic acid, and those formed with organic acids such as maleic acid and methanesulphonic acid.

Salts of amine groups may also comprise quarternary ammonium salts in which the amino nitrogen carries a suitable organic group such as a lower (for example, C₁₋₄) alkyl, substituted lower alkyl, lower (for example, C₁₋₄) alkenyl, substituted lower alkenyl, lower (for example, C₁₋₄) alkynyl, substituted lower alkynyl, or aralkyl moiety.

Base addition salts include those derived from inorganic bases, such as ammonium or alkali or alkaline earth metal hydroxides, carbonates, bicarbonates, and the like. Bases useful in preparing pharmaceutically acceptable salts thus include sodium hydroxide, potassium hydroxide, ammonium hydroxide, potassium carbonate, sodium carbonate, sodium bicarbonate, potassium bicarbonate, calcium hydroxide, calcium carbonate, and the like.

One skilled in the art will understand that the particular counterion forming a part of a pharmaceutically acceptable salt is usually not of a critical nature, so long as the salt as a whole is pharmacologically acceptable and as long as the counterion does not contribute undesired qualities to the salt as a whole.

Certain embodiments relate to pharmaceutically acceptable solvates of a compound of general formula I. One skilled in the art will appreciate that certain compounds of general formula I may combine with solvents such as water, methanol, ethanol and acetonitrile to form pharmaceutically acceptable solvates such as the corresponding hydrate, methanolate, ethanolate and acetonitrilate. Other examples of solvents that may be used to prepare solvates include isopropanol, dimethyl sulfoxide, ethyl acetate, acetic acid, ethanolamine, or acetone, as well as miscible formulations of solvate mixtures as would be known to the skilled artisan.

One skilled in the art will appreciate that certain compounds of general formula I may exhibit tautomerism. It is therefore to be understood that the structural formulae herein are intended to represent any tautomeric form of the depicted compound and should not to be limited to any specific compound form depicted by the structural formulae.

In addition, the skilled person will appreciate that certain compounds of general formula I may have one or more asymmetric (chiral) centres and/or one or more unsaturated bonds. As a consequence, these compounds can be present as racemates, individual enantiomers, mixtures of enantiomers, individual diastereomers, mixtures of diastereomers, individual isomers (for example, E and Z isomers) and mixtures of isomers. Certain embodiments of the invention thus relate to compounds of general formula I in a substantially pure enantiomeric, diastereomeric or isomeric form. By “substantially pure” it is meant that the compound is in a form that is at least 80% optically pure, that is, a form that comprises at least 80% of a single isomer. In certain embodiments, chiral compounds may be in a form that is at least 85% optically pure, for example, at least 90%, at least 95%, at least 97.5%, or at least 99% optically pure. Certain embodiments relate to compounds of general formula I in the form of mixtures of enantiomers, diastereomers or isomers, including racemic mixtures.

The preparation of salts and solvates and the separation of enantiomers, diastereomers or isomers may be carried out by methods known in the art. It will be appreciated that non-pharmaceutically acceptable salts or solvates also fall within the scope of certain embodiments since these may be useful for example in the preparation of pharmaceutically acceptable salts or solvates.

### Preparation of Compounds of General Formula I

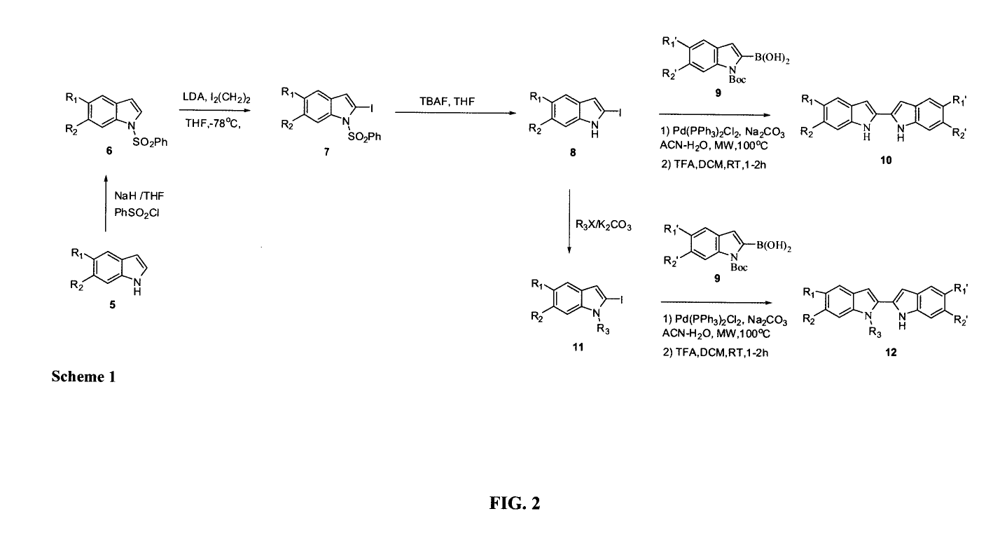

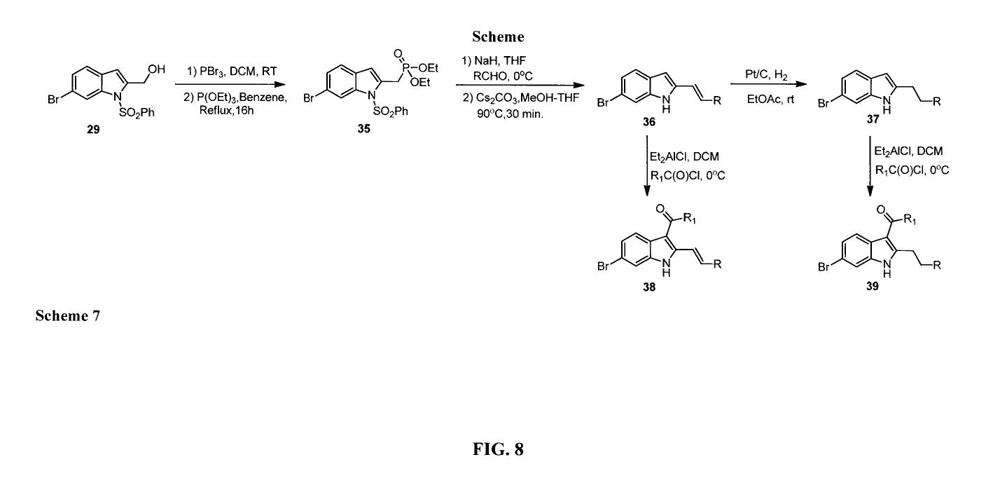

Compounds of general formula I may be prepared from known starting materials by standard synthetic chemistry methods. Representative examples of suitable synthetic routes are described in detail in the Examples provided herein (see also FIGS. 2-10). One skilled in the art will recognize that alternative methods may be employed to synthesize compounds of general formula I, and that the approaches described herein are therefore not intended to be exhaustive, but rather to provide the skilled person with examples of some broadly applicable and practical routes to representative compounds.

### Activity of Compounds of General Formula I

### Inhibition of Bacterial Pyruvate Kinase

The ability of candidate compounds of general formula I to selectively inhibit bacterial pyruvate kinase (PK) may be determined as described, for example, in Zoraghi, et al., 2011, Antimicrob. Agents Chemother., 55:20142-2053, and in the Examples provided herein.

In general, relevant recombinant PK proteins are expressed in E. coli and purified. The constructs encoding the recombinant PK proteins may be obtained from commercial sources or may be cloned using standard techniques. The gene sequences for various bacterial PK enzymes, as well as those of various human PK isoforms which may be used to ensure the specificity of the test compound, are available from public databases, such as the GenBank database maintained by the National Center for Biotechnology Information (NCBI).

Non-limiting examples of pyruvate kinase sequences from pathogenic species include, for example, Leishmania mexicana (X74944 Genomic DNA (CAA52898.2)); Chlamydia pneumoniae (AE001363 Genomic DNA (AAD18250.1) and ref seq. NP_224305.1); Mycoplasma genitalium (L43967 Genomic DNA (AAC71435.1) U01798 Genomic DNA (AAD12324.1) and ref seq. NP_072881.1); Mycobacterium tuberculosis (BX842577 Genomic DNA (CAB08894.1) ref seq. NP_216133.1); Candida albicans (S65775 mRNA); Escherichia coli O157:H7 (AE005174 Genomic DNA (AAG56663.1) and ref seq. NP_288110.1); Salmonella typhi (AL627271 Genomic DNA (CAD01987.1) and ref seq. NP_456147.1); Trypanosoma brucei brucei (X57950 Genomic DNA (CAA41018.1)); Staphylococcus aureus (strain MRSA252) BX571856 Genomic DNA (CAG40767.1) and ref seq. YP_041163.1)).

Mammalian pyruvate kinase has four isoforms: L, R, M1 and M2. The PK L isozyme is major isozyme in the liver, the R isozyme is found in red blood cells, the M1 isozyme is the main form in muscle, heart and brain, and M2 is found in early fetal tissues. Pyruvate kinase isozymes M1/M2 are encoded by the PKM2 gene (alternative references include M23725 mRNA (AAA36449.1); M26252 mRNA (AAA36672.1); X56494 Genomic DNA (CAA39849.1); AK092369 mRNA (BAG52542.1); AK222927 mRNA (BAD96647.1); AK294315 mRNA (BAG57589.1 note different initiation); AK312253 mRNA (BAG35185.1); AY352517 Genomic DNA (AAQ15274.1); ACO20779 Genomic DNA; CH471082 Genomic DNA (EAW77884.1); CH471082 Genomic DNA (EAW77888.1); BC000481 mRNA (AAH00481.3); BC007640 mRNA (AAH07640.1); BC007952 mRNA (AAH07952.3); BC012811 mRNA (AAH12811.3); BC035198 mRNA (AAH35198.1); AF025439 mRNA (AAC39559.1); and reference sequences NP_002645.3; NP_872270.1; NP_872271.1), and are alternative splicing variants. The pyruvate kinase isozymes R/L are encoded by the PKLR gene (alternative references include AB015983 mRNA (BAA31706.1); M15465 mRNA (AAA60104.1); AY316591 Genomic DNA (AAP69527.1); BC025737 mRNA (AAH25737.1); S60712 mRNA (AAB26262.1); and reference sequences NP_000289.1; NP_870986.1).

PK activity in the presence and absence of the candidate compound may be determined using a continuous assay coupled to lactate dehydrogenase (LDH). Briefly, an appropriate reaction mixture containing buffer, salts, NADH, L-LDH, ADP and PEP is prepared and the reaction is initiated by addition of a suitable amount of one of the PK enzymes. The change in absorbance at 340 nm owing to the oxidation of NADH is measured using a spectrophotometer. PK activity proportional to the rate of the change in absorbance at 340 nm can be expressed as specific activity (μmol/min/mg), which is defined as the amount of PK that catalyzes the formation of 1 μmol of either product per minute. IC₅₀ and/or EC₅₀ values may be calculated by standard curve fitting procedures.

In accordance with certain embodiments of the invention, a candidate compound of general formula I is considered to exhibit PK inhibitory activity when the compound demonstrates an IC₅₀ of ≦1000 nM in the above assay. In some embodiments, a candidate compound of general formula I is considered to exhibit PK inhibitory activity when the compound demonstrates an IC₅₀ of ≦100 nM in the above assay. In some embodiments, a candidate compound of general formula I is considered to exhibit PK inhibitory activity when the compound demonstrates an IC₅₀ of ≦50 nM in the above assay. Certain compounds of general formula I may exhibit a non-classical inhibition curve in the above assay, but still be inhibitory. Accordingly, in some embodiments, a candidate compound of general formula I is considered to exhibit PK inhibitory activity when the compound demonstrates at least 25% inhibition at a concentration of 10 μM or less in the above assay, for example, at least 50% inhibition, at least 60% inhibition or at least 70% inhibition.

### Anti-Bacterial Activity

The anti-bacterial activity of a candidate compound of general formula I may be tested using standard techniques known in the art. As is known in the art, anti-bacterial activity of a compound may result in the killing of bacteria (i.e. bactericidal activity), or it may result in the slowing or arrest of the growth of bacteria (i.e. bacteriostatic activity). Certain embodiments of the invention relate to compounds of general formula I that exhibit bactericidal activity. Certain embodiments relate to compounds of general formula I that exhibit bacteriostatic activity. Compounds that exhibit bacteriostatic activity can be useful, for example, in combination treatments with other known anti-microbial agents.

### In Vitro Testing

In vitro methods of determining the ability of candidate compounds of general formula I to inhibit the growth of bacteria are well-known in the art. In general, these methods involve contacting a culture of the cells of interest with various concentrations of the candidate compound and monitoring the growth of the cell culture relative to an untreated control culture. A second control culture comprising cells contacted with a known anti-bacterial agent may also be included in such tests, if desired.

For example, the ability of a candidate compound of general formula I to inhibit the growth of microbial cells may be determined by measurement of the minimum inhibitory concentration (MIC) for the compound. The MIC is defined as the lowest concentration that inhibits growth of the organism to a pre-determined extent. For example, a MIC₁₀₀ value is defined as the lowest concentration that completely inhibits growth of the organism, whereas a MIC₉₀ value is defined as the lowest concentration that inhibits growth by 90% and a MIC₅₀ value is defined as the lowest concentration that inhibits growth by 50%. MIC values are sometimes expressed as ranges, for example, the MIC₁₀₀ for a compound may be expressed as the concentration at which no growth is observed or as a range between the concentration at which no growth is observed and the concentration of the dilution which immediately follows.

Typically, anti-bacterial MICs for candidate compounds are measured using a broth macro- or microdilution assay (see, for example, Amsterdam, D. (1996) “Susceptibility testing of antimicrobials in liquid media,” pp. 52-111. In Loman, N., ed. Antibiotics in Laboratory Medicine, 4th ed. Williams and Wilkins, Baltimore, Md.). A standardized anti-bacterial susceptibility test is provided by the National Committee for Clinical Laboratory Standards (NCCLS) as NCCLS, 2000; document M7-A58.

In the classical broth microdilution method, the candidate anti-bacterial compound is diluted in culture medium in a sterile, covered 96-well microtiter plate. An overnight culture of a single bacterial colony is diluted in sterile medium such that, after inoculation, each well in the microtiter plate contains an appropriate number of colony forming units (CFU)/ml (typically, approximately 5×10⁵ CFU/ml). Culture medium only (containing no bacteria) is also included as a negative control for each plate and known antibiotics are often included as positive controls. The inoculated microtiter plate is subsequently incubated at an appropriate temperature (for example, 35° C.-37° C. for 16-48 hours). The turbidity of each well is then determined by visual inspection and/or by measuring the absorbance, or optical density (OD), at 595 nm or 600 nm using a microplate reader and is used as an indication of the extent of bacterial growth. An exemplary MIC testing protocol is also described in the Examples herein.

In accordance with certain embodiments of the invention, candidate compounds of general formula I are considered to exhibit anti-bacterial activity if they demonstrate an MIC in a standard broth dilution assay of ≦64 μg/mL against at least one bacterial strain, wherein the MIC is defined as ≧98% inhibition. In some embodiments, the at least one bacterial strain comprises S. aureus. In some embodiments, the at least one bacterial strain comprises a methicillin sensitive S. aureus (MSSA) strain.

One skilled in the art will appreciate that compounds that exhibit poor anti-bacterial activity when used alone (for example, a compound that has a MIC of >128 μg/ml) may still be capable of good anti-bacterial activity when used in combination with one or more known anti-bacterial agents. For example, the compound may sensitize bacteria to the action of the other agent(s), may act in synergy with other agent(s), or otherwise potentiate the activity of the other agent(s).

As such, some anti-bacterial compounds may show maximal effects when used in combination with a second drug. Such effects may be simply additive, or they may be synergistic. For example, a compound that exhibits only bacteriostatic effects when used in isolation can become bacteriocidal when used in combination with a second anti-bacterial compound. In certain embodiments, therefore, it is contemplated that the antibacterial activity of a compound of general formula I may be enhanced by the presence of another compound such as a known anti-bacterial agent, and/or that a compound of general formula I may enhance the anti-bacterial effect of other anti-bacterial agents.

Methods of testing for synergistic and/or additive effects between two or more compounds are well-known in the art. For example, the fractional inhibitory concentration (FIC) may be used to assess the presence or absence of synergy between two anti-bacterial compounds (see, for example, H. D. Isenberg, “Synergism testing: broth microdilution checkerboard and broth macrodilution methods,” in J. Hinton (ed.), Microbiology ASM, Clinical Microbiology Procedures Handbook (1992)). FICs are determined in microtiter plates in a similar manner to MICs, except that FICs are performed using a checkerboard titration of, for example, candidate compounds in one dimension and known antibiotics in the other dimension. The FIC is calculated by evaluating the impact of one antibiotic on the MIC of the other and vice versa.

In certain embodiments, candidate compounds of general formula I are considered to exhibit anti-bacterial activity if they enhance the anti-bacterial effect of at least one other anti-bacterial agent.

### In Vivo Testing

The ability of a compound of general formula I to act as an anti-bacterial agent may also be tested in vivo using standard techniques. A number of animal models suitable for testing the anti-bacterial activity of compounds are known in the art (see, for example, “Handbook of Animal Models of Infection: Experimental Models in Antimicrobial Chemotherapy,” O. Zak and M. A. Sande (eds.), 1999, Elsevier Ltd.). Representative examples include various immunocompromised or neutropenic mouse models as well as suckling mouse models. An exemplary protocol for testing compounds in a neutropenic mouse thigh infection model is provided in the Examples section.

Typically, in vivo testing comprises introducing a selected bacterium into the appropriate animal model in a sufficient amount to cause infection, followed by administration of one or more doses of the test compound. Methods of administration will vary depending on the compound being employed, but can be, for example, by way of bolus infusion into a suitable vein (such as the tail vein of mice or rats), by intraperitoneal administration, intramuscular administration, intranasal administration or by oral administration. Animals treated with a known anti-bacterial agent and/or with a saline or buffer control solution may be used as controls. Repeat doses of the test compound may be administered to the animal, if necessary, at appropriate time intervals. The animals are subsequently monitored for mortality. Animals may be sacrificed after an appropriate period of time and bacterial counts in the infected tissue may also be evaluated.

### Pharmaceutical Compositions

Compounds of general formula I are typically formulated for therapeutic use. Certain embodiments of the invention thus relate to pharmaceutical compositions comprising a compound of general formula I and a pharmaceutically acceptable carrier, diluent, or excipient. The pharmaceutical compositions may be prepared by known procedures using well-known and readily available ingredients.

Pharmaceutical compositions comprising compounds of general formula I may be formulated for administration to a subject by one of a variety of standard routes, for example, orally (including, for example, buccally or sublingually), topically, parenterally, by inhalation or spray, ocularly, rectally or vaginally, in dosage unit formulations containing conventional non-toxic pharmaceutically acceptable carriers, diluents or excipients. The term parenteral as used herein may include subcutaneous injection, intradermal injection or infusion, intra-articular injection or infusion, intravenous injection or infusion, intramuscular injection or infusion, intravascular injection or infusion, intrasternal injection or infusion, and intrathecal injection or infusion. The pharmaceutical composition is formulated in a suitable format for administration by the selected route to the subject, for example, as a syrup, elixir, tablet, troche, lozenge, hard or soft capsule, pill, suppository, eye drops, ointment, gel, oily or aqueous suspension, dispersible powder or granule, emulsion, injectable or solution. In certain embodiments, pharmaceutical compositions comprising a compound of general formula I are formulated for parenteral, oral or topical administration.

Compositions intended for oral use may be prepared in either solid or fluid unit dosage forms. Fluid unit dosage form can be prepared according to procedures known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations. An elixir is prepared by using a hydroalcoholic (for example, ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like.

Solid formulations such as tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate: granulating and disintegrating agents for example, corn starch, or alginic acid: binding agents, for example starch, gelatin or acacia, and lubricating agents, for example magnesium stearate, stearic acid or talc and other conventional ingredients such as dicalcium phosphate, magnesium aluminum silicate, calcium sulfate, starch, lactose, methylcellulose, and functionally similar materials. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Soft gelatin capsules are prepared by machine encapsulation of a slurry of the compound with an acceptable vegetable oil, light liquid petrolatum or other inert oil.

Aqueous suspensions contain active materials in admixture with excipients suitable for the manufacture of aqueous suspensions. Such excipients are suspending agents, for example sodium carboxylmethylcellulose, methyl cellulose, hydropropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl-p-hydroxy benzoate, one or more colouring agents, one or more flavouring agents or one or more sweetening agents, such as sucrose or saccharin.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

The pharmaceutical compositions may be in the form of a sterile injectable aqueous or oleaginous suspension. This suspension may be formulated according to known art using those suitable dispersing or wetting agents and suspending agents that have been mentioned above. The sterile injectable preparation may also be a sterile injectable solution or a suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. Adjuvants such as local anaesthetics, preservatives and buffering agents can also be included in the injectable solution or suspension.

In certain embodiments, other agents may be included in the pharmaceutical composition in combination with the compound of general formula I, for example, to aid uptake or metabolism, and/or delay dispersion within the subject. For example, the composition may be formulated as a controlled release formulation, which may be formed by microencapsulation using suitable agents, by embolism within a carbohydrate or polymer matrix, or the like.

Other pharmaceutical compositions and methods of preparing pharmaceutical compositions are known in the art and are described, for example, in “Remington: The Science and Practice of Pharmacy” (formerly “Remington Pharmaceutical Sciences”); Gennaro, A., Lippincott, Williams & Wilkins, Philadelphia, Pa. (2000).

In certain embodiments, the pharmaceutical composition may comprise one or more additional active agents, such as one or more of another antibiotic, an anti-protozoal agent, an anti-fungal agent, an anti-proliferative agent, an analgesics, an anti-inflammatory agent, or other compound commonly used to treat bacterial infections and/or diseases and disorders associated with bacterial infections.

Examples of commonly used antibiotics include, but are not limited to, penicillin, cloxacillin, dicloxacillin, methicillin, nafcillin, oxacillin, ampicillin, amoxicillin, bacampicillin, azlocillin, carbenicillin, mezlocillin, piperacillin, ticarcillin, azithromycin, clarithromycin, clindamycin, erythromycin, lincomycin, daptomycin, demeclocycline, doxycycline, minocycline, oxytetracycline, tetracycline, quinolone, cinoxacin, nalidixic acid, fluoroquinolone, ciprofloxacin, enoxacin, grepafloxacin, levofloxacin, lomefloxacin, norfloxacin, ofloxacin, sparfloxacin, trovafloxacin, bacitracin, colistin, polymyxin B, sulfonamide, trimethoprim-sulfamethoxazole, co-amoxyclav, cephalothin, cefuroxime, ceftriaxone, cefotaxime, vancomycin, gentamicin, amikacin, metronidazole, chloramphenicol, nitrofurantoin, co-trimoxazole, rifampicin, isoniazid, pyrazinamide, kirromycin, thiostrepton, micrococcin, fusidic acid, thiolactomycin, fosmidomycin, imipenem, cilastatin, aztreonam, linezolid, tedizolid phosphate, televancin, dalvance, oritavancin, tigecyclin, and the like.

Examples of commonly used anti-protozoal agents include, but are not limited to, chloroquine, doxycycline, mefloquine, metronidazole, eplornithine, furazolidone, hydroxychloroquine, iodoquinol, pentamidine, mebendazole, piperazine, halofantrine, primaquine, pyrimethamine sulfadoxine, doxycycline, clindamycin, quinine sulfate, quinidine gluconate, quinine dihydrochloride, hydroxychloroquine sulfate, proguanil, quinine, clindamycin, atovaquone, azithromycin, suramin, melarsoprol, eflornithine, nifurtimox, amphotericin B, sodium stibogluconate, pentamidine isethionate, trimethoprim-sulfamethoxazole, pyrimethamine, sulfadiazine, and the like.

Examples of commonly used anti-fungal agents include, but are not limited to, amphotericin B, fluconazole, itraconazole, ketoconazole, potassium iodide, flucytosine, and the like.

Examples of commonly used anti-proliferative agents include, but are not limited to, altretamine, amifostine, anastrozole, arsenic trioxide, bexarotene, bleomycin, busulfan, capecitabine, carboplatin, carmustine, celecoxib, chlorambucil, cisplatin, cisplatin-epinephrine gel, cladribine, cytarabine liposomal, daunorubicin liposomal, daunorubicin daunomycin, dexrazoxane, docetaxel, doxorubicin, doxorubicin liposomal, epirubicin, estramustine, etoposide phosphate, etoposide VP-16, exemestane, fludarabine, fluorouracil 5-FU, fulvestrant, gemicitabine, gemtuzumab-ozogamicin, goserelin acetate, hydroxyurea, idarubicin, ifosfamide, imatinib mesylate, irinotecan, letrozole, leucovorin, levamisole, liposomal daunorubicin, melphalan L-PAM, mesna, methotrexate, methoxsalen, mitomycin C, mitoxantrone, paclitaxel, pamidronate, pegademase, pentostain, porfimer sodium, streptozocin, talc, tamoxifen, temozolamide, teniposide VM-26, topotecan, toremifene, tretinoin, ATRA, valrubicin, vinorelbine, zoledronate, steroids, and the like.

Examples of commonly used analgesics include, but are not limited to, acetaminophen, aspirin, diflunisal, ibuprofen, naproxen, fenoprofen, fenbuten, flurbiprofen, indoprofen, ketoprofen, indomethacin, ketorolac, diclofenac, nabumetone, piroxicam, meloxicam, tenoxicam, mefenamic acid, tolfenamic acid, meclofenamic acid, tolfenamic acid, celecoxib, rofecoxib, valdecoxib, parecoxib, lumiracoxib, nimesulide, licofenole, phenylbutazone, oxphenbutazone, antipyrine, aminopyrine, thiocolchicoside, duloxetine, milnacipran, amitriptylene, desipramine, imipramine, bupropion, lefetamine, methylphenidate, pregabalin, paroxetine, citalopram, clonidine, guanfacine, tizaidine morphine, oxycodone, hydromorphone, hydrocodone and the like.

Examples of commonly used anti-inflammatory agents include non-steroidal anti-inflammatory drugs (NSAIDs) such as aspirin salsalate, diflunisal, ibuprofen, ketoprofen, nabumetone, piroxicam, naproxen, diclofenac, indomethacin, sulindac, tolmetin, etodolac, ketorolac, oxaprozin and celecoxib, and corticosteroid drugs such as cortisone, hydrocortisone and prednisone.

### Methods and Uses

One aspect of the invention relates to the use of compounds of general formula I as anti-bacterial agents. In this context, the compounds may, for example, exert an effect by inhibiting PK activity in one or more bacterial strains, inhibiting the growth of one or more bacterial strains, ameliorating a condition associated with a bacterial infection, or combinations thereof.

Certain embodiments of the invention thus relate to a method of treating a bacterial infection or contamination with a compound of general formula I. Certain embodiments of the invention relate to a method of inhibiting bacterial PK activity with a compound of general formula I. Certain embodiments of the invention relate to a method of inhibiting bacterial growth with a compound of general formula I. Certain embodiments of the invention relate to a method of ameliorating a condition associated with a bacterial infection using a compound of general formula I. Certain embodiments of the invention relate to a method of treating a disease or disorder associated with a bacterial infection using a compound of general formula I. In some embodiments, the invention relates to a method of inhibiting bacterial growth with a compound of general formula I, wherein the compound inhibits PK activity in the bacteria.

When a compound of general formula I are used in a therapeutic context, for example, for one or more of treating a bacterial infection in an animal, inhibiting a bacterial PK in vivo, inhibiting bacterial growth in vivo, ameliorating a condition associated with a bacterial infection, or treating a disease or disorder associated with a bacterial infection, the compound is typically formulated as a medicament. Accordingly, certain embodiments of the invention relate to the use of a compound of general formula I in the manufacture of a medicament for one or more of the foregoing therapeutic uses.

Certain embodiments of the invention relate to the use of compounds of general formula I as broad-spectrum anti-bacterial agents. Accordingly, in certain embodiments, therefore, the compounds may be used as anti-bacterial agents against one or more of a wide range of bacterial strains including, for example, bacterial strains belonging to the genus Acinetobacter, Aeromonas, Bacteroides, Bordetella, Borrelia, Burkholderia, Campylobacter, Citrobacter, Clostridium, Corynebacterium, Enterobacter, Enterococcus, Escherichia, Francisella, Gardnerella, Haemophilus, Helicobacter, Kingella, Klebsiella, Legionella, Listeria, Moraxella, Morganella, Mycobacterium, Neisseria, Pasteurella, Proteus, Providencia, Pseudomonas, Salmonella, Serratia, Shigella, Stenotrophomonas, Streptococcus, Vibrio or Yersinia. For example, in various embodiments, the bacterial strain may be Acinetobacter baumannii, Acinetobacter calcoaceticus, Acinetobacter haemolyticus, Aeromonas hydrophilia, Bacillus anthracis, Bacillus cereus, Bacteroides 3452A homology group, Bacteroides distasonis, Bacteroides eggerthii, Bacteroides fragilis, Bacteroides ovalus, Bacteroides splanchnicus, Bacteroides thetaiotaomicron, Bacteroides uniformis, Bacteroides vulgatus, Bordetella bronchiseptica, Bordetella parapertussis, Bordetella pertussis, Borrelia burgdorferi, Burkholderia cepacia, Campylobacter coli, Campylobacter fetus, Campylobacter jejuni, Citrobacter freundii, Clostridium difficile, Corynebacterium diphtheriae, Corynebacterium ulcerans, Enterobacter aerogenes, Enterobacter cloacae, Enterococcus faecalis, Enterococcus faecium, Escherichia coli, Francisella tularensis, Gardnerella vaginalis, Haemophilus ducreyi, Haemophilus haemolyticus, Haemophilus influenzae, Haemophilus parahaemolyticus, Haemophilus parainfluenzae, Helicobacter pylori, Klebsiella oxytoca, Klebsiella pneumoniae, Legionella pneumophila, Listeria monocytogenes, Morganella morganii, Morexella catarrhalis (formerly Branhamella catarrhalis), Mycobacterium avium, Mycobacterium intracellulare, Mycobacterium leprae, Mycobacterium tuberculosis, Neisseria gonorrhoeae, Neisseria meningitidis, Pasteurella haemolytica, Pasteurella multocida, Proteus mirabilis, Proteus vulgaris, Providencia alcalifaciens, Providencia rettgeri, Providencia stuartii, Pseudomonas aeruginosa, Pseudomonas acidovorans, Pseudomonas alcaligenes, Pseudomonas fluorescens, Pseudomonas putida, Salmonella enteritidis, Salmonella paratyphi, Salmonella typhi, Salmonella typhimurium, Serratia marcescens, Shigella dysenteriae, Shigella flexneri, Shigella sonnei, Staphylococcus aureus, Staphylococcus epidermidis, Staphylococcus intermedius, Staphylococcus haemolyticus, Staphylococcus hominis, Staphylococcus hyicus subsp. hyicus, Staphylococcus saccharolyticus, Staphylococcus saprophyticus, Stenotrophomonas maltophilia, Streptococcus agalactiae, Streptococcus pneumoniae, Streptococcus pyogenes, Vibrio cholerae, Vibrio parahaemolyticus, Yersinia enterocolitica, Yersinia intermedia, Yersinia pestis, Yersinia pseudotuberculosis, or a drug resistant strain thereof.

In some embodiments, the compounds of general formula I may be used as anti-bacterial agents against one or more gram positive bacterial strains. Examples of gram positive bacterial strains include strains belonging to the genus Bacillus, Clostridium, Corynebacterium, Enterococcus, Listeria, Staphylococcus and Streptococcus, such as Bacillus anthracis, Bacillus cereus, Clostridium difficile, Corynebacterium diphtheriae, Corynebacterium ulcerans, Enterococcus faecalis, Enterococcus faecium, Listeria monocytogenes, Staphylococcus aureus, Staphylococcus epidermidis, Staphylococcus intermedius, Staphylococcus haemolyticus, Staphylococcus hominis, Staphylococcus hyicus subsp. hyicus, Staphylococcus saccharolyticus, Staphylococcus saprophyticus, Streptococcus agalactiae, Streptococcus pneumonia, Streptococcus pyogenes, and drug resistant strains thereof.

In some embodiments, the compounds of general formula I may be used as anti-bacterial agents against one or more gram negative bacterial strains. Examples of gram negative bacterial strains include strains belonging to the genus Acinetobacter, Aeromonas, Bacteroides, Bordetella, Burkholderia, Campylobacter, Citrobacter, Enterobacter, Escherichia, Francisella, Haemophilus, Helicobacter, Kingella, Klebsiella, Legionella, Morexella, Morganella, Neisseria, Pasteurella, Proteus, Providencia, Pseudomonas, Salmonella, Serratia, Shigella, Stenotrophomonas, Vibrio and Yersinia, such as Acinetobacter baumannii, Acinetobacter calcoaceticus, Acinetobacter haemolyticus, Aeromonas hydrophilia, Bacteroides 3452A homology group, Bacteroides distasonis, Bacteroides eggerthii, Bacteroides fragilis, Bacteroides ovalus, Bacteroides splanchnicus, Bacteroides thetaiotaomicron, Bacteroides uniformis, Bacteroides vulgatus, Bordetella bronchiseptica, Bordetella parapertussis, Bordetella pertussis, Borrelia burgdorferi, Burkholderia cepacia, Campylobacter coli, Campylobacter fetus, Campylobacter jejuni, Citrobacter freundii, Enterobacter aerogenes, Enterobacter cloacae, Escherichia coli, Francisella tularensis, Gardnerella vaginalis, Haemophilus ducreyi, Haemophilus haemolyticus, Haemophilus influenzae, Haemophilus parahaemolyticus, Haemophilus parainfluenzae, Helicobacter pylori, Klebsiella oxytoca, Klebsiella pneumoniae, Legionella pneumophila, Morganella morganii, Morexella catarrhalis (formerly Branhamella catarrhalis), Mycobacterium avium, Mycobacterium intracellulare, Mycobacterium leprae, Mycobacterium tuberculosis, Neisseria gonorrhoeae, Neisseria meningitidis, Pasteurella haemolytica, Pasteurella multocida, Proteus mirabilis, Proteus vulgaris, Providencia alcalifaciens, Providencia rettgeri, Providencia stuartii, Pseudomonas aeruginosa, Pseudomonas acidovorans, Pseudomonas alcaligenes, Pseudomonas fluorescens, Pseudomonas putida, Salmonella enteritidis, Salmonella paratyphi, Salmonella typhi, Salmonella typhimurium, Serratia marcescens, Shigella dysenteriae, Shigella flexneri, Shigella sonnei, Stenotrophomonas maltophilia, Vibrio cholerae, Vibrio parahaemolyticus, Yersinia enterocolitica, Yersinia intermedia, Yersinia pestis, Yersinia pseudotuberculosis, and drug resistant strains thereof.

In certain embodiments, the compounds of general formula I may be used as anti-bacterial agents against both gram positive bacterial strains and gram negative bacterial strains, such as those described above. In certain embodiments, the compounds of general formula I may be used as anti-bacterial agents against strains of bacteria from one or more of Acinetobacter, Enterococcus, Klebsiella and/or Staphylococcus, for example, one or more of Acinetobacter baumannii, Acinetobacter calcoaceticus, Acinetobacter haemolyticus, Enterococcus faecalis, Enterococcus faecium, Klebsiella oxytoca, Klebsiella pneumoniae, Staphylococcus aureus, Staphylococcus epidermidis, Staphylococcus intermedius, Staphylococcus haemolyticus, Staphylococcus hominis, Staphylococcus hyicus subsp. hyicus, Staphylococcus saccharolyticus and/or Staphylococcus saprophyticus, or drug resistant strains thereof. In some embodiments, the compounds of general formula I may be used as anti-bacterial agents against at least one of A. baumannii, K pneumoniae, S. aureus, E. faecalis or E. faecium, or drug resistant strains thereof.

In a certain embodiments, the compounds of general formula I may be used to treat infections caused by one or more hospital-acquired ESKAPE pathogens (Enterococcus faecium, Staphylococcus aureus, Klebsiella pneumoniae, Acinetobacter baumannii, Pseudomonas aeruginosa and Enterobacter spp.).

In certain embodiments, the compounds of general formula I may be used to treat an infection caused by a drug resistant strain of bacteria. In this context, it is contemplated that a compound of general formula I may be used as a first-line therapy to treat a subject infected with, or suspected of being infected with, a drug resistant strain of bacteria, or the compound may be used as a second or third line therapy to treat a subject infected with, or suspected of being infected with, a drug resistant strain of bacteria, who has not responded to treatment with one or more known antibiotics. Accordingly, certain embodiments of the invention relate to a method of treating a subject known or suspected of having an infection caused by a drug resistant bacterium with a compound of general formula I. Some embodiments relate to a method of treating a subject known or suspected of having an infection caused by a drug resistant bacterium with a compound of general formula I, where the subject has not responded to treatment with a first-line antibiotic. Some embodiments relate to a method of treating a subject known or suspected of having an infection caused by a drug resistant bacterium with a compound of general formula I, where the subject has not responded to treatment with a first-line antibiotic and a second-line antibiotic.

In certain embodiments, compounds of general formula I may be used to treat infections caused by methicillin-resistant S. aureus (MRSA) or vancomycin-resistant Enterococcus (VRE).

In certain embodiments, the compounds of general formula I may be used in methods of treating a localized bacterial infection in a subject or a disease, disorder or condition associated therewith. For example, in certain embodiments, the compounds of general formula I may be used to treat an infection of the upper respiratory tract and/or an associated condition such as otitis media, bacterial tracheitis, acute epiglottitis, or thyroiditis. In some embodiments, the compounds of general formula I may be used to treat an infection of the lower respiratory tract and/or an associated condition such as empyema, or lung abscesses. In some embodiments, the compounds of general formula I may be used to treat a cardiac infection and/or an associated condition such as infective endocarditis or bacterial pericarditis. In some embodiments, the compounds of general formula I may be used to treat an infection of the gastrointestinal tract and/or an associated condition such as bacterial diarrhoea, splenic abscesses, or retroperitoneal abscesses. In some embodiments, the compounds of general formula I may be used to treat a CNS infection and/or an associated condition such as a cerebral abscess. In some embodiments, the compounds of general formula I may be used to treat an eye infection and/or an associated condition such as blepharitis, conjunctivitis, keratitis, endophthalmitis, preseptal cellulitis, orbital cellulitis or darcryocystitis. In some embodiments, the compounds of general formula I may be used to treat an infection of the kidney and/or urinary tract and/or an associated condition such as epididymitis, cystitis, intrarenal abscesses, perinephric abscesses or toxic shock syndrome. In some embodiments, the compounds of general formula I may be used to treat an infection of the skin and/or an associated condition such as impetigo, folliculitis, cutaneous abscesses, cellulitis, wound infection or bacterial myositis. In some embodiments, the compounds of general formula I may be used to treat an infection of bone and/or joint and/or an associated condition such as septic arthritis or osteomyelitis.

Other diseases and disorders associated with bacterial infection that be treated with compounds of general formula I in certain embodiments may include, for example, tuberculosis, meningitis, ulcers, septicaemia, bacteremia, cystic fibrosis, pneumonia, typhoid fever, gonorrhoea, impetigo, bacterial ear infections, bacterial vaginitis, food poisoning, hemolytic uremic syndrome, botulism, leprosy, gangrene, tetanus, Lyme disease, Legionnaire's disease, listeriosis, plague, anthrax and/or chancroid.

Certain embodiments of the invention contemplate the use of a compound of general formula I as part of a combination therapy to treat a bacterial infection or associated condition, disease or disorder such as those described above. For example, the compound of general formula I may be used in combination with one or more other antibiotics and/or with one or more of an anti-protozoal agent, anti-fungal agent, anti-proliferative agent, analgesic, anti-inflammatory or other compound commonly used to treat bacterial infections and/or diseases and disorders associated with bacterial infection. Representative examples of antibiotics, anti-protozoal agents, anti-fungal agents, anti-proliferative agents, analgesics and anti-inflammatory agents that are commonly used in the treatment of bacterial infections or diseases and disorders associated with bacterial infections are provided above (see “Pharmaceutical Compositions”). Other examples would be known to the skilled person. When a compound of general formula I is used as part of a combination therapy, the compound and the one or more other drugs may be administered together or may be administered separately. When administered together, they may be formulated as a single composition, or they may be formulated separately but administered together.

The dosage of the compounds of general formula I may vary depending on the route of administration (for example, oral, intravenous, inhalation, or the like) and the form in which the composition or compound is administered (for example, solution, controlled release or the like). Determination of appropriate dosages is within the ability of one of skill in the art. As used herein, a “therapeutically effective amount,” or a “pharmacologically effective amount” of a medicament may refer to an amount of a medicament present in such a concentration to result in a therapeutic level of drug delivered over the term that the drug is used. This may be dependent on mode of delivery, time period of the dosage, age, weight, general health, sex and diet of the subject receiving the medicament. Methods of determining effective amounts are known in the art. It will also be appreciated that the effective dose of a particular compound may increase or decrease over the course of a particular treatment.

Certain embodiments of the invention relate to the use of compounds of general formula I in a non-therapeutic context, for example, as the active ingredient in anti-bacterial cleansers, polishes, paints, sprays, soaps, detergents, and the like. In some embodiments, the compounds may be included as an anti-bacterial agent in cosmetic, personal care, household and industrial products, for example, to improve shelf-life by inhibiting the growth of spoilage bacteria within the products. In some embodiments, it is contemplated that the compounds may be formulated for application to surfaces to inhibit the growth of a bacterial species thereon, for example, surfaces such as countertops, desks, chairs, laboratory benches, tables, floors, sinks, showers, toilets, bathtubs, bed stands, tools or equipment, doorknobs and windows. In some embodiments, the compounds may be formulated for laundry applications, for example, for washing clothes, towels, sheets and other bedlinen, washcloths or other cleaning articles. The cleansers, polishes, paints, sprays, soaps, or detergents comprising an anti-bacterial compound of general formula I may optionally contain one or more suitable solvents, carriers, thickeners, pigments, fragrances, deodorisers, emulsifiers, surfactants, wetting agents, waxes, oils, or the like, as would be known to those skilled in the art. In certain embodiments, compounds of general formula I may be included in formulations for external use, for example as a pharmaceutically acceptable skin cleanser. The non-therapeutic formulations comprising compounds of general formula I may find use for example in hospitals for the prevention of nosocomial infections, in schools and in recreational facilities, as well as in other institutional and home settings.

In certain embodiments, the invention contemplates the use of compounds of general formula I in formulations to assist in the sterilization of surgical and other medical equipment and implantable devices, including prosthetic joints. In some embodiments, the compounds may be formulated for use in the in situ sterilization of indwelling invasive devices such as intravenous lines and catheters, which are often foci of infection.

In certain embodiments, the invention contemplates the use of the compounds of general formula I as the active ingredient in personal care items, such as soaps, deodorants, shampoos, mouthwashes, toothpastes, and the like. Many compositions used in personal care applications are susceptible to bacterial growth and it is thus desirable to incorporate into these compositions an effective anti-bacterial agent. The anti-bacterial agent may be incorporated into the personal care formulation using techniques known in the art. For example, it may be added to the personal care formulation as a solution, emulsion or dispersion in a suitable liquid medium, or it may be added, undiluted, to the personal care formulation or it may be added with a solid carrier or diluent. In this context, the anti-bacterial agent may be added to a pre-prepared personal care formulation or it may be added during the preparation of the personal care formulation, either separately or premixed with one of the other components of the formulation.

It is also contemplated that the compounds described herein may be used for in vivo or in vitro research uses (i.e. non-clinical) to investigate alternative treatments for microbial infection. Furthermore, these compounds may be used individually or as part of a kit for in vivo or in vitro research to investigate mechanisms of microbial resistance or microbial infection using recombinant proteins, cells maintained in culture, and/or animal models.

### Assays

Certain embodiments of the invention relate to assay methods for identifying compounds that inhibit bacterial PK. As described herein, and without being limited to any particular method or mechanism of action, it is proposed that compounds of general formula I may inhibit bacterial PK by binding to the PK tetramer at the minor interface. The assay methods therefore identify compounds which bind to the PK tetramer at the minor interface and inhibit PK activity, possibly through rigidification of the complex.

In some embodiments, the assay method determines whether a candidate compound selectively binds to a pathogen PK (for example, a MRSA PK) by combining a candidate compound with (a) pathogen PK monomeric subunits, and (b) one or more of the human PK monomeric subunits (i.e. the human isoenzymes monomers for M1, M2, L and R), then determining PK tetramer and/or dimer formation by each of the pathogen and the human PK monomers in the presence of the candidate compound.

Assaying for pyruvate kinase tetramer and/or dimer formation may be accomplished, for example, through the use of monomer-specific monoclonal antibodies which may be used to quantify monomer by immunocytochemistry (see for example, Ashizawa et al. 1991, J Biol. Chem., 266:16842-16846). Alternatively, dimer and/or tetramer formation may be assayed via pyruvate kinase activity assays (for example, using Abcam™ Pyruvate-Kinase-PK-Assay-Kit (catalog# ab83432); Sigma Aldrich Pyruvate Kinase Activity Assay Kit (catalog# MAK072); BioVision™ Pyruvate Kinase Assay Kit (catalog# K709-100), or by gel filtration and immunodetection (see for example, Adachi et al., 1977, Proc Natl Acad Sci USA, 74:501-504; Zwerschke et al., 1999, Proc Natl Acad Sci USA, 96(4):1291-1296; and Gupta et al., 2010, J Biol Chem., 285(22):16864-73). Dimer and/or tetramer formation may also be assayed through the use of mass spectrometry (MS) coupled with the soft ionization processes of either matrix-assisted laser desorption (MALDI) or electrospray (ES) ionization (for example, Hernandez & Robinson, 2007, Nature Protocols 2:715-726), MALDI-TOF spectroscopy (for example, Farmer & Caprioli, 1991, Biological Mass Spectrometry 20:796-800; and Moniatte et al., 1997, Int. J Mass Spectrometry and Ion Processes, 169-170:179-199), or using other assays for tetramer formation (for example, Ashizawa et al., 1991, Biochemistry, 30:7105-7111; and Desmaret et al., 2005, Biol. Chem., 386:1137-1147).

Constructs encoding recombinant PK proteins for preparation of the monomeric subunits may be obtained from commercial sources or may be cloned using the known gene sequences for various bacterial PK enzymes and human PK isoforms (as described above and available for example from the NCBI GenBank database) and standard techniques.

### Pharmaceutical Kits

Certain embodiments of the invention relate to pharmaceutical kits or packs containing a compound of general formula I or a pharmaceutical composition comprising a compound of general formula I. In those embodiments in which the compounds of general formula I are intended for use as part of a combination therapy, the kit may optionally contain the other therapeutic(s) that makes up the combination.

Individual components of the kit would typically be packaged in separate containers and, associated with such containers, can be a notice in the form prescribed by a governmental agency regulating the manufacture, use or sale of pharmaceuticals or biological products, which notice reflects approval by the agency of manufacture, for use or sale for human or animal administration. If appropriate, one or more components of the kit may be lyophilized or provided in a dry form, such as a powder or granules, and the kit can additionally contain a suitable solvent for reconstitution of the lyophilized or dried component(s).

In those embodiments in which the compound of general formula I is included in the kit in the form of a pharmaceutical composition suitable for administration to a subject, the container may optionally be itself in a form a allowing for administration to a subject, for example, an inhaler, syringe, pipette, eye dropper, pre-soaked gauze or pad, or other such like apparatus, from which the composition may be administered to the subject.

To gain a better understanding of the invention described herein, the following examples are set forth. It will be understood that these examples are intended to describe illustrative embodiments of the invention and are not intended to limit the scope of the invention in any way.

### EXAMPLES

### General Methodologies

Bacterial Strains.

Epidemic methicillin resistant S. aureus (MRSA) strain sequenced at the Sanger Centre (MRSA252, NRS71), S. aureus RN4220 (NCTC8325 NRS144), hyper-virulent community-acquired MSSA sequenced at the Sanger Centre (MSSA476, NRS72), MRSA strain sequenced at TIGR, (COL, NRS100) and community-acquired MRSA strain sequenced at the National Institute of Technology and Evaluation, Tokyo (USA400, MW2, NRS123) were obtained from NARSA (Network on Antimicrobial Resistance in S. aureus). Methicillin sensitive S. aureus (ATCC 29213 and 25923) were from ATCC, The Global Bioresourse Center. Streptococcus pyogenes (ATCC 700294), Acinetobacter baumannii (ATCC 19606), Klebsiella pneumonia (C238), Escherichia coli (DAS 1-IMP) and Pseudomonas aeruginosa (PAO-1) were obtained from the laboratory of Dr B. B. Finlay at the University of British Columbia (Vancouver, Canada).

Generation of Pyruvate Kinase (PK) Constructs.

Genomic DNA of MRSA strain Sanger 252 extracted using DNeasy Tissue Kit™ (Qiagen™) was used as a template to generate the His-tagged MRSA PK. Human cDNA from MCF-7 breast cancer cell line (courtesy of Dr. J Wong, BC Cancer Research Center (Vancouver, Canada) was used as a template to generate the full-length human M2 PK enzyme. The following primer sets were used creating appropriate restriction sites (NdeI and XhoI sites underlined): For cloning of MRSA PK: M27F 5′-CTACATATGAGAAAAACTAAAATTGTATG-3′ and M27R 5′-GTTCTCGAGTTATAGTACGTTTGCATATCCTTC-3′, for cloning of human M2 PK isoform: hM2F 5′-GATCATATGATGTCGAAGCCCCATAGTGAAGCC-3′ and hM2R 5′-GTTCTCGAGTCACGGCACAGGAACAACACGCATG-3′. The resulting PCR fragments for each construct were cloned into the NdeI and XhoI unique sites of the bacterial expression vector pET-28a (+) (Novagen™). This step resulted in plasmids pET-28a-MRSA and pET-28M2, which generated N-terminally His-tagged recombinant MRSA and human M2 PKs. The sequence and the correct reading frame of all constructs were verified by sequencing. Human M1, R and L PK constructs in pET-28-a(+) vectors (courtesy of Dr. L. Cantley, Harvard Medical, School (Boston, USA)) were used to generate relevant recombinant His-tagged human PK isoforms.

Expression and Purification of Recombinant his-Tagged MRSA and Human PKs.

MRSA and human constructs in pET-28a(+) were used to express relevant recombinant PK proteins in E. coli BL-21 (DE3). The proteins were expressed and purified using Ni-NTA agarose (Qiagen™) according to the manufacturer's protocol. Briefly, cells were grown to an absorbance of 0.4-0.5 at 600 nm in 2×YT medium, then induced with 0.1 mM IPTG for 3 h at 20° C. Cells were lysed by sonication on ice (3×10-s bursts with a 30-s recovery between bursts) in lysis buffer (0.2 mg/ml lysozyme, 50 mM Tris pH 7.5, 10 mM MgCl₂, 200 mM NaCl, 100 mM KCl, 10% glycerol, 10 mM imidazole, 0.5% NP-40 and 1 mM DTT containing Complete™ protease inhibitor). Cell lysates were cleared by centrifugation (18,000×g in a Beckman™ JA-20 rotor) for 20 min at 4° C. and PK isoforms were purified by batch binding to Ni-NTA resin. The resins were then packed in columns (1×2 cm) and washed with 400 column volumes lysis buffer containing 30 mM imidazole. His-tagged PK isoforms were eluted with the same buffer containing 300 mM imidazole. The proteins were dialyzed overnight at 4° C. against 2000 volumes of ice-cold 30 mM Tris pH 7.5, 25 mM KCl, 5 mM MgCl₂, 10% glycerol and 1 mM DTT to remove imidazole. All purification steps were done at 4° C.; enzymes were flash-frozen and stored at −70° C. Enzymatic activity of frozen protein preparations was stable for at least 10 months and up to 5 freeze/thaw cycles. Purity and physical integrity of proteins were assessed using SDS-polyacrylamide gel electrophoresis (SDS-PAGE) followed by coomassie blue staining. Protein concentration was estimated by Bradford assay (Bio-Rad Protein Assay™) using bovine serum albumin as a standard.

Measurement of PK Activity.

Candidate MRSA PK inhibitors were assayed for their ability to inhibit enzymatic activities of MRSA and human PKs. PK activity was determined using a continuous assay coupled to lactate dehydrogenase (LDH) in which the change in absorbance at 340 nm owing to oxidation of NADH was measured using a Benchmark Plus™ microplate spectrophotometer (Bio-Rad Laboratories, Hercules, Calif.). The reaction contained 60 mM Na⁺-HEPES, pH 7.5, 5% glycerol, 67 mM KCl, 6.7 mM MgCl₂, 0.24 mM NADH, 5.5 units L-LDH from rabbit muscle (Sigma-Aldrich, St. Louis, Mo.), 2 mM ADP and 10 mM PEP (i.e. close to the Kₘ of MRSA PK, so that the IC₅₀ values should approximate the Kᵢ) in a total volume of 200 μl. Reactions were initiated by the addition of 15 nM of one of the PK enzymes. PK activity proportional to the rate of change at 340 nm was expressed as specific activity (μmol/min/mg), which is defined as the amount of PK that catalyzes the formation of one micromole of either product per minute. Inhibitors were dissolved in DMSO with the final concentration of the solvent never exceeding 1% of the assay volume. IC₅₀ values were calculated by curve fitting on a four-parameter dose-response model with variable slope using Graphpad Prism 5.0™ (GraphPad™ Software Inc., La Jolla, Calif.). In all studies, less than 10% of total PEP was exhausted during the reaction. Reactions were performed at 30° C. for up to 5 min. All values determined represent at least two measurements, in triplicate (Tables 1-6) or duplicate unless mentioned otherwise.

In Vitro Susceptibility Testing.

The antimicrobial activities of PK inhibitor candidates were determined using the 96-well microtiter standard 2-fold serial broth microdilution method as described by CLSI (formerly NCCLS) with the various gram-positive and gram-negative bacteria species mentioned above. Bacteria from a single colony were grown, overnight in either BHI Broth (VRE), mueller hinton broth (S. aureus 29213; MRSA USA400) or L-broth (E. coli, P. aeruginosa, S. typhimuriu, K. Pneumonia and A. baumannii). Each compound was prepared in DMSO with 2-fold serial dilutions to give a final concentration of 64 to 0.031 μg/ml. 10 μl of the compound solution was then added, in duplicate, to either, 190 μl of cation adjusted mueller hinton broth (CAMHB) or 190 μl CAMHB containing ˜2.5×10⁵ CFU/ml of bacteria (final compound concentration 64 to 0.031 μg/ml). Culture plates were incubated for 18-24 h at 37° C., and optical density at 600 nm (OD₆₀₀) was measured using a Benchmark Plus™ microplate spectrophotometer (Bio-Rad™). The absorbance control values for the series containing CAMHB and inhibitor were subtracted as background from the corresponding infected wells. The MIC was defined as the lowest concentration of test compound leading to complete inhibition of cell growth in relationship to compound-free control wells as determined by optical density. Minimal inhibitory concentration (MIC) was defined as the lowest concentration of test compound leading to complete inhibition of cell growth in relation to compound-free control wells as determined by optical density. Vancomycin, methicillin and ciprofloxicin were used as reference compounds. All assays were run in triplicate (Tables 1-6) or duplicate. Experiments were replicated at least twice to verify reproducibility using the above conditions.

Other Methods:

¹H and ¹³C NMR spectra were recorded with Bruker Avance II™ 600 MHz, Bruker Avance III™ 500 MHz, Bruker Avance III™ 400 MHz or Bruker Avance II+. Processing of the spectra was performed with MestRec™ software. The high-resolution mass spectra were recorded either in positive or negative ion-mode with an ESI or multimode ESI/APCI ion source on an Agilent™ 6210 Time-of-Flight LC/MS mass spectrometer. Low resolution mass spectra were recorded using a Waters Micromass ZQ mass spectrometer. Analytical thin-layer chromatography (TLC) was performed on aluminum plates pre-coated with silica gel 60E-254 as the absorbent. The developed plates were air-dried, exposed to UV light and/or dipped in KMnO₄ solution and heated. Column chromatography was performed with silica gel 60 (230-400 mesh). Automated flash chromatography was carried out on Biotage Isolera Flash Purification Systems using commercial 50 μm silica gel cartridges. Purity (>90%) for all final compounds was confirmed by analytical reverse-phase HPLC utilizing either a Dikma Technologies™ Inspire® C18 reverse-phase analytical column (4.6×150 mm) or Waters Symmetry C18 reverse-phase analytical column (4.6×75 mm). All HPLC purifications were carried out using an Agilent™ C18 reverse-phase preparatory column (21.2×250 mm).

### Example 1: Synthesis of Compounds 10a-m, 12a-c, 14, 15, 17, 20a & b, 22a-g, 25a-c, 26a & b, 27a-c, 28a & b, AND 33a-f

The syntheses of the title compounds were carried out as generally shown in Schemes 1 through 6 and described in detail below. Briefly, the indole NH was first protected with a phenylsulfonyl group to give intermediate 6 which was subsequently iodinated at the 2-position to give 2-iodo-indole 7 by treating 6 with LDA followed by the addition of diiodoethane (Scheme 1). An attempt to couple 7 with the boronic acid 9 under standard Suzuki-Miyura conditions did not result in isolation of the desired product. However, the coupling reaction of boronic acid 9 with the unprotected indole 8, (obtained by hydrolysis of compound 7), provided the desired adduct. Finally, removing the Boc protecting group with TFA gave the desired compound 10.

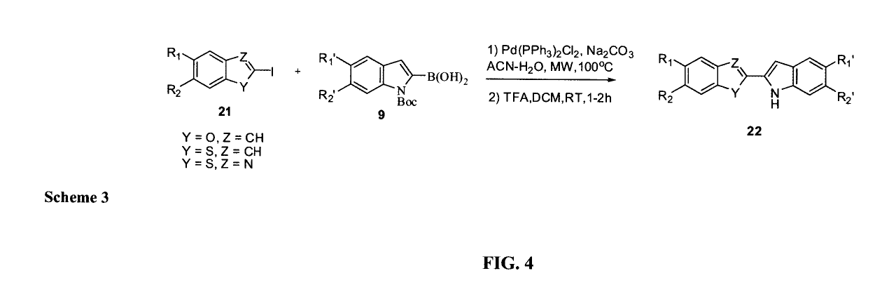

In order to prepare the alkylated bis-indole 12, 8 was first alkylated with alkyl halide to give intermediate 11 which was subsequently coupled with boronic acid 9 Scheme 1; FIG. 2). Compound 22 was prepared in a similar manner where an appropriate 2-iodo-hetrocycle 21 was coupled with boronic acid 9 under standard Suzuki-Miyura conditions and finally the Boc protecting group was removed with TFA (Scheme 3; FIG. 4).

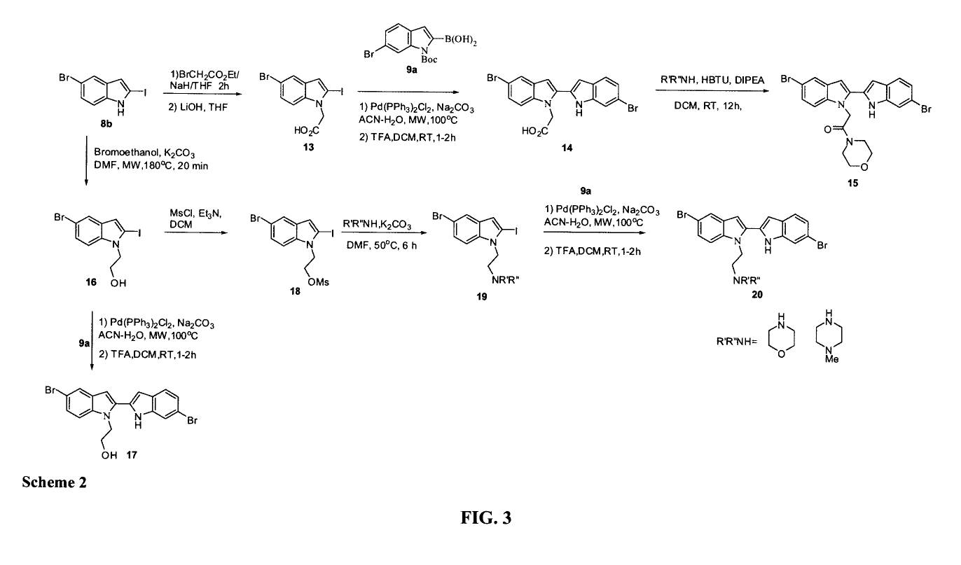

Compound 14 was prepared from 8b by treatment with an alkyl bromide, which was following by hydrolysis of the ester with LiOH to give the corresponding carboxylic acid derivative 13. Derivative 13 was coupled with 9a to provide 14 (Scheme 2; FIG. 3). The carboxylic acid on 14 was then reacted with morpholine and HBTU to give compound 15. Treating intermediate 8b with 2-bromoethanol gave alcohol 12 which was then coupled with boronic acid 9a and removal of Boc protecting group with TFA gave compound 17. Compound 20 was prepared from alcohol 12 which was first converted to the mesylate and then displaced by an amine to give intermediate 19 which was subsequently coupled with 9a followed by the removal of the Boc protecting group.

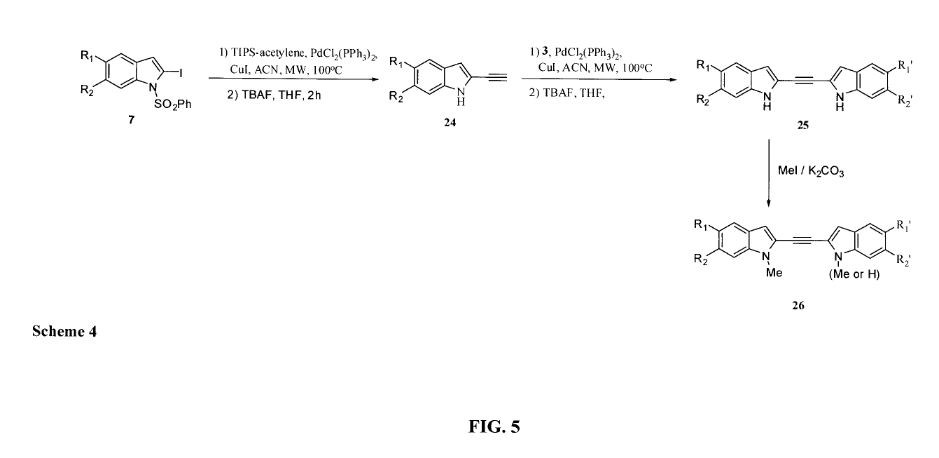

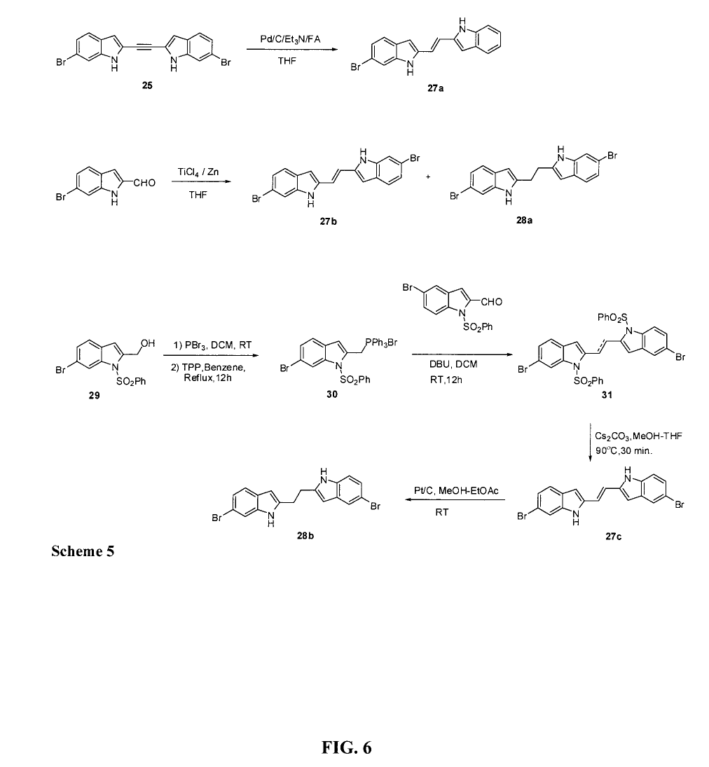

2-Acetylene-indole 24 was prepared by coupling 2-iodo-indole 7 with TIPS-acetylene using Sonogashira coupling condition with PdCl₂(PPh₃)₂ and CuI, and then the phenylsulfonyl protecting group was removed with TBAF (Scheme 4; FIG. 5). A second Sonogashira coupling of intermediate 24 with 7 followed by removal of the phenylsulfonyl group gave compound 25. Treating 25 with MeI gave a mixture of mono-methylated compound 26a and dimethylated compound 26b. Attempts were made to reduce the acetylene linker of compound 25 as a route to synthesize 27b, however, only the mono-brominated compound 27a (Scheme 5; FIG. 6). Compound 27b was however successfully synthesized by cross-coupling two molecules of 6-bromo-1H-indole-2-carbaldehyde using titanium tetrachloride and zinc dust which also gave 28a as a bi-product. Compound 27c was prepared from alcohol 29 where it was first converted to Wittig salt 30 (Scheme 5). Compound 30 was then coupled with the corresponding aldehyde to give 31 and finally the phenylsulfonyl protecting groups were removed with Cs₂CO₃ to give the desired adduct 27c. The double bond of compound 27c was reduced by hydrogenation over Pt/C to give compound 28b.

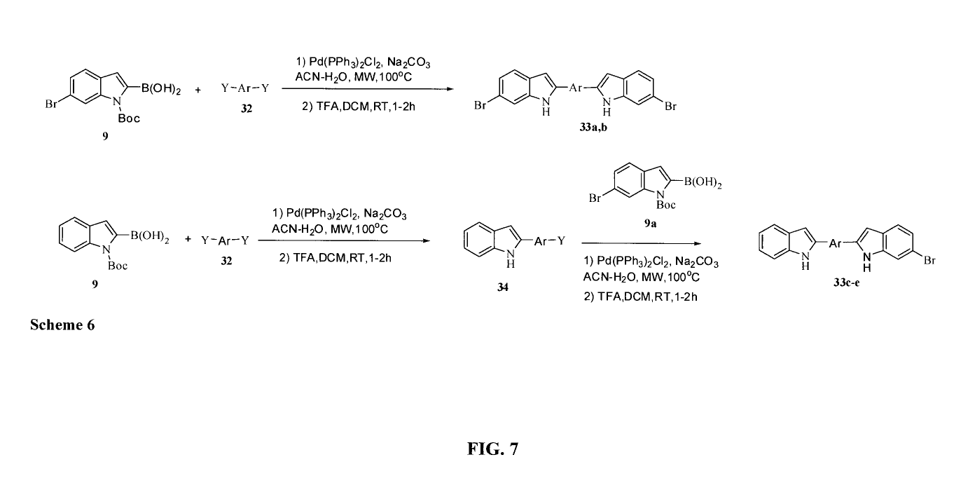

Symmetrical bis-indoles 33a and 33b were prepared by double Suzuki-Miyura reaction of boronic acid 9 with aryl di-halide 32 followed by the removal of the Boc protecting group with TFA (Scheme 6; FIG. 7). In order to prepare unsymmetrical bisindoles 33c-e, aryl di-halide was first coupled with one equivalent of boronic acid 9 to give intermediate 34 which was consequently coupled with a different boronic acid 9a and finally the Boc group was cleaved with TFA to give the desired compounds.

### General procedure for the synthesis of 1-(phenylsulfonyl)-1H-indole (6)

To a stirred solution of an appropriate indole (1 mmol) in THF (25 ml) at 0° C. was added NaH (60% in oil, 2 mmol) gradually. After stirring at room temperature for 10 minutes benzenesulphonyl chloride was added and the mixture was further stirred for 2h. The reaction was quenched with saturated ammonium chloride solution and extracted with EtOAc (2×50 ml). The combined organic phases were washed with brine, dried over anhydrous Na₂SO₄, filtered and concentrated in vacuo. The crude product was purified by automated flash chromatography to give the desired product.

### General procedure for the synthesis of 2-iodo-(phenylsulfonyl)-1H-indole (7)

To a stirred solution of 6 (1 mmol) in anhydrous THF (10 mL) at −78° C. was added a solution of LDA (1.5 mmol) in THF (5 mL). The mixture was stirred for at −78° C. for 100 min and then warmed to 0° C. for 30 min. The solution was re-cooled to −78° C. and then either a solution of 1,2-diiodo ethane or molecular iodine (1.5 mmol) in THF (10 mL) was added. The reaction mixture was stirred at 0° C. for 15 minutes and then allowed for warm to room temperature for 1 h. The reaction was quenched with saturated NH₄Cl solution and extracted with EtOAc (2×50 ml). The combined organic phases were washed with brine, dried over anhydrous Na₂SO₄, filtered and concentrated in vacuo. The crude product was purified by automated flash chromatography to give the desired product.

### 6-Bromo-2-iodo-1-(phenylsulfonyl)-1H-indole (7a)

Yield=53%, white solid. ¹H NMR (CDCl₃, 400 MHz): δ 8.16 (d, J=9.0 Hz, 1H), 7.87 (d, J=7.6 Hz, 2H), 7.58 (t, J=7.5 Hz, 1H), 7.5 (d, J=1.6 Hz, 1H), 7.46 (t, J=7.8 Hz, 2H), 7.37 (dd, J=9.0, 1.9 Hz, 1H), 6.93 (s, 1H). HRMS calcd for (C₁₄H₉BrINO₂S—H)⁻ 460.8582, found 460.8598.

### 5-Bromo-2-iodo-1-(phenylsulfonyl)-1H-indole (7b)

Yield=54%, white solid. ¹H NMR (CDCl₃, 400 MHz): δ 8.15 (d, J=9.0 Hz, 1H), 7.87 (d, J=8.5 Hz, 2H), 7.56 (t, J=8.1 Hz, 1H), 7.53 (d, J=1.7 Hz, 1H), 7.44 (t, J=7.5 Hz, 2H), 7.37 (dd, J=2.0, 9.0 Hz, 1H), 6.91 (s, 1H).

### 5-Chloro-2-iodo-1-(phenylsulfonyl)-1H-indole (7c)

Yield=75%, white Solid. ¹H NMR (CDCl₃, 400 MHz): δ 8.21 (d, J=9.0 Hz, 1H), 7.84-7.90 (m, 2H), 7.55-7.61 (m, 1H), 7.46 (dt, J=7.4, 1.8 Hz, 2H), 7.38 (d, J=1.9 Hz, 1H), 7.24 (dd, J=9.0, 2.2 Hz, 1H), 6.93 (d, J=0.7 Hz, 1H). ¹³C NMR (CDCl₃, 125 MHz): 138.12, 136.95, 134.44, 132.87, 129.82, 129.42, 127.31, 126.80, 125.21, 123.43, 119.36, 116.55 ppm. HRMS calcd for (C₁₄H₉ClINO₂S—H)⁻ 416.9087, found 416.9094.

### 5-Fluoro-2-iodo-1-(phenylsulfonyl)-1H-indole (7d)

Yield=94%, white solid. ¹H NMR (CDCl₃, 400 MHz): δ 8.23 (dd, J=9.2, 4.4 Hz, 1H), 7.84-7.89 (m, 2H), 7.54-7.61 (m, 1H), 7.42-7.48 (m, 2H), 6.97-7.08 (m, 2H), 6.95 (d, J=0.5 Hz, 1H). HRMS calcd for (C₁₄H₉FINO₂S—H)⁻ 400.9383, found 400.9392.

### General Procedure for the Synthesis of Substituted 2-iodo-1H-indole (8)

To a stirred solution of compound 7 (1 mmol) in THF (20 mL) at room temperature was added a solution of TBAF (1 mL, 1M in THF, 1 mmol). The mixture was stirred at ambient temperature for 5h and then partitioned between partitioned between EtOAc (100 mL) and H₂O (50 mL). The organic phase was washed with brine (50 mL), dried over anhydrous Na₂SO₄ and concentrated. The residue was purified by automated flash chromatography to give compound 8.

### 6-Bromo-2-iodo-1H-indole (8a)

Yield=48%, white solid. ¹H NMR (CDCl₃, 400 MHz): δ 8.07 (brs, 1H), 7.46-7.50 (m, 1H), 7.39 (d, J=8.4 Hz, 1H), 7.19 (dd, J=8.4, 1.7 Hz, 1H), 6.69 (dd, J=2.0, 0.9 Hz, 1H). ¹³C NMR (DMSO-d₆, 125 MHz): 139.38, 128.33, 122.17, 120.23, 114.01, 112.90, 110.84, 79.73. HRMS calc for (C₈H₅BrIN—H)⁻ 320.8650, found 320.8658.

### 6-Bromo-2-iodo-1H-indole (8b)

Yield=92%, white solid. ¹H NMR (DMSO-d₆, 400 MHz): δ 11.9 (brs, 1H), 7.65 (d, J=1.6 Hz, 1H), 7.28 (d, J=8.6 Hz, 1H), 7.15 (dd, J=1.9, 8.6 Hz, 1H), 6.6 (s, 1H).

### 5-Chloro-2-iodo-1H-indole (8c)

Yield=96%, pale brown solid. ¹H NMR (CDCl₃, 400 MHz): δ 8.12 (brs, 1H), 7.51 (d, J=2.0 Hz, 1H), 7.24 (d, J=8.7 Hz, 1H), 7.09 (dd, J=8.6, 2.0, 1H), 6.67 (d, J=1.2 Hz, 1H). ¹³C NMR (DMSO-d₆, 125 MHz): δ 137.16, 130.31, 123.94, 121.09, 117.68, 111.88, 110.33, 80.64. HRMS calc for (C₈H₅ClIN—H)⁻ 276.9155, found 276.9158.

### 5-fluoro-2-iodo-1H-indole (8d)

Yield=85%, white solid. ¹H NMR (CDCl₃, 400 MHz): δ 8.08 (brs, 1H), 7.22-7.25 (m, 1H), 7.19 (dd, J=9.4, 2.5 Hz, 1H), 6.89 (td, J=9.1, 2.5 Hz, 1H), 6.68 (dd, J=2.0, 0.8 Hz, 1H).

### 5,6-Dibromo-2-iodo-1H-indole

Yield=89%, white solid. ¹H NMR (CDCl₃, 400 MHz): δ 8.09 (brs, 1H), 7.80 (s, 1H), 7.69 (d, J=0.7 Hz, 1H), 6.64 (dd, J=0.8, 2.3 Hz, 1H).

### General procedure for the synthesis of substituted 2-iodo-1-methyl-1H-indole (11)

A mixture of an appropriate 2-iodo-1H-indole 8 (1 mmol), K₂CO₃ (2 mmol) and methyl iodide (1.5 mmol) in DMF was stirred at room temperature for 3d. The reaction mixture was partitioned between partitioned between EtOAc (100 mL) and H₂O (50 mL). The organic phase was washed with brine (50 mL), dried over anhydrous Na₂SO₄ and concentrated. The residue was purified by automated flash chromatography to afford compound 11.

### 6-Bromo-2-iodo-1-methyl-1H-indole (11a)

Yield=97%, white solid. ¹H NMR (CDCl₃, 400 MHz): δ 7.46-7.47 (m, 1H), 7.37 (d, J=8.4 Hz, 1H), 7.17 (dd, J=8.4, 1.7 Hz, 1H), 6.76 (d, J=0.8 Hz, 1H), 3.72 (s, 3H).

### 5-Bromo-2-iodo-1-methyl-1H-indole (11b)

Yield=99%, white solid. ¹H NMR (CDCl₃, 500 MHz): δ 7.63 (d, J=1.6 Hz, 1H), 7.22 (dd, J=8.7, 1.8 Hz, 1H), 7.16 (d, J=8.7 Hz, 1H), 6.72 (s, 1H), 3.73 (s, 3H). HRMS calcd for (C₉H₇BrIN)⁻ 334.8807, found 334.8780.

### Synthesis of 5-bromo-2-iodo-1-(methoxymethyl)-1H-indole (11c)

To a stirred slurry of NaH (22 mg, 60% in oil, 0.94 mmol) in THF (5 mL) and DMF (1 mL) at 0° C. was added a solution of 8b (200 mg, 0.63 mmol) in THF (5 mL). After stirring for 1 h methoxymethyliodide (64 μL, 0.75 mmol) was added and the mixture was further stirred for 2h. The reaction mixture was partitioned between Et₂O (100 mL) and H₂O (50 mL). The organic phase was washed with brine (50 mL), dried over anhydrous Na₂SO₄ and concentrated. The residue was purified by automated flash chromatography to afford 11c (178 mg, 77%) as yellow solid.

¹H NMR (DMSO-d₆, 500 MHz): δ 7.70 (d, J=1.8 Hz, 1H), 7.60 (d, J=8.7 Hz, 1H), 7.26 (dd, J=8.7, 1.9 Hz, 1H), 6.85 (s, 1H), 5.52 (s, 2H), 3.19 (s, 3H). ¹³C NMR (DMSO-d₆, 125 MHz): 136.66, 131.25, 124.32, 121.35, 112.96, 112.51, 112.31, 88.25, 76.62, 55.46. Yield: 77%. HRMS calc for (C₁₀H₉BrINO)⁻ 364.8192, found 364.8923.

### Synthesis of 2-(5-bromo-2-iodo-1H-indol-1yl)acetic acid (13)

To a stirred slurry of NaH (144 mg, 60% in oil, 6.0 mmol) in DMF (5 mL) at 0° C. was added a solution of 8b (960 mg, 3.0 mmol) in DMF (15 mL). The mixture was stirred at 0° C. for 30 minutes and then at rt for an additional 30 minutes. Ethyl 2-iodoacetate (225 μL, 3.6 mmol) was added and the mixture was stirred at rt for 16h. The reaction mixture was diluted with EtOAc and then washed with 1M HCl followed by brine, dried over anhydrous Na₂SO₄ and concentrated. The residue was dissolved in a mixture of THF and LiOH solution at 0° C. and stirred at rt until the completion of the reaction as indicated by TLC. The reaction mixture was acidified and extracted with EtOAc. The organic phase was washed with brine, dried over anhydrous Na₂SO₄ and concentrated. The crude product was purified by automated flash chromatography to give white solid (1.01g, 89%).

¹H NMR (DMSO-d₆, 500 MHz): δ 13.17 (bs, 1H), 7.69 (d, J=1.8 Hz, 1H), 7.50 (d, J=8.8 Hz, 1H), 7.21 (dd, J=8.7, 2.0 Hz, 1H), 6.81 (s, 1H), 5.0 (s, 2H). ¹³C NMR (DMSO-d₆, 125 MHz): 169.46, 136.74, 130.82, 123.92, 121.11, 112.36, 112.26, 111.22, 89.24, 48.26. HRMS calc for (C₁₀H₇BrINO₂—H)⁻ 378.8705, found 378.8691.

### Synthesis of 2-(5-bromo-2-iodo-1H-indol-1-yl)ethanol (16)

To a solution of 8b (38 mg, 0.11 mmol) in DMF (1 mL) was added bromo ethanol (14 mg, 0.12 mmol) and K₂CO₃ (49 mg, 0.36 mmol). The reaction heated at 180° C. with microwave for 30 minutes and then partitioned between EtOAc and H₂O. The organic phase was washed with brine, dried over anhydrous Na₂SO₄ and concentrated. The residue was purified by automated flash chromatography to afford 16 (24 mg, 59%) as white solid.

¹H NMR (CDCl₃, 500 MHz): δ 7.65 (s, 1H), 7.28 (d, J=7.7 Hz, 1H), 7.23 (dd, J=8.7, 1.8 Hz, 1H), 6.75 (s, 1H), 4.32 (t, J=5.6 Hz, 2H), 3.9-3.97 (m, 2H).

### Synthesis of 2-(5-bromo-2-iodo-1H-indol-1-yl)ethylmethanesulfonate (18)

To a stirred solution of 16 (3.0g, 8.2 mmol) in DCM (20 mL) at 0° C. was added Et3N (1.8 mL, 12.4 mmol) and methane sulphonyl chloride (0.64 mL, 8.2 mmol). The reaction mixture was stirred at rt for 1 h and then quenched by the addition of ice. The reaction mixture was extracted into DCM (3×10 mL) and the combined extract was washed with brine, dried over anhydrous Na₂SO₄ and concentrated to give essentially pure compound 14 as brown solid (2.98g, 82%).

¹H NMR (CDCl₃, 500 MHz): δ 7.66 (m. 1H), 7.24-7.28 (m, 2H), 6.78 (s, 1H), 4.45-4.55 (m, 4H), 2.71 (s, 3H). HRMS calc for (C₁₁H₁₁BrINO₃S)⁻ 442.8688, found 442.8686.

### General Procedure for the Synthesis of 2-Iodoindole Derivative 19

To a stirred solution of the intermediate 18 (1 mmol) in DMF (3 mL) was added K₂CO₃ (1.5 mmol) and the corresponding amine (3 mL). The mixture was heated at 50° C. for 12-20h. and then partitioned between EtOAc and H₂O. The organic phase was washed with saturated NH₄Cl, brine, dried over anhydrous Na₂SO₄ and concentrated. The residue was purified by automated flash chromatography to afford the desired product 19.

### 5-bromo-2-iodo-1-(2-(4-methylpiperazin-1-yl)ethyl)-1H-indole (19a)

Yield=89%, white solid. ¹H NMR (CDCl₃, 500 MHz): δ 7.61-7.64 (m, 1H), 7.19-7.21 (m, 2H), 6.69 (s, 1H), 4.20-4.30 (m, 2H), 2.35-2.65 (m, 10H), 2.29 (s, 3H). ¹³C NMR (125 MHz): 136.02, 131.22, 124.66, 122.06, 113.30, 111.53, 111.01, 103.91, 57.18, 55.01, 53.46, 46.14, 45.21. HRMS calc for (C₁₅H₁₉BrIN₃)⁻ 446.9807, found 446.9814.

### 4-(2-(5-bromo-2-iodo-1H-indol-1-yl)ethyl)morpholine (19b)

Yield=55%, brown solid. ¹H NMR (CDCl₃, 400 MHz): δ 7.65-7.64 (m, 1H), 7.26-7.22 (m, 2H), 6.72 (s, 1H), 4.27 (t, J=7.2 Hz, 2H), 3.70 (bs, 4H), 2.63 (t, J=7.0 Hz, 2H), 2.53 (bs, 4H).

### General Procedure for the Synthesis of Compounds 10, 12, 14, 17, 20, 22, 33 and 24

A solution of boronic acid 9 (1 mmol), iodo-heterocycle (8, 11, 21, 32 or 34) (1 mmol), Na₂CO₃ (1M aqueous solution, 3.5 mmol) in ACN (5 mL) was purged with argon for 10 minutes followed by the addition of Pd(PPh₃)₂Cl₂ catalyst (10 mol %). The mixture was heated in a sealed tube with microwave at 110° C. until all the staring material was consumed as indicated by TLC (typically in about 40-60 minutes). The reaction mixture was partitioned between partitioned between EtOAc (100 mL) and H₂O (50 mL). The organic phase was washed with brine (50 mL), dried over anhydrous Na₂SO₄ and concentrated. The residue was taken up in DCM (10 mL) and then TFA (1 mL) was added. After stirring at room temperature for 2 h, solvent was removed and the crude product was purified by automated flash chromatography to give the desired adduct.

### 6-Bromo-1H,1′H-2,2′-biindole (10a)

10a was prepared from (6-bromo-1-(tert-butoxycarbonyl)-1H-indol-2-yl)boronic acid 9a and tert-butyl 2-iodo-1H-indole-1-carboxylate.

Yield=35%, pink solid. mp=199-201° C. ¹H NMR (DMSO-d₆, 400 MHz): δ 11.71 (s, 1H), 11.59 (s, 1H), 7.51-7.59 (m, 3H), 7.41 (d, J=8.1 Hz, 1H), 7.09-7.16 (m, 2H), 7.02 (t, J=7.4 Hz, 1H), 6.92-6.95 (m, 2H). ¹³C NMR (DMSO-d₆, 150 MHz): 137.04, 136.85, 132.34, 130.69, 128.20, 127.42, 122.21, 121.92, 121.65, 120.15, 119.48, 114.14, 113.35, 111.13, 98.79, 98.37. HRMS calc for (C₁₆H₁₁BrN₂—H)⁻ 310.0106, found 310.0107.

### 6,6′-Dibromo-1H,1′H-2,2′-biindole (10b)

10b was prepared from (6-bromo-1-(tert-butoxycarbonyl)-1H-indol-2-yl)boronic acid 9a and 6-bromo-2-iodo-1H-indole 8a.

Yield=63%, White Solid. mp=266-267° C. ¹H NMR (500 MHz, DMSO-d₆): δ 11.77 (s, 2H), 7.56 (s, 2H), 7.54 (d, J=8.4 Hz, 2H), 7.15 (dd, J=1.6 Hz, 8.4 Hz, 2H), 6.95 (d, J=1.1 Hz, 2H). ¹³C NMR (125 MHz, DMSO-d₆): δ 137.8 (2C), 131.8 (2C), 127.4 (2C), 122.4 (2C), 121.9 (2C), 114.4 (2C), 113.5 (2C), 99.0 (2C). HRMS calcd for (C₁₆H₁₀Br₂N₂—H)⁻ 388.9118, found 388.9123.

### 5,6-Bibromo-1H,1′H-2,2′-biiindole (10c)

10c was prepared from (6-bromo-1-(tert-butoxycarbonyl)-1H-indol-2-yl)boronic acid 9a and 5-bromo-2-iodo-1H-indole 8b.

Yield=45%, pale brown solid. mp=270-272° C. ¹H NMR (DMSO-d₆, 400 MHz): δ 11.82 (s, 1H), 11.78 (s, 1H), 7.79 (s, 1H), 7.53-7.57 (m, 2H), 7.37 (d, J=8.5 Hz, 1H), 7.23 (d, J=8.6 Hz, 1H), 7.15 (d, J=8.4 Hz, 1H), 6.97 (s, 1H), 6.93 (s, 1H). ¹³C NMR (DMSO-d₆, 100 MHz): 137.86, 135.68, 132.28, 131.74, 130.26, 127.38, 124.30, 122.43, 122.28, 121.88, 114.47, 113.58, 113.01, 111.98, 99.11, 98.43. HRMS calc for (C₁₆H₁₀Br₂N₂—H)⁻ 387.9211, found 387.9227.

### 6′-Bromo-5-chloro-1H,1′H-2,2′-biindole (10d)

10d was prepared from (6-bromo-1-(tert-butoxycarbonyl)-1H-indol-2-yl)boronic acid 9a and 5-chloro-2-iodo-1H-indole 8c.

Yield=60%, white solid. mp=257-259° C. ¹H NMR (DMSO-d₆, 500 MHz): δ 11.80 (s, 1H), 11.77 (s, 1H), 7.64 (d, J=1.8 Hz, 1H), 7.52-7.55 (m, 2H), 7.40 (d, J=8.6 Hz, 1H), 7.14 (dd, J=8.4, 1.7 Hz, 1H), 7.11 (dd, J=8.6, 2.0 Hz, 1H), 6.95 (d, J=1.5 Hz, 1H), 6.92 (d, J=1.5 Hz, 1H). ¹³C NMR (DMSO-d₆, 100 MHz): 137.83, 135.44, 132.44, 131.77, 129.51, 127.36, 123.99, 122.41, 121.89, 121.77, 119.23, 114.43, 113.55, 112.54, 99.04, 98.53. HRMS calc for (C₁₆H₁₀BrClN₂—H)⁻ 343.9716, found 343.9727.

### 6′-Bromo-5-fluoro-1H,1′H-2,2′-biindole (10e)

10e was prepared from (6-bromo-1-(tert-butoxycarbonyl)-1H-indol-2-yl)boronic acid 9a and 5-fluoro-2-iodo-1H-indole 8d.

Yield=61%, white solid. mp=246-248° C. ¹H NMR (DMSO-d₆, 600 MHz): δ 11.73 (s, 1H), 11.68 (s, 1H), 7.51-7.56 (m, 2H), 7.38 (dd, J=8.7, 4.6 Hz, 1H), 7.34 (dd, J=9.9, 2.3 Hz, 1H), 7.14 (dd, J=8.4, 1.8 Hz, 1H), 6.90-6.97 (m, 3H). HRMS calc for (C₁₆H₁₀BrFN₂—H)⁻ 328.0011, found 328.0019.

### 6′-Bromo-5-methoxy-1H,1′H-2,2′-biindole (10f)

10f was prepared from (6-bromo-1-(tert-butoxycarbonyl)-1H-indol-2-yl)boronic acid 9a and tert-butyl 2-iodo-5-methoxy-1H-indole-1-carboxylate.

Yield=16%, pale brown solid. mp=240-241° C. ¹H NMR (DMSO-d₆, 500 MHz): δ 11.67 (s, 1H), 11.43 (s, 1H), 7.50-7.54 (m, 2H), 7.28 (d, J=8.7 Hz, 1H), 7.12 (dd, J=8.4, 1.7 Hz, 1H), 7.07 (d, J=2.4 Hz, 1H), 6.89 (s, 1H), 6.84 (s, 1H), 6.76 (dd, J=8.7, 2.4 Hz, 1H), 3.77 (s, 3H). ¹³C NMR (DMSO-d₆, 150 MHz): 153.68, 137.69, 132.51, 132.10, 131.19, 128.73, 127.50, 122.24, 121.61, 113.99, 113.40, 112.20, 111.75, 101.67, 98.80, 98.17, 55.30. HRMS calc for (C₁₇H₁₃BrN₂O—H) 340.0211, found 340.0217.

### 6′-Bromo-5-phenyl-1H,1′H-2,2′-biindole (10g)

10g was prepared from (6-bromo-1-(tert-butoxycarbonyl)-1H-indol-2-yl)boronic acid 9a and tert-butyl 2-iodo-5-phenyl-1H-indole-1-carboxylate.

Yield=17%, pale yellow solid. mp=237-239° C. ¹H NMR (DMSO-d₆, 600 MHz): δ 11.78 (s, 1H), 11.69 (s, 1H), 7.85-7.86 (m, 1H), 7.68-7.70 (m, 2H), 7.51-7.56 (m, 2H), 7.42-7.50 (m, 3H), 7.31 (t, J=7.4 Hz, 1H), 7.14 (dd, J=8.4, 1.7 Hz, 1H), 7.0 (s, 1H), 6.96 (s, 1H). ¹³C NMR (DMSO-d₆, 151 MHz): 141.62, 137.79, 136.60, 132.25, 132.00, 131.52, 128.95, 128.76 (2C), 127.46, 126.63 (2C), 126.27, 122.31, 121.74, 121.36, 118.17, 114.19, 113.45, 111.48, 99.30, 98.60. HRMS calc for (C₂₂H₁₅BrN₂—H)⁻ 386.0419, found 386.0429.

### 5-Bromo-1H,1′H-2,2′-biindole (10h)

10h was prepared from (5-bromo-1-(tert-butoxycarbonyl)-1H-indol-2-yl)boronic acid 9b and tert-butyl 2-iodo-1H-indole-1-carboxylate.

Yield=50%, white solid. ¹H NMR (DMSO-d₆, 400 MHz): δ 11.77 (s, 1H), 11.61 (s, 1H), 7.77 (d, J=1.7 Hz, 1H), 7.57 (d, J=7.8 Hz, 1H), 7.41 (d, J=8.0 Hz, 1H), 7.37 (d, J=8.6 Hz, 1H), 7.22 (dd, J=8.6, 1.9 Hz, 1H), 7.10-7.15 (m, 1H), 7.0-7.04 (m, 1H), 6.95 (d, J=1.3 Hz, 1H), 6.92 (d, J=1.4 Hz, 1H). ¹³C NMR (DMSO-d₆, 100 MHz): 137.0, 135.60, 132.91, 130.73, 130.37, 128.30, 124.03, 122.12, 121.96, 120.18, 119.50, 112.92, 111.87, 111.14, 99.02, 97.94. HRMS calc for (C₁₆H₁₁BrN₂—H)⁻ 310.0106, found 310.0107.

### 5,5′-Bibromo-1H,1′H-2,2′-biindole (10i)

10i was prepared from (5-bromo-1-(tert-butoxycarbonyl)-1H-indol-2-yl)boronic acid 9b and 5-bromo-2-iodo-1H-indole 8b.

Yield=65%, brown solid. mp=304-306° C. ¹H NMR (DMSO-d₆, 400 MHz): δ 11.83 (s, 2H), 7.79 (d, J=1.7 Hz, 2H), 7.36 (d, J=8.6 Hz, 2H), 7.23 (dd, J=8.6, 1.9 Hz, 2H), 6.94 (d, J=1.4 Hz, 2H). ¹³C NMR (DMSO-d₆, 100 MHz): 135.68, 132.22, 130.22, 124.33, 122.30, 113.03, 111.96, 98.52. HRMS calc for (C₁₆H₁₁BrN₂—H)⁻ 387.9211, found 387.9221.

### 5,6-Dibromo-1H,1′H-2,2′-biindole (10j)

10j was prepared from (1-(tert-butoxycarbonyl)-1H-indol-2-yl)boronic acid 9c and 5,6-dibromo-2-iodo-1H-indole 8e.

Yield=53%, white solid. mp=233° C.—(decompose). ¹H NMR (400 MHz, DMSO-d₆): δ 11.86 (s, 1H), 11.66 (s, 1H), 8.00 (s, 1H), 7.74 (s, 1H), 7.59 (d, J=7.7 Hz, 1H), 7.42 (d, J=8.0 Hz, 1H), 7.14 (t, J=7.2 Hz, 1H), 7.02 (t, J=7.4 Hz, 1H), 6.97 (s, 1H), 6.94 (s, 1H). ¹³C NMR (100 MHz, DMSO-d₆): δ 137.0, 136.7, 133.8, 130.2, 129.6, 128.2, 124.1, 122.1, 120.3, 119.5, 115.4, 115.3, 113.5, 111.2, 99.4, 97.8. HRMS calcd for (C₁₆H₁₀Br₂N₂—H)⁻ 388.9118, found 388.9108.

### 5,6,6′-Tribromo-1H,1′H-2,2′-biindole (10k)

10k was prepared from (6-bromo-1-(tert-butoxycarbonyl)-1H-indol-2-yl)boronic acid 9a and 5,6-dibromo-2-iodo-1H-indole 8e.

Yield=24%, pale yellow solid. ¹H NMR (400 MHz, DMSO-d₆): δ 11.95 (s, 1H), 11.87 (s, 1H), 8.02 (s, 1H), 7.75 (s, 1H), 7.75 (s, 1H), 7.56 (s, 1H), 7.55 (d, J=8.6 Hz, 1H), 7.15 (dd, J=1.7 Hz, 8.4 Hz, 1H), 6.98 (bs, 1H), 6.94 (s, 1H). ¹³C NMR (100 MHz, DMSO-d₆): δ 137.9, 136.7, 133.2, 131.3, 129.5, 127.3, 124.3, 122.5, 122.0, 115.6, 115.5, 114.6, 113.64, 113.60, 99.5, 98.3. HRMS calcd for (C₁₆H₉Br₃N₂—H)⁻ 466.8223, found 466.8223.

### 5,5′,6,6′-Tetrabromo-1H,1′H-2,2′-biindole (101)

101 was isolated as a minor bi-product during the synthesis of 10k from 9a and 8e.

Yield=7%, pale yellow solid. ¹H NMR (400 MHz, DMSO-d₆): δ 11.98 (bd, J=0.8 Hz, 2H), 8.04 (s, 2H), 7.75 (d, J=0.6 Hz, 2H), 6.97 (d, J=1.2 Hz, 2H). ¹³C NMR (100 MHz, DMSO-d₆): δ 136.8 (2C), 132.6 (2C), 129.3 (2C), 124.5 (2C), 115.9 (2C), 115.6 (2C), 113.8 (2C), 98.9 (2C). HRMS calcd for (C₁₆H₈Br₄N₂—H)⁻ 546.7308, found 546.7292.

### 5,5′,6-Tribromo-1H,1′H-2,2′-biindole (10m)

10m was prepared from (5-bromo-1-(tert-butoxycarbonyl)-1H-indol-2-yl)boronic acid 9b and 5,6-dibromo-2-iodo-1H-indole 8e.

Yield=49%, white solid. ¹H NMR (400 MHz, DMSO-d₆): δ 12.04 (s, 1H), 11.97 (s, 1H), 8.02 (s, 1H), 7.79 (d, J=1.8 Hz, 1H), 7.75 (s, 1H), 7.37 (d, J=8.6 Hz, 1H), 7.23 (dd, J=1.9 Hz, 8.6 Hz, 1H), 6.95 (bs, 2H). ¹³C NMR (100 MHz, DMSO-d₆): δ 136.8, 135.7, 133.2, 131.8, 130.1, 129.5, 124.5, 124.3, 122.4, 115.58, 115.55, 113.6, 113.1, 112.0, 98.9, 98.4. HRMS calcd for (C₁₆H₉Br₃N₂—H)⁻ 466.8223, found 466.8211.

### 5,6′-Dibromo-1-methyl-1H,1′H-2,2′-biindole (12a)

12a was prepared from (6-bromo-1-(tert-butoxycarbonyl)-1H-indol-2-yl)boronic acid 9a and 5-bromo-2-iodo-1-methyl-1H-indole 11b.

Yield=53%, white solid; mp=180-182° C. ¹H NMR (DMSO-d₆, 400 MHz): δ 11.78 (s, 1H), 7.82 (d, J=1.9 Hz, 1H), 7.51-7.61 (m, 3H), 7.31 (dd, J=8.7, 2.0 Hz, 1H), 7.18 (dd, J=8.4, 1.8 Hz, 1H), 6.91 (s, 1H), 6.89 (s, 1H), 3.96 (s, 3H). ¹³C NMR (DMSO-d₆, 125 MHz): 137.58, 137.0, 133.90, 130.03, 128.85, 127.41, 124.25, 122.45, 122.31, 122.06, 114.75, 113.68, 112.35, 112.14, 101.76, 100.61, 31.78. HRMS calc for (C₁₇H₁₂Br₂N₂—H)⁻ 401.9367, found 401.9384.

### 5′,6-Dibromo-1-methyl-1H,2′H-2,2′-biindole (12b)

12b was prepared from (5-bromo-1-(tert-butoxycarbonyl)-1H-indol-2-yl)boronic acid 9b and 6-bromo-2-iodo-1-methyl-1H-indole 8a.

Yield=48%, pale yellow solid. mp=192-194° C. ¹H NMR (DMSO-d₆, 400 MHz): δ 11.76 (s, 1H), 7.84 (s, 1H), 7.78 (d, J=1.9 Hz, 1H), 7.57 (d, J=8.4, 1H), 7.39 (d, J=8.6 Hz, 1H), 7.26 (dd, J=8.6, 1.9 Hz, 1H), 7.21 (dd, J=8.4, 1.7 Hz, 1H), 6.92 (d, J=0.6 Hz, 1H), 6.86 (d, J=1.5 Hz, 1H). ¹³C NMR (DMSO-d₆, 100 MHz): 139.13, 135.39, 133.41, 130.59, 130.25, 126.04, 124.57, 122.71, 122.37, 121.92, 114.76, 113.16, 112.97, 111.92, 101.48, 101.06, 31.78. HRMS calc for (C₁₇H₁₂Br₂N₂—H)⁻ 401.9367, found 401.9376.

### 5,6′-Dibromo-1-(methoxymethyl)-1H,1′H-2,2′-biindole (12c)

12c was prepared from (6-bromo-1-(tert-butoxycarbonyl)-1H-indol-2-yl)boronic acid 9a and 5-bromo-2-iodo-1-(methoxymethyl)-1H-indole 11c.

Yield=65%, white solid. mp=199-201° C. ¹H NMR (DMSO-d₆, 500 MHz): δ 11.74 (s, 1H), 7.87 (d, J=1.9 Hz, 1H), 7.74 (d, J=8.8 Hz, 1H), 7.55-7.61 (m, 2H), 7.36 (dd, J=8.3, 2.4 Hz, 1H), 7.17 (dd, J=8.4, 1.8 Hz, 1H), 6.98 (s, 1H), 6.95 (s, 1H), 5.71 (s, 2H) 3.30 (s, 3H). ¹³C NMR (DMSO-d₆, 125 MHz): 137.69, 137.46, 133.97, 129.50, 129.18, 127.40, 124.95, 122.55, 122.45, 122.26, 114.87, 113.67, 113.14, 112.51, 102.23, 101.93, 74.19, 55.55. HRMS calc for (C₁₈H₁₄Br₂N₂O—H)⁻ 431.9473, found 431.9485.

### 2-(5,6′-Dibromo-1H,1′H-[2,2′-biindol]-1-yl)acetic acid (14)

14 was prepared from (6-bromo-1-(tert-butoxycarbonyl)-1H-indol-2-yl)boronic acid 9a and 2-(5-bromo-2-iodo-1H-indol-1yl)acetic acid 13.

Yield=33%, white solid. mp=321-323° C. ¹H NMR (DMSO-d₆, 400 MHz): δ 11.77 (s, 1H), 7.84 (d, J=1.9 Hz, 1H), 7.52-7.60 (m, 3H), 7.31 (dd, J=8.7, 2.0 Hz, 1H), 7.17 (dd, J=8.5, 1.7 Hz, 1H), 6.91 (s, 1H), 6.69 (d, J=1.5 Hz, 1H), 5.24 (s, 2H). ¹³C NMR (DMSO-d₆, 150 MHz): 170.07, 137.48, 137.29, 133.85, 129.69, 129.03, 127.34, 124.57, 122.51, 122.39, 122.13, 114.84, 113.67, 112.71, 112.34, 101.64, 100.79, 46.05. HRMS calc for (C₁₈H₁₂Br₂N₂O₂—H)⁻ 445.9266, found 445.9272.

### 2-(5,6′-Dibromo-1H, 1′H-[2,2′-biindol]-1-yl)ethanol (17)

17 was prepared from (6-bromo-1-(tert-butoxycarbonyl)-1H-indol-2-yl)boronic acid 9a and 2-(5-bromo-2-iodo-1H-indol-1-yl)ethanol 16.

Yield=8%, pale white solid. ¹H NMR (DMSO-d₆, 500 MHz): δ 11.71 (s, 1H), 7.81 (d, J=1.9 Hz, 1H), 7.54-7.59 (m, 3H), 7.30 (dd, J=8.7, 2.0 Hz, 1H), 7.18 (dd, J=8.4, 1.8 Hz, 1H), 6.91 (s, 1H), 6.86 (s, 1H), 5.17 (t, J=5.1 Hz, 1H), 4.47 (t, J=5.9 Hz, 2H), 3.78 (q, J=5.6 Hz, 2H). ¹³C NMR (DMSO-d₆, 100 MHz): 137.48, 136.82, 133.84, 130, 129.06, 127.36, 124.22, 122.42, 122.26, 122.07, 114.66, 113.70, 112.74, 112.39, 101.74, 101.32, 59.97, 46.44. HRMS calc for (C₁₈H₁₄Br₂N₂O—H)⁻ 431.9473, found 431.9483.

### 5,6′-Dibromo-1-(2-(4-methylpiperazin-1-yl)ethyl)-1H,1′H-2,2′-biindole (20a)

20a was prepared from (6-bromo-1-(tert-butoxycarbonyl)-1H-indol-2-yl)boronic acid 9a and 5-bromo-2-iodo-1-(2-(4-methylpiperazin-1-yl)ethyl)-1H-indole 19a.

Yield=57%, pale white solid. mp: 140° C. ¹H NMR (CDCl₃, 500 MHz): 11.94 (s, 1H), 7.81 (s, 1H), 7.52-7.61 (m, 3H), 7.31 (d, J=8.7 Hz, 1H), 7.18 (d, J=8.4 Hz, 1H), 6.89 (s, 1H), 6.84 (s, 1H), 4.51 (t, J=6.4 Hz, 2H), 2.62 (t, J=6.4 Hz, 2H), 2.15-2.45 (m, 8H), 2.11 (s, 3H). ¹³C NMR (150 MHz): 137.47, 136.46, 133.66, 130.04, 129.12, 127.38, 124.34, 122.45, 122.41, 122.10, 114.65, 113.72, 112.54, 112.49, 101.71, 101.50, 56.77, 54.39, 52.79, 45.38, 42.41. HRMS calc for (C₂₃H₂₄Br-2N₄—H)⁻ 514.0368, found 514.0372.

### 4-(2-(5,6′-dibromo-1H,1′H-[2,2′-biindol]-1-yl)ethyl)morpholine (20b)

20b was prepared from (6-bromo-1-(tert-butoxycarbonyl)-1H-indol-2-yl)boronic acid 9a and 4-(2-(5-bromo-2-iodo-1H-indol-1-yl)ethyl)morpholine 19b.

Yield=22%, white form. ¹H NMR (400 MHz, DMSO-d₆): δ 11.89 (s, 1H), 7.81 (d, J=1.3 Hz, 1H), 7.59-7.56 (m, 3H), 7.31 (dd, J=1.4 Hz, 8.7 Hz, 1H), 7.18 (dd, J=1.2 Hz, 8.4 Hz, 1H), 6.89 (s, 1H), 6.85 (s, 1H), 4.54 (t, J=6.4 Hz, 2H), 3.43 (bs, 4H), 2.60 (t, J=6.3 Hz, 2H), 2.30 (bs, 4H). ¹³C NMR (100 MHz, DMSO-d₆): δ 137.5, 136.5, 133.7, 130.0, 129.1, 127.3, 124.3, 122.4, 122.3, 122.1, 114.6, 113.7, 122.5, 122.4, 101.7, 101.4, 66.0, 57.3, 53.5, 42.0. HRMS calcd for (C₂₂H₂₁Br₂ON₃—H)⁻ 504.0105, found 504.0116.

### 2-(6-Bromo-1H-indol-2-yl)benzo[d]thiazole (22a)

22a was prepared from (6-bromo-1-(tert-butoxycarbonyl)-1H-indol-2-yl)boronic acid 9a and 2-iodobenzo[d]thiazole 21a.

Yield=43%, pink solid. ¹H NMR (DMSO-d₆, 600 MHz): δ 12.35 (s, 1H), 8.16 (ddd, J=8.0, 1.1, 0.6 Hz, 1H), 8.04 (ddd, J=8.2, 1.1, 0.6 Hz, 1H), 7.63-7.64 (m, 1H), 7.62 (d, J=8.5 Hz, 1H), 7.57 (ddd, J=8.2, 7.2, 1.2 Hz, 1H), 7.48 (ddd, J=8.2, 7.2, 1.2 Hz, 1H), 7.29 (dd, J=2.2, 0.8 Hz, 1H), 7.21 (dd, J=8.4, 1.8 Hz, 1H). ¹³C NMR (DMSO-d₆, 150 MHz): 159.40, 153.21, 150.64, 138.43, 134.23, 131.92, 126.85, 126.78, 125.60, 123.21, 123.10, 122.50, 122.41, 116.61, 114.63, 105.08. HRMS calc for (C₁₅H₉BrN₂S—H)⁻ 327.9670, found 327.9674.

### 2-(Benzofuran-2-yl)-6-bromo-1H-indole (22b)

22b was prepared from (6-bromo-1-(tert-butoxycarbonyl)-1H-indol-2-yl)boronic acid 9a and 2-iodobenzofuran 21b.

Yield=40%, pale white solid. mp=196-198° C. ¹H NMR (DMSO-d₆, 400 MHz): δ 12.02 (s, 1H), 7.71 (d, J=7.1 Hz, 1H), 7.64 (d, J=8.1 Hz, 1H), 7.60 (s, 1H), 7.56 (d, J=8.5 Hz, 1H), 7.25-7.37 (m, 3H), 7.18 (dd, J=8.4, 1.7 Hz, 1H), 7.0 (s, 1H). ¹³C NMR (DMSO-d₆, 100 MHz): 154.0, 149.12, 137.97, 129.34, 128.55, 127.11, 124.75, 123.44, 122.75, 122.26, 121.55, 115.11, 113.92, 111.0, 102.28, 100.45. HRMS calc for (C₁₆H₁₀BrNO—H)⁻ is 310.9946, found 310.9948.

### 2-(Benzo[b]thiophen-2-yl)-6-bromo-1H-indole (22c)

22c was prepared from (6-bromo-1-(tert-butoxycarbonyl)-1H-indol-2-yl)boronic acid 9a and 2-iodobenzo[b]thiophene 21c.

Yield=57%, white solid. mp=250-252° C. ¹H NMR (DMSO-d₆, 400 MHz): δ 11.91 (s, 1H), 7.94-7.98 (m, 1H), 7.86 (dd, J=7.1, 1.5 Hz, 1H), 7.82 (s, 1H), 7.54-7.57 (m, 1H), 7.50 (d, J=8.4 Hz, 1H), 7.33-7.43 (m, 2H), 7.14 (dd, J=8.4, 1.8 Hz, 1H), 6.83 (d, J=1.4 Hz). ¹³C NMR (DMSO-d₆, 150 MHz): 139.98, 138.35, 138.03, 135.14, 132.93, 127.38, 124.99, 124.86, 123.75, 122.67, 122.47, 122.01, 119.87, 114.87, 113.67, 100.86. HRMS calc for (C₁₆H₁₀BrNS—H)⁻ 326.9717, found 326.9708.

### 2-(5-Bromo-1H-indol-2-yl)benzo[d]thiazole (22d)

22d was prepared from (5-bromo-1-(tert-butoxycarbonyl)-1H-indol-2-yl)boronic acid 9b and 2-iodobenzo[d]thiazole 21a.

Yield=35%, colorless solid. mp=208-210° C. ¹H NMR (DMSO-d₆, 400 MHz): δ 12.43 (s, 1H), 8.15 (d, J=7.9 Hz, 1H), 8.05 (d, J=8.1 Hz, 1H), 7.84 (d, J=1.5 Hz, 1H), 7.57 (t, J=7.7 Hz, 1H), 7.42-7.50 (m, 2H), 7.34 (dd, J=8.7, 1.8 Hz, 1H), 7.22 (s, 1H). ¹³C NMR (DMSO-d₆, 100 MHz): 159.35, 153.22, 136.32, 134.22, 132.31, 129.50, 126.80, 126.44, 125.58, 123.30, 122.44 (2C), 114.22, 112.59, 104.24. HRMS calc for (C₁₅H₉BrN₂S—H)⁻ 327.9670, found 327.9676.

### 2-(Benzofuran-2-yl)-5-bromo-1H-indole (22e)

22e was prepared from (5-bromo-1-(tert-butoxycarbonyl)-1H-indol-2-yl)boronic acid 9b and 2-iodobenzofuran 21b.

Yield=38%, white solid. mp=236-238° C. ¹H NMR (DMSO-d₆, 400 MHz): δ 12.06 (s, 1H), 7.80 (d, J=1.9 Hz, 1H), 7.70-7.73 (m, 1H), 7.61-7.66 (m, 1H), 7.40 (d, J=8.6 Hz, 1H), 7.25-7.38 (m, 4H), 6.97 (d, J=1.5 Hz, 1H). ¹³C NMR (DMSO-d₆, 150 MHz): 154.09, 154.08, 149.06, 149.04, 135.82, 129.91, 129.81, 128.50, 124.98, 124.80, 123.44, 122.64, 121.29, 113.42, 112.20, 111.01, 102.41, 99.81. HRMS calc for C₁₆H₁₀BrNO is 310.9946, found 310.9954.

### 2-(Benzo[b]thiophen-2-yl)-5-bromo-1H-indole (221)

22f was prepared from (5-bromo-1-(tert-butoxycarbonyl)-1H-indol-2-yl)boronic acid 9b and 2-iodobenzo[b]thiophene 21c.

Yield=57%, white solid. mp=269-271° C. ¹H NMR (DMSO-d₆, 400 MHz): δ 12.0 (s, 1H), 7.98-8.01 (m, 1H), 7.86-7.90 (m, 1H), 7.85 (s, 1H), 7.76 (d, J=1.8 Hz, 1H), 7.35-7.44 (m, 3H), 7.25 (dd, J=8.6, 1.9 Hz, 1H), 6.82 (d, J=1.6 Hz, 1H). ¹³C NMR (DMSO-d₆, 100 MHz): 139.97, 138.37, 135.90, 134.64, 133.38, 130.18, 124.97, 124.89, 124.83, 123.75, 122.48, 122.38, 120.03, 113.24, 112.15, 100.26. HRMS calc for (C₁₆H₁₀BrNS—H)⁻ 326.9717, found 326.9722.

### 6-Bromo-2-(5-bromo-1H-indol-2-yl)benzo[d]thaizole (22g)

22g was prepared from (5-bromo-1-(tert-butoxycarbonyl)-1H-indol-2-y 1)boronic acid 9b and 6-bromo-2-iodobenzo[d]thiazole 21c.

Yield=48%, yellow solid. mp=246-248° C. ¹H NMR (DMSO-d₆, 400 MHz): δ 12.45 (s, 1H), 8.48 (d, J=2.0 Hz, 1H), 7.97 (d, J=8.7 Hz, 1H), 7.87 (d, J=1.8 Hz, 1H), 7.72 (dd, J=8.7, 2.0 Hz, 1H), 7.44 (d, J=8.7 Hz, 1H), 7.36 (dd, J=8.7, 1.9 Hz, 1H), 7.28 (d, J=1.5 Hz, 1H). ¹³C NMR (DMSO-d₆, 150 MHz): 160.26, 152.30, 136.42, 136.23, 131.87, 129.93, 129.45, 126.64, 125.01, 123.85, 123.38, 118.00, 114.26, 112.66, 104.67. HRMS calc for (C₁₅H₈Br₂N₂S—H)⁻ 405.8775, found 405.8765.

### 2-(4-bromo-3-nitrophenyl)-1H-indole (34a)

34a was prepared from (1-(tert-butoxycarbonyl)-1H-indol-2-yl)boronic acid and 1,4-dibromo-2-nitrobenzene.

Yield=38%, brown form. ¹H NMR (400 MHz, CDCl₃): δ 8.45 (bs, 1H), 7.96 (d, J=2.0 Hz, 1H), 7.76 (dd, J=2.0 Hz, 8.4 Hz, 1H), 7.65 (dd, J=0.9 Hz, 7.9 Hz, 1H), 7.58 (d, J=8.4 Hz, 1H) 7.42 (dd, J=0.8 Hz, 8.2 Hz, 1H), 7.28-7.24 (m, 1H), 7.17-7.13 (m, 1H), 6.73-6.72 (m, 1H).

### 2-(4-Bromo-3-methoxyphenyl)-1H-indole (34b)

34b was prepared from (1-(tert-butoxycarbonyl)-1H-indol-2-yl)boronic acid and 1,4-dibromo-2-methoxybenzene.

Yield=57%, white solid. ¹H NMR (DMSO-d₆, 600 MHz): δ 11.59 (s, 1H), 7.63 (d, J=8.2 Hz, 1H), 7.57 (d, J=2.0 Hz, 1H), 7.54 (d, J=8.0 Hz, 1H), 7.42-7.39 (m, 2H), 7.14-7.11 (m, 1H), 7.02-7.00 (m, 2H). ¹³C NMR (DMSO-d₆, 150 MHz): 156.2, 137.6, 137.2, 133.7, 133.6, 129.0, 122.4, 120.7, 120.0, 119.0, 111.8, 109.8, 109.5, 100.1, 56.9.

### 1,4-bis(6-bromo-1H-indol-2-yl)benzene (33a)

33a was prepared from (6-bromo-1-(tert-butoxycarbonyl)-1H-indol-2-yl)boronic acid 9a (2 equivalence) and 1,4-diiodobenzene.

Yield=67%, white solid. ¹H NMR (600 MHz, DMSO-d₆): δ 11.75 (s, 2H), 7.96 (s, 4H), 7.55 (s, 2H), 7.51 (d, J=8.4 Hz, 2H), 7.14 (dd, J=1.7 Hz, 8.4 Hz, 2H), 7.02 (d, J=1.5 Hz, 2H). ¹³C NMR (150 MHz, DMSO-d₆): δ 138.2 (2C), 138.0 (2C), 130.8 (2C), 127.7 (2C), 125.5 (4C), 122.3 (2C), 121.8 (2C), 114.2 (2C), 113.6 (2C), 99.2 (2C). HRMS calcd for (C₂₂H₁₄Br₂N₂—H)⁻ 464.9432, found 464.9441.

### 2,5-bis(6-bromo-1H-indol-2-yl)thiophene (33b)

33b was prepared from (6-bromo-1-(tert-butoxycarbonyl)-1H-indol-2-yl)boronic acid 9a (2 equivalence) and 2,5-diiodothiophene.

Yield=88%, yellow solid. Decomposes >228° C. ¹H NMR (600 MHz, DMSO-d₆): δ 11.81 (s, 2H), 7.55 (s, 2H), 7.53 (bs, 2H), 7.50 (d, J=8.4 Hz, 2H), 7.15 (dd, J=1.7 Hz, 8.4 Hz, 2H), 6.76 (d, J=1.2 Hz, 2H). ¹³C NMR (150 MHz, DMSO-d₆): δ 137.8 (2C), 133.8 (2C), 132.8 (2C), 127.5 (2C), 124.9 (2C), 122.6 (2C), 121.7 (2C), 114.4 (2C), 113.5 (2C), 99.3 (2C). HRMS calcd for (C₂₀H₁₂Br₂N₂S—H)⁻ 470.8995, found 470.9004.

### 2-(4-(1H-indol-2-yl)phenyl)-6-bromo-1H-indole (33c)

33c was prepared from (6-bromo-1-(tert-butoxycarbonyl)-1H-indol-2-yl)boronic acid 9a and tert-butyl 2-(4-iodophenyl)-1H-indole-1-carboxylate.

Yield=89%, Yellow solid. mp=>300° C. ¹H NMR (400 MHz, DMSO-d₆): δ 11.74 (s, 1H), 11.57 (s, 1H), 7.97 (d, J=8.7 Hz, 1H), 7.94 (d, J=9.1 Hz, 1H), 7.55 (s, 1H), 7.54 (d, J=7.8 Hz, 1H), 7.50 (d, J=8.4 Hz, 1H), 7.42 (d, J=8.1 Hz, 1H), 7.15-7.09 (m, 2H), 7.03-6.98 (m, 3H). ¹³C NMR (150 MHz, DMSO-d₆): δ 138.3, 138.0, 137.2, 137.1, 131.3, 130.4, 128.6, 127.7, 125.5, 125.3 (2C), 125.31 (2C), 122.3, 121.7, 120.0, 119.4, 114.1, 113.6, 111.2, 99.1, 99.0. HRMS calcd for (C₂₂H₁₅BrN₂—H)⁻ 387.0328, found 387.0333.

### 2-(4-(1H-indol-2-yl)-3-nitrophenyl)-6-bromo-1H-indole (33d)

33d was prepared from 9a and 34a.

Yield=57%, brown solid. (Mixture of isomers ˜5:1) ¹H NMR (400 MHz, DMSO-d₆): δ 11.97 (d, J=1.3 Hz, 1H), 11.60 (d, J=1.3 Hz, 1H), 8.45 (d, J=1.8 Hz, 1H), 8.25 (dd, J=1.8 Hz, 8.2 Hz, 1H), 7.90 (d, J=8.2 Hz, 1H), 7.60-7.55 (m, 3H), 7.43 (d, J=8.1 Hz, 1H), 7.15-7.06 (m, 3H), 7.03 (dd, J=7.0 Hz, 7.9 Hz, 1H), 6.59 (d, J=1.4 Hz, 1H). ¹³C NMR (150 MHz, DMSO-d₆): δ 148.9, 138.3, 137.1, 135.9, 132.1, 131.9, 131.4, 128.3, 128.1, 127.4, 124.4, 122.8, 122.4, 122.3, 120.5, 119.9, 119.6, 115.2, 113.9, 111.5, 101.7, 101.2. HRMS calcd for (C₂₂H₁₄BrN₃O₂—H)⁻ 432.0179, found 432.0195.

### 2-(4-(1H-indol-2-yl)-2-methoxyphenyl)-6-bromo-1H-indole (33e)

33e was prepared from 9a and 34b.

Yield=11%, yellow solid. ¹H NMR (600 MHz, DMSO-d₆): δ 11.62 (d, J=1.0 Hz, 1H), 11.37 (d, J=1.0 Hz, 1H), 7.87 (d, J=8.1 Hz, 1H), 7.65 (d, J=1.3 Hz, 1H), 7.63 (s, 1H), 7.59 (dd, J=1.6 Hz, 8.1 Hz, 1H), 7.56 (d, J=7.8 Hz, 1H), 7.51 (d, J=8.4 Hz, 1H), 7.44 (d, J=8.1 Hz, 1H), 7.14-7.11 (m, 2H), 7.06-7.01 (m, 3H), 4.09 (s, 3H). ¹³C NMR (150 MHz, DMSO-d₆): δ 156.4, 137.3, 137.2, 137.2, 135.4, 132.6, 128.6, 128.0, 127.2, 122.0, 121.8, 121.5, 120.1, 119.5, 118.9, 117.6, 113.8, 113.7, 111.2, 108.4, 101.7, 99.5, 55.9. HRMS calcd for (C₂₃H₁₇BrN₂O—H)⁻ 417.0434, found 417.0442.

### 2-(3-(1H-indol-2-yl)phenyl)-6-bromo-1H-indole (33f)

33f was prepared from 9a and tert-butyl 2-(3-iodophenyl)-1H-indole-1-carboxylate.

Yield=86%, white solid. mp=224-226° C. ¹H NMR (600 MHz, DMSO-d₆): δ 11.78 (s, 1H), 11.60 (s, 1H), 8.38 (s, 1H), 7.82 (d, J=7.8 Hz, 1H), 7.78 (d, J=7.8 Hz, 1H), 7.58-7.54 (m, 4H), 7.44 (d, J=7.4 Hz, 1H), 7.17-7.01 (m, 5H). ¹³C NMR (150 MHz, DMSO-d₆): δ 138.5, 138.0, 137.3, 137.1, 132.9, 132.3, 129.5, 128.6, 127.6, 124.2, 123.9, 122.3, 121.9, 121.8, 121.7, 120.1, 119.4, 114.2, 113.7, 111.3, 99.3, 99.2. HRMS calcd for (C₂₂H₁₅BrN₂—H)⁻ 387.0328, found 387.0335.

### Specific Procedure for the Synthesis of 2-(5,6′-dibromo-1H,1′H-[2,2′-biindol]-1-yl)-1-morpholinoethanone (15)

To a stirred solution of compound 14 (50 mg, 0.11 mmol), morpholine (10 μL, 0.12 mmol) and HBTU (44 mg, 0.12 mmol) in DMF (1 mL) at 0° C. was added DIPEA (61 μL, 0.35 mmol) and the resulting solution stirred at rt for 12h. The mixture was diluted with DCM (10 mL) and washed with 1N HCl, saturated aqueous NaHCO₃, brine and concentrated. The crude product was purified by automated flash chromatography to give compound 15 as white solid (38 mg, 68%). Mp=320-322° C.

¹H NMR (DMSO-d₆, 400 MHz): δ 11.17 (s, 1H), 7.65 (s, 1H), 7.50 (s, 1H), 7.39 (d, J=8.4 Hz, 1H), 7.19 (d, J=8.7 Hz, 1H), 7.11 (d, J=8.5 Hz, 1H), 7.07 (d, J=8.5 Hz, 1H), 6.71 (s, 1H), 6.37 (s, 1H), 5.10 (s, 2H), 3.62 (brs, 2H), 3.54 (brs, 2H). HRMS calc for (C₂₂H₁₉Br₂N₃O₂—H)⁻ 514.9844, found 514.9867.

### General Procedure for the Synthesis of Substituted 2-ethynyl-1H-indole (24) and Substituted bisindole (25)

A solution of 2-iodo indole 7 (1 mmol) and acetylene derivative (1 mmol) in THF (5 mL) was purged with argon for 10 minutes followed by triethylamine (3.5 mmol), CuI (0.2 mmol) and Pd(Ph₃P)₂Cl₂ as catalyst (10 mol %). The mixture was heated in microwave at 100° C. for 30 minutes. After completion of the reaction as monitored by TLC, water was added and the mixture extracted with EtOAc (2×20 mL). Combined organic layer was washed with brine, dried over anhydrous Na₂SO₄ and evaporated in vacuo. The residue was purified by automated flash chromatography and then taken up in THF (5 mL) followed by the addition if TBAF (1M in THF, 1 mmol). The mixture was stirred at rt for 2h and then partitioned between partitioned between EtOAc (50 mL) and H₂O (50 mL). The organic phase was washed with brine (50 mL), dried over anhydrous Na₂SO₄ and concentrated. The crude product was purified by automated flash chromatography to give the desired product.

### 6-Bromo-2-ethynyl-1H-indole (24a)

24a was prepared from 7a and TIPS-acetylene.

Yield=52%, dark brown solid. ¹H NMR (CDCl₃, 400 MHz): δ 8.20 (s, 1H), 7.48 (s, 1H), 7.44 (d, J=8.3, 1.0 Hz, 2H), 7.23 (dd, J=8.5, 1.6 MHz, 1H), 6.68 (s, 1H), 3.33 (s, 1H). ¹³C NMR (CDCl₃, 100 MHz):136.59, 126.23, 123.98, 122.17, 118.29, 117.47, 113.54, 109.61, 81.31, 75.55. HRMS calc for (C₁₀H₆Br₂N—H)⁻ 218.9684, found 218.9687.

### 5-Bromo-2-ethynyl-1H-indole (24b)

24b was prepared from 7b and TIPS-acetylene.

Yield=63%, brown solid. ¹H NMR (CDCl₃, 400 MHz): δ 8.23 (s, 1H), 7.72 (d, J=1.32 Hz, 1H), 7.32 (dd, J=8.7, 1.8 Hz, 1H), 7.19 (d, J=8.7 Hz, 1H), 6.75 (d, J=1.5 Hz, 1H), 3.33 (s, 1H). ¹³C NMR (126 MHz, CDCl₃): 134.59, 129.20, 126.82, 123.52, 118.93, 113.96, 112.38, 109.15, 81.54, 75.77. HRMS calc for (C₁₀H₆BrN—H)⁻ 218.9684, found 218.9685.

### 2-((1H-indol-2-yl)ethynyl)-6-bromo-1H-indole (25a)

25a was prepared from 24a and 2-iodo-1-(phenylsulfonyl)-1H-indole.

Yield=64%, pale brown solid; mp=240-242° C.; ¹H NMR (DMSO-d₆, 600 MHz): δ 11.94 (s, 1H), 11.78 (s, 1H), 7.57 (dd, J=7.9, 0.6 Hz, 1H), 7.52-7.54 (m, 2H), 7.36 (dd, J=8.2, 0.9 Hz, 1H), 7.17-7.22 (m, 2H), 7.06 (ddd, J=8.0, 7.0, 1.0 Hz, 1H), 6.89 (dd, J=2.0, 0.7 Hz, 1H), 6.88 (dd, J=2.0, 0.9 Hz, 1H). ¹³C NMR (DMSO-d₆, 151 MHz): δ 150.63, 137.31, 136.61, 127.15, 126.22, 123.23, 122.91, 122.22, 120.50, 119.97, 118.80, 117.50, 115.92, 113.78, 113.39, 108.33, 108.16, 85.62, 84.50. HRMS calc for (C₁₈H₁₁BrN₂—H)⁻ 334.0106, found 334.0113.

### 1,2-Bis(6-bromo-1H-indol-2-yl)ethyne (25b)

25b was prepared from coupling 7a with 24a.

Yield=50%, Yellow Solid. ¹H NMR (600 MHz, CDCl₃): δ 8.24 (bs, 2H), 7.52 (s, 2H), 7.47 (d, J=8.2 Hz, 2H), 7.26 (m, 2H), 6.84 (s, 2H). ¹³C NMR (150 MHz, CDCl₃): δ 137.0 (2C), 126.5 (2C), 124.3 (2C), 122.2 (2C), 118.6 (2C), 117.7 (2C), 113.8 (2C), 109.7 (2C), 85.1 (2C). HRMS calcd for (C₁₈H₁₀Br₂N₂—H)⁻ 412.9118, found 412.9126.

### 6-Bromo-2-((5-bromo-1H-indol-2-yl)ethynyl)-1H-indole (25c)

25c was prepared from 7b and 24a.

Yield=32%, pale yellow solid; mp=236-237° C.; ¹H NMR (CDCl₃, 600 MHz): δ 12.03 (s, 1H), 11.96 (s, 1H), 7.77 (d, J=1.8 Hz, 1H), 7.52-7.55 (m, 2H), 7.33 (d, J=8.7 Hz, 1H), 7.30 (dd, J=8.6, 1.9 Hz, 1H), 7.19 (dd, J=8.5, 1.6 Hz, 1H), 6.93 (m, 1H), 6.86 (m, 1H). ¹³C NMR (CDCl₃, 150 MHz): 137.78, 135.60, 129.35, 126.61, 126.12, 123.37, 122.97, 122.72, 119.45, 118.88, 116.50, 114.26, 113.75, 112.90, 108.93, 108.14, 85.50, 85.38. HRMS calc for (C₁₈H₁₀Br₂N₂—H)⁻ 412.9118, found 412.9102.

### Synthesis of Compounds 26a and 26b

A mixture of 25b (70 mg, 0.17 mmol), K₂CO₃ (94 mg, 0.65 mmol) and methyl iodide (19 μL, 0.30 mmol) in DMF (1.5 mL) was stirred at 30° C. for 3d and then partitioned between EtOAc (20 mL) and H₂O (10 mL). The organic phase was washed with brine (10 mL), dried over anhydrous Na₂SO₄ and concentrated. The crude product was purified by automated flash chromatography to give compounds 26a and 26b.

### 6-bromo-2-((6-bromo-1H-indol-2-yl)ethynyl)-1-methyl-1H-indole (26a)

Yield=40%, yellow solid. ¹H NMR (600 MHz, DMSO-d₆): δ 11.99 (s, 1H), 7.79 (s, 2H), 7.56 (s, 1H), 7.54 (d, J=8.4 Hz, 2H), 7.23 (dd, J=1.6 Hz, 8.4 Hz, 1H), 7.19 (dd, J=1.7 Hz, 8.5 Hz, 1H), 6.96 (s, 2H), 3.89 (s, 6H). ¹³C NMR (150 MHz, DMSO-d₆): δ 138.0, 137.4, 126.2, 125.6, 123.2, 123.0, 122.4, 122.3, 121.7, 118.4, 116.4, 116.1, 113.8, 113.1, 108.5, 107.6, 88.4, 83.6, 30.9. HRMS calcd for (C₁₉H₁₂Br₂N₂—H)⁻ 426.9275, found 426.9279.

### 1,2-bis(6-bromo-1-methyl-1H-indol-2-yl)ethyne (26b)

Yield=47%, pale brown solid. ¹H NMR (600 MHz, DMSO-d₆): δ 7.82 (s, 2H), 7.55 (d, J=8.3 Hz, 2H), 7.24 (d, J=8.3 Hz, 2H), 7.03 (s, 2H), 3.90 (s, 6H). ¹³C NMR (150 MHz, DMSO-d₆): δ 138.0 (2C), 125.6 (2C), 123.2 (2C), 122.5 (2C), 121.5 (2C), 116.5 (2C), 113.1 (2C), 108.1 (2C), 86.9 (2C), 31.0 (2C). HRMS calcd for (C₂₀H₁₄Br₂N₂+H)⁺ 442.9577, found 442.9562.

### Synthesis of (E)-2-(2-(1H-indol-2-yl)vinyl)-6-bromo-1H-indole (27a)

To a stirred solution of 25b (72 mg, 0.17 mmol) and Et₃N (0.34 mL, 2.4 mmol) in THF (2 mL) at 50° C. under Ar was added formic acid (88%, 774, 1.8 mmol) followed by addition of 10% Pd/C (8 mg). More 10% Pd/C (8 mg) was added every 15 minutes to a total of 40 mg and the mixture was heated for an additional 2h, filtered through a pad of silica which was thoroughly washed with EtOAc (2×10 mL. The filtrate was washed with H₂O (10 mL), brine (10 mL), dried over anhydrous Na₂SO₄ and concentrated. The crude product was purified by automated flash chromatography to give compounds 27a as yellow solid (4 mg, 7%).

¹H NMR (600 MHz, DMSO-d₆): δ 11.57 (s, 1H), 11.42 (s, 1H), 7.50 (d, J=7.9 Hz, 1H), 7.49 (bs, 1H), 7.45 (d, J=8.4 Hz, 1H), 7.34 (d, J=8.1 Hz, 1H), 7.20 (d, J=16.5 Hz, 1H), 7.18 (d, J=16.5 Hz, 1H), 7.11-7.09 (m, 2H), 6.96 (dd, J=7.1 Hz, 7.8 Hz), 6.57 (s, 2H). ¹³C NMR (150 MHz, DMSO-d₆): δ 138.2, 137.7, 137.5, 136.5, 128.4, 127.6, 122.18, 122.17, 121.6, 120.0, 119.3, 119.1, 117.7, 114.5, 113.3, 110.9, 103.2, 102.6. HRMS calcd for (C₁₈H₁₃BrN₂—H)⁻ 335.0189, found 335.0195.

### Synthesis of Compounds 27b and 28a

To a stirred solution of 6-bromo-1H-indole-2-carbaldehyde (80 mg, 0.36 mmol) and TiCl₄ (58 μL, 0.53 mmol) in THF (5 mL) under Ar was added Zn dust (70 mg, 1.1 mmol) gradually over 15 minutes and the resulting misture was refluxed for 3h. After cooling to rt 10% aqueous solution of K₂CO₃ (1 mL) was added and the reaction mixture was stirred at rt for 16h. The mixture was partitioned between EtOAc (50 mL) and H₂O (20 mL). The organic phase was washed with brine (20 mL), dried over anhydrous Na₂SO₄ and concentrated. The crude product was purified by automated flash chromatography to give compounds 27b and 28a.

### (E)-1,2-bis(6-bromo-1H-indol-2-yl)ethane (27b)

Yield=20%, yellow solid. Decomposes >300° C. ¹H NMR (600 MHz, DMSO-d₆): δ 11.59 (s, 2H), 7.49 (s, 2H), 7.46 (d, J=8.4 Hz, 2H), 7.20 (s, 2H), 7.11 (dd, J=1.7 Hz, 8.4 Hz, 2H), 6.60 (d, J=0.9 Hz, 2H). ¹³C NMR (150 MHz, DMSO-d₆): δ 138.2 (2C), 137.4 (2C), 127.5 (2C), 122.2 (2C), 121.7 (2C), 118.5 (2C), 114.6 (2C), 113.3 (2C), 103.0 (2C). HRMS calcd for (C₁₈H₁₂Br₂N₂—H)⁻ 414.9275, found 414.9280.

### 1,2-bis(6-bromo-1H-indol-2-yl)ethane (28a)

Yield=53%, Yellow solid. ¹H NMR (600 MHz, DMSO-d₆): δ 11.18 (s, 2H), 7.46 (s, 2H), 7.46 (d, J=8.3 Hz, 2H), 7.04 (d, J=8.3 Hz, 2H), 6.21 (s, 2H), 3.11 (s, 4H). ¹³C NMR (150 MHz, DMSO-d₆): δ 140.4 (2C), 136.8 (2C), 127.3 (2C), 121.4 (2C), 120.8 (2C), 113.1 (2C), 112.7 (2C), 98.7 (2C), 27.0 (2C). HRMS calcd for (C₁₈H₁₄Br₂N₂—H)⁻ 416.9431, found 416.9435.

### Synthesis of (6-Bromo-1-(phenylsulfonyl)-1H-indol-2-yl)methanol (29)

6-Bromo-N-benzenesulphonate (1.79 g, 5.3 mmol) in THF (20 mL) was cooled to −78° C. and LDA 1.8M in THF (4.46 mL, 8.0 mmol) was added. After 30 min at −78° C. and 30 min at rt the mixture was cooled to −78° C. and paraformaldehyde (206 mg, 6.89 mmol) was added all at once. The reaction was warmed to room temperature (rt) and stirred 12 h and then quenched by addition of saturated NH₄Cl. The mixture was extracted with EtOAc, the organics were washed with brine, dried over Na₂SO₄, concentrated and purified by automated flash chromatography to give compound 29.

Yield=72%, white solid; ¹H NMR (CDCl₃, 500 MHz): δ 8.25 (d, J=0.6 Hz, 1H), 7.83 (dd, J=1.1, 8.5 Hz, 1H), 7.61-7.56 (m, 1H), 7.47 (dd, J=5.0, 10.8 Hz, 1H), 7.38-7.33 (m, 1H), 6.62 (s, 1H), 4.88 (s, 1H). ¹³C NMR (CDCl₃, 126 MHz): 140.72, 138.27, 137.70, 134.47, 129.72, 128.01, 127.39, 126.51, 122.44, 118.88, 117.48, 111.12, 58.53. Yield: 72%.

### Synthesis of (E)-6-bromo-2-(2-(5-bromo-1-(phenulsulfonyl)-1H-indol-2-yl)vinyl-1-(phenulsulfonyl)-1H-indole (31)

Compound 29 (1.0 g, 2.54 mmol) was treated in dry ether (15 mL) at 0° C. with PBr3 (240 μL, 2.4 mmol) added slowly and then the reaction was stirred at room temperature for 30 min. After addition of aq. KBr, the mixture was extracted with ether and the organics were washed with brine, dried over Na₂SO₄, concentrated to provide the crude bromide as a brown solid which was dissolved in dichloromethane (DCM) (20 mL) and triphenylphosphine (744 mg, 2.84 mmol) was added and the mixture was stirred at rt overnight. The solvent was removed in vacuo and the residue was suspended in EtOAc (15 mL), sonicated and the solid collected by filtration. The crude Wittig reagent (1.0 g, 1.49 mmol) was dissolved in 1:1, THF:MeOH (20 mL) and DBU (285 μL, 2.02 mmol) was added followed by 5-bromo-N-benzenesulphonylindole (490 mg, 1.35 mmol). The mixture was stirred at rt for 3 h and then the solvent was removed under reduced pressure and the residue was partitioned between water and EtOAc, and the aqueous layers were washed with EtOAc, the organic extracts were combined, washed with brine dried over Na₂SO₄, concentrated and purified by automated flash chromatography to give compound 31 as a yellow solid.

Yield 36%; ¹H NMR (DMSO-d₆, 500 MHz): δ 8.23 (s, 1H), 8.04 (d, J=8.9 Hz, 1H), 7.85 (d, J=1.9 Hz, 1H), 7.83-7.48 (m, 15H), 7.35 (s, 1H), 7.27 (s, 1H). ¹³C NMR (DMSO-d₆, 151 MHz): 139.40, 138.69, 137.45, 137.14, 136.81, 136.72, 135.60, 135.10, 135.02, 131.62, 130.20, 130.09, 130.03, 129.92, 128.77, 128.05, 127.65, 126.45, 126.24, 126.21, 123.81, 123.25, 121.68, 121.58, 118.16, 117.13, 117.02, 116.50, 110.27, 109.61. HRMS calc for (C₃₀H₂OBr₂N₂O₄S₂—H)⁻ 692.9153, found 692.9158.

### Synthesis of (E)-6-bromo-2-(2-(5-bromo-1H-indol-2-yl)vinyl)-1H-indole (27c)

Compound 31 (20 mg, 0.29 mmol) was dissolved in 1 mL of THF: MeOH, (2:1) and Cs₂CO₃ (28 mg, 0.86 mmol) was added and the mixture has heated in a μwave reactor at 90° C. for 30 min. The reaction was cooled, solvents were removed and the residue was stirred with water 1 ml) for 10 min and then the mixture was extracted with DCM. The extracts were dried (Na₂SO₄), concentrated to dryness and the residue purified by automated flash chromatography to give compound 27c as a yellow solid.

Yield=88%, yellow solid; ¹H NMR (DMSO-d₆, 500 MHz): δ 11.65 (s, 1H), 11.60 (s, 1H), 7.68 (d, J=1.6 Hz, 1H), 7.50 (s, 1H), 7.46 (d, J=8.4 Hz, 1H), 7.31 (d, J=8.5 Hz, 1H), 7.22-7.17 (m, 3H), 7.11 (dd, J=1.7, 8.4 Hz, 1H), 6.60 (s, 1H), 6.56 (s, 1H). ¹³C NMR (DMSO-d₆, 126 MHz): 138.27, 137.96, 137.38, 136.11, 130.33, 127.52, 124.44, 122.23, 122.05, 121.72, 118.86, 118.47, 114.69, 113.29, 112.84, 111.74, 103.12, 102.3. HRMS calc for (C₁₈H₁₂Br₂N₂—H)⁻ 413.9367, found 412.9251.

### Synthesis of 6-bromo-2-(2-(5-bromo-1H-indol-2-yl)ethyl)-1H-indole (28b)

Compound 27c (48 mg) was dissolved in 1:1 EtOAc:MeOH (2 mL) and 5 mg 10% PT-C was added and the mixture was stirred under a H2 atmosphere at room temperature following the reaction by TLC. When complete, the mixture was filtered, concentrated to dryness and the residue was purified by automated flash chromatography to give compound 28b as a white solid.

Yield=81%; Mp=208-210° C.; ¹H NMR (DMSO-d₆, 500 MHz): δ 11.23 (s, 1H), 11.17 (s, 1H), 7.58 (d, J=1.7 Hz, 1H), 7.45 (s, 1H), 7.35 (d, J=8.4 Hz, 1H), 7.25 (d, J=8.6 Hz, 1H), 7.10 (dd, J=1.9, 8.5 Hz, 1H), 7.04 (dd, J=1.8, 8.3 Hz, 1H), 6.20 (s, 1H), 6.18 (s, 1H), 3.14 (s, 4H). ¹³C NMR (DMSO-d₆, 126 MHz): 141.00, 140.39, 136.83, 134.61, 130.18, 127.27, 122.51, 121.41, 121.32, 120.86, 113.14, 112.69, 112.53, 111.15, 98.68, 98.22, 27.04 (2C). HRMS calc for (C₁₈H₁₄Br₂N₂—H) 414.9445; found 414.9470.

### Example 2: Synthesis of Compounds 36a-c

### Synthesis of diethyl ((6-bromo-1-(phenylsulfonyl)-1H-indol-2-yl)methyl)phosphonate (35)

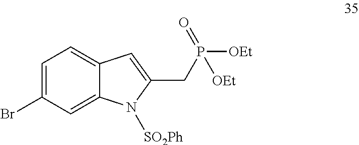

To a stirred solution of 29 (300 mg, 0.82 mmol) (see Example 1) in DCM (5 mL) at 0° C. under Ar was added PBr₃ (90 μL, 0.96 mmol) and the mixture was stirred at rt for 1 h. The mixture was re-cooled to 0° C. and then quenched with saturated NaHCO₃ (5 mL). The mixture was partitioned between EtOAc and H₂O and the organic layer was washed with brine, dried over anhydrous Na₂SO₄ and concentrated to give crude bromide. The residue was dissolved in benzene (1 mL) and triethyl phosphite and the resulting mixture was refluxed for 16h. All the volatiles were removed by distillation and the residue was purified by automated flash chromatography to give compound 35 was brown solid (295 mg, 74%).

¹H NMR (500 MHz, CDCl₃) δ 8.31 (s, 1H), 7.76 (d, J=7.7 Hz, 2H), 7.56 (t, J=7.5 Hz, 1H), 7.45 (t, J=7.9 Hz, 2H), 7.35 (dd, J=8.3, 1.4 Hz, 1H), 7.30 (d, J=8.3 Hz, 1H), 6.81 (d, J=3.3 Hz, 1H), 4.15-4.08 (m, 4H), 3.71 (d, J=22.0 Hz, 2H), 1.29 (t, J=7.1 Hz, 6H).

### General Procedure for the Synthesis of Compound 36

To a stirred solution of 35 (1 mmol) in THF (7 mL) at −10° C. was added NaH (60% in oil, 1.5 mmol) and the mixture was stirred for 15 min followed by the addition of the corresponding aldehyde (1.5 mmol) in THF (2 mL). The mixture was stirred at −10° C. for 2h and then partitioned between EtOAc and H₂O. The organic layer was washed with brine, dried over anhydrous Na₂SO₄ and then concentrated. The residue was partially purified by automated flash chromatography and then dissolved in THF (6 mL) and MeOH (12 mL). Cs₂CO₃ (2 mmol) was added and the mixture was heated with microwave at 90° C. for 30 min. The reaction mixture was partitioned between EtOAc and H₂O and the organic layer was washed with brine, dried over anhydrous Na₂SO₄ and then concentrated. The crude product was purified by automated flash chromatography to give the desired product 36a, b or c.

### (E)-6-bromo-2-styryl-1H-indole (36a)

Yield=71%, yellow solid; ¹H NMR (500 MHz, CDCl₃) δ 8.25 (s, 1H), 7.55-7.49 (m, 3H), 7.45 (d, J=8.4 Hz, 1H), 7.41 (t, J=7.6 Hz, 2H), 7.31 (t, J=7.3 Hz, 1H), 7.23 (dd, J=8.4, 1.2 Hz, 1H), 7.12 (d, J=16.5 Hz, 1H), 6.95 (d, J=16.5 Hz, 1H), 6.60 (s, 1H). ¹³C NMR (126 MHz, CDCl₃) 137.63, 136.94, 136.57, 128.84, 127.98, 127.87, 126.36, 124.71, 123.57, 121.75, 118.52, 116.20, 113.49, 103.71. HRMS calc for (C₁₆H₁₂BrN—H)⁻ 296.008, found 296.0076.

### (E)-6-bromo-2-(4-bromostyryl)-1H-indole (36b)

Yield=40%, yellow solid; ¹H NMR (500 MHz, CDCl₃) δ 8.22 (s, 1H), 7.55-7.49 (m, 3H), 7.46 (d, J=8.4 Hz, 1H), 7.38 (d, J=8.4 Hz, 2H), 7.23 (dd, J=8.4, 1.4 Hz, 1H), 7.10 (d, J=16.5 Hz, 1H), 6.86 (d, J=16.5 Hz, 1H), 6.62 (s, J=16.7 Hz, 1H). ¹³C NMR (126 MHz, CDCl₃) δ 137.70, 136.55, 135.55, 131.96, 127.80, 127.77, 126.48, 123.68, 121.84, 121.68, 119.20, 116.44, 113.54, 104.17. HRMS calc for (C₁₆H₁₁Br₂N—H)⁻ 375.9166, found 375.9158.

### (E)-6-bromo-2-(4-chlorostyryl)-1H-indole (36c)

Yield=50%, yellow solid; ¹H NMR (500 MHz, CDCl₃) δ 8.23 (s, 1H), 7.56-7.41 (m, 4H), 7.36 (d, J=8.0 Hz, 2H), 7.23 (d, J=8.0 Hz, 1H), 7.08 (d, J=16.4 Hz, 1H), 6.88 (d, J=16.2 Hz, 1H), 6.61 (s, 1H). ¹³C NMR (126 MHz, CDCl₃) δ 137.68, 136.58, 135.11, 133.54, 129.02, 127.81, 127.48, 126.46, 123.67, 121.83, 119.10, 116.42, 113.53, 104.09. HRMS calc for (C₁₆H₁₁BrClN—H)⁻ 331.9669, found 331.9665.

### Example 3: Synthesis of Compounds 37a-C

### General Procedure for the Synthesis of Compound 37

To a stirred solution of compound 36 (1 mmol) (see Example 2) in EtOAc (25 mL) was added Pt/C (10% dry on C, 50 mg) and the mixture was stirred under H₂ atmosphere for 16h, filtered through a pad of celite and concentrated. The crude product was purified by automated flash chromatography to give the desired product 37a, b or c.

### 6-bromo-2-phenethyl-1H-indole (37a)

Yield=62%, white solid; ¹H NMR (500 MHz, CDCl₃) δ 7.71 (s, 1H), 7.43-7.38 (m, 3H), 7.34 (t, J=7.3 Hz, 2H), 7.28-7.25 (m, 1H), 7.23 (d, J=7.1 Hz, 2H), 7.19 (dd, J=8.4, 1.7 Hz, 1H), 6.27 (s, 1H), 3.12 3.02 (m, 4H). ¹³C NMR (126 MHz, CDCl₃) δ 140.93, 139.73, 136.59, 128.61, 128.39, 127.54, 126.39, 122.89, 121.03, 114.43, 113.25, 100.04, 35.50, 30.05. HRMS calc for (C₁₆H₁₄BrN—H) 398.0237, found 298.0235.

### 6-bromo-2-(4-bromophenethyl)-1H-indole (37b)

Yield=87%, yellow solid; ¹H NMR (500 MHz, CDCl₃) δ 7.75 (s, 1H), 7.47-7.42 (m, 3H), 7.40 (d, J=8.4 Hz, 1H), 7.29 (s, 1H), 7.20 (dd, J=8.4, 1.4 Hz, 1H), 7.08 (d, J=8.2 Hz, 2H), 6.24 (s, 1H), 3.09-2.97 (m, 4H). ¹³C NMR (126 MHz, CDCl₃) δ 139.80, 139.14, 136.60, 131.63, 130.13, 127.53, 123.00, 121.10, 120.16, 114.57, 113.31, 100.23, 34.88, 29.88. HRMS calc for (C₁₆H₁₃Br₂N—H)⁻ 377.9322, found 377.932.

### 6-bromo-2-(4-chlorophenethyl)-1H-indole (37c)

Yield=90%, white solid; ¹H NMR (500 MHz, CDCl₃) δ 7.75 (s, 1H), 7.44 (d, J=1.4 Hz, 1H), 7.40 (d, J=8.4 Hz, 1H), 7.29 (d, J=8.3 Hz, 2H), 7.20 (dd, J=8.4, 1.7 Hz, 1H), 7.13 (d, J=8.3 Hz, 2H), 6.24 (s, 1H), 3.10-2.97 (m, 41-1). ¹³C NMR (126 MHz, CDCl₃) δ 142.58, 139.28, 139.16, 136.59, 132.15, 129.73, 128.68, 127.52, 123.00, 121.09, 113.29, 100.22, 34.83, 29.97. HRMS calc for (C₁₆H₁₃BrClN—H)⁻ 333.9825, found 333.9825.

### Example 4: Synthesis of Compounds 38a and 39a-C

### General Procedure for the Synthesis of Compounds 38 and 39

To a stirred solution of either 36 or 37 (0.1 mmol) (see Examples 2 and 3) in DCM (1.6 mL) under Ar at 0° C. was added Et₂AlCl (1M in hexanes, 0.45 mmol) and the mixture was stirred at 0° C. for 30 min followed by the addition of the corresponding acid chloride (0.45 mmol) in DCM (1 mL). The mixture was stirred at 0° C. for 3h, quenched with saturated NaHCO₃ and partitioned between EtOAc and H₂O. The organic layer was washed with brine, dried over anhydrous Na₂SO₄ and concentrated. The crude product was purified by automated flash chromatography to give the desired adduct 38a or 39a, b, c or d.

### (E)-1-(6-bromo-2-styryl-1H-indol-3-yl)ethanone (38a)

Yield=56%, yellow solid; ¹H NMR (500 MHz, DMSO) δ 12.38 (s, 1H), 7.97 (d, J=1.7 Hz, 1H), 7.95 (d, J=5.9 Hz, 1H), 7.65 (d, J=7.5 Hz, 2H), 7.60 (d, J=1.2 Hz, 1H), 7.52 (s, 1H), 7.50-7.44 (m, 2H), 7.39 (t, J=7.3 Hz, 1H), 7.32 (dd, J=8.6, 1.4 Hz, 1H), 2.66 (s, 3H). ¹³C NMR (126 MHz, DMSO) δ 194.27, 142.29, 137.60, 136.54, 134.32, 129.55, 129.35, 127.39, 126.21, 124.81, 123.29, 118.56, 116.21, 114.76, 114.44, 32.11. HRMS calc for (C₁₈H₁₄BrNO—H)⁻ 338.0186, found 338.0183.

### 1-(6-bromo-2-phenethyl-1H-indol-3-yl)ethanone (39a)

Yield=57%, white solid; ¹H NMR (500 MHz, CDCl₃) δ 8.30 (s, 1H), 7.86 (d, J=8.6 Hz, 1H), 7.43 (d, J=1.5 Hz, 1H), 7.37 (dd, J=8.6, 1.7 Hz, 1H), 7.31 (d, J=7.0 Hz, 2H), 7.27-7.23 (m, 1H), 7.19 (d, J=7.0 Hz, 2H), 3.46 (t, J=7.4 Hz, 2H), 3.08 (t, J=7.4 Hz, 2H), 2.70 (s, 3H). ¹³C NMR (126 MHz, CDCl₃) δ 194.33, 147.59, 140.57, 135.25, 128.70, 128.48, 126.54, 125.60, 125.20, 122.07, 115.77, 114.13, 113.98, 35.04, 31.45, 30.89. HRMS calc for (C₁₈H₁₆BrNO—H)⁻ 340.0343, found 340.0338.

### 1-(6-bromo-2-(4-chlorophenethyl)-1H-indol-3-yl)ethanone (39b)

Yield=50%, colorless syrup; ¹H NMR (500 MHz, DMSO) δ 12.00 (s, 1H), 7.90 (d, J=8.6 Hz, 1H), 7.57 (d, J=1.8 Hz, 1H), 7.35 (d, J=8.4 Hz, 2H), 7.29 (dd, J=8.6, 1.8 Hz, 1H), 7.25 (d, J=8.4 Hz, 2H), 3.32 (dd, J=8.7, 7.3 Hz, 2H), 2.97 (dd, J=9.1, 6.9 Hz, 2H), 2.54 (s, 3H). ¹³C NMR (126 MHz, DMSO) δ 193.54, 148.33, 140.29, 136.24, 131.20, 130.60, 128.78, 126.03, 124.55, 122.80, 114.82, 114.51, 113.53, 34.39, 31.46, 30.27. HRMS calc for (C₁₈H₁₅BrClNO—H)⁻ 375.9931, found 375.9924.

### 1-(6-bromo-2-(4-chlorophenethyl)-1H-indol-3-yl)-2-methylpropan-1-one (39c)

Yield=58%, colorless syrup; ¹H NMR (500 MHz, DMSO) δ 12.03 (s, 1H), 7.79 (d, J=8.7 Hz, 1H), 7.58 (d, J=1.8 Hz, 1H), 7.33 (d, J=8.3 Hz, 2H), 7.29 (dd, J=8.6, 1.8 Hz, 1H), 7.23 (d, J=8.4 Hz, 2H), 3.41-3.27 (m, 3H), 2.98-2.91 (m, 2H), 1.11 (d, J=6.8 Hz, 6H). ¹³C NMR (126 MHz, DMSO) δ 200.24, 148.79, 140.33, 136.36, 131.17, 130.55, 128.74, 125.26, 124.56, 122.47, 114.65, 112.11, 38.43, 34.41, 30.40, 19.22. HRMS calc for (C₂₀H₁₉BrClNO—H)⁻ 404.02451, found 404.0246.

### Example 5: Synthesis of Compound 39d

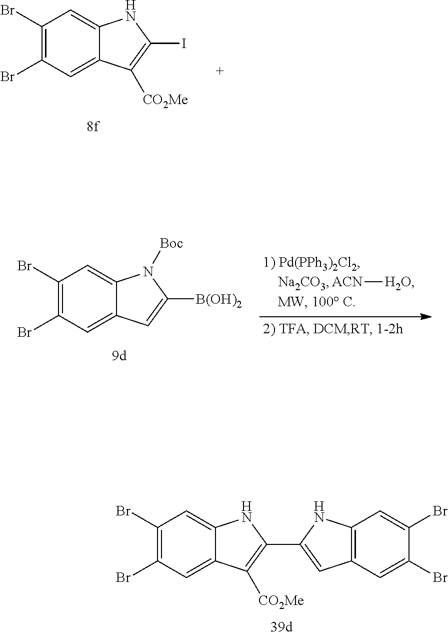

### Ethyl 5,6-dibromo-2-iodo-1H-indole-3-carboxylate (8f)

8f was prepared using general procedure as for the synthesis of substituted 2-iodo-1H-indole (8) (see Example 1). Yield=66%, white solid. ¹H NMR (500 MHz, CDCl₃) δ 8.60 (s, 1H), 8.42 (s, 1H), 7.68 (s, 1H), 4.00 (s, 3H).

### Specific Procedure for the Synthesis of (5,6-dibromo-1-(tert-butoxycarbonyl)-1H-indol-2-yl)boronic acid (9d)

To a stirred solution of tert-butyl 5,6-dibromo-1H-indole-1-carboxylate (0.6 g, 1.6 mmol) and triisopropylborate (0.5 mL, 2.2 mmol) in THF (6 mL) at 0° C. under N₂ was added LDA (1.8 M in THF, 1.1 mL, 2.0 mmol) dropwise. The mixture was stirred at 0° C. for 10 min and then at rt for 1 h. The mixture was acidified to pH ˜2 with 1M HCl and extracted with EtOAc. The organic phase was washed with brine, dried over anhydrous Na₂SO₄ and concentrated. The residue was suspended in Hexanes/EtOAc (6/1, 5 mL) and sonicated for 5 min and the resulting solid was collected by filtration to give the desired product (0.52g, 78%).

¹H NMR (500 MHz, DMSO) δ 8.42 (s, 1H), 8.35 (bs, 2H), 8.02 (s, 1H), 6.63 (s, J=9.4 Hz, 1H), 1.60 (s, 9H).

### Ethyl 5,5′,6,6′-tetrabromo-1H,1′H-[2,2′-biindole]-3-carboxylate (39d)

Compound 39d was prepared using the general procedure as for the synthesis of compounds 10, 12, 14, 17, 20, 22, 33 and 24 (see Example 1).

Yield=15%, brown solid. ¹H NMR (400 MHz, DMSO) δ 12.73 (s, 1H), 12.22 (s, 1H), 8.33 (s, 1H), 8.14 (s, 1H), 8.11 (s, 1H), 7.81 (s, 1H), 7.27 (s, 1H), 3.97 (s, 3H). ¹³C NMR (101 MHz, DMSO) δ 166.07, 137.77, 136.73, 130.58, 128.66, 128.21, 126.09, 126.07, 125.40, 118.16, 117.73, 117.57, 116.99, 116.81, 114.86, 104.00, 102.89, 52.41. HRMS calc for (C₁₈H₁₀Br₄N₂O₂—H)⁻ 604.7363, found 604.7343.

### Example 6: Synthesis of Compounds 90 and 91

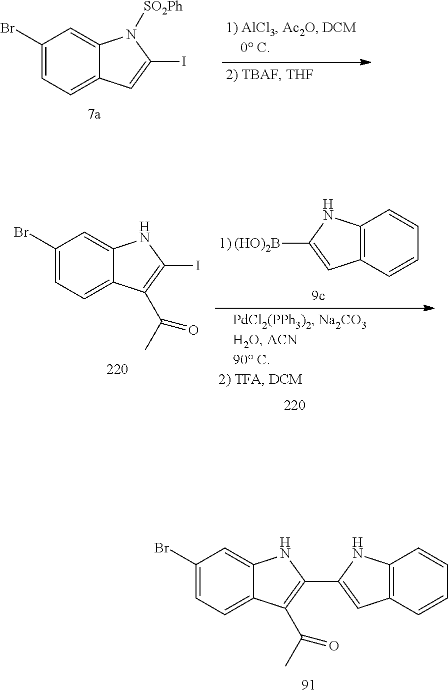

### Synthesis of Compound 220

Acetic anhydride (3.0 mmol) was added to a stirred suspension of AlCl₃ (6.0 mmol) in DCM at 0° C. and the mixture was stirred at 0° C. for 15 min. A solution of 7a (1.0 mmol) in DCM was added and the mixture was stirred at rt for 1h. The reaction was quenched with ice water and then extracted with DCM. The organic phase was washed saturated aqueous NaHCO₃, H₂O, brine, dried over anhydrous Na₂SO₄ and concentrated. The residue was dissolved in THF followed by the addition of TBAF (1M in THF, 1.0 mmol) and the mixture was stirred at rt for 3h. The reaction mixture was diluted with EtOAc and washed with H₂O, brine, dried over anhydrous Na₂SO₄ and concentrated. The crude product was purified by automated flash chromatography to give the desired product 220 as colorless solid (56%).

### Synthesis of 1-(6-bromo-1H,1′H-[2,2′-biindol]-3-yl)ethan-1-one (91)

Compound 91 was prepared from 220 and 9c by using the procedure described to synthesize compounds 10, 12, 14, 17, 20, 22, 33 and 24 (see Scheme 1; FIG. 2). Yield=56%, yellow solid. HRMS calculated for (C₁₈H₁₃BrN₂O—H)⁻ 353.0120, found 353.0129.

### Synthesis of methyl 6′-bromo-1H,1′H-[2,2′-biindole]-3-carboxylate (90)

Compound 41 was prepared by coupling 9a with 1-(tert-butyl)-3-methyl 2-iodo-1H-indole-1,3-dicarboxylate using the procedure described to synthesize compounds 10, 12, 14, 17, 20, 22, 33 and 24 (see Scheme 1; FIG. 2). ¹³C NMR (151 MHz, DMSO) δ 166.65, 137.16, 135.94, 135.88, 129.70, 126.84, 126.37, 123.46, 122.90, 122.26, 121.79, 121.66, 115.54, 114.79, 111.69, 103.26, 102.55, 51.62. HRMS calculated for (C₁₈H₁₃BrN₂O₂—H)⁻ 367.0087, found 367.0095.

### Example 7: Synthesis of Compounds 42 and 87

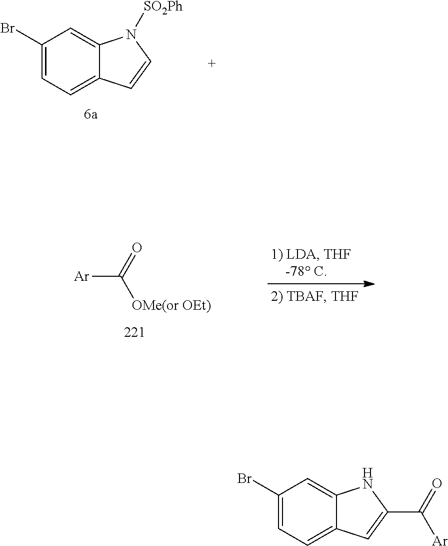

To a stirred solution of 6a (1 mmol) in THF (16 mL) under argon at −78° C. was added LDA (1.7M in THF, 1.3 mmol) and the mixture was stirred at 0° C. for 30 min. The mixture was re-cooled to −78° C. followed by the addition of the corresponding ester (221) (1.1 mmol) in THF (2 mL). The mixture was stirred at −78° C. for 30 min and then slowly warmed to rt over 1h. The reaction was quenched with ice water and then extracted with EtOAc. The organic phase was washed with H₂O, brine, dried over anhydrous Na₂SO₄ and concentrated. The residue was dissolved in THF followed by the addition of TBAF (1M in THF, 3.0 mmol) and the mixture was stirred at rt for 3h. The reaction mixture was diluted with EtOAc and washed with H₂O, brine, dried over anhydrous Na₂SO₄ and concentrated. The crude product was purified by automated flash chromatography to give the desired product.

### (6-Bromo-1H-indol-2-yl)(3-hydroxynaphthalen-2-yl)methanone (42)

Compound 42 was prepared from 6a and methyl 3-((tert-butyldimethylsilyl)oxy)-2-naphthoate. ¹H NMR (600 MHz, DMSO-d₆) δ 12.13 (s, 1H), 10.21 (s, 1H), 8.11 (s, 1H), 7.93 (d, J=8.2 Hz, 1H), 7.79 (d, J=8.3 Hz, 1H), 7.69-7.62 (m, 2H), 7.55-7.49 (m, 1H), 7.39-7.33 (m, 1H), 7.32 (s, 1H), 7.23 (dd, J=8.5, 1.8 Hz, 1H), 6.99 (s, 1H). 13C NMR (151 MHz, DMSO) δ 186.98, 152.95, 138.74, 135.36, 129.74, 128.59, 128.54, 127.75, 126.72, 125.91, 125.85, 124.83, 123.58, 123.43, 118.58, 115.11, 112.50, 110.04.

### (6-Bromo-1H-indol-2-yl)(5-bromo-2-hydroxyphenyl)methanone (87)

Compound 87 was prepared from 6a and ethyl 5-bromo-2-((triisopropylsilyl)oxy)benzoate. Yield=44%, yellow solid. ¹H NMR (400 MHz, CDCl₃) δ 11.46 (s, 1H), 9.18 (s, 1H), 8.25 (d, J=2.4 Hz, 1H), 7.67-7.63 (m, 2H), 7.62 (dd, J=8.9, 2.4 Hz, 1H), 7.32 (dd, J=8.5, 1.7 Hz, 1H), 7.29 (dd, J=2.2, 0.9 Hz, 1H), 7.00 (d, J=8.9 Hz, 1H).

### Example 8: Synthesis of Compounds 43 and 49

Compounds 43 and 49 were prepared following the general procedure described to synthesize compounds 33a and b (Example 1).

### 2,5-bis(5-Bromo-1H-indol-2-yl)thiophene (43)

Compound 43 was prepared from 9b and 2,5-diiodothiophene. ¹H NMR (600 MHz, DMSO-d₆): δ 11.86 (d, J=1.5 Hz, 2H), 7.72 (d, J=1.8 Hz, 2H), 7.56 (s, 2H), 7.34 (d, J=8.5 Hz, 2H), 7.23 (dd, J=1.9 Hz, 8.5 Hz, 2H), 6.73 (d, J=1.5 Hz, 2H). ¹³C NMR (150 MHz, DMSO-d₆): δ 135.6 (2C), 133.9 (2C), 133.3 (2C), 130.3 (2C), 125.0 (2C), 124.4 (2C), 122.0 (2C), 113.0 (2C), 112.1 (2C), 98.7 (2C).

### 3,6-bis(6-Bromo-1H-indol-2-yl)pyridazine (49)

49 was prepared from 9a and 3,6-diiodopyridazine. ¹H NMR (400 MHz, DMSO-d₆): δ 12.13 (s, 2H), 8.35 (s, 2H), 7.69 (bs, 2H), 7.62 (d, J=8.5 Hz, 2H), 7.41 (d, J=1.4 Hz, 2H), 7.19 (dd, J=1.7 Hz, 8.5 Hz, 2H). ¹³C NMR (100 MHz, DMSO-d₆): δ 151.0 (2C), 138.5 (2C), 135.0 (2C), 127.1 (2C), 124.4 (2C), 122.74 (2C), 122.68 (2C), 115.8 (2C), 114.6 (2C), 103.1 (2C).

### Example 9: Synthesis of Compounds 47, 81, 59, 60, 61 and 86

Compounds 47, 81, 59, 60, 61 and 86 were prepared following the procedure described to synthesize compound 33c-e.

### 6-Bromo-2-(5-(5-bromo-1H-indol-2-yl)thiophen-2-yl)-1H-indole (47)

¹H NMR (500 MHz, DMSO-d₆): δ 11.87 (s, 1H), 11.82 (s, 1H), 7.71 (d, J=1.7 Hz, 1H), 7.56-7.53 (m, 3H), 7.49 (d, J=8.4 Hz, 1H), 7.35 (d, J=8.6 Hz, 1H), 7.23 (dd, J=1.9 Hz, 8.6 Hz, 1H), 7.15 (dd, J=1.7 Hz, 8.4 Hz, 1H), 6.76 (d, J=1.4 Hz, 1H), 6.72 (d, J=1.4 Hz, 1H). ¹³C NMR (125 MHz, DMSO-d₆): δ 137.8, 135.7, 134.0, 133.8, 133.3, 132.8, 130.4, 127.5, 125.1, 124.9, 124.4, 122.6, 122.1, 121.7, 114.5, 113.6, 113.1, 122.1, 99.4, 98.7.

### 2-(5-(1H-Pyrazol-5-yl)thiophen-2-yl)-6-bromo-1H-indole (81)

Yield=35%, yellow solid. ¹H NMR (400 MHz, DMSO-d₆) δ 12.96 (s, 1H), 11.76 (d, J=2.2 Hz, 1H), 7.79 (d, J=2.4 Hz, 1H), 7.55-7.45 (m, 3H), 7.42 (d, J=3.7 Hz, 1H), 7.14 (dd, J=8.4, 1.8 Hz, 1H), 6.73 (dd, J=2.1, 0.9 Hz, 1H), 6.67 (d, J=2.3 Hz, 1H).

### 2-(4′-Bromo-[1,1′-biphenyl]-3-yl)-1H-indole (59)

Yield=75%, pale mauve solid. ¹H NMR (400 MHz, DMSO-d₆) δ 7.02 (1H, tm, J₅₋₄=J₅₋₆=7.8 Hz, H5), 7.03 (1H, m, H3), 7.13 (1H, tm, J₆₋₅=J₆₋₇=7.8 Hz, H6), 7.43 (1H, d, J₇₋₆=7.8 Hz, H7), 7.55 (1H, t, J_{5′-4′}=J_{5′-6′}=7.6 Hz, H5′), 7.56 (1H, d, J₄₋₅=7.8 Hz, H4), 7.62 (1H, d, J_{4′-5′}=7.6 Hz, H4′), 7.72 (2H, d, J_{2″-3″}=J_{6″-5″}=8.6 Hz, H2″ and H6″), 7.78 (2H, d, J_{3″-2″}=J_{5″-6″}=8.6 Hz, H3″ and H5″), 7.89 (1H, d, J_{6′-5′}=7.6 Hz, H6′), 8.19 (1H, s, H2′), 11.64 (1H, s, indolic H). ¹³C NMR (100 MHz, DMSO-d₆): δ 99.7 (C3), 111.8 (C7), 119.9 (C6), 120.6 (C4), 121.6 (C4″), 122.2 (C5), 123.4 (C2′), 125.1 (C6′), 126.0 (C4′), 129.1 (C3a), 129.4 (C2″ and C6″), 130.1 (C5′), 132.3 (C3″ and C5″), 133.4 (C2 or C1′), 137.6 (C7a), 137.8 (C2 or C1′), 139.6 (C1″), 139.9 (C3′). HRMS calculated for (C₂₀H₁₄⁷⁹BrN+H)⁺ 348.0382, found 348.0397.

### 2-(6-(4-Bromophenyl)pyrazin-2-yl)-1H-indole (60)

Yield=14%, yellow solid. ¹H NMR (400 MHz, DMSO-d₆) δ 7.07 (1H, tm, J₅₋₄=J₅₋₆=7.2 Hz, H5), 7.22 (1H, tm, J₆₋₅=J₆₋₇=7.2 Hz, H6), 7.43 (1H, m, H3), 7.56 (1H, d, J₇₋₆=7.2 Hz, H7), 7.65 (1H, d, J₄₋₅=7.2 Hz, H4), 7.80 (2H, d, =J_{5″-6″}=8.4 Hz, H3″ and H5″), 8.39 (2H, d, J_{2″-3″}=J_{6″-5″}=8.4 Hz, H2″ and H6″), 9.16 (1H, s, H3′), 9.26 (1H, s, H5′), 11.85 (1H, s, indolic H). ¹³C NMR (100 MHz, DMSO-d₆): δ 103.1 (C3), 112.6 (C7), 120.4 (C5), 121.5 (C7), 123.7 (C6), 124.4 (C4″), 128.8 (C3a), 129.5 (C2″ and C6″), 132.3 (C3″ and C5″), 134.6 (C2), 135.4 (C1″), 138.1 (C7a), 139.4 (C3′), 141.0 (C5′), 145.8 (C2′), 149.4 (C6′). HRMS calculated for (C₁₈H₁₂⁷⁹BrN₃+H)⁺ 350.0287, found 350.0301.

### 2-(6-(4′-Bromo-[1,1′-biphenyl]-4-yl)pyrazin-2-yl)-1H-indole (61)

61 was obtained as a side product during the synthesis of compound 60. Yield=14%, yellow solid. ¹H NMR (400 MHz, DMSO-d₆) δ 7.07 (1H, tm, J₅₋₄=J₅₋₆=7.2 Hz, H5), 7.23 (1H, tm, J₆₋₅=J₆₋₇=7.2 Hz, H6), 7.44 (1H, m, H3), 7.58 (1H, d, J₇₋₆=7.2 Hz, H7), 7.66 (1H, d, J₄₋₅=7.2 Hz, H4), 7.71 (2H, d, J_{8″-9″}=J_{12″-11″}=8.8 Hz, H8″ and H12″), 7.78 (2H, d, J_{9″-8″}=J_{11″-12″}=8.8 Hz, H9″ and H11″), 7.90 (2H, d, J_{3″-2″}=J_{5″-6″}=8.4 Hz, H3″ and H5″), 8.53 (2H, d, J_{2″-3″}=J_{6″-5″}=8.4 Hz, H2″ and H6″), 9.20 (1H, s, H3′), 9.25 (1H, s, H5′), 11.87 (1H, s, indolic H). ¹³C NMR (100 MHz, DMSO-d₆): δ 102.9 (C3), 112.6 (C7), 120.3 (C5), 121.5 (C7), 121.9 (C10″), 123.7 (C6), 127.5 (C2″ and C6″), 128.1 (C3″ and C5″), 128.8 (C3a), 129.4 (C9″ and C11″), 132.4 (C8″ and C12″), 134.7 (C2), 135.5 (C1″), 138.1 (C7a), 139.0 (C4″), 139.6 (C3′), 140.8 (C5′), 140.8 (C7″), 145.9 (C2′), 150.0 (C6′). HRMS calculated for (C₂₄H₁₆⁷⁹BrN₃+H)⁺ 426.0600, found 426.0617.

### 4-bromo-2-(2-(6-bromo-1H-indol-2-yl)pyridin-4-yl)phenol (86)

Yield=45%, dark yellow solid.

### Example 10: Synthesis of Compounds 45 and 48

Compound 45 was synthesized following the general procedure described to prepare compound 25 (Example 1), and compound 48 was synthesized following the general procedure described to prepare compound 26 (Example 1).

### 1,2-bis(5-Bromo-1H-indol-2-yl)ethyne (45)

Mass calculated for (C₁H₁₁Br₂N₂—H)⁻ 412.9, found 412.8.

### 5-Bromo-2-((5-bromo-1H-indol-2-yl)ethynyl)-1-methyl-1H-indole (48)

¹H NMR (500 MHz, DMSO-d₆) δ 12.05 (bs, 1H), 7.81-7.76 (m, 2H), 7.50 (d, J=8.8 Hz, 1H), 7.41-7.29 (m, 3H), 6.94-6.90 (m, 2H), 3.90 (s, 3H).

### Example 11: Synthesis of Compounds 50, 51 and 78

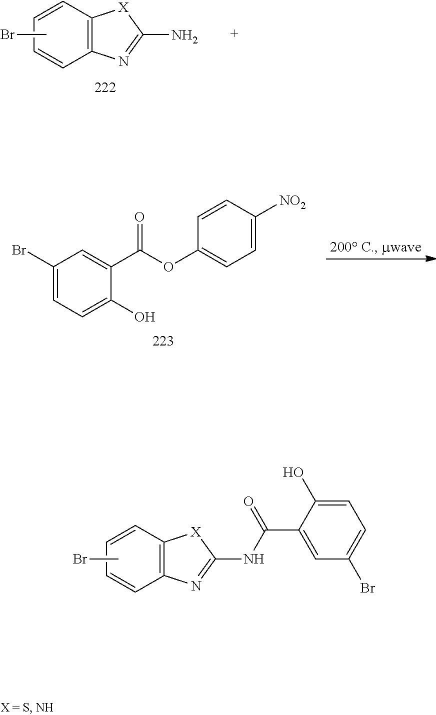

### General Procedure

A mixture of the corresponding amine 222 (1 mmol) and 4-nitrophenyl 5-bromo-2-hydroxybenzoate 223 (1.1 mmol) was heated at 200° C. by microwave for 80 minutes. The solid was taken up in ethanol and heated at reflux temperature for 20 minutes. The precipitate was collected by filtration and then triturated with diethyl ether to give the desired product.

### 5-Bromo-N-(5-bromobenzo[d]thiazol-2-yl)-2-hydroxybenzamide (50)

Yield=43%, off-white solid. ¹HNMR (600 MHz, DMSO-d₆) δ 7.03 (d, J=8.7 Hz, 1H), 7.52 (dd, J=8.5 Hz, J=1.4 Hz, 1H), 7.64 (dd, J=8.7 Hz, J₄₋₆=2.3 Hz, 1H), 7.94 (bs, 1H), 8.00 (d, J=8.5 Hz, 1H), 8.04 (d, J=2.3 Hz, 1H), 12.25 (bs, 1H). ¹³C NMR (125 MHz, DMSO-d₆): δ 111.0 119.7, 119.9, 120.1, 124.5, 127.1, 132.8, 137.3. HRMS calculated for (C₁₄H₈BrBrN₂O₂S+H)⁺ 428.8726, found 428.8774.

### 5-Bromo-N-(6-bromobenzo[d]thiazol-2-yl)-2-hydroxybenzamide (51)

Yield=63%, off-white solid. ¹HNMR (600 MHz, DMSO-d₆) δ 7.02 (d, J=8.7 Hz, 1H), 7.62 (dd, J=8.5 Hz, J=1.9 Hz, 1H), 7.64 (dd, J=8.7 Hz, J=2.6 Hz, 1H), 7.68 (bm, 1H), 8.05 (d, J=2.6 Hz, 1H), 8.30 (1H, d, J=1.9 Hz, 1H), 12.32 (1H, bs, 1H)¹³C NMR (125 MHz, DMSO-d₆): δ 110.5, 115.9, 119.3, 119.6, 122, 124.6, 129.5, 132.3, 136.8, 157.0, 165. HRMS calculated for (C₁₄H₈BrBr N₂O₂S+H)⁺ 428.8726, found 428.8768.

### 5-Bromo-N-(6-bromo-1H-benzo[d]imidazol-2-yl)-2-hydroxybenzamide (78)

Yield=41%, brown solid. ¹HNMR (600 MHz, DMSO-d₆) δ 6.88 (d, J=8.8 Hz, 1H), 7.42 (m, 2H), 7.51 (dd, J=8.8 Hz, J=2.7 Hz, 1H), 7.65 (bm, 1H), 8.05 (d, J=2.7 Hz, 1H), 12.32 (bs, 1H). ¹³C NMR (125 MHz, DMSO-d₆): δ 109.6, 114.2, 115.1, 115.3, 120.2, 121.8, 126.3, 132.2, 136.3, 160.1.

### Example 12: Synthesis of Compounds 53, 54, 62-64, 69, 77, 79, 80, 82 and 84

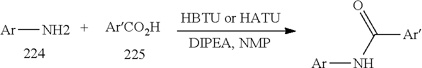

### General Procedure

To a mixture of 225 (1 mmol) and HBTU or HATU (1 mmol) in NMP (12 mL) were added 224 (1 mmol) and DIPEA (2-3 mmol). The reaction mixture was stirred at rt for 12-24h, quenched with H₂O and extracted with EtOAc. The organic layer was washed with H₂O, brine, dried over anhydrous Na₂SO₄ and concentrated. The crude product was purified by automated flash chromatography to give the desired product.

### N-(5-Bromobenzo[d]thiazol-2-yl)-1H-indole-2-carboxamide (53)

Yield=13%, white solid. ¹HNMR (500 MHz, DMSO-d₆) δ 7.11 (t, J=7.6 Hz, 1H), 7.29 (t, J=7.6 Hz, 1H), 7.50 (d, J=7.6 Hz, 1H), 7.51 (dd, J=8.5 Hz, J=1.8 Hz, 1H), 7.71 (d, J=7.6 Hz, 1H), 7.77 (s, 1H), 8.00 (bs, 1H), 8.02 (d, J=8.5 Hz, 1H), 12.03 (s, 1H), 13.11 (s, 1H). ¹³C NMR (150 MHz, DMSO-d₆): δ 107.3, 113.1, 119.4, 120.8, 122.9, 123.2, 124.1, 125.4, 126.7, 127.4, 129.2, 131.4, 138.1, 150.7, 160.4, 160.6. HRMS calculated for (C₁₆H₁₀BrN₃OS+H)⁺ 371.9806, found 371.9839.

### N-(6-Bromobenzo[d]thiazol-2-yl)-1H-indole-2-carboxamide (54)

Yield=10%, white solid. ¹HNMR (500 MHz, DMSO-d₆) δ 7.11 (t, J=7.6 Hz, 1H), 7.29 (t, J=7.6 Hz, 1H), 7.50 (d, J=7.6 Hz, 1H), 7.62 (dd, J=8.6 Hz, J=2.0 Hz, 1H), 7.71 (d, J=7.6 Hz, 1H), 7.74 (m, 1H), 7.76 (s, 1H), 8.32 (d, J=2.0 Hz, 1H), 12.02 (s, 1H), 13.05 (s, 1H). ¹³C NMR (150 MHz, DMSO-d₆): δ 107.2, 113.1, 116.0, 120.7, 122.5, 122.8, 124.7, 125.4, 127.4, 129.7, 138.1, 148.0. HRMS calculated for (C₁₆H₁₀BrN₃OS+H)⁺ 371.9806, found 371.9841.

### 6-Bromo-N-(5-bromo-2-hydroxyphenyl)-1H-indole-2-carboxamide (62)

Yield=15%, pink solid. ¹HNMR (400 MHz, DMSO-d₆) δ 6.91 (d, J=8.6 Hz, 1H), 7.21 (m, 2H), 7.39 (m, 1H), 7.64 (m, H1), 7.65 (d, J=8.6 Hz, 1H), 7.94 (d, J=2.5 Hz, 1H), 9.55 (s, 1H), 10.26 (s, 1H), 11.94 (s, 1H). ¹³C NMR (125 MHz, DMSO-d₆): δ 104.7, 110.0, 115.3, 117.2, 117.9, 123.5, 124.2, 126.5, 126.6, 127.5, 128.4, 132.3, 138.1, 149.1, 159.8. HRMS calculated for (C₅H₁₁Br₂N₂O₂+H)⁺ 410.9162, found 410.9175.

### 6-Bromo-N-(4-bromophenyl)-1H-indole-2-carboxamide (63)

Yield=31%, white solid. ¹HNMR (400 MHz, DMSO-d₆) δ 7.08 (dd, J=8.6 Hz, J=1.8 Hz, 1H), 7.46 (bs, 1H), 7.57 (2H, d, J=8.7 Hz, 2H), 7.64 (d, J=1.8 Hz, 1H), 7.68 (d, J=8.6 Hz, 1H), 7.79 (d, J=8.7 Hz, 2H), 10.40 (s, 1H), 11.92 (s, 1H). ¹³C NMR (125 MHz, DMSO-d₆): δ 104.7, 115.3, 115.8, 117.2, 122.5, 123.5, 124.2, 126.4, 132.0, 132.5, 138.0, 138.7, 159.9. HRMS calculated for (C₁₅H₁₀⁷⁹Br⁸¹BrN₂O+H)⁺ 394.9213, found 394.9232.

### 6-Bromo-N-(3-bromo-2-hydroxyphenyl)-1H-indole-2-carboxamide (64)

Yield=26%, pink solid. ¹HNMR (400 MHz, DMSO-d₆) δ 6.86 (1H, t, 8.0 Hz, H5′), 7.22 (1H, dd, J₅₋₆=8.6 Hz, J₅₋₃=1.8 Hz, H5), 7.43 (1H, s, H3), 7.45 (1H, dm, H4′), 7.47 (1H, dm, H6′), 7.65 (1H, d, J₇₋₅=1.8 Hz, H7), 7.68 (1H, d, J₄₋₅=8.6 Hz, H4), 9.80 (1H, s, amide H), 10.11 (1H, s, phenolic H), 11.97 (1H, s, indolic H). ¹³C NMR (125 MHz, DMSO-d₆): δ 105.1 (C3), 112.2 (C3′), 115.3 (C7), 117.2 (C6), 121.1 (C5′), 123.5 (C5), 124.2 (C4), 125.7 (C6′), 126.5 (C3a), 127.5 (C1′), 130.5 (C4′), 132.1 (C2), 138.0 (C7a), 148.0 (C2′), 160.8 (C8). HRMS calculated for (C₁₅H₁₀⁷⁹Br⁸¹BrN₂O₂+H)⁺ 410.9162, found 410.9176.

### N-(6-Bromobenzo[d]thiazol-2-yl)-1-methyl-1H-indole-2-carboxamide (69)

Yield=10%, white solid. ¹HNMR (500 MHz, DMSO-d₆) δ 4.09 (3H, s, methyl), 7.17 (1H, t, J₅₋₄=J₅₋₆=7.6 Hz, H5), 7.38 (1H, t, J₆₋₅=J₆₋₇=7.6 Hz, H6), 7.61 (1H, dd, J_{5′-4′}=8.5 Hz, J_{5′-7′}=2.1 Hz, H5′), 7.62 (1H, d, J₄₋₅=7.6 Hz, H4), 7.72-7.74 (3H, m, H3, H7 & H4′), 8.30 (1H, d, J_{7′-5′}=2.1 Hz, H7′), 12.99 (1H, s, amide H). ¹³C NMR (150 MHz, DMSO-d₆): δ 109.1 (C3), 111.3 (C4), 116.0 (C6′), 121.1 (C5), 122.9 (C7), 124.7 (C7′), 125.6 (C6), 125.8 (C3a), 129.7 (C5′), 140.0 (C7a). HRMS calculated for (C₁₇H₁₂⁷⁹BrN₃OS+Na)⁺ 407.9777, found 407.9781.

### 5-Bromo-N-(5-bromobenzo[d]thiazol-2-yl)-1H-indole-2-carboxamide (77)

Yield=2%, pale green solid. ¹HNMR (400 MHz, DMSO-d₆) δ 7.39 (1H, dd, J₆₋₇=8.8 Hz, J₆₋₄=1.4 Hz, H6), 7.45 (1H, d, J₇₋₆=8.8 Hz, H7), 7.29 (1H, t, J₅₋₄=J₅₋₆=7.6 Hz, H5), 7.51 (1H, dd, J_{6′-7′}=8.8 Hz, J_{6′-4′}=1.4 Hz, H6′), 7.72 (1H, s, H3), 7.95 (1H, s, H4), 7.99 (1H, s, H4′), 8.01 (1H, d, J_{7′-6′}=8.8 Hz, H7′), 12.21 (1H, s, indolic H), 13.17 (1H, s, amide H). ¹³C NMR (100 MHz, DMSO-d₆): δ 106.5 (C3), 113.2 (C5), 115.1, 119.4 (C5′), 124.1 (C7′), 124.9, 126.7 (C6′), 127.9, 129.1 (C3a), 136.6 (C7a). HRMS calculated for (C₁₆H₉⁷⁹Br⁸¹BrN₃OS—H)⁻ 449.8734, found 449.8657.

### 5-bromo-N-(6-bromo-1H-benzo[d]imidazol-2-yl)-1H-indole-2-carboxamide (79)

Yield=9%, beige solid. ¹HNMR (400 MHz, DMSO-d₆) δ 7.28 (1H, dd, J_{5′-4′}=8.4 Hz, J_{5′-7′}=2.0 Hz, H5′), 7.38 (1H, dd, J₆₋₇=8.8 Hz, J₆₋₄=1.8 Hz, H6), 7.45 (1H, d, J_{4′-5′}=8.8 Hz, H4′), 7.48 (1H, d, J₇₋₆=8.8 Hz, H7), 7.60 (1H, bs, H3), 7.67 (1H, d, J_{7′-5′}=2.0 Hz, H7′), 7.92 (1H, d, J₄₋₆=1.8 Hz, H4). ¹³C NMR (100 MHz, DMSO-d₆): δ 105.9 (C3), 113.0 (C5), 113.8 (C3a′), 115.0 (C7), 124.5 (C5′), 124.7 (C4), 127.4 (C6), 129.2 (C3a), 136.3 (C7a).

### 6-Bromo-N-(6-bromo-1H-benzo[d]imidazol-2-yl)-1H-indole-2-carboxamide (80)

Yield=7%, brown solid. ¹HNMR (400 MHz, DMSO-d₆) δ 7.23 (11-1, dd, J₅₋₄=8.8 Hz, J₅₋₇=1.8 Hz, H5), 7.29 (1H, dd, J_{5′-4′}=8.4 Hz, J_{5′-7′}=2.0 Hz, H5′), 7.45 (1H, d, J_{4′-5′}=8.8 Hz, H4′), 7.66-7.69 (4H, m, H3, H4, H7, H7′), 11.92 (1H, s, indolic H), 12.32 (2H, bs, amide H & benzimidazolic H). ¹³C NMR (100 MHz, DMSO-d₆): δ 106.6 (C3), 115.4 (C7), 117.7 (C6), 123.7 (C5), 124.6 (C4 & C5′), 126.5 (C3a), 138.4 (C7a).

### 5-Bromo-N-(6-bromobenzo[d]thiazol-2-yl)-1H-indole-2-carboxamide (82)

Yield=3%, beige solid. ¹HNMR (400 MHz, DMSO-d₆) δ 7.40 (1H, dd, J₆₋₇=8.8 Hz, J₆₋₄=1.4 Hz, H6), 7.46 (1H, d, J₇₋₆=8.8 Hz, H7), 7.62 (1H, dd, J_{5′-4′}=8.8 Hz, J_{5′-7′}=1.8 Hz, H5′), 7.73 (2H, m, H3 & H4′), 7.95 (1H, d, J₄₋₆=1.4 Hz, H4), 8.31 (1H, d, 5=1.8 Hz, H7′), 12.22 (1H, s, indolic H), 13.09 (1H, s, amide H). ¹³C NMR (100 MHz, DMSO-d₆): δ 106.5 (C3), 113.2 (C5), 115.1 (C7), 116.1 (C6′), 122.4 (C4′), 124.8 (C7′), 124.9 (C4), 127.9 (C6), 129.1 (C3a), 129.7 (C5′), 130.6 (C3a′), 134.2 (C7a′), 136.6 (C7a).

### 3-Bromo-N-(6-bromobenzo[d]thiazol-2-yl)-2-methoxybenzamide (84)

Yield=23%, white solid. ¹HNMR (400 MHz, DMSO-d₆) δ 3.83 (3H, s, methoxy), 7.24 (1H, t, J₅₋₄=J₅₋₆=7.8 Hz, H5), 7.62 (1H, dd, J_{5′-4′}=8.6 Hz, J_{5′-7′}=2.0 Hz, H5′), 7.66 (1H, dd, J₄₋₅=7.6 Hz, J₄₋₆=1.6 Hz, H4), 7.74 (1H, d, J_{4′-5′}=8.6 Hz, H4′), 7.86 (1H, dd, J₆₋₅=7.6 Hz, J₆₋₄=1.6 Hz, H6), 8.32 (1H, d, J_{7′-5′}=2.0 Hz, H7′), 12.85 (1H, s, amide H). ¹³C NMR (100 MHz, DMSO-d₆): δ 62.6 (methoxy), 116.3 (C6′), 117.5 (C5), 122.9 (C4′), 124.8 (C7′), 126.2 (C5), 129.6 (C4), 129.8 (C5′), 130.6 (C1), 134.2 (C7a′), 148.2 (C3a′), 136.6 (C6), 154.9 (C2), 165.7 (C7).

### Example 13: Synthesis of Compound 71

To a mixture of 6-bromo-benzothiophene-2-carboxylic acid (500 mg, 1.95 mmol, 1.0 eq.) and EDCI.HCl (1.12 g, 5.85 mmol, 3.0 eq.) in NMP (30 mL) were added 4-bromoaniline (335 mg, 1.95 mmol, 1.0 eq.). The reaction mixture was stirred at room temperature for 2d, quenched with H₂O and extracted with EtOAc. The organic layer was washed successively with 1M aqueous NaOH, 1M aqueous HCl and H₂O, and then dried over Na₂SO₄. The solvent was evaporated under reduced pressure and the residue was triturated with methyl tert-butyl ether to afford compound 71 (600 mg, 31%) as an orange solid.

### 6-bromo-N-(4-bromophenyl)benzo[b]thiophene-2-carboxamide (71)

¹HNMR (400 MHz, DMSO-d₆) δ 7.57 (2H, d, J_{3′-2′}=J_{5′-6′}=8.9 Hz, H3′ and H5′), 7.63 (1H, dd, J₅₋₄=8.5 Hz, J₅₋₇=1.8 Hz, H5), 7.74 (2H, d, J_{2′-3′}=J_{6′-5′}=8.9 Hz, H2′ and H6′), 7.97 (1H, d, J₄₋₅=8.5 Hz, H4), 8.34 (1H, bs, H3), 8.38 (1H, d, J₇₋₅=1.8 Hz, H7), 10.66 (1H, s, amide H). ¹³C NMR (125 MHz, DMSO-d₆): δ 115.8 (C4′), 119.9 (C6), 122.2 (C2′ and C6′), 125.4 (C7), 125.7 (C3), 127.1 (C4), 128.3 (C5), 131.6 (C3′ and C5′), 137.9 (C1′), 138.0 (C3a), 140.5 (C2), 142.1 (C7a), 160.1 (C8). HRMS calculated for (C₁₅H₉⁷⁹Br⁸¹BrNOS+H)⁺ 411.8824, found 411.8827.

### Example 14: Synthesis of Compounds 67, 68, 70 and 83

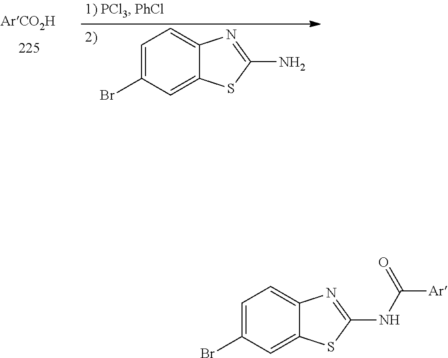

### General Procedure

A mixture of compound 225 (1.0 mmol) and PCl₃ (0.5 mmol) in chlorobenzene was refluxed for 15 min and then cooled to rt. 6-bromobenzo[d]thiazol-2-amine (1.0 mmol) was added and the reaction mixture was refluxed for 3 h. After cooling to rt, the mixture was diluted with EtOH, filtered and the solid was triturated with methyl tert-butylether to afford the desired adduct.

### 4-Bromo-N-(6-bromobenzo[d]thiazol-2-yl)-2-hydroxybenzamide (67)

Yield=31%, beige solid. ¹HNMR (600 MHz, DMSO-d₆) δ 7.20 (11-1, d, J₅₋₆=8.1 Hz, H5), 7.27 (1H, s, H3), 7.62 (1H, dm, J_{5′-4′}=8.5 Hz, H5′), 7.68 (1H, bm, H4′), 7.88 (1H, d, J₆₋₅=8.1 Hz, H6), 8.30 (1H, bs, H7′), 12.25 (1H, bs, amide H). ¹³C NMR (150 MHz, DMSO-d₆): δ 116.3 (C6′), 117.4 (C4), 120.3 (C3), 122 (C4′), 123.2 (C5), 125.1 (C7′), 127.8 (C1), 130.0 (C5′), 132.6 (C6), 168.2 (C7). HRMS calculated for (C₁₄H₈⁷⁹Br⁸¹BrN₂O₂S+H)⁺ 428.8726, found 428.8745.

### 5-Bromo-N-(6-bromobenzo[d]thiazol-2-yl)-2-methoxybenzamide (68)

Yield=61%, yellow solid. ¹HNMR (600 MHz, DMSO-d₆) δ 3.92 (3H, s, methoxy), 7.21 (1H, d, J₃₋₄=8.9 Hz, 1-13), 7.61 (1H, dd, J_{5′-4′}=8.6 Hz, J_{5′-7′}=1.9 Hz, H5′), 7.73 (1H, d, J_{4′-5′}=8.9 Hz, H4′), 7.75 (1H, dd, J₄₋₃=8.9 Hz, J₄₋₆=2.6 Hz, H4), 7.82 (1H, d, J₆₋₄=2.6 Hz, H6), 8.31 (1H, d, J_{7′-5′}=1.9 Hz, H7′), 12.36 (1H, bs, amide H). ¹³C NMR (150 MHz, DMSO-d₆): δ 57.0 (methoxy), 112.3 (C5), 115.2 (C3), 116.2 (C6′), 122.8 (C4′), 124.6 (C1), 124.9 (C7′), 129.8 (C5′), 132.6 (C6), 134.2 (C7a′), 136.1 (C4), 148.2 (C3a′), 156.8 (C2), 158.9 (C2′), 164.5 (bs, C7). HRMS calculated for (C₁₅H₁₀⁷⁹Br⁸¹BrN₂O₂S+H)⁺ 442.8882, found 442.8909.

### 3-Bromo-N-(6-bromobenzo[d]thiazol-2-yl)-2-hydroxybenzamide (70)

Yield=9%, beige solid. ¹HNMR (400 MHz, DMSO-d₆) δ 6.92 (1H, t, J₅₋₄=J₅₋₆=7.8 Hz, H5), 7.62 (1H, d, J_{4′-5′}=8.6 Hz, H4′), 7.67 (1H, dd, J_{5′-4′}=8.6 Hz, J_{5′-7′}=1.9 Hz, H5′), 7.80 (1H, J₄₋₅=7.8 Hz, H4), 8.31 (1H, d, J_{7′-5′}=1.9 Hz, H7′). ¹³C NMR (150 MHz, DMSO-d₆): δ 111.1 (C3), 116.0 (C6′), 119.9 (C5), 125.2 (C7′), 129.3 (C4), 130.1 (C5′), 137.5 (C5). HRMS calculated for (C₁₄H₈⁷⁹Br⁸¹BrN₂O₂S+H)⁺ 428.8726, found 428.8726.

### N-(6-bromobenzo[d]thiazol-2-yl)-3-chloro-2-hydroxybenzamide (83)

Yield=26%, beige solid. ¹HNMR (400 MHz, DMSO-d₆) δ 6.97 (1H, t, J₅₋₄=J₅₋₆=7.8 Hz, H5), 7.62 (1H, d, J_{4′-5′}=8.8 Hz, H4′), 7.65 (1H, dd, J₄₋₅=7.8 Hz, J₄₋₆=1.6 Hz, H4), 7.67 (1H, dd, J_{5′-4′}=8.8 Hz, J_{5′-7′}=2.0 Hz, H5′), 7.99 (1H, dd, J₆₋₅=7.8 Hz, J₆₋₄=1.6 Hz, H6), 8.29 (1H, d, J_{7′-5′}=2.0 Hz, H7′). ¹³C NMR (100 MHz, DMSO-d₆): δ 116.5 (C6′), 119.7 (C3), 119.9 (C5), 121.8 (C1), 125.6 (C7′), 129.2 (C4), 130.5 (C5′), 134.9 (C5), 155.9 (C2).

### Example 15: Synthesis of Compound 85

To a mixture of 3,5-dibromo-2-hydroxybenzoic acid (651 mg, 2.2 mmol) in SOCl₂ (2 mL) were added several drops of DMF. The reaction was stirred at reflux temperature for 8 h and then concentrated under reduced pressure. The residue was taken up in chlorobenzene (6 mL) and 6-bromo-1,3-benzothiazol-2-amine (458 mg, 2.0 mmol) was added. The reaction mixture was refluxed for 4 h. After cooling to rt, the mixture was diluted with ethanol and stirred at rt for 10 min. The mixture was filtered and the solid was triturated with methyl tert-butyl ether to afford compound 85 as a dark green solid (600 mg, 54%).

### 3,5-dibromo-N-(6-bromobenzo[d]thiazol-2-3/1)-2-hydroxybenzamide (85)

¹HNMR (400 MHz, DMSO-d₆) δ 7.59 (1H, d, J_{4′-5′}=8.8 Hz, H4′), 7.67 (1H, dd, J_{5′-4′}=8.8 Hz, J_{5′-7′}=2.1 Hz, H5′), 7.96 (1H, d, J₄₋₆=2.3 Hz, H4), 8.09 (11-1, J₆₋₄=2.3 Hz, H6), 8.28 (1H, d, 2.1 Hz, H7′). ¹³C NMR (100 MHz, DMSO-d₆): δ 109.9 (C5), 113.1 (C3), 116.6 (C6′), 120.8, 125.8 (C7′), 130.7 (C5′), 131.9 (C6), 139.1 (C4), 159.9 (C2).

### Example 16: Synthesis of Compound 55

A mixture of 6-bromoindole (0.60 g, 3.06 mmol), 1-bromo-4-iodobenzene (1.04 g, 3.67 mmol) and potassium acetate (0.90 g, 9.18 mmol) in H₂O (6 mL) was purged with argon for 10 min followed by the addition of PdCl₂(PPh₃)₄ (0.17 g, 0.24 mmol). The reaction mixture was heated at 110° C. for 2d, cooled to rt and then extracted with ethyl acetate. The organic layer was dried over Na₂SO₄ and evaporated under reduced pressure. The residue was purified by silica gel flash-column chromatography to afford 55 as a white solid (0.11 g, 10%).

### 6-bromo-2-(4-bromophenyl)-1H-indole (55)

¹HNMR (400 MHz, DMSO-d₆) δ 6.98 (1H, bs, H3), 7.14 (1H, dd, J₅₋₄=8.4 Hz, J₅₋₇=1.8 Hz, H5), 7.51 (1H, d, J₅₋₄=8.4 Hz, H5), 7.55 (1H, d, J₇₋₅=1.8 Hz, H7), 7.68 (1H, dm, J_{3′-2′}=J_{5′-6′}=8.8 Hz, H3′ and H5′), 7.81 (1H, dm, J_{2′-3′}=J_{6′-5′}=8.8 Hz, H2′ and H6′), 11.77 (1H, s, indolic H). ¹³C NMR (100 MHz, DMSO-d₆): δ 100.0 (C3), 114.3 (C7), 114.9 (C6), 121.2 (C4′), 122.4 (C4), 122.9 (C5), 127.5 (C2′ and C6′), 128.0 (C3a), 131.4 (C1′), 132.4 (C3′ and C5′), 137.9 (C2), 138.5 (C7a). HRMS calculated for (C₁₄H₉⁷⁹Br⁸¹BrN—H)⁻ 349.9009, found 349.9046.

### Example 17: Synthesis of Compound 57

To a mixture of 6-bromoindole (0.60 g, 3.06 mmol), 1,4-dibromobenzene (0.87 g, 3.67 mmol) and lithium hydroxide (0.22 g, 9.18 mmol in water (6 mL) was purged with argon for 10 min followed by the addition of PdCl₂(PPh₃)₄ (0.17 g, 0.24 mmol). The reaction mixture was heated at 110° C. for 38h, quenched with 1M aqueous HCl solution and then extracted with ethyl acetate. The organic layer was dried over Na₂SO₄ and evaporated under reduced pressure. The residue was purified by silica gel flash-column chromatography to afford compound 57 as a white solid (0.07 g, 7%).

### 6-bromo-3-(4-bromophenyl)-1H-indole (57)

¹HNMR (400 MHz, DMSO-d₆) δ 7.23 (1H, dd, J₅₋₄=8.4 Hz, J₅₋₇=1.8 Hz, H6), 7.61 (1H, d, J_{3′-2′}=J_{5′-6′}=8.8 Hz, H3′ and H5′), 7.64 (1H, d, J₇₋₅=1.8 Hz, H4), 7.65 (1H, d, J_{2′-3′}=J_{6′-5′}=8.8 Hz, H2′ and H6′), 7.79 (1H, bs, H2), 7.80 (1H, d, J₅₋₄=8.4 Hz, H5), 11.59 (1H, s, indolic H). ¹³C NMR (100 MHz, DMSO-d₆): δ 114.7 (C6), 115.1 (C7), 115.1 (C3), 118.8 (C4′), 121.2 (C4), 123.1 (C5), 124.2 (C3a), 125.4 (C2), 128.9 (C2′ and C6′), 132.1 (C3′ and C5′), 134.9 (C1′), 138.3 (C7a). HRMS calculated for (C₁₄H₉⁷⁹Br⁸¹BrN—H)⁻ 349.9009, found 349.8943.

### Example 18: Synthesis of Compound 65

To a mixture of 6-bromo-1,3-benzothiazol-2-amine (120 mg, 0.52 mmol) and 4-bromophtalic anhydride (660 mg, 2.92 mmol) in toluene (3 mL) was added Et₃N (1 mL). The reaction mixture was irradiated with microwaves for 2h at 200° C. The residue was filtered and the solid was triturated with ethanol and methyl tert-butylether to afford compound 65 as an off pink solid (190 mg, 50%).

### 5-bromo-2-(6-bromobenzo[d]thiazol-2-yl)isoindoline-1,3-dione (65)

¹HNMR (400 MHz, DMSO-d₆) δ 7.70 (1H, dd, J_{5′-4′}=8.8 Hz, J_{5′-7′}=2.1 Hz, H5′), 7.98 (1H, d, J_{4′-5′}=8.8 Hz, H4′), 8.00 (1H, d, J₇₋₆=8.0 Hz, H7), 8.17 (1H, dd, J₆₋₇=8.0 Hz, J₆₋₄=1.6 Hz, H6), 8.30 (1H, d, J₄₋₆=1.6 Hz, H4), 8.46 (1H, d, J_{7′-5′}=2.1 Hz, H7′). ¹³C NMR (100 MHz, DMSO-d₆): δ 118.0 (C6′), 124.5 (C4′), 125.0 (C7′), 126.4 (C7), 127.4 (C4), 129.6 (C5), 130.2 (C5′), 130.5 (C7a), 133.5 (C3a), 134.9 (C7a′), 138.7 (C6), 148.5 (C3a′), 153.2 (C2′), 163.6 & 164.3 (C1 & C3). HRMS calculated for (C₁₅H₆⁷⁹Br⁸¹BrN₂O₂S+H)⁺ 438.8569, found 438.8589.

### Example 19: Synthesis of Compound 72

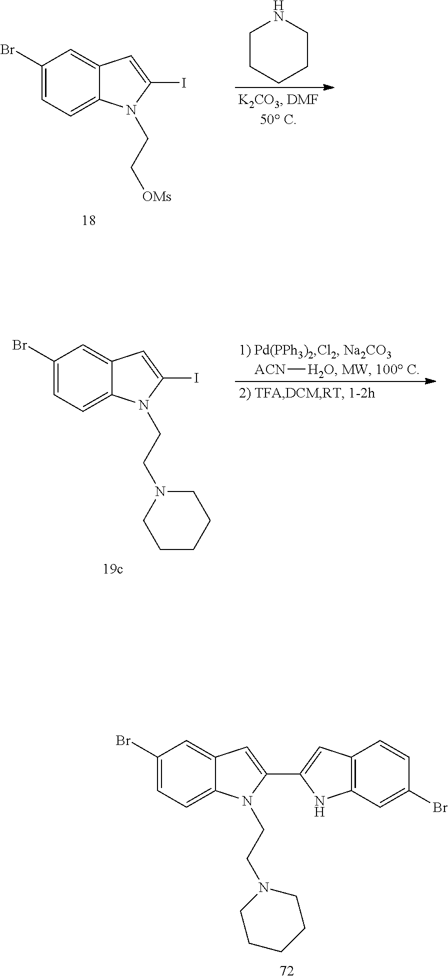

### 5-bromo-2-iodo-1-(2-(piperidin-1-yl)ethyl-1H-indole 19c

Compound 19c was prepared following the protocol described to prepare compound 19 (see Scheme 2; FIG. 3). Yield=70%, Brown oil; ¹H NMR (CDCl₃, 500 MHz): δ 7.64 (s, 1H), 7.17-7.30 (m, 2H), 6.71 (s, 1H), 4.21-4.4.39 (m, 2H), 2.36-2.71 (m, 6H), 1.39-1.73 (m, 6H).

### 5,6′-dibromo-1-(2-(piperidin-1-yl)ethyl)-1H,1′H-2,2′-biindole (72)

Compound 72 was prepared following the procedure described for the synthesis of compound 20. Yield=21%, white solid; mp 102-104° C.; ¹H NMR (DMSO-d₆, 500 MHz): δ 12.10 (s, 1H), 7.79 (d, J=1.9 Hz, 1H), 7.59-7.60 (m, 1H), 7.56 (d, J=8.4 Hz, 1H), 7.52 (d, J=8.8 Hz, 1H), 7.29 (dd, J=8.7, 1.9 Hz, 1H), 7.17 (dd, J=8.4, 1.8 Hz, 1H), 6.87 (d, J=1.2 Hz, 1H), 6.83 (s, 3H), 4.48 (t, J=6.5 Hz, 2H), 2.59 (t, J=6.5 Hz, 2H), 2.20-2.32 (m, 4H), 1.23-1.42 (m, 6H). ¹³C (DMSO-d₆, 150 MHz): 137.47, 136.42, 133.69, 130.10, 129.14, 127.40, 124.32, 122.44, 122.40, 122.08, 114.60, 113.71, 112.61, 112.48, 101.66, 101.54, 57.56, 54.50 (2C), 42.64, 25.44 (2C), 23.68 ppm. Yield: 20.7%. HRMS calculated for (C₂₃H₂₄Br₂N₃—H)⁻ 499.0259, found 499.0262.

### Example 20: Synthesis of Compound 88

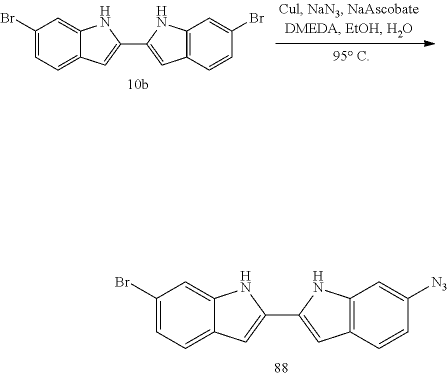 [SMILES: Brc1ccc2cc(-c3cc4ccc(Br)cc4[nH]3)[nH]c2c1 ; [N-]=[N+]=Nc1ccc2cc(-c3cc4ccc(Br)cc4[nH]3)[nH]c2c1]

10b (142 mg, 0.364 mmol), NaN₃ (24 mg, 0.364 mmol), CuI (7 mg, 0.0364 mmol) and sodium ascorbate (43 mg, 0.22 mmol) were combined in 1 mL Et0H/H₂O (7:3) and degassed under N₂ for 10 minutes. DMEDA (11.8 uL, 0.11 mmol) was added and the reaction was heated at 100° C. for 90 min in the microwave reactor. The reaction mixture was filtered through a pad of celite and then extracted with EtOAc. The organic phase was washed with brine, dried over Na₂SO₄ and concentrated. The crude product was purified via reverse phase HPLC to give compound 88 as a pale brown solid.

### 6-azido-6′-bromo-1H,1′H-2,2′-biindole (88)

¹H NMR (DMSO, 500 MHz) δ 11.76 (s, 1H), 11.70 (s, 1H), 7.63 (d, J=8.4 Hz, 1H), 7.55 (s, 1H), 7.54 (d, J=11.8 Hz, 1H) 7.15 (dd, J=8.4, 1.8 Hz, 1H), 7.08 (d, J=2.4 Hz, 1H), 6.93 (dd, J=12.3, 2.0 Hz, 2H), 6.82 (dd, J=8.4, 2.1 Hz, 1H). ¹³C NMR (DMSO, 151 MHz) δ 137.80, 137.50, 133.22, 132.02, 131.52, 127.43, 126.31, 122.33, 121.74, 121.54, 114.23, 113.47, 111.68, 101.03, 99.10, 98.61. MS (ESI, m/z).

### Example 21: Synthesis of Compounds 89 and 93

Compounds 89 and 93 were prepared using the general procedure described to synthesize compounds 10, 12, 14, 17, 20, 22, 33 and 24 (Example 1).

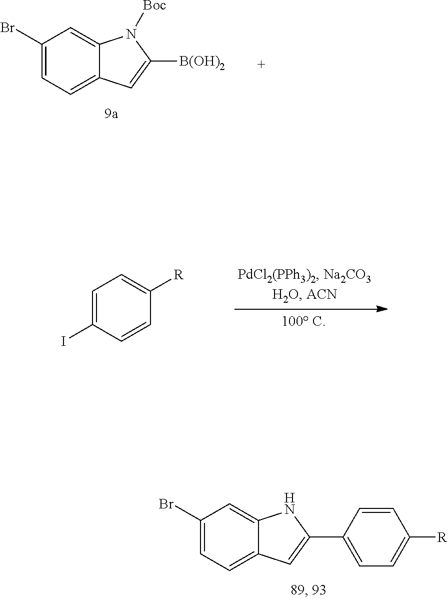

### 6-Bromo-2-(4-(3-(trifluoromethyl)diaziridin-3-yl)phenyl)-1H-indole (89)

Prepared from 9a and 3-(4-iodophenyl)-3-(trifluoromethyl)diaziridine. Yield=46%. ¹H NMR (DMSO, 500 MHz) δ 11.79 (s, 1H), 7.93 (d, J=8.4 Hz, 2H), 7.64 (d, J=8.2 Hz, 2H), 7.56 (s, 1H), 7.52 (d, J=8.4 Hz, 1H), 7.15 (dd, J=8.4, 1.7 Hz, 1H), 7.01 (d, J=1.3 Hz, 1H), 4.17 (d, J=8.2 Hz, 1H), 4.05 (d, J=7.5 Hz, 1H). ¹³C (DMSO, 150 MHz) δ 138.07, 137.71, 132.88, 131.26, 129.14, 127.53, 124.93, 124.16 (q, J=275 Hz), 122.44, 121.97, 114.42, 113.80, 99.74, 57.20 (q, J=35 Hz).

### 2-(4-azidophenyl)-6-bromo-1H-indole (93)

Prepared from 9a and 1-azido-4-iodobenzene. mp 173-175° C. ¹H NMR (DMSO, 600 MHz) δ 11.72 (s, 1H), 7.90 (d, J=8.4 Hz, 2H), 7.54 (s, 1H), 7.49 (d, J=8.4 Hz, 1H), 7.24 (d, J=8.3 Hz, 2H), 7.14 (d, J=8.4 Hz, 1H), 6.93 (s, 1H). ¹³C NMR (DMSO, 151 MHz) δ 138.60, 137.95, 137.89, 128.65, 127.65, 126.64, 122.33, 121.71, 119.72, 114.07, 113.65, 98.85.

### Example 22: Synthesis of Compound 94

A mixture of 9a (114 mg, 0.36 mmol), 3-(4-iodophenyl)-3-(trifluoromethyl)-3H-diazirine (122 mg, 0.36 mmol) and Na₂CO₃ (1M in H₂O, 1 mL) in CAN (2 mL) was purged with N₂ for 10 min. PdCl₂(PPh₃)₂ (15 mg) was added in one portion and the mixture was degassed again under N₂ for 10 min. Mixture was heated overnight in a sealed tube at 82° C. and then filtered through a pad of celite. The filtrate was extracted with EtOAc, washed with brine, dried over Na₂SO₄ and concentrated. The residue was partially purified by flash silica chromatography and then by reverse phase HPLC to give compound 94 as beige powder.

### 6-bromo-2-(4-(3-(trifluoromethyl)-3H-diazirin-3-yl)phenyl)-1H-indole (94)

¹H NMR (DMSO, 500 MHz) δ 11.85 (s, 1H), 7.98 (d, J=8.5 Hz, 2H), 7.56 (s, 1H), 7.53 (d, J=8.4 Hz, 1H), 7.38 (d, J=8.3, 2H), 7.16 (d, J=8.4, 1.7 Hz, 1H), 7.05 (s, 1H). ¹³C NMR (DMSO, 150 MHz) δ 138.21, 137.03, 133.48, 127.46, 127.07, 126.50, 125.73, 122.58, 121.98 (q, J=275 Hz), 120.98, 114.75, 113.89, 100.43, 28.35 (q, J=40 Hz).

### Example 23: Synthesis of Compound 92

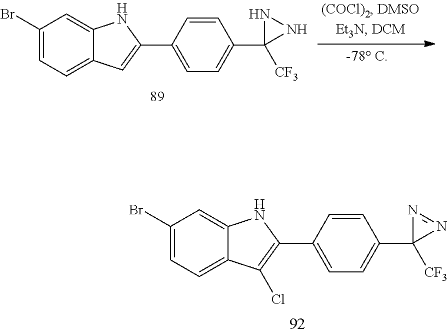 [SMILES: FC(F)(F)C1(c2ccc(-c3cc4ccc(Br)cc4[nH]3)cc2)NN1 ; FC(F)(F)C1(c2ccc(-c3[nH]c4cc(Br)ccc4c3Cl)cc2)N=N1]

Oxalyl chloride (13 μL, 0.149 mmol) was added at −78° C. to a stirred solution of DMSO (13 μL, 0.183 mmol) in dry DCM (3 mL) and stirred for 5 min. 89 (43.7 mg, 0.115 mmol) in DMSO (0.5 mL) and DCM (2 mL) was added at −78° C. in the dark and stirred for 15 min. Et₃N (80 uL, 0.573 mmol) was added at −78° C. and the reaction was warmed slowly to rt over 2 h. The reaction was quenched with H₂O, extracted with DCM and the organic phase was washed with brine, dried over Na₂SO₄ and concentrated. The crude product was purified via flash silica chromatography to give compound 92 as bright green-yellow powder.

### 6-bromo-3-chloro-2-(4-(3-(trifluoromethyl)-3H-diazirin-3-yl)phenyl)-1H-indole (92)

¹H NMR (DMSO, 600 MHz) δ 12.09 (s, 1H), 8.04 (d, J=8.5 Hz, 2H), 7.63 (d, J=1.7 Hz, 1H), 7.51 (d, J=8.5 Hz, 1H), 7.49 (d, J=8.3 Hz, 2H), 7.31 (dd, J=8.5, 1.7 Hz, 1H). ¹³C (DMSO, 151 MHz) δ 135.66, 131.78, 131.67, 127.89, 127.33, 126.98, 125.08, 123.45, 121.82 (q, J=275, 550 Hz), 119.44, 116.09, 114.49, 102.22, 28.11 (q, J=40 Hz).

### Example 24: Antimicrobial Activity and Pyruvate Kinase Inhibitory Activity

The antimicrobial activity against S. aureus ATCC 29213 and the IC₅₀ for inhibition of MRSA PK for the various compounds described in Examples 1-5 was tested according to the procedures provided under “General Methodologies” above. The results are presented below in TABLES 1-6.

TABLE 1
Antimicrobial Activity and PK Inhibitory Activity for Compounds 10a-m,
12a-c, 14, 15, 17, 20a and 20b

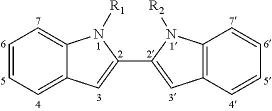

| | | MIC | | | |
|---|---|---|---|---|---|
| Compound | IC₅₀ (nM)^{a} | (μg/mL)^{b} | R₁ | R₂ | Substitutions |
| 10a | 21.4 | 2.0 | H | H | 6-Bromo |
| 10b | 7.0 | 16.0 | H | H | 6,6′-Dibromo |
| 10c | 2.2 | 0.3 | H | H | 6,5′-Dibromo |
| | | (3) | | | |
| 10d | 2.0 | 0.3 | H | H | 6-Bromo-5′-chloro |
| 10e | 2.8 | 0.3 | H | H | 6-Bromo-5′-fluoro |
| 10f | 2.5 | >64 | H | H | 6-Bromo-5′-methoxy |
| 10g | 2.2 | >64 | H | H | 6-Bromo-5′-phenyl |
| 10h | 36% @ 10 μM | ND | H | H | 5-Bromo |
| 10i | 3.0 | >64 | H | H | 5,5′-Dibromo |
| | | (2) | | | |
| 10j | 39% @ 10 μM | ND | H | H | 5,6-Dibromo |
| 10k | 1.5 | 0.5 | H | H | 5,6,6′-Tribromo |
| 10l | 2.0 | >64 | H | H | 5,5′,6,6′-Tetrabromo |
| 10m | 1.6 | 1.0 | H | H | 5,6,5′-Tribromo |
| 12a | 1.0 | >64 | H | CH₃ | 6,5′-Dibromo |
| | | (2) | | | |
| 12b | 25% @ 10 μM | ND | CH₃ | H | 6,5′-Dibromo |
| 12c | 1.9 | >64 | H | CH₂OCH₃ | 6,5′-Dibromo |
| 14 | 11.1 | >64 | H | CH₂COOH | 6,5′-Dibromo |
| 15 | 6.0 | >64 | H | 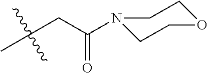 [SMILES: *CC(=O)N1CCOCC1] | 6,5′-Dibromo |
| 17 | 1.3 | 2.0 | H | CH₂CH₂OH | 6,5′-Dibromo |
| 20b | 1.2 | >64 | H | 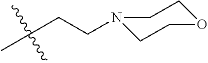 [SMILES: *CCN1CCOCC1] | 6,5′-Dibromo |
| 20a | 2.0 | 4.0 | H | 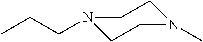 | 6,5′-Dibromo |

^{a}IC₅₀ values are calculated from a triplicate 15 point titration. Alternatively the % inhibition at the highest concentration tested is presented.
^{b}Minimum concentration to give >98% inhibition of growth of S. aureus ATCC 29213 (single determination or average of (n) determinations). Control MIC (vancomycin) is 1 μg/ml.

TABLE 2
Antimicrobial Activity and PK Inhibitory Activity for Compounds 22a-g

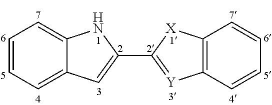

| | | MIC | | | |
|---|---|---|---|---|---|
| Compound | IC₅₀ (nM)^{a} | (μg/mL)^{b} | X | Y | Substitutions |
| 22a | 151.7 | >64 | S | N | 6-Bromo |
| 22b | 107.6 (2) | >64 | O | CH | 6-Bromo |
| 22c | 14.0 | >64 | S | CH | 6-Bromo |
| 22d | 358.1 | >64 | S | N | 5-Bromo |
| 22e | 45% @ 10 μM | ND | O | CH | 5-Bromo |
| 22f | 42% @ 1 μM | >64 | S | CH | 5-Bromo |
| 22g | 4.1 | >64 | S | N | 5,6′-Dibromo |

^{a}IC₅₀ values are calculated from a triplicate 15 point titration or are an average of (n) such determinations as indicated. Alternatively the % inhibition at the highest concentration tested is presented.
^{b}Minimum concentration to give >98% inhibition of growth of ATCC 29213 (single determination). Control MIC (vancomycin) is 1 μg/ml.

TABLE 3
Antimicrobial Activity and PK Inhibitory Activity for Compounds 25a-c,
26a & b, 27a-c, 28a & b, and 33a-f

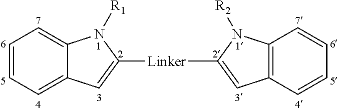

| | | MIC | | | | |
|---|---|---|---|---|---|---|
| Compound | IC₅₀ (nM)^{a} | (μg/mL)^{b} | Linker | R₁ | R₂ | Substitutions |
| 25a | 7.1 | >64 | 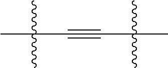 [SMILES: *C#C*] | H | H | 6-Bromo |
| 25b | 2.4 | >64 | 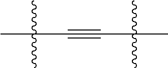 [SMILES: *C#C*] | H | H | 6,6′-Dibromo |
| 25c | 4.7 | 16 | 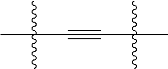 [SMILES: *C#C*] | H | H | 6,5′-Dibromo |
| 26a | 13.7 | >64 | 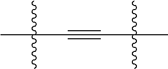 [SMILES: *C#C*] | CH₃ | H | 6,6′-Dibromo |
| 26b | 27% @ 10 μM | ND | 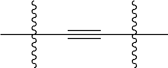 [SMILES: *C#C*] | CH₃ | CH₃ | 6,6′-Dibromo |
| 27a | 4.5 | >64 | 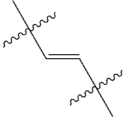 [SMILES: */C=C/*] | H | H | 6-Bromo |
| 27b | 3.9 | >64 | 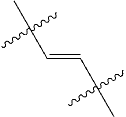 [SMILES: */C=C/*] | H | H | 6,6′-Dibromo |
| 27c | 4.1 | >64 | 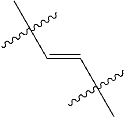 [SMILES: */C=C/*] | H | H | 6,5′-Dibromo |
| 28a | 6.5 | >64 | 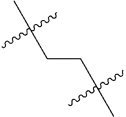 [SMILES: *CC*] | H | H | 6,6′-Dibromo |
| 28b | 6.3 | >64 | 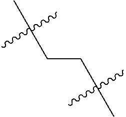 [SMILES: *CC*] | H | H | 6,5′-Dibromo |
| 33a | 54% @ 1 μM | >64 | 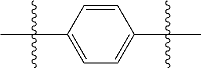 [SMILES: *c1ccc(*)cc1] | H | H | 6,6′-Dibromo |
| 33c | 5.6 | >64 | 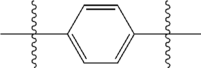 [SMILES: *c1ccc(*)cc1] | H | H | 6-Bromo |
| 33d | 1.8 | 2.0 | 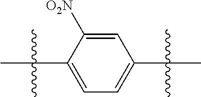 [SMILES: *c1ccc(*)c([N+](=O)[O-])c1] | H | H | 6-Bromo |
| 33e | 2.1 | >64 | 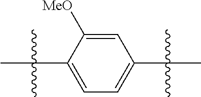 [SMILES: *c1ccc(*)c(OC)c1] | H | H | 6-Bromo |
| 33f | 36% @ 10 μM | ND | 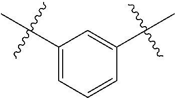 [SMILES: *c1cccc(*)c1] | H | H | 6-Bromo |
| 33b | 28% @ 1 μM | >64 | 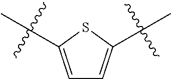 [SMILES: *c1ccc(*)s1] | H | H | 6,6′-Dibromo |

^{a}IC₅₀ values are calculated from a triplicate 15 point titration. Alternatively the % inhibition at the highest concentration tested is presented.
^{b}Minimum concentration to give >98% inhibition of growth of S. aureus ATCC 29213 (single determination). Control MIC (vancomycin) is 1 μg/ml.

TABLE 4
Antimicrobial Activity and PK Inhibitory Activity for
Compounds 36a-c and 37a-c

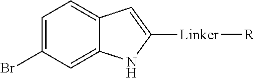

| | | MIC | | |
|---|---|---|---|---|
| Compound | IC₅₀ (nM) | (μg/mL) | Linker | R |
| 36a | 25 | >64 | 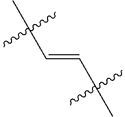 [SMILES: */C=C/*] | 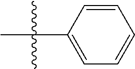 [SMILES: *c1ccccc1] |
| 36b | 1 | >64 | 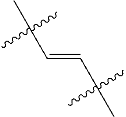 [SMILES: */C=C/*] | 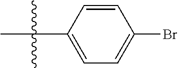 [SMILES: *c1ccc(Br)cc1] |
| 36c | — | — | 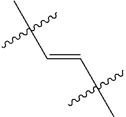 [SMILES: */C=C/*] | 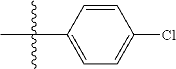 [SMILES: *c1ccc(Cl)cc1] |
| 37a | 56% @ 4 μM | — | 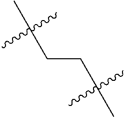 [SMILES: *CC*] | 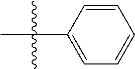 [SMILES: *c1ccccc1] |
| 37b | 187 | — | 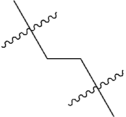 [SMILES: *CC*] | 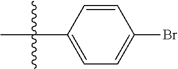 [SMILES: *c1ccc(Br)cc1] |
| 37c | — | — | 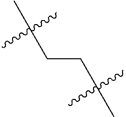 [SMILES: *CC*] | 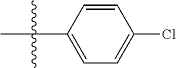 [SMILES: *c1ccc(Cl)cc1] |

TABLE 5
Antimicrobial Activity and PK Inhibitory Activity for Compounds 38a and
39a-d

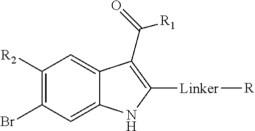

| | | MIC | | | | |
|---|---|---|---|---|---|---|
| Compound | IC₅₀ (nM) | (μg/mL) | Linker | R | R₁ | R₂ |
| 38a | >20 μM | >64 | 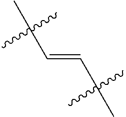 [SMILES: */C=C/*] | 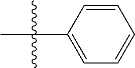 [SMILES: *c1ccccc1] | CH₃ | H |
| 39a | 608 | >64 | 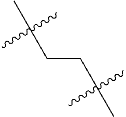 [SMILES: *CC*] | 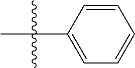 [SMILES: *c1ccccc1] | CH₃ | H |
| 39b | 28 | 4 | 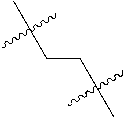 [SMILES: *CC*] | 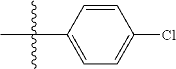 [SMILES: *c1ccc(Cl)cc1] | CH₃ | H |
| 39c | 28 | 4 | 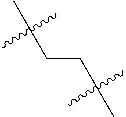 [SMILES: *CC*] | 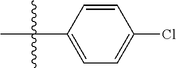 [SMILES: *c1ccc(Cl)cc1] | CH(CH₃)₂ | H |
| 39d | 2.1 | 4 | bond | 5′,6′- | OCH₃ | Br |
| | | | | dibromo-2- | | |
| | | | | indolyl | | |

TABLE 6
Antimicrobial Activity and PK Inhibitory Activity for Compounds 42-72,
74 and 77-94

| Compound | IC₅₀ (nM) | MIC (μg/mL) |
|---|---|---|
| 42 | 72% @ 10 μM | >64 |
| 43 | 50% @ 10 μM | >64 |
| 44 | 63% @ 10 μM | >64 |
| 45 | 60% @ 10 μM | >64 |
| 46 | 52% @ 10 μM | >16 |
| 47 | 52% @ 10 μM | >64 |
| 48 | 60% @ 10 μM | >64 |
| 49 | 70% @ 10 μM | >32 |
| 50 | 60% @ 10 μM | >64 |
| 51 | 3.0 | >64 |
| 52 | 67% @ 10 μM | >32 |
| 53 | 50.9 | >64 |
| 54 | 11.1 | >64 |
| 55 | 56.9 | >64 |
| 56* | 76% @ 10 μM | >64 |
| 57 | 102.3 | 2 |
| 58 | 77% @ 10 μM | 64 |
| 59 | 54% @ 10 μM | >64 |
| 60 | 79% @ 10 μM | >64 |
| 61 | 61% @ 0.1 μM | >64 |
| 62 | 12.3 | >64 |
| 63 | 9.7 | >64 |
| 64 | 115.9 | >64 |
| 65 | 76% @ 10 μM | >64 |
| 66 | 59% @ 10 μM | >64 |
| 67 | 213.8 | >64 |
| 67 | 213.8 | >64 |
| 68 | 8.3 | >64 |
| 69 | 61% @ 10 μM | >64 |
| 70 | 42.9 | 4 |
| 71 | 55% @ 10 μM | >64 |
| 72 | 3.7 | >64 |
| 72 | 3.7 | >64 |
| 74* | 56% @ 10 μM | >64 |
| 77 | 8.2 | >64 |
| 78 | 11.3 | 4 |
| 79 | 3.5 | >64 |
| 80 | 1.5 | >64 |
| 81 | 74.5 | 0.5 |
| 82 | 22.5 | >64 |
| 83 | 89% @ 1 μM | 4 |
| 84 | 84% @ 10 μM | >64 |
| 85 | 4.1 | 1.0 |
| 86 | 62% @ 10 μM | >64 |
| 87 | 58% @ 10 μM | >64 |
| 88 | 22.6 | — |
| 89 | 84% @ 10 μM | — |
| 90 | 2.4 | — |
| 91 | 7.7 | 4 |
| 92 | 110.0 | — |
| 93 | 40.5 | — |
| 94 | 78.3 | — |

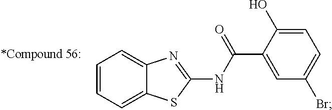 [SMILES: O=C(Nc1nc2ccccc2s1)c1cc(Br)ccc1O]

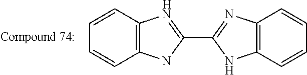

### Example 25: Selectivity of Exemplary Compounds

Exemplary compounds were screened in an in vitro enzyme assay to assess selectivity using the mammalian PK isoforms M1, M2, R and L using the protocols described in the “General Methodologies.” The results are provided in Table 7.

TABLE 7
Inhibition of enzymatic activities of MRSA PK and human PK at 10 μM

| | % Inhibition | | | | |
|---|---|---|---|---|---|
| | | Human | | | |
| Compound | MRSA PK | M1 | Human M2 | Human R | Human L |
| 10b | 84.9 (2) | 10.3 (2) | 0.1 (2) | 18.3 (2) | 7.9 (2) |
| 10c | 97.8 | 6.9 | 8.7 | 13.6 | 6.1 |
| 10i | 71 | 12.3 | 0.5 | 24.4 | 9.8 |
| 10k | 94.1 | 16.1 | 20.8 | 41.0 | 26.8 |
| 10l | 97.6 | 11.1 | 2.3 | 20.7 | 8.2 |
| 12a | 99.0 | 13.9 | 9.1 | 2.7 | 2.8 |
| 17 | 100.9 | 14.6 | 12.1 | 25.2 | 6.8 |
| 20a | 100.1 | 3.2 | 1.7 | 26.1 | 16.8 |
| 20b | 100.6 | 13.1 | 3.5 | 50.7 | 32.1 |
| 22a | 89.8 | 12.4 | 3.0 | 21.6 | 7.8 |
| 22b | 95.9 | 5.3 | 0.4 | −5.5 | −6.0 |
| 25c | 86.3 | 19.5 | 8.7 | 27.8 | 14.0 |
| 27a | 93.4 | 16.1 | −0.9 | 23.8 | 12.9 |
| 28a | 93.4 | 9.9 | 1.3 | 24.1 | 9.7 |
| 33d | 86.2 | 9.5 | 6.8 | 0.0 | −8.9 |
| 33e | 62.2 | 7.3 | 14.6 | 13.5 | 4.3 |

Of those compounds tested for selectivity none showed inhibition of the mammalian PK isoforms greater than 50% and the compounds generally showed no significant inhibition at the highest concentration tested (10 μM). Cytotoxicity was evaluated for selected compounds with HEK 293 and they were found not to be significantly cytotoxic at concentration up to 100 μg/mL.

### Example 26: SAR for In Vitro Inhibition of MRSA PK

To further improve antibacterial activity, SAR for pyruvate kinase enzyme was initially determined using MRSA PK as an example. The SAR derived for the MRSA PK enzyme is discussed separately below from the cellular antibacterial activity.

The effect of substitution on the bis-indole scaffold (Table 1) was systematically evaluated by replacing one indole ring with a number of heterocycles (Table 2) and modifying the central linking moiety (Table 3).

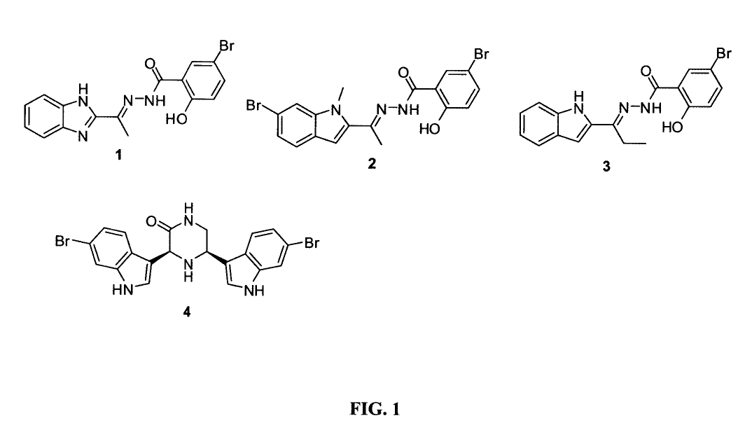

The directly linked 6,6′-dibromo-1H, 1′H-2,2′-biindole (10b) was prepared and was shown to be more potent than the naturally occurring bisindole 4 (FIG. 1) with an IC₅₀ of 7.0 nM. Compound 10b also gave a comparable MIC against S. aureus (Table 1).

To evaluate the role of the two bromines, the mono-brominated compound 10a was made and found to be about 3-fold less active. However, the asymmetrically 6,5′-dibrominated compound 10c was even more potent with IC₅₀ of 2.2 nM. It was found that the 5′-bromine could be substituted with chloro (10d), fluoro (10e), methoxy (10f) or even with a relatively bulky group such as phenyl (10g) without significant loss of potency suggesting that there is still some room in the binding pocket which might be further exploited to improve activity.

The 5′-mono brominated bis-indole analogue (10h) was next prepared and a drop in activity was noted. This suggested that at least one bromine in the 6-position may be important for activity. On the other hand, however, the 5,5′dibromo bisindole (10i) was found to be very potent with an IC₅₀ of 3 nM. One possible explanation why 10h was not active whereas 10i is very potent could be that one of the bromines of 10i is oriented towards the interior of the binding pocket and the other bromine is facing outwards, thus placing the indole NH towards the interior to provide the necessary hydrogen bonding with Ser362. Compound 10j was not active in this particular screen, whereas the two tribromo compounds 10k and 10m and tetrabromo bis-indole 101 were found to be very potent, suggesting again that there are still more space in the binding pocket to be exploited (Table 3).

The effect of substitution on the NH of indole was next investigated. The N-methyl bis-indole 12a was very active with an IC₅₀ of 1.0 nM whereas N-methyl bis-indole 12b was much less active. This again suggests that the bromine at a 6-position relative to the indole NH may be important for activity. It appears that the bromine atom and NH of 6-bromo-indole fragment binds very tightly and options for further substitution in that region of the molecule are limited. The methyl group in 12a is most likely oriented towards the outside (water side) of the binding site and that explains why compounds 12c, 11, 15, 17, 20a and 20b with bulky groups attached to nitrogen atom of 5-bromo indole are still very potent. There was no further improvement in activity by either introducing polar (12c, 15, 17, 20b), acidic (14) or basic (20a) groups at NH of the second indole.

A next step was to investigate whether both indoles are required for binding or if one can be replaced with other heterocycles. Keeping the 6-bromo indole element constant and replacing the second indole with benzothiazole (22a) or benzoxazole (22b) led to a 5-8 fold decrease in activity (compared to 10a). However, the benzothiophene derivative (22c) is slightly more potent than 10a suggesting that one indole can indeed be replaced with benzothiophene. In the case where the 5-bromo indole was kept constant and the other indole replaced with benzothiazole (22d), benzoxazole (22e) or benzothiophene (22f), there was a drop in activity as expected from earlier observation. Nevertheless, 6-bromothiazole analog (22g) was about 5-fold more potent than 10a and comparable in potency to 5,5′-dibromo-bisindole 10i, and hence might have a similar mode of binding.

The option of a spacer moiety placed between the two indoles was investigated. Compound 25a with an acetylene linker was found to be 3-fold more potent than direct linked compound (10a) suggesting that the binding pocket has more linear space. The 6,6′-dibromo analog (25b) and 6,5′-dibromo analog (25c) with an acetylene linker did not show significant improvement in potency. One indole NH could be methylated (26a) without significant loss in activity, but as expected, methylation of both NH (26b) led to a poorly active compound again suggesting the potential importance of both a 6-bromo and free NH proton on at least one indole for activity. Compounds 27a and 27b with an ethylene linker were found to be almost equipotent to compounds with the corresponding acetylene linker. Compounds 28a and 28b with a fully saturated ethane linker moiety were still very potent, both with IC₅₀ of 6 nM. Compounds 28a and 28b presumably are able to orient in a linear and planar conformation to bind in the flat lipophilic pocket similarly to the naturally product 4. In order to make the spacer slightly longer, the 1,4-phenyl linked compound 33a was synthesized. There was a significant drop in activity observed for 33a, suggesting that this linker may be too long to fit in the binding pocket. A slightly shorter analogue 33c prepared by removing one bromine was found to be very potent (IC₅₀ 5.6 nM). Hence it appears that 33c is occupying most of the binding pocket and may define the breadth of the site. It was noted that if 33 was modelled into the binding site, the central ring was flanked above and below by the His-365 residues. Considering that these interactions might lead to some charge transfer, the effect of placing an electron withdrawing group (33d) and electron releasing group (33e) on the phenyl linker was investigated. Both 33d and 33e were found to be about 2-3 fold more potent than 33c so it is not clear if such an interaction exists. 1,3-phenyl linked (331) and 2,5-thiophene linked (33b) compounds were very poorly active suggesting that both indoles may need to adopt a linear attitude for significant binding.

The results described above demonstrate that the PK SAR data establishes utility for all compounds active against PK, not only those with good MICs.

### Example 27: SAR for Anti-MRSA Activity

The MIC of the initial lead compound 10b was similar to that reported for cis-3-4-dihydrohamacanthin B (4) (FIG. 1). However, compound 10a, which contains one less bromine, was about 8 fold more potent than 10b in the MIC assay despite being 3 fold less potent in the MRSA PK enzyme assay. Direct linked bis-indole compounds 6,5′-dibromo (10c), 6-bromo-5-chloro (10d) and 6-bromo-5-fluoro (10e) were most potent with MICs of 0.3 μg/mL. The 5,5′-dibromo 10i and tetrabromo 101 derivatives were equipotent in vitro to 10b-10e in the MRSA PK enzyme assay, but showed much poorer MICs (>64 μg/ml). Both tribromo substituted compounds 10k and 10m likewise showed poor MICs in this assay. It was noted that many of these compounds had limited solubility which may limit their ability to penetrate cells and may account for the poorer MICs. A number of potentially solubilizing groups were installed on one of the indole NHs in compounds 12c, 14, 15, 17, 20a, 20b, of these the hydroxyethyl analog 17 and basic piperaziylethyl derivative 20a showed improved MICs. These results suggest that the correlation between the in vitro enzyme inhibition potency and anti-MRSA activity may not be straightforward and polarity of solubility may be just one contributing factor. In those compounds in which one indole was replaced with a heterocycle (22a-g), MICs were poor despite the compounds being potent in the enzyme assay (e.g. 22g). Of all the compounds made with a spacer between the two indoles, 25c and 33d gave the best MIC values and, of all the phenyl linked compounds made, 33d gave the best MIC value. From these results, it appears that the presence of an electron withdrawing group may improve performance in the M1C assay. Accordingly, the SAR data assists in selecting the best compounds for use in the treatment of MRSA specifically.

### Example 28: Synthesis of Compounds 95-100 and 104-118

Compounds 95-100 and 104-118 were prepared using intermediate 35 and the corresponding aldehydes following the procedure described in Example 2 to synthesize compound 36.

### (E)-6-Bromo-2-(3,5-dichlorostyryl)-1H-indole (95)

Yield=37%, yellow solid. ¹H NMR (400 MHz, DMSO) δ 11.57 (s, 1H), 7.63 (d, J=1.8 Hz, 2H), 7.54-7.49 (m, 3H), 7.47 (d, J=11.5 Hz, 1H), 7.15-7.08 (m, 2H), 6.66 (s, 1H). Mass calculated for (C₁₆H₁₀BrCl₂N+H)⁺ 365.9, found 365.8.

### (E)-6-Bromo-2-(3-chlorostyryl)-1H-indole (96)

Yield=61%, yellow solid. ¹H NMR (600 MHz, DMSO) δ 11.56 (s, 1H), 7.66 (s, 1H), 7.52 (s, 1H), 7.52 (d, J=8.3 Hz, 1H), 7.48 (d, J=8.4 Hz, 1H), 7.43 (t, J=7.8 Hz, 1H), 7.37 (d, J=16.5 Hz, 1H), 7.35-7.32 (m, 1H), 7.17 (d, J=16.5 Hz, 1H), 7.12 (dd, J=8.4, 1.7 Hz, 1H), 6.65 (s, 1H). ¹³C NMR (151 MHz, DMSO) δ 139.58, 138.64, 137.79, 134.19, 131.13, 127.79, 127.76, 126.72, 125.93, 125.58, 122.71, 122.35, 121.39, 115.30, 113.90, 104.11. Mass calculated for (C₁₆H₁₁BrClN—H)⁻ 332.0, found 332.0.

### (E)-6-Bromo-2-(4-chloro-3-nitrostyryl)-1H-indole (97)

Yield=52%, orange solid. ¹H NMR (400 MHz, DMSO) δ 11.18 (s, 1H), 7.51-7.47 (m, 1H), 7.40 (d, J=8.4 Hz, 1H), 7.13-7.02 (m, 3H), 6.70-6.61 (m, 2H), 6.38-6.31 (m, 2H), 4.37 (d, J=5.6 Hz, 2H). Mass calculated for (C₁₆H₁₀BrClN₂O₂+H)⁺ 377.0, found 376.9.

### (E)-6-Bromo-2-(2-(6-chloropyridin-3-yl)vinyl)-1H-indole (98)

Yield=60%, pale yellow solid. ¹H NMR (400 MHz, DMSO) δ 11.63 (s, 1H), 8.56 (d, J=2.5 Hz, 1H), 8.12 (dd, J=8.5, 2.5 Hz, 1H), 7.57-7.47 (m, 3H), 7.44 (d, J=16.5 Hz, 1H), 7.19 (d, J=16.6 Hz, 1H), 7.13 (dd, J=8.4, 1.8 Hz, 1H), 6.66 (s, 1H). ¹³C NMR (100 MHz, DMSO) δ 148.60, 147.93, 138.21, 137.08, 135.95, 132.16, 127.24, 124.40, 122.75, 122.29, 122.07, 121.98, 115.02, 113.47, 103.94. Mass calculated for (C₁₅H₁₀BrClN₂+H)⁺ 335.0, found 334.9.

### (E)-6-Bromo-2-(2-(5-bromopyridin-2-yl)vinyl)-1H-indole (99)

Yield=74%, orange solid. ¹H NMR (400 MHz, DMSO) δ 11.68 (s, 1H), 8.69 (d, J=2.3 Hz, 1H), 8.04 (dd, J=8.4, 2.4 Hz, 1H), 7.68 (d, J=16.1 Hz, 1H), 7.57-7.43 (m, 3H), 7.24 (d, J=16.2 Hz, 1H), 7.13 (dd, J=8.4, 1.7 Hz, 1H), 6.76 (s, 1H). ¹³C NMR (101 MHz, DMSO) δ 153.63, 150.24, 139.35, 138.32, 136.83, 127.25, 126.11, 123.48, 123.44, 122.35, 122.06, 118.15, 115.23, 113.52, 104.76. Mass calculated for (C₁₅H₁₀Br₂N₂+H)⁺378.9, found 378.9.

### (E)-6-Bromo-2-(2-(5-bromopyridin-3-yl)vinyl)-1H-indole (100)

Yield=53%, yellow solid. ¹H NMR (400 MHz, DMSO) δ 11.64 (s, 1H), 8.69 (d, J=1.8 Hz, 1H), 8.58 (d, J=2.1 Hz, 1H), 8.36 (t, J=2.0 Hz, 1H), 7.56-7.47 (m, 3H), 7.19-7.11 (m, 2H), 6.67 (s, 1H). ¹³C NMR (101 MHz, DMSO) δ 148.63, 146.57, 138.27, 136.97, 134.76, 134.43, 127.19, 123.02, 122.41, 122.33, 122.05, 120.75, 115.15, 113.51, 104.34. Mass calculated for (C₁₅H₁₀Br₂N₂+H)⁺ 378.9, found 379.0.

### (E)-2-(4-(1H-Imidazol-1-yl)styryl)-6-bromo-1H-indole (104)

Yield=43%, yellow solid. ¹H NMR (400 MHz, DMSO) δ 11.57 (s, 1H), 8.32 (s, 1H), 7.81 (s, 1H), 7.74-7.66 (m, 4H), 7.52 (s, 1H), 7.48 (d, J=8.4 Hz, 1H), 7.34 (d, J=16.5 Hz, 1H), 7.24 (d, J=16.5 Hz, 1H), 7.16-7.07 (m, 2H), 6.64 (s, 1H). ¹³C NMR (101 MHz, DMSO) δ 138.61, 138.14, 136.46, 135.91, 135.83, 130.43, 128.04, 127.89, 127.26, 122.66, 122.25, 121.02, 120.11, 118.30, 115.12, 113.84, 103.61. Mass calculated for (C₁₉H₁₄BrN₃+H)⁺ 364.0, found 364.0.

### (E)-3-(2-(6-Bromo-1H-indol-2-yl)vinyl)-5-phenylisoxazole (105)

Yield=12%, yellow solid. ¹H NMR (400 MHz, DMSO) δ 11.70 (s, 1H), 7.96-7.88 (m, 2H), 7.77 (s, 1H), 7.64-7.50 (m, 4H), 7.20-7.13 (m, 2H), 7.03-6.96 (m, 2H), 6.51 (d, J=12.9 Hz, 1H). ¹³C NMR (101 MHz, DMSO) δ 169.35, 160.52, 137.87, 135.15, 131.15, 129.83, 127.18, 127.04, 126.20, 123.09, 122.84, 116.09, 114.90, 113.79, 107.16, 102.19. Mass calculated for (C₁₉H₁₃BrN₂O—H)⁻ 363.0, found 363.0.

### (E)-6-Bromo-2-(2-(6-methylpyridin-3-yl)vinyl)-1H-indole (106)

Yield=57%, yellow solid. ¹H NMR (400 MHz, DMSO) δ 11.57 (s, 1H), 8.59 (d, J=2.1 Hz, 1H), 7.94 (dd, J=8.1, 2.3 Hz, 1H), 7.51 (s, 1H), 7.48 (d, J=8.4 Hz, 1H), 7.33 (d, J=16.6 Hz, 1H), 7.29 (d, J=8.1 Hz, 1H), 7.18 (d, J=16.6 Hz, 1H), 7.12 (dd, J=8.4, 1.8 Hz, 1H), 6.62 (d, J=1.3 Hz, 1H), 2.49 (s, 3H). ¹³C NMR (101 MHz, DMSO) δ 157.51, 147.89, 138.60, 138.01, 133.15, 130.18, 127.84, 124.90, 123.73, 122.67, 122.29, 120.55, 115.17, 113.86, 103.62, 24.32. Mass calculated for (C₁₆H₁₃BrN₂+H)⁺ 313.0, found 313.0.

### (E)-6-Bromo-2-(2-(5-methoxypyridin-2-yl)vinyl)-1H-indole (107)

Yield=63%, yellow solid. ¹H NMR (400 MHz, DMSO) δ 11.58 (s, 1H), 8.32 (d, J=2.9 Hz, 1H), 7.56-7.44 (m, 4H), 7.41 (dd, J=8.7, 3.0 Hz, 1H), 7.23 (d, J=16.2 Hz, 1H), 7.11 (dd, J=8.4, 1.8 Hz, 1H), 6.66 (d, J=1.3 Hz, 1H), 3.87 (s, 3H). ¹³C NMR (101 MHz, DMSO) δ 155.00, 147.92, 138.61, 138.11, 138.01, 127.90, 127.71, 122.98, 122.64, 122.23, 121.34, 120.71, 115.12, 113.83, 103.78, 56.14. Mass calculated for (C₁₆H₁₃BrN₂O+H)⁺ 329.0, found 329.1.

### (E)-6-Bromo-2-(2-(5-chloropyridin-2-yl)vinyl)-1H-indole (108)

Yield=28%, yellow solid. ¹H NMR (400 MHz, DMSO) δ 11.67 (s, 1H), 8.62 (d, J=2.5 Hz, 1H), 7.93 (dd, J=8.4, 2.6 Hz, 1H), 7.67 (d, J=16.2 Hz, 1H), 7.59 (d, J=8.4 Hz, 1H), 7.53 (s, 1H), 7.50 (d, J=8.4 Hz, 1H), 7.27 (d, J=16.2 Hz, 1H), 7.14 (dd, J=8.4, 1.8 Hz, 1H), 6.76 (s, 1H). ¹³C NMR (101 MHz, DMSO) δ 153.93, 148.63, 138.82, 137.33, 137.08, 129.55, 127.75, 126.56, 123.93, 123.52, 122.85, 122.57, 115.72, 114.02, 105.23. Mass calculated for (C₁₅H₁₀BrClN₂+H)⁺ 335.0, found 334.9.

### (E)-6-Bromo-2-(2-(5-methylpyrazin-2-yl)vinyl)-1H-indole (109)

Yield=75%, yellow solid. ¹H NMR (400 MHz, DMSO) δ 11.69 (s, 1H), 8.65 (d, J=1.2 Hz, 1H), 8.54 (s, 1H), 7.71 (d, J=16.2 Hz, 1H), 7.53 (s, 1H), 7.50 (d, J=8.4 Hz, 1H), 7.29 (d, J=16.2 Hz, 1H), 7.14 (dd, J=8.4, 1.8 Hz, 1H), 6.77 (s, 1H), 3.33 (s, 3H). ¹³C NMR (101 MHz, DMSO) δ 152.26, 148.04, 144.58, 142.64, 138.81, 137.37, 127.74, 124.30, 123.85, 122.86, 122.60, 115.74, 114.03, 105.17, 21.45. Mass calculated for (C₁₅H₁₂BrN₃+H)⁺ 314.0, found 314.1.

### (E)-2-(2-(6-Bromo-1H-indol-2-yl)vinyl)imidazo[1,2-a]pyridine (110)

Yield=76%, yellow solid. ¹H NMR (400 MHz, DMSO) δ 11.56 (s, 1H), 8.50 (d, J=6.8 Hz, 1H), 8.08 (s, 1H), 7.55-7.48 (m, 2H), 7.46 (d, J=8.4 Hz, 1H), 7.42 (d, J=16.1 Hz, 1H), 7.29-7.21 (m, 2H), 7.11 (dd, J=8.4, 1.8 Hz, 1H), 6.88 (td, J=6.7, 1.1 Hz, 1H), 6.64 (d, J=1.3 Hz, 1H). ¹³C NMR (101 MHz, DMSO) δ 145.39, 143.37, 138.54, 138.27, 127.98, 127.26, 125.77, 122.59, 122.10, 121.07, 120.31, 116.73, 114.89, 113.75, 112.46, 112.06, 103.08. Mass calculated for (C₁₇H₁₂BrN₃+H)⁺ 338.0, found 338.1.

### (E)-6-Bromo-2-(2-(6-methoxypyridin-3-yl)vinyl)-1H-indole (111)

Yield=66%, yellow solid. ¹H NMR (400 MHz, MeOD) δ 8.22 (d, J=2.3 Hz, 1H), 7.97 (dd, J=8.7, 2.5 Hz, 1H), 7.49 (s, 1H), 7.40 (d, J=8.4 Hz, 1H), 7.16-7.08 (m, 2H), 7.06 (d, J=16.6 Hz, 1H), 6.84 (d, J=8.7 Hz, 1H), 6.54 (s, 1H), 3.95 (s, 3H). ¹³C NMR (101 MHz, MeOD) δ 163.70, 145.13, 138.36, 137.58, 135.34, 127.82, 126.92, 123.29, 122.16, 121.06, 118.46, 114.98, 113.10, 110.64, 102.53, 52.80. Mass calculated for (C₁₆H₁₃BrN₂O—H)⁻ 327.0, found 327.0.

### (E)-4-(2-(6-Bromo-1H-indol-2-yl)vinyl)-2-methyloxazole (112)

Yield=68%, yellow solid. ¹H NMR (400 MHz, MeOD) δ 7.84 (s, 1H), 7.48 (s, 1H), 7.40 (d, J=8.4 Hz, 1H), 7.19 (d, J=16.2 Hz, 1H), 7.10 (dd, J=8.4, 1.7 Hz, 1H), 6.91 (d, J=16.2 Hz, 1H), 6.54 (s, 1H), 2.50 (s, 3H). ¹³C NMR (101 MHz, MeOD) δ 162.69, 138.70, 138.39, 137.15, 135.83, 127.76, 122.20, 121.14, 120.16, 115.27, 115.08, 113.14, 102.65, 12.15. Mass calculated for (C₁₄H₁₁BrN₂O—H)⁻ 301.0, found 301.0.

### (E)-2-(2-(6-Bromo-1H-indol-2-yl)vinyl)-4-methylthiazole (113)

Yield=84%, yellow solid. ¹H NMR (400 MHz, MeOD) δ 7.52 (s, 1H), 7.47-7.37 (m, 2H), 7.21 (d, J=16.3 Hz, 1H), 7.13 (dd, J=8.4, 1.7 Hz, 1H), 7.08 (s, 1H), 6.70 (s, 1H), 2.45 (d, J=0.7 Hz, 3H). ¹³C NMR (101 MHz, MeOD) δ 166.90, 153.08, 138.87, 135.84, 127.58, 124.36, 122.59, 121.63, 118.93, 116.17, 113.41, 113.16, 105.50, 15.34. Mass calculated for (C₁₄H₁₁BrN₂S—H)⁻ 319.0, found 319.0.

### (E)-6-Bromo-2-(2-(5-methylfuran-2-yl)vinyl)-1H-indole (114)

Yield=45%, yellow solid. ¹H NMR (400 MHz, DMSO) δ 11.46 (s, 1H), 7.47 (s, 1H), 7.43 (d, J=8.4 Hz, 1H), 7.09 (dd, J=8.4, 1.7 Hz, 1H), 7.00 (d, J=16.3 Hz, 1H), 6.88 (d, J=16.3 Hz, 1H), 6.58 (s, 1H), 6.46 (d, J=3.1 Hz, 1H), 6.19 (dd, J=3.1, 0.9 Hz, 1H), 2.34 (s, 3H). ¹³C NMR (101 MHz, DMSO) δ 152.74, 151.35, 138.49, 138.13, 128.01, 122.59, 121.97, 116.72, 116.02, 114.81, 113.65, 111.10, 108.93, 102.86, 14.01. Mass calculated for (C₁₅H₁₂BrNO—H)⁻ 300.0, found 300.0.

### (E)-5-(2-(6-Bromo-1H-indol-2-yl)vinyl)-2-methylthiazole (115)

Yield=78%, yellow solid. ¹H NMR (400 MHz, MeOD) δ 7.59 (s, 1H), 7.48 (s, 1H), 7.40 (d, J=8.4 Hz, 1H), 7.21 (d, J=16.2 Hz, 1H), 7.11 (dd, J=8.4, 1.7 Hz, 1H), 6.87 (d, J=16.2 Hz, 1H), 6.55 (s, 1H), 2.70 (s, 3H). ¹³C NMR (101 MHz, MeOD) δ 165.72, 139.61, 138.47, 138.13, 136.69, 127.70, 122.35, 121.41, 121.25, 116.95, 115.38, 113.18, 103.25, 17.58. Mass calculated for (C₁₄H₁₁BrN₂S—H)⁻ 319.0, found 319.0.

### (E)-6-Bromo-2-(2-(5-methylthiophen-2-yl)vinyl)-1H-indole (116)

Yellow solid. ¹H NMR (400 MHz, DMSO-d₆) δ 11.47 (s, 1H), 7.49-7.46 (m, 1H), 7.43 (d, J=8.4 Hz, 1H), 7.30 (d, J=16.2 Hz, 1H), 7.10 (dd, J=8.4, 1.8 Hz, 1H), 7.01 (d, J=3.5 Hz, 1H), 6.84-6.77 (m, 2H), 6.58 (d, J=1.9 Hz, 1H), 2.47 (s, 3H). ¹³C NMR (101 MHz, DMSO) δ 140.35, 139.66, 138.48, 137.95, 127.96, 127.28, 126.97, 122.60, 122.28, 122.01, 117.66, 114.84, 113.69, 102.75, 15.78. Mass calculated for (C₁₅H₁₂BrNS—H)⁻ 315.9, found 316.0.

### (E)-6-Bromo-2-(2-(4-methylthiophen-2-yl)vinyl)-1H-indole (117)

Yellow solid. ¹H NMR (400 MHz, DMSO-d₆) δ 11.50 (s, 1H), 7.48 (t, J=1.2 Hz, 1H), 7.44 (d, J=8.4 Hz, 1H), 7.32 (d, J=16.3 Hz, 1H), 7.10 (dd, J=8.4, 1.8 Hz, 1H), 7.06 (d, J=12.5 Hz, 2H), 6.91 (d, J=16.3 Hz, 1H), 6.62-6.59 (m, 1H), 2.22 (d, J=1.1 Hz, 3H). ¹³C NMR (101 MHz, DMSO) δ 142.10, 138.53, 137.80, 129.07, 122.64, 122.10, 121.97, 121.11, 118.49, 114.98, 113.73, 103.14, 99.96, 15.86. Mass calculated for (C₁₅H₁₂BrNS—H)⁻ 315.9, found 316.2.

### (E)-6-Bromo-2-(2-(5-chloropyrazin-2-yl)vinyl)-1H-indole (118)

Yield=48%, yellow solid. ¹H NMR (400 MHz, DMSO-d₆) δ 11.75 (s, 1H), 8.75 (d, J=1.3 Hz, 1H), 8.64 (d, J=1.4 Hz, 1H), 7.77 (d, J=16.2 Hz, 1H), 7.54 (s, 1H), 7.51 (d, J=8.5 Hz, 1H), 7.30 (d, J=16.2 Hz, 1H), 7.14 (dd, J=8.4, 1.8 Hz, 1H), 6.81 (d, J=1.9 Hz, 1H). ¹³C NMR (101 MHz, DMSO) δ 149.84, 146.25, 144.56, 143.04, 138.97, 136.97, 127.66, 125.81, 123.02, 122.82, 116.14, 114.15, 106.11. Mass calculated for (C₁₄H₉BrClN₃—H)⁻ 332.0, found 331.9.

### Example 29: Synthesis of Compound 101

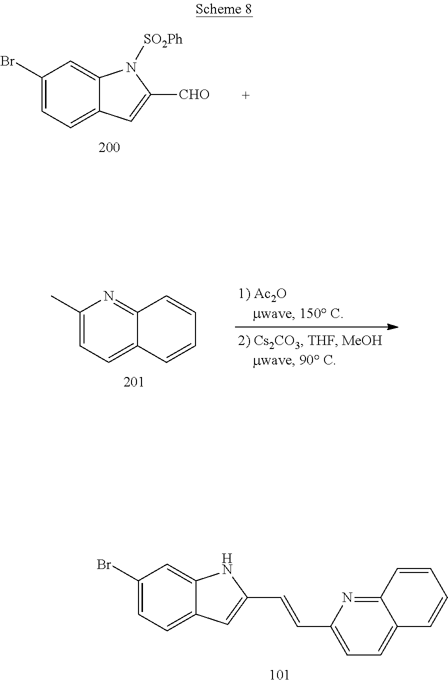

### Specific Procedure for the Synthesis of (E)-2-(2-(6-bromo-1H-indol-2-yl)vinyl)quinoline (101)

A mixture of compound 200 (1 mmol) and 201 (1.2 mmol) in Ac₂O (30 mmol) was heated by microwave at 150° C. for 30 min. The reaction mixture was diluted with EtOAc and washed with H₂O, saturated aqueous NaHCO₃ and brine. The organic phase was dried over anhydrous Na₂SO₄ and concentrated. The residue was dissolved in THF (6 mL) and MeOH (12 mL). Cs₂CO₃ (2 mmol) was added and the mixture was heated by microwave at 90° C. for 30 min. The reaction mixture was partitioned between EtOAc and H₂O and the organic layer was washed with brine, dried over anhydrous Na₂SO₄ and then concentrated. The crude product was purified by automated flash chromatography to give the desired product 101 as yellow solid (34%). ¹H NMR (500 MHz, DMSO) δ 11.76 (s, 1H), 8.37 (d, J=8.6 Hz, 1H), 7.99 (d, J=8.4 Hz, 1H), 7.96 (d, J=7.5 Hz, 1H), 7.86 (d, J=3.9 Hz, 1H), 7.84 (d, J=11.8 Hz, 1H), 7.77 (ddd, J=8.3, 6.9, 1.4 Hz, 1H), 7.61-7.45 (m, 4H), 7.15 (dd, J=8.4, 1.8 Hz, 1H), 6.82 (d, J=1.2 Hz, 1H). Mass calculated for (C₁₉H₁₃BrN₂—H)⁻ 347.0, found 347.0.

### Example 30: Synthesis of Compounds 102, 103 and 161

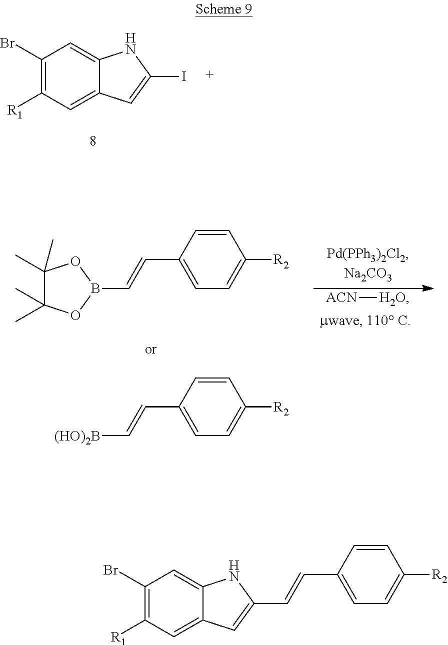

### General Procedure for the Synthesis of Compounds 102, 103 and 161

A solution of compound 8 (1 mmol), Na₂CO₃ (1M aqueous solution, 3.5 mmol) and the corresponding Pinacol boronate ester or boronic acid (1.1 mmol) in ACN (5 mL) was purged with argon for 10 min followed by the addition of Pd(PPh₃)₂Cl₂ catalyst (10 mol %). The mixture was heated by microwave at 110° C. for 90 min. The reaction mixture was diluted with EtOAc (100 mL) and washed with H₂O (2×50 mL) and brine (50 mL). The organic phase was dried over anhydrous Na₂SO₄ and concentrated. The crude product was purified by automated flash chromatography to give the desired product.

### (E)-6-Bromo-2-(4-(trifluoromethyl)styryl)-1H-indole (102)

Yield=39%, yellow solid. ¹H NMR (600 MHz, DMSO) δ 11.62 (s, 1H), 7.79 (d, J=8.2 Hz, 2H), 7.74 (d, J=8.4 Hz, 2H), 7.53 (s, 1H), 7.50 (d, J=8.4 Hz, 1H), 7.45 (d, J=16.5 Hz, 1H), 7.28 (d, J=16.5 Hz, 1H), 7.13 (dd, J=8.4, 1.8 Hz, 1H), 6.70 (s, 1H). ¹³C NMR (151 MHz, DMSO) δ 141.36, 138.71, 137.64, 127.92 (q, J_{C,F}=31.8 Hz), 127.76, 127.21, 126.62, 126.18 (q, J_{C,F}=3.6 Hz), 124.80 (q, J_{C,F}=271.7 Hz), 122.78, 122.49, 122.47, 115.51, 113.95, 104.60. Mass calculated for (C₁₇H₁₁BrF₃N—H)⁻ 364.0, found 363.9.

### (E)-6-Bromo-2-(4-fluorostyryl)-1H-indole (103)

Yield=56%, white solid. ¹H NMR (400 MHz, DMSO) δ 11.53 (s, 1H), 7.63 (dd, J=8.5, 5.7 Hz, 2H), 7.50 (s, 1H), 7.46 (d, J=8.4 Hz, 1H), 7.28-7.16 (m, 4H), 7.11 (dd, J=8.4, 1.6 Hz, 1H), 6.60 (s, 1H). ¹³C NMR (101 MHz, DMSO) δ 162.14 (d, J_{C,F}=245.2 Hz), 138.52, 138.14, 133.82 (d, J_{C,F}=3.1 Hz), 128.61 (d, J_{C,F}=8.1 Hz), 127.88, 127.25, 122.61, 122.17, 119.60 (d, J_{C,F}=2.3 Hz), 116.24 (d, J_{C,F}=21.6 Hz), 115.00, 113.80, 103.30. Mass calculated for (C₁₆H₁₁BrFN—H)⁻ 314.0, found 314.0.

### (E)-6-Bromo-2-(4-chlorostyryl)-5-fluoro-1H-indole (161)

Yield=44%, white solid. ¹H NMR (600 MHz, DMSO) δ 11.61 (s, 1H), 7.62-7.58 (m, 3H), 7.49 (d, J=9.7 Hz, 1H), 7.46 (d, J=8.5 Hz, 2H), 7.30 (d, J=16.5 Hz, 1H), 7.22 (d, J=16.5 Hz, 1H), 6.64 (d, J=1.2 Hz, 1H). ¹³C NMR (151 MHz, DMSO) δ 152.61 (d, J_{C,F}=233.2 Hz), 139.12, 135.58, 134.36, 132.10, 128.85, 128.03, 127.97, 127.24, 119.84, 114.48, 105.89 (d, J_{C,F}=24.4 Hz), 103.16 (d, J_{C,F}=4.7 Hz), 101.91 (d, J_{C,F}=24.6 Hz). Mass calculated for (C₁₆H₁₀BrClFN—H)⁻ 350.0, found 350.0.

### Example 31: Synthesis of Compounds 119-121, 125 and 126

Compounds 119-121 and 126 were prepared following the procedure described in Example 3 for the synthesis of compound 37. Compound 125 was prepared using a similar protocol except the mixture was stirred under an H₂ atmosphere for 2h instead of 16h.

### 6-Bromo-2-(3,5-dichlorophenethyl)-1H-indole (119)

Yield=88%, pale yellow solid. ¹H NMR (400 MHz, DMSO) δ 11.13 (s, 1H), 7.47 (s, 1H), 7.42 (t, J=1.9 Hz, 1H), 7.37 (d, J=8.4 Hz, 1H), 7.35 (d, J=1.9 Hz, 2H), 7.05 (dd, J=8.4, 1.8 Hz, 1H), 6.18 (d, J=1.4 Hz, 1H), 3.03 (s, 4H). Mass calculated for (C₁₆H₁₂BrCl₂N—H)⁻ 366.0, found 365.9.

### 6-Bromo-2-(2-(6-chloropyridin-3-yl)ethyl)-1H-indole (120)

Yield=84%, yellow solid. ¹H NMR (400 MHz, DMSO) δ 11.15 (s, 1H), 8.26 (d, J=2.1 Hz, 1H), 7.71 (dd, J=8.2, 2.5 Hz, 1H), 7.47-7.39 (m, 2H), 7.36 (d, J=8.4 Hz, 1H), 7.05 (dd, J=8.4, 1.8 Hz, 1H), 6.16 (d, J=1.3 Hz, 1H), 3.03 (s, 4H). ¹³C NMR (101 MHz, DMSO) δ 149.58, 147.93, 139.84, 139.59, 136.83, 136.08, 127.19, 123.81, 121.42, 120.86, 113.13, 112.74, 99.07, 30.57, 28.70. Mass calculated for (C₁₅H₁₂BrClN₂+H)⁺ 337.0, found 337.0.

### 6-Bromo-2-(2-(5-bromopyridin-3-yl)ethyl)-1H-indole (121)

Yield=8%. ¹H NMR (400 MHz, DMSO) δ 11.15 (s, 1H), 8.52 (d, J=2.2 Hz, 1H), 8.42 (d, J=1.7 Hz, 1H), 7.97 (t, J=2.0 Hz, 1H), 7.46 (s, 1H), 7.37 (d, J=8.4 Hz, 1H), 7.05 (dd, J=8.4, 1.8 Hz, 1H), 6.17 (s, 1H), 3.05 (s, 3H). ¹³C NMR (101 MHz, DMSO) δ 148.65, 148.39, 140.34, 139.41, 138.87, 137.31, 127.69, 121.93, 121.36, 120.48, 113.63, 113.24, 99.53, 31.41, 29.06. Mass calculated for (C₁₅H₁₂Br₂N₂+H)⁺ 380.9, found 380.9.

### 2-(2-(6-Bromo-1H-indol-2-yl)ethyl)-5,6,7,8-tetrahydroimidazo[1,2-a]pyridine (126)

Yield=46%, brown solid. ¹H NMR (400 MHz, DMSO) δ 11.22 (s, 1H), 7.44 (s, 1H), 7.37 (d, J=8.3 Hz, 1H), 7.05 (dd, J=8.3, 1.8 Hz, 1H), 6.71 (s, 1H), 6.20 (s, 1H), 3.85 (t, J=5.7 Hz, 2H), 2.98 (t, J=7.8 Hz, 2H), 2.81 (t, J=7.8 Hz, 2H), 2.68 (t, J=6.1 Hz, 2H), 1.91-1.75 (m, 4H). ¹³C NMR (101 MHz, DMSO) δ 143.58, 141.88, 139.54, 137.31, 127.85, 121.80, 121.24, 114.65, 113.57, 112.97, 98.92, 44.40, 28.11, 28.03, 24.52, 23.08, 21.22. Mass calculated for (C₁₇H₁₈BrN₃+H)⁺ 344.1, found 344.1.

### 2-(2-(6-Bromo-1H-indol-2-yl)ethyl)imidazo[1,2-a]pyridine (125)

Yield=23%. ¹H NMR (400 MHz, DMSO) δ 11.24 (s, 1H), 8.46 (d, J=6.7 Hz, 1H), 7.71 (s, 1H), 7.51-7.43 (m, 2H), 7.36 (d, J=8.4 Hz, 1H), 7.22-7.13 (m, 1H), 7.05 (dd, J=8.3, 1.8 Hz, 1H), 6.83 (td, J=6.7, 1.0 Hz, 1H), 6.22 (s, 1H), 3.20-3.07 (m, 4H). ¹³C NMR (101 MHz, DMSO) δ 146.35, 144.53, 141.48, 137.34, 127.81, 126.99, 124.54, 121.84, 121.29, 116.60, 113.61, 113.06, 111.99, 110.05, 99.11, 28.44, 27.83. Mass calculated for (C₁₇H₁₄BrN₃+H)⁺ 340.0, found 340.0.

### Example 32: Synthesis of Compounds 122 and 127

Compounds 122 and 127 were prepared following the procedure described in Example 4 for the synthesis of compound 38.

### (E)-1-(6-Bromo-2-(4-chlorostyryl)-1H-indol-3-yl)ethanone (122)

Yield=30%, yellow solid. ¹H NMR (400 MHz, DMSO) δ 12.33 (s, 1H), 7.97 (s, 1H), 7.94 (d, J=8.9 Hz, 1H), 7.67 (d, J=8.5 Hz, 2H), 7.60 (d, J=1.7 Hz, 1H), 7.52 (d, J=8.5 Hz, 2H), 7.46 (d, J=16.7 Hz, 1H), 7.33 (dd, J=8.6, 1.9 Hz, 1H), 2.65 (s, 3H). ¹³C NMR (151 MHz, DMSO) δ 194.31, 141.90, 137.58, 135.48, 133.67, 132.88, 129.56, 129.05, 126.16, 124.90, 123.35, 119.37, 116.36, 114.99, 114.45, 32.12. Mass calculated for (C₁₈H₁₃BrClNO—H)⁻ 374.0, found 374.0.

### 1-(6-Bromo-2-(2-(5,6,7,8-tetrahydroimidazo[1,2-a]pyridin-2-yl)ethyl)-1H-indol-3-yl)ethan-1-one (127)

Yield=45%, white solid. ¹H NMR (400 MHz, DMSO-d₆) δ 12.13 (s, 1H), 7.95 (d, J=8.6 Hz, 1H), 7.57 (d, J=1.8 Hz, 1H), 7.29 (dd, J=8.6, 1.9 Hz, 1H), 6.74 (s, 1H), 3.86 (t, J=5.7 Hz, 2H), 3.38-3.34 (m, 2H), 2.86-2.78 (m, 2H), 2.70 (t, J=6.1 Hz, 2H), 2.55 (s, 3H), 1.93-1.74 (m, 4H). ¹³C NMR (101 MHz, DMSO) δ 193.53, 149.16, 143.77, 139.05, 136.32, 126.26, 124.49, 122.92, 114.79, 114.72, 114.42, 113.50, 44.46, 31.37, 28.61, 28.10, 24.50, 23.05, 21.18. Mass calculated for (C₁₉H₂₀BrN₃O+H)⁺ 386.1, found 386.0.

### Example 33: Synthesis of Compounds 123, 150-153 and 167-176

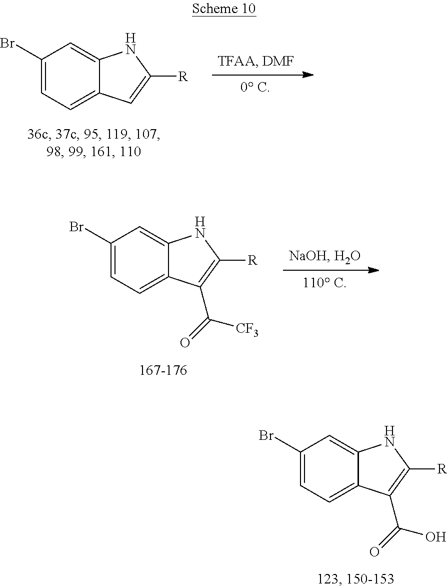

### General Procedure for the Synthesis of Compounds 167-176

To a stirred solution of the corresponding starting material (1 mmol) in DMF (7 mL) at 0° C. under argon was added TFAA (1.5 mmol) and the mixture was stirred at 0° C. for 5h. The reaction mixture was diluted with EtOAc (100 mL) and washed with H₂O (2×50 mL) and brine (50 mL). The organic phase was dried over anhydrous Na₂SO₄ and concentrated. The crude product was purified by automated flash chromatography to give the desired product.

### 1-(6-Bromo-2-(4-chlorophenethyl)-1H-indol-3-yl)-2,2,2-trifluoroethanone (167)

Yield=69%. ¹H NMR (600 MHz, DMSO) δ 7.75 (d, J=8.5 Hz, 1H), 7.70 (d, J=1.8 Hz, 1H), 7.43 (dd, J=8.7, 1.9 Hz, 1H), 7.37 (d, J=8.3 Hz, 2H), 7.26 (d, J=8.4 Hz, 2H), 3.40-3.33 (m, 2H), 3.01-2.95 (m, 2H). ¹³C NMR (151 MHz, DMSO) δ 173.94 (q, J=35.2 Hz), 155.41, 139.87, 136.81, 131.35, 130.58, 128.85, 126.11, 124.26, 121.90 (q, J_{C,F}=4.4 Hz), 117.31 (q, J_{C,F}=290.4 Hz), 116.10, 115.51, 106.49, 33.72, 30.79. Mass calculated for (C₁₈H₁₂BrClF₃NO—H)⁻ 430.0, found 430.0.

### (E)-1-(6-Bromo-2-(4-chlorostyryl)-1H-indol-3-yl)-2,2,2-trifluoroethanone (168)

Yield=80%, yellow solid. ¹H NMR (600 MHz, CDCl₃) δ 9.18 (s, 1H), 7.96-7.89 (m, 2H), 7.59 (d, J=1.5 Hz, 1H), 7.51 (d, J=8.4 Hz, 2H), 7.42 (dd, J=8.7, 1.8 Hz, 1H), 7.39 (d, J=8.4 Hz, 2H), 7.20 (d, J=16.7 Hz, 1H). ¹³C NMR (151 MHz, CDCl₃) δ 176.06 (q, J_{C,F}=36.7 Hz), 145.67, 136.40, 135.56, 134.20, 133.75, 129.29, 128.49, 126.72, 124.74, 122.76 (q, J_{C,F}=4.4 Hz), 118.38, 117.71, 116.83 (q, J_{C,F}=289.6 Hz), 114.17, 108.72. Mass calculated for (C₁₈H₁₀BrClF₃NO—H)⁻ 428.0, found 427.9.

### 1-(6-Bromo-2-(pyrimidin-2-ylethynyl)-1H-indol-3-yl)-2,2,2-trifluoroethanone (169)

Orange solid. ¹H NMR (400 MHz, DMSO-d₆) δ 13.83 (s, 1H), 8.96 (d, J=5.0 Hz, 2H), 8.11 (d, J=8.7 Hz, 1H), 7.74 (d, J=1.8 Hz, 1H), 7.64 (t, J=4.9 Hz, 1H), 7.55 (dd, J=8.7, 1.8 Hz, 1H). ¹³C NMR (101 MHz, DMSO-d₆) δ 174.43 (q, J=36.7 Hz), 158.62, 151.52, 137.59, 127.80, 125.53, 125.32, 123.58, 122.24, 119.10, 116.69 (q, J=289.9 Hz), 115.74, 112.41, 96.27, 75.79. Mass calculated for (C₁₆H₇BrF₃N₃O+H)⁺ 394.0, found 393.9.

### (E)-1-(6-Bromo-2-(3,5-dichlorostyryl)-1H-indol-3-yl)-2,2,2-trifluoroethanone (170)

Yield=89%, yellow solid. ¹H NMR (500 MHz, DMSO) δ 13.14 (s, 1H), 7.87 (d, J=16.5 Hz, 1H), 7.80 (d, J=8.6 Hz, 1H), 7.73 (d, J=1.8 Hz, 1H), 7.72 (d, J=1.8 Hz, 2H), 7.67 (t, J=1.8 Hz, 1H), 7.54 (d, J=16.6 Hz, 1H), 7.47 (dd, J=8.8, 1.8 Hz, 1H). ¹³C NMR (151 MHz, DMSO) δ 174.78 (q, J_{C,F}=35.0 Hz), 147.05, 139.73, 137.92, 135.31, 134.16, 128.93, 126.54, 126.09, 124.47, 122.33, 120.88, 118.11 (q, J_{C,F}=290.5 Hz), 117.55, 115.52, 107.87. Mass calculated for (C₁₈H₉BrCl₂F₃NO—H)⁻ 461.9, found 461.9.

### 1-(6-Bromo-2-(3,5-dichlorophenethyl)-1H-indol-3-yl)-2,2,2-trifluoroethanone (171)

Yield=57%, white solid. ¹H NMR (600 MHz, DMSO) δ 7.74 (d, J=8.6 Hz, 1H), 7.72 (d, J=1.7 Hz, 1H), 7.47 (t, J=1.8 Hz, 1H), 7.44 (dd, J=8.7, 1.9 Hz, 1H), 7.30 (d, J=1.8 Hz, 2H), 3.38 (dd, J=8.9, 6.9 Hz, 2H), 3.03-2.99 (m, 2H). ¹³C NMR (151 MHz, DMSO) δ 173.99 (q, J_{C,F}=35.1 Hz), 155.02, 145.15, 136.70, 134.39, 127.65, 126.49, 126.19, 124.09, 121.86 (q, J_{C,F}=4.8 Hz), 118.25 (q, J_{C,F}=290.3 Hz), 116.17, 115.54, 106.57, 33.55, 30.30. Mass calculated for (C₁₈H₁₁BrCl₂F₃NO—H)⁻ 463.9, found 463.9.

### (E)-1-(6-Bromo-2-(2-(5-methoxypyridin-2-yl)vinyl)-1H-indol-3-yl)-2,2,2-trifluoroethanone (172)

Yield=50%, yellow solid. ¹H NMR (400 MHz, DMSO) δ 13.05 (s, 1H), 8.44 (d, J=2.9 Hz, 1H), 8.16 (d, J=16.1 Hz, 1H), 7.82 (d, J=8.7 Hz, 1H), 7.71-7.63 (m, 2H), 7.59 (d, J=8.6 Hz, 1H), 7.51-7.42 (m, 2H), 3.90 (s, 3H). ¹³C NMR (101 MHz, DMSO) δ 174.69 (q, J=35.9 Hz), 156.15, 147.52, 146.22, 139.18, 137.91, 136.27, 126.32, 125.25, 124.92, 122.43 (q, J=4.3 Hz), 121.11, 118.01, 117.38, 117.22 (q, J=290.4 Hz), 115.23, 107.49, 56.28. Mass calculated for (C₁₈H₁₂BrF₃N₂O₂+H)⁺ 425.0, found 425.0.

### 1-(6-Bromo-2-(2-(6-chloropyridin-3-yl)ethyl)-1H-indol-3-yl)-2,2,2-trifluoroethanone (173)

Yield=68%, pale brown solid. ¹H NMR (600 MHz, DMSO) δ 8.24 (d, J=2.5 Hz, 1H), 7.74 (d, J=8.6 Hz, 1H), 7.70 (d, J=1.9 Hz, 1H), 7.67 (dd, J=8.2, 2.5 Hz, 1H), 7.47-7.42 (m, 2H), 3.38 (dd, J=8.4, 7.0 Hz, 2H), 3.06-3.01 (m, 2H). ¹³C NMR (151 MHz, DMSO) δ 173.99 (q, J_{C,F}=35.5 Hz), 154.90, 149.99, 148.76, 140.14, 136.70, 135.79, 126.18, 124.47, 124.09, 121.89 (q, J_{C,F}=4.6 Hz), 117.27 (q, J_{C,F}=290.4 Hz), 116.18, 115.54, 106.61, 30.58, 30.28. Mass calculated for (C₁₇H₁₁BrClF₃N₂O+H)⁺ 433.0, found 432.7.

### (E)-1-(6-Bromo-2-(2-(5-bromopyridin-2-yl)vinyl)-1H-indol-3-yl)-2,2,2-trifluoroethanone (174) TFA salt

Yield=60%, brown solid. ¹H NMR (600 MHz, DMSO) δ 8.82 (d, J=2.3 Hz, 1H), 8.31 (d, J=16.1 Hz, 1H), 8.15 (dd, J=8.3, 2.4 Hz, 1H), 7.83 (d, J=8.6 Hz, 1H), 7.72 (d, J=1.8 Hz, 1H), 7.67 (d, J=16.1 Hz, 1H), 7.59 (d, J=8.3 Hz, 1H), 7.47 (dd, J=8.7, 1.8 Hz, 1H). ¹³C NMR (151 MHz, DMSO) δ 174.94 (q, J_{C,F}=35.4 Hz), 158.70 (q, J_{C,F}=37.2 Hz), 152.51, 151.39, 146.44, 140.34, 137.96, 135.08, 126.53, 125.79, 124.75, 122.51 (d, J_{C,F}=4.1 Hz), 121.25, 120.61, 117.70, 117.12 (q, J_{C,F}=290.2 Hz), 115.84 (q, J_{C,F}=290.2 Hz), 115.42, 108.16. Mass calculated for (C₁₉H₉Br₂F₆N₂O₂+H)⁺ 474.9, found 474.8.

### (E)-1-(6-Bromo-2-(4-chlorostyryl)-5-fluoro-1H-indol-3-yl)-2,2,2-trifluoroethanone (175)

Yield=23%, yellow solid. ¹H NMR (400 MHz, DMSO) δ 13.16 (s, 1H), 7.83-7.76 (m, 2H), 7.71-7.66 (m, 3H), 7.63 (d, J=16.6 Hz, 1H), 7.56 (d, J=8.5 Hz, 2H). Mass calculated for (C₁₈H₉BrClF₄NO—H)⁻ 446.0, found 445.8.

### (E)-1-(6-Bromo-2-(2-(imidazo[1,2-a]pyridin-2-yl)vinyl)-1H-indol-3-yl)-2,2,2-trifluoroethanone (176)

Yellow solid. ¹H NMR (400 MHz, DMSO-d₆) δ 13.04 (s, 1H), 8.54 (d, J=6.8 Hz, 1H), 8.29 (s, 1H), 8.14 (d, J=16.0 Hz, 1H), 7.81 (d, J=8.6 Hz, 1H), 7.75 (d, J=16.1 Hz, 1H), 7.68 (d, J=1.9 Hz, 1H), 7.61 (d, J=9.1 Hz, 1H), 7.43 (dd, J=8.7, 1.9 Hz, 1H), 7.31 (ddd, J=8.9, 6.8, 1.3 Hz, 1H), 6.96-6.90 (m, 1H). Mass calculated for (C₁₉H₁₁BrF₃N₃O—H)⁻ 432.0, found 431.8.

### General Procedure for the Synthesis of Compounds 123 and 150-153

A suspension of the appropriate starting compound (167-176) (1 mmol) in an aqueous solution of NaOH (20%, 20 mL) was heated at 110° C. for 5h. After cooling to rt the reaction mixture was acidified with 15% aqueous HCl solution to pH of 1-2. The resulting mixture was extracted with EtOAc (×2) and the organic phase was dried over anhydrous Na₂SO₄ and concentrated. The residue was recrystallized with EtOAc and hexanes to give the desired product.

### (E)-6-Bromo-2-(4-chlorostyryl)-1H-indole-3-carboxylic acid (123)

Yield=83%, green solid. ¹H NMR (400 MHz, DMSO) δ 12.52 (s, 1H), 12.26 (s, 1H), 8.04 (d, J=16.9 Hz, 1H), 7.95 (d, J=8.6 Hz, 1H), 7.64-7.56 (m, 3H), 7.54-7.50 (m, 2H), 7.45 (d, J=16.8 Hz, 1H), 7.28 (dd, J=8.6, 1.8 Hz, 1H). Mass calculated for (C₁₇H₁₁BrClNO₂—H)⁻ 374.0, found 374.0.

### (E)-6-Bromo-2-(3,5-dichlorostyryl)-1H-indole-3-carboxylic acid (150)

Yield=73%, yellow solid. ¹H NMR (600 MHz, DMSO) δ 12.31 (s, 1H), 8.06 (d, J=16.8 Hz, 1H), 7.96 (d, J=8.6 Hz, 1H), 7.62-7.58 (m, 4H), 7.38 (d, J=16.7 Hz, 1H), 7.30 (dd, J=8.6, 1.8 Hz, 1H). ¹³C NMR (151 MHz, DMSO) δ 166.37, 141.51, 140.49, 137.57, 135.21, 129.81, 128.13, 126.72, 125.52, 124.71, 123.65, 121.39, 116.65, 114.40, 106.81. Mass calculated for (C₁₇H₁₀BrCl₂NO₂—H)⁻ 407.9, found 408.1.

### (E)-6-Bromo-2-(2-(5-methoxypyridin-2-yl)vinyl)-1H-indole-3-carboxylic acid (151)

Yield=58%, yellow solid. ¹H NMR (400 MHz, DMSO) δ 12.48 (bs, 1H), 12.25 (s, 1H), 8.40 (d, J=2.8 Hz, 1H), 8.33 (d, J=16.5 Hz, 1H), 7.96 (d, J=8.6 Hz, 1H), 7.57 (d, J=1.6 Hz, 1H), 7.53-7.41 (m, 3H), 7.28 (dd, J=8.6, 1.7 Hz, 1H), 3.89 (s, 3H). ¹³C NMR (101 MHz, DMSO) δ 166.53, 155.57, 147.16, 142.42, 138.76, 137.57, 131.77, 127.02, 124.45, 124.25, 123.50, 121.16, 119.26, 116.26, 114.18, 106.17, 56.19. Mass calculated for (C₁₇H₁₃BrN₂O₃—H)⁻ 371.0, found 371.0.

### (E)-6-Bromo-2-(2-(6-chloropyridin-3-yl)vinyl)-1H-indole-3-carboxylic acid (152)

Yield=53%, yellow solid. ¹H NMR (400 MHz, DMSO) δ 12.52 (s, 1H), 12.34 (s, 1H), 8.57 (d, J=2.5 Hz, 1H), 8.14-8.06 (m, 2H), 7.96 (d, J=8.6 Hz, 1H), 7.62-7.57 (m, 2H), 7.45 (d, J=16.9 Hz, 1H), 7.30 (dd, J=8.6, 1.8 Hz, 1H). ¹³C NMR (151 MHz, DMSO) δ 165.90, 149.60, 148.53, 141.20, 137.07, 136.24, 131.56, 127.31, 126.23, 124.71, 124.18, 123.14, 120.38, 116.09, 113.85, 106.03. Mass calculated for (C₁₆H₁₀BrClN₂O₂+H)⁺ 379.0, found 379.1.

### (E)-6-Bromo-2-(2-(5-bromopyridin-2-yl)vinyl)-1H-indole-3-carboxylic acid (153)

Yield=56%, yellow solid. ¹H NMR (400 MHz, DMSO) δ 12.58 (s, 1H), 12.34 (s, 1H), 8.77 (d, J=2.4 Hz, 1H), 8.50 (d, J=16.4 Hz, 1H), 8.09 (dd, J=8.4, 2.4 Hz, 1H), 7.96 (d, J=8.6 Hz, 1H), 7.58 (d, J=1.8 Hz, 1H), 7.50 (d, J=2.9 Hz, 1H), 7.46 (d, J=11.0 Hz, 1H), 7.29 (dd, J=8.6, 1.8 Hz, 1H). ¹³C NMR (101 MHz, DMSO) δ 165.85, 152.87, 150.63, 140.96, 139.64, 137.20, 130.25, 126.38, 124.59, 124.20, 123.19, 121.82, 119.17, 116.26, 113.88, 106.62. Mass calculated for (C₁₆H₁₀Br₂N₂O₂—H)⁻ 420.9, found 420.9.

### Example 34: Synthesis of Compound 124

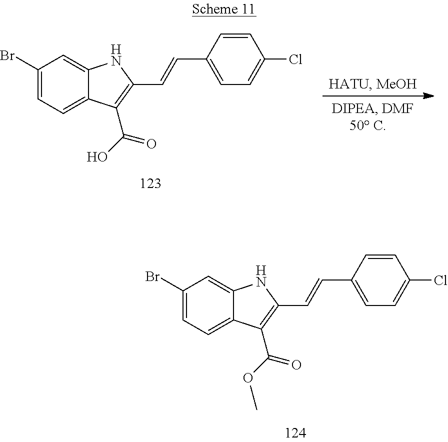 [SMILES: O=C(O)c1c(/C=C/c2ccc(Cl)cc2)[nH]c2cc(Br)ccc12 ; COC(=O)c1c(/C=C/c2ccc(Cl)cc2)[nH]c2cc(Br)ccc12]

To a stirred solution of 123 (50 mg, 0.13 mmol), HATU (55 mg, 0.14 mmol) and DIPEA (0.11 mL, 0.63 mmol) in DMF (2 mL) at rt was added MeOH (0.1 mL) and the mixture was heated at 50° C. overnight. The reaction mixture was diluted with EtOAc (50 mL) and washed with H₂O (2×20 mL) and brine (20 mL). The organic phase was dried over anhydrous Na₂SO₄ and concentrated. The crude product was purified by automated flash chromatography to give compound 124 as yellow solid (27 mg, 45%).

### (E)-methyl 6-bromo-2-(4-chlorostyryl)-1H-indole-3-carboxylate (124)

¹H NMR (400 MHz, DMSO) δ 12.39 (s, 1H), 8.00 (d, J=16.8 Hz, 1H), 7.92 (d, J=8.6 Hz, 1H), 7.64 (d, J=8.5 Hz, 2H), 7.60 (d, J=1.7 Hz, 1H), 7.53 (d, J=8.5 Hz, 2H), 7.49 (d, J=16.9 Hz, 1H), 7.32 (dd, J=8.6, 1.8 Hz, 1H), 3.90 (s, 3H). ¹³C NMR (101 MHz, DMSO) δ 165.36, 142.61, 137.53, 135.44, 133.65, 132.12, 129.63, 128.96, 126.24, 124.81, 123.37, 118.44, 116.55, 114.42, 104.98, 51.55. Mass calculated for (C₁₈H₁₃BrClNO₂—H)⁻ 490.0, found 489.9.

### Example 35: Synthesis of Compounds 162 and 177-179

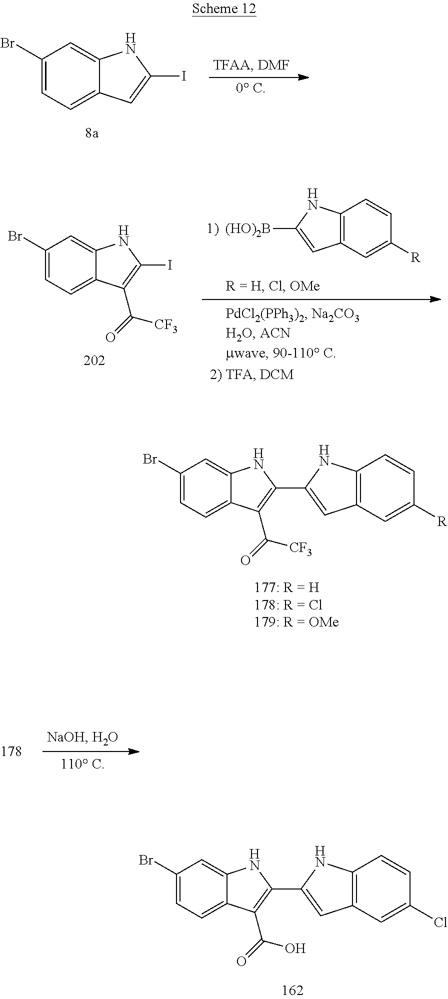

Specific procedure for the synthesis of intermediate 202

To a stirred solution of compound 8a (1.0 g, 3.1 mmol) in DMF (20 mmol) at 0° C. under argon was added TFAA (0.8 mL, 5.8 mmol) and the mixture was stirred at 0° C. for 6h. The reaction mixture was diluted with EtOAc (100 mL) and washed with H₂O (2×50 mL) and brine (50 mL). The organic phase was dried over anhydrous Na₂SO₄ and concentrated. The crude product was purified by automated flash chromatography to give compound 202 as white solid (1.21g, 93%). ¹H NMR (400 MHz, Chloroform-d) δ 9.15 (s, 1H), 7.89 (d, J=8.8 Hz, 1H), 7.60 (d, J=1.8 Hz, 1H), 7.43 (dd, J=8.8, 1.8 Hz, 1H). Mass calculated for (C₁₀H₄BrF₃INO—H)⁻ 415.9, found 415.7.

### Specific Procedure for the Synthesis of 1-(6-bromo-1H,1′H-[2,2′-biindol]-3-yl)-2,2,2-trifluoroethan-1-one (177)

A solution of 202 (100 mg, 0.24 mmol), Na₂CO₃ (1M aqueous solution, 0.9 mL, 0.9 mmol)) and (1H-indol-2-yl)boronic acid (68 mg, 0.26 mmol) in ACN (2 mL) was purged with argon for 10 min followed by the addition of Pd(PPh₃)₂Cl₂ catalyst (15 mg, 0.02 mmol). The mixture was heated by microwave at 110° C. for 90 min. The reaction mixture was diluted with EtOAc (50 mL) and washed with H₂O (2×20 mL) and brine (20 mL). The organic phase was dried over anhydrous Na₂SO₄ and concentrated. The residue was dissolved in TFA/DCM mixture (1:3, 4 mL) and stirred at rt for 2h. The reaction mixture was concentrated and then purified by automated flash chromatography to give 177 as yellow solid (52%). ¹H NMR (600 MHz, CDCl₃) δ 11.84 (s, 1H), 9.29 (s, 1H), 7.88 (d, J=8.7 Hz, 1H), 7.71 (d, J=8.0 Hz, 1H), 7.62 (d, J=1.8 Hz, 1H), 7.56 (d, J=8.3 Hz, 1H), 7.47-7.43 (m, 1H), 7.37 (t, J=7.6 Hz, 1H), 7.22 (t, J=7.5 Hz, 1H), 7.15 (d, J=1.8 Hz, 1H). ¹³C NMR (151 MHz, CDCl₃) δ 176.42 (q, J=36.7 Hz), 142.24, 136.76, 136.24, 127.64, 126.93, 126.82, 125.07, 124.23, 122.74 (q, J=6.4 Hz), 121.26, 121.09, 118.31, 117.56 (q, J=289.0 Hz), 114.22, 112.52, 107.35, 104.33. Mass calculated for (C₁₈H₁₀BrF₃N₂O—H)⁻ 405.0, found 404.9.

### Specific Procedure for the Synthesis of 1-(6-bromo-5′-chloro-1H,1′H-[2,2′-biindol]-3-yl)-2,2,2-trifluoroethan-1-one (178)

A solution of 202 (500 mg, 1.2 mmol), Na₂CO₃ (1.5M aqueous solution, 4.0 mL, 6.0 mmol)) and (5-chloro-1H-indol-2-yl)boronic acid (500 mg, 1.7 mmol) in ACN (12 mL) was purged with argon for 10 min followed by the addition of Pd(PPh₃)₂Cl₂ catalyst (100 mg, 0.14 mmol). The mixture was heated by microwave at 90° C. for 2h. The reaction mixture was diluted with EtOAc (100 mL) and washed with H₂O (2×50 mL) and brine (50 mL). The organic phase was dried over anhydrous Na₂SO₄ and concentrated. The residue was dissolved in TFA/DCM mixture (1:3, 20 mL) and stirred at rt for 1 h. The reaction mixture was concentrated and then purified by automated flash chromatography to give 178 as yellow solid (132 mg, 25%). ¹H NMR (400 MHz, DMSO) δ 13.17 (s, 1H), 11.94 (s, 1H), 7.86 (d, J=8.6 Hz, 1H), 7.78 (d, J=2.1 Hz, 1H), 7.74 (d, J=1.7 Hz, 1H), 7.58 (d, J=8.7 Hz, 1H), 7.51 (dd, J=8.7, 1.9 Hz, 1H), 7.25 (dd, J=8.7, 2.1 Hz, 1H), 7.15 (d, J=1.3 Hz, 1H). ¹³C NMR (151 MHz, DMSO) δ 175.17 (q, J_{C,F}=35.6 Hz), 141.58, 137.52, 135.90, 129.23, 128.73, 126.60, 125.04, 124.93, 123.95, 122.42 (q, J_{C,F}=3.9 Hz), 120.46, 117.17, 117.17 (q, J_{C,F}=290.5 Hz), 115.60, 114.14, 107.32, 105.88. Mass calculated for (C₁₈H₉BrClF₃N₂O—H)⁻ 441.0, found 441.0.

### Specific procedure for the synthesis of 1-(6-bromo-5′-methoxy-1H,1′H-[2,2′-biindol]-3-yl)-2,2,2-trifluoroethan-1-one (179)

A solution of 202 (100 mg, 0.24 mmol), Na₂CO₃ (1M aqueous solution, 1.2 mL, 1.2 mmol)) and (5-methoxy-1H-indol-2-yl)boronic acid (100 mg, 0.34 mmol) in ACN (2 mL) was purged with argon for 10 min followed by the addition of Pd(PPh₃)₂Cl₂ catalyst (25 mg, 0.036 mmol). The mixture was heated by microwave at 100° C. for 2h. The reaction mixture was diluted with EtOAc (50 mL) and washed with H₂O (2×20 mL) and brine (20 mL). The organic phase was dried over anhydrous Na₂SO₄ and concentrated. The residue was dissolved in TFA/DCM mixture (1:3, 4 mL) and stirred at rt for 1 h. and then concentrated. The residue was partially purified by automated flash chromatography and the recrystallized with EtOAc/hexanes to give 179 as yellow solid (16 mg, 15%). ¹H NMR (400 MHz, DMSO) δ 13.05 (s, 1H), 11.66 (s, 1H), 7.84 (d, J=8.6 Hz, 1H), 7.72 (d, J=1.7 Hz, 1H), 7.52-7.42 (m, 2H), 7.17 (d, J=2.2 Hz, 1H), 7.14 (d, J=1.6 Hz, 1H), 6.91 (dd, J=8.9, 2.3 Hz, 1H), 3.80 (s, 3H). ¹³C NMR (101 MHz, DMSO) δ 174.84 (q, J=35.6 Hz), 154.50, 142.54, 137.56, 132.84, 128.14, 127.90, 126.43, 125.00, 122.35 (q, J=4.2 Hz), 117.33 (q, J=290.7 Hz), 117.00, 115.45, 115.26, 113.50, 106.76, 106.29, 101.97, 55.78. Mass calculated for (C₁₉H₁₂BrF₃N₂O₂—H)⁻ 435.0, found 435.0.

### Specific Procedure for the Synthesis of 6-bromo-5′-chloro-1H,1′H-[2,2′-biindole]-3-carboxylic acid (162)

A suspension of compound 179 (530 mg, 1.2 mmol) in an aqueous solution of NaOH (20%, 10 mL) was heated at 110° C. for 30 min. After cooling to rt the reaction mixture was acidified with 15% aqueous HCl solution to pH of 1-2. The resulting mixture was extracted with EtOAc (×2) and the organic phase was dried over anhydrous Na₂SO₄ and concentrated. The residue was partially purified by automated flash chromatography and then recrystallized with EtOAc and hexanes to give compound 162 as a brown solid (160 mg, 34%). ¹H NMR (600 MHz, DMSO) δ 12.34 (s, 1H), 8.13 (d, J=8.5 Hz, 1H), 7.73 (d, J=1.8 Hz, 1H), 7.63-7.56 (m, 2H), 7.30 (d, J=8.7 Hz, 1H), 7.23 (s, 1H), 7.17 (dd, J=8.6, 1.9 Hz, 1H). ¹³C NMR (151 MHz, DMSO) δ 168.38, 163.56, 137.19, 135.76, 135.18, 131.64, 129.21, 127.64, 124.68, 124.45, 122.95, 119.95, 115.93, 114.15, 114.12, 101.88. Mass calculated for (C₁₇H₁₀BrClN₂O₂—H)⁻ 389.0, found 388.9.

### Example 36: Synthesis of Compound 164

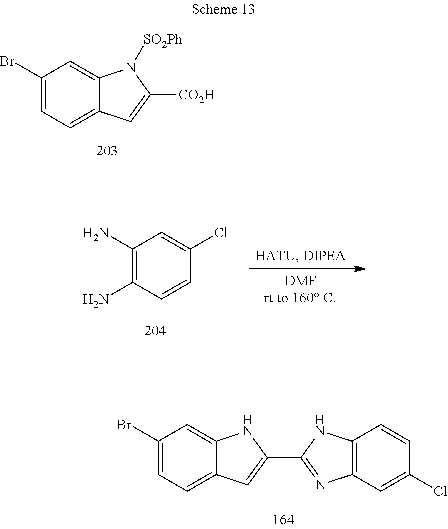

A mixture of 203 (60 mg, 0.16 mmol), 204 (50 mg, 0.35 mmol), HATU (90 mg, 0.24 mmol) and DIPEA (0.1 mL, 0.57 mmol) in DMF (2 mL) was stirred at rt for 3h and then heated by microwave at 160° C. for 5h. The reaction mixture was diluted with EtOAc (50 mL) and washed with H₂O (2×20 mL) and brine (20 mL). The organic phase was dried over anhydrous Na₂SO₄ and concentrated. The crude product was purified by automated flash chromatography to give compound 164 as a pale brown solid (27 mg, 49%).

### 2-(6-bromo-1H-indol-2-yl)-5-chloro-1H-benzo[d]imidazole (164)

¹H NMR (400 MHz, DMSO-d₆) δ 13.24 (d, J=15.6 Hz, 1H), 12.19 (d, J=12.2 Hz, 1H), 7.92-7.52 (m, 4H), 7.31-7.23 (m, 2H), 7.20 (dd, J=8.4, 1.9 Hz, 1H). Mass calculated for (C₁₅H₉BrClN₃+H)⁺ 346.0, found 346.0.

### Example 37: General Procedure for the Synthesis of Amide Linked Compounds

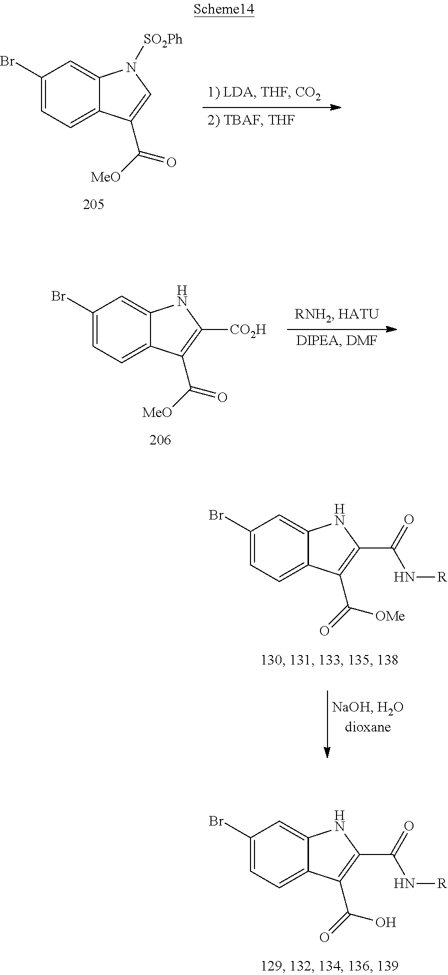

### Synthesis of Compound 206

To a stirred solution of 205 (6.61 g, 16.8 mmol) in anhydrous THF (125 mL) at −78° C. was added a solution of LDA (2.0 M in THF/heptane/ethylbenzene, 12.0 ml, 24 mmol) dropwise. The mixture was stirred at 0° C. for 15 min and then CO₂ gas was bubbled through for 30 min. The reaction was quenched with H₂O and then diluted with EtOAc. The resulting mixture was washed with 0.5M aqueous HCl, brine, dried over anhydrous Na₂SO₄, filtered and concentrated in vacuo. The residue was dissolved in THF (150 ml) followed by the addition of TBAF (50.0 mL, 1 M in THF, 50 mmol). The mixture was stirred at rt for 18h and then diluted with EtOAc. The mixture was washed with H₂O, brine, dried over anhydrous Na₂SO₄ and concentrated. The crude product was partially purified by automated flash chromatography and then recrystallized with EtOAc/hexanes to give compound 206 as a brown solid (2.85 g, 57%). ¹H NMR (400 MHz, DMSO-d₆) δ 14.42 (s, 1H), 12.91 (s, 1H), 7.95 (d, J=8.7 Hz, 1H), 7.72 (d, J=1.8 Hz, 1H), 7.44 (dd, J=8.7, 1.8 Hz, 1H), 3.97 (s, 3H).

### General Procedure for the Synthesis of Compounds 130, 131, 133, 135 and 138

To a stirred solution of 206 (1 mmol), DIPEA (3.4 mmol) and the corresponding amine (4.0 mmol) in DMF (3 mL) at rt was added HATU (1.4 mmol) and the mixture was stirred overnight. The precipitate was collected by filtration and the solid was washed with EtOAc (×2) to give the desired product. In the preparation of compound 138 only 0.9 mmol of DIPEA was used.

### Methyl 6-bromo-2-((4-chlorophenyl)carbamoyl)-1H-indole-3-carboxylate (130)

Yield=37%, white solid. ¹H NMR (400 MHz, DMSO-d₆) δ 12.95 (s, 1H), 12.39 (s, 1H), 8.06 (d, J=8.8 Hz, 1H), 7.80 (d, J=8.9 Hz, 2H), 7.77 (d, J=1.8 Hz, 1H), 7.51 (d, J=8.8 Hz, 2H), 7.44 (dd, J=8.8, 1.9 Hz, 1H), 4.00 (s, 3H).

### Methyl 6-bromo-2-((4-bromophenyl)carbamoyl)-1H-indole-3-carboxylate (131)

Yield=49%, yellow solid. ¹H NMR (400 MHz, DMSO-d₆) δ 12.94 (s, 1H), 12.40 (s, 1H), 8.06 (d, J=8.8 Hz, 111), 7.80-7.70 (m, 3H), 7.63 (d, J=8.5 Hz, 2H), 7.44 (dd, J=8.7, 1.8 Hz, 1H), 4.00 (s, 3H). Mass calculated for (C₁₇H₁₂Br₂N₂O₃—H)⁻ 450.9, found 450.9.

### Methyl 6-bromo-2-((4-methoxyphenyl)carbamoyl)-1H-indole-3-carboxylate (133)

Yield=75%, white solid. ¹H NMR (400 MHz, DMSO-d₆) δ 12.90 (s, 1H), 12.27 (s, 1H), 8.05 (d, J=8.7 Hz, 1H), 7.76 (d, J=1.8 Hz, 1H), 7.70 (d, J=9.0 Hz, 2H), 7.43 (dd, J=8.7, 1.8 Hz, 1H), 7.01 (d, J=9.0 Hz, 2H), 4.01 (s, 3H), 3.78 (s, 3H). ¹³C NMR (101 MHz, DMSO) δ 167.51, 157.33, 156.52, 138.05, 135.96, 131.68, 126.09, 125.84, 124.99, 121.68, 117.69, 115.97, 114.80, 104.96, 55.75, 52.96. Mass calculated for (C₁₈H₁₅BrN₂O₄—H)⁻ 401.0, found 400.9.

### Methyl 6-bromo-2-((6-methoxypyridin-3-yl)carbamoyl)-1H-indole-3-carboxylate (134)

Yield=58%, white solid. ¹H NMR (400 MHz, DMSO-d₆) δ 12.95 (s, 1H), 12.18 (s, 1H), 8.58 (d, J=2.7 Hz, 1H), 8.14-7.96 (m, 2H), 7.76 (d, J=1.8 Hz, 1H), 7.45 (dd, J=8.8, 1.8 Hz, 1H), 6.93 (d, J=8.9 Hz, 1H), 4.00 (s, 3H), 3.88 (s, 3H). ¹³C NMR (101 MHz, DMSO) δ 167.18, 160.81, 158.12, 138.71, 137.51, 136.07, 132.39, 129.58, 126.16, 125.72, 124.94, 117.79, 115.98, 111.14, 105.32, 53.82, 52.92. Mass calculated for (C₁₇H₁₄BrN₃O₄—H)⁻ 404.0, found 403.9.

### Methyl 6-bromo-2-((6-chloropyridin-3-yl)carbamoyl)-1H-indole-3-carboxylate (138)

Yield=71%, white solid. ¹H NMR (400 MHz, DMSO-d₆) δ 13.00 (s, 1H), 12.39 (s, 1H), 8.78 (d, J=2.7 Hz, 1H), 8.25 (dd, J=8.7, 2.8 Hz, 1H), 8.06 (d, J=8.8 Hz, 1H), 7.77 (d, J=1.8 Hz, 1H), 7.61 (d, J=8.7 Hz, 1H), 7.45 (dd, J=8.8, 1.8 Hz, 1H), 4.00 (s, 3H). Mass calculated for (C₁₆H₁₁BrClN₃O₃)⁺ 408.0, found 407.7.

### General Procedure for the Synthesis of Compounds 129, 132, 134, 136 and 139

NaOH (2M in H₂O, 20 mmol) was added to a stirred solution/suspension of the corresponding starting material (1 mmol) in dioxane (16 mL) and the mixture was heated at 60° C. overnight. The mixture was acidified (pH 1-2) and the solid was collected by filtration. When there was no/low solid formation, the acidified mixture was extracted with EtOAc and then recrystallized with EtOAc and hexanes.

### 6-Bromo-2-((4-chlorophenyl)carbamoyl)-1H-indole-3-carboxylic acid (129)

Yield=74%, pale yellow solid. ¹H NMR (400 MHz, DMSO-d₆) δ 13.98 (s, 1H), 13.29 (s, 1H), 12.86 (s, 1H), 8.10 (d, J=8.8 Hz, 1H), 7.84-7.69 (m, 3H), 7.50 (d, J=8.8 Hz, 2H), 7.42 (dd, J=8.8, 1.9 Hz, 1H). ¹³C NMR (101 MHz, DMSO) δ 169.30, 157.94, 137.77, 137.14, 135.96, 129.64, 128.38, 126.54, 125.99, 125.37, 121.64, 117.84, 115.91, 106.64.

### 6-Bromo-2-((4-bromophenyl)carbamoyl)-1H-indole-3-carboxylic acid (132)

Yield=61%, white solid. ¹H NMR (400 MHz, DMSO) δ 13.93 (bs, 1H), 13.32 (s, 1H), 12.83 (s, 1H), 8.11 (d, J=8.8 Hz, 1H), 7.77 (d, J=1.5 Hz, 1H), 7.73 (d, J=8.8 Hz, 2H), 7.63 (d, J=8.8 Hz, 2H), 7.42 (dd, J=8.8, 1.7 Hz, 1H). ¹³C NMR (101 MHz, DMSO) δ 169.28, 157.94, 138.16, 137.11, 135.99, 132.53, 126.51, 125.99, 125.39, 122.03, 117.81, 116.42, 115.90. Mass calculated for (C₁₆H₁₀Br₂N₂O₃—H)⁻ 436.9, found 436.8.

### 6-Bromo-2-((4-methoxyphenyl)carbamoyl)-1H-indole-3-carboxylic acid (134)

Yield=73%, yellow solid. ¹H NMR (400 MHz, DMSO-d₆) δ 13.90 (s, 1H), 12.65 (s, 1H), 8.15 (d, J=8.7 Hz, 1H), 7.85-7.57 (m, 3H), 7.38 (dd, J=8.7, 1.9 Hz, 1H), 7.13-6.83 (m, 2H), 3.78 (s, 3H). Mass calculated for (C₁₇H₁₃BrN₂O₄—H)⁻ 387.0, found 386.9.

### 6-Bromo-2-((6-methoxypyridin-3-yl)carbamoyl)-1H-indole-3-carboxylic acid (136)

Yield=80%, pale yellow solid. ¹H NMR (400 MHz, DMSO-d₆) δ 13.97 (s, 1H), 13.09 (s, 1H), 12.87 (s, 1H), 8.54 (d, J=2.7 Hz, 1H), 8.21-8.00 (m, 2H), 7.76 (d, J=1.8 Hz, 1H), 7.42 (dd, J=8.7, 1.9 Hz, 1H), 6.94 (d, J=8.9 Hz, 1H), 3.88 (s, 3H). ¹³C NMR (101 MHz, DMSO) δ 169.33, 160.73, 157.84, 138.50, 137.17, 135.96, 132.20, 129.75, 126.45, 126.03, 125.32, 117.79, 115.92, 111.17, 106.37, 53.79. Mass calculated for (C₁₆H₁₂BrN₃O₄—H)⁻ 388.0, found 388.0.

### 6-Bromo-2-((6-chloropyridin-3-yl)carbamoyl)-1H-indole-3-carboxylic acid (139)

Yield=48%, white solid. ¹H NMR (400 MHz, DMSO-d₆) δ 14.05 (s, 1H), 13.59 (s, 1H), 12.87 (s, 1H), 8.73 (d, J=2.7 Hz, 1H), 8.26 (dd, J=8.7, 2.8 Hz, 1H), 8.12 (d, J=8.8 Hz, 1H), 7.76 (d, J=1.8 Hz, 1H), 7.60 (d, J=8.6 Hz, 1H), 7.42 (dd, J=8.7, 1.9 Hz, 1H). ¹³C NMR (101 MHz, DMSO) δ 169.20, 158.54, 144.95, 141.27, 136.45, 136.09, 135.22, 130.84, 126.45, 126.05, 125.43, 125.15, 117.97, 115.91. Mass calculated for (C₁₅H₉BrClN₃O₃—H)⁻ 392.0, found 391.9.

### Example 38: Synthesis of Compound 137

To a stirred solution of 206 (50 mg, 0.17 mmol) (Example 19) and the corresponding amine (100 mg, 0.78 mmol) in DMF (2 mL) at rt was added HATU (90 mg, 0.24 mmol) and the mixture was stirred overnight. The precipitate was collected by filtration and the solid was washed with EtOAc (×2) to give the desired product 137 as a white solid (51 mg, 74%).

### Methyl 6-bromo-2-((5-chloropyridin-2-yl)carbamoyl)-1H-indole-3-carboxylate (137)

¹HNMR (400 MHz, DMSO-d₆) δ 13.25 (s, 1H), 13.04 (s, 1H), 8.50 (d, J=2.6 Hz, 1H), 8.38 (d, J=8.9 Hz, 1H), 8.21-7.94 (m, 2H), 7.80 (s, 1H), 7.46 (d, J=8.8 Hz, 1H), 4.06 (s, 3H). Mass calculated for (C₁₆H₁₁BrClN₃O₃)⁺ 408.0, found 408.0.

### Example 39: Synthesis of Compound 128

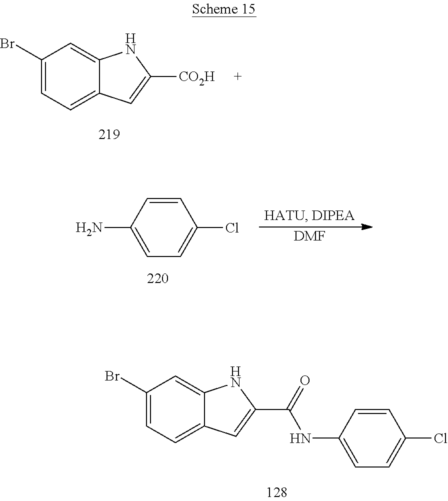

To a stirred solution of 6-bromo-1H-indole-2-carboxylic acid 219 (100 mg, 0.41 mmol), HATU (300 mg, 0.78 mmol) and DIPEA (0.25 mL, 1.44 mmol) in DMF (5 mL) at rt was added amine 220 (65 mg, 0.51 mmol) and the mixture was stirred overnight. The reaction mixture was diluted with EtOAc (100 mL) and washed with H₂O (2×50 mL) and brine (50 mL). The organic phase was dried over anhydrous Na₂SO₄ and concentrated. The crude product was purified by automated flash chromatography to give compound 128 as a brown solid (81 mg, 56%).

### 6-bromo-N-(4-chlorophenyl)-1H-indole-2-carboxamide (128)

¹H NMR (400 MHz, DMSO) δ 11.93 (s, 1H), 10.40 (s, 1H), 7.85 (d, J=8.9 Hz, 2H), 7.68 (d, J=8.5 Hz, 1H), 7.64 (d, J=0.7 Hz, 1H), 7.49-7.42 (m, 3H), 7.22 (dd, J=8.5, 1.8 Hz, 1H). ¹³C NMR (101 MHz, DMSO) δ 159.89, 138.25, 138.03, 132.53, 129.12, 127.75, 126.44, 124.22, 123.48, 122.15, 117.15, 115.28, 104.66. Mass calculated for (C₁₅H₁₀BrClN₂O—H)⁻ 349.0, found 348.9.

### Example 40: Synthesis of Compounds 146 and 147

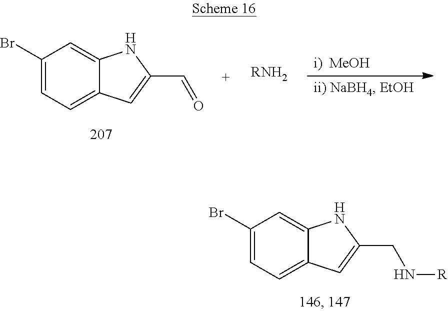

A mixture of 207 (1 mmol) and the corresponding amine (2 mmol) in MeOH (10 mL) was stirred at rt for 3 h and then concentrated. The residue was dissolved in EtOH (10 mL) followed by the addition of NaBH₄ (1.5 mmol) and the mixture was stirred at rt overnight. The reaction mixture was diluted with EtOAc (100 mL) and washed with H₂O (50 mL) and brine (50 mL). The organic phase was dried over anhydrous Na₂SO₄ and concentrated. The crude product was purified by automated flash chromatography to give the desired product.

### N-Benzyl-1-(6-bromo-1H-indol-2-yl)methanamine (146)

Yield=94%, brown oil. ¹H NMR (400 MHz, DMSO) δ 11.12 (s, 1H), 7.51-7.48 (m, 1H), 7.40 (d, J=8.4 Hz, 1H), 7.38-7.30 (m, 4H), 7.27-7.21 (m, 1H), 7.07 (dd, J=8.4, 1.8 Hz, 1H), 6.30 (d, J=1.1 Hz, 1H), 3.80 (s, 2H), 3.71 (s, 2H), 2.70 (bs, 1H). Mass calculated for (C₁₆H₁₅BrN₂+H)⁺ 315.0, found 315.0.

### N-((6-Bromo-1H-indol-2-yl)methyl)-4-chloroaniline (147)

Yield=29%, white solid. ¹H NMR (400 MHz, DMSO) δ 11.18 (s, 1H), 7.49 (bs, 1H), 7.40 (d, J=8.4 Hz, 1H), 7.12-7.04 (m, 3H), 6.68-6.62 (m, 2H), 6.35 (t, J=4.0 Hz, 2H), 4.37 (d, J=5.6 Hz, 2H). Mass calculated for (C₁₅H₁₂BrClN₂+H)⁺ 335.0, found 334.9.

### Example 41: Synthesis of Compounds 148 and 149

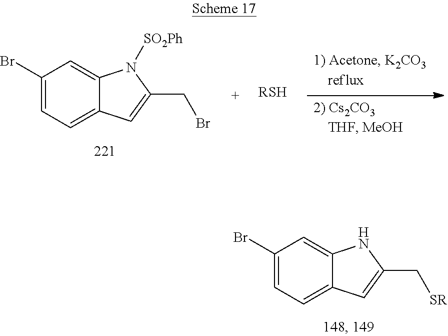

A mixture of bromide 221 (1.0 mmol), the corresponding thiol (1.1 mmol) and K₂CO₃ (2.0) in acetone (25 mL) was refluxed for 2 h, filtered through a pad of celite and then concentrated. The residue was dissolved in THF: MeOH, (2:1, 9 mL) and Cs₂CO₃ (2.0 mmol) was added. The mixture has heated in a microwave reactor at 90° C. for 30 min. The reaction mixture was diluted with EtOAc (100 mL) and washed with H₂O (50 mL) and brine (50 mL). The organic phase was dried over anhydrous Na₂SO₄ and concentrated. The crude product was purified by automated flash chromatography to give the desired product.

### 2-(((6-Bromo-1H-indol-2-yl)methyl)thio)-1H-benzo[d]imidazole (148)

Yield=57%, white solid. ¹H NMR (400 MHz, DMSO) δ 12.63 (s, 1H), 11.41 (s, 1H), 7.58-7.34 (m, 4H), 7.18-7.11 (m, 2H), 7.08 (dd, J=8.4, 1.8 Hz, 1H), 6.41 (s, 1H), 4.72 (s, 2H). Mass calculated for (C₁₆H₁₂BrN₃S+H)⁺ 360.0, found 360.0.

### 2-((Benzylthio)methyl)-6-bromo-1H-indole (149)

Yield=61%, white solid.

¹H NMR (400 MHz, DMSO) δ 11.25 (s, 1H), 7.52-7.48 (m, 1H), 7.43 (d, J=8.4 Hz, 1H), 7.36-7.23 (m, 5H), 7.09 (dd, J=8.4, 1.8 Hz, 1H), 6.36 (d, J=1.2 Hz, 1H), 3.79 (s, 2H), 3.70 (s, 2H). ¹³C NMR (151 MHz, DMSO) δ 138.63, 137.82, 137.31, 129.36, 128.89, 127.37, 127.34, 122.17, 121.74, 113.98, 113.89, 101.38, 35.62, 28.31. Mass calculated for (C₁₆H₁₄BrNS+H)⁺ 334.0, found 334.0.

### Example 42: Synthesis of Compound 154

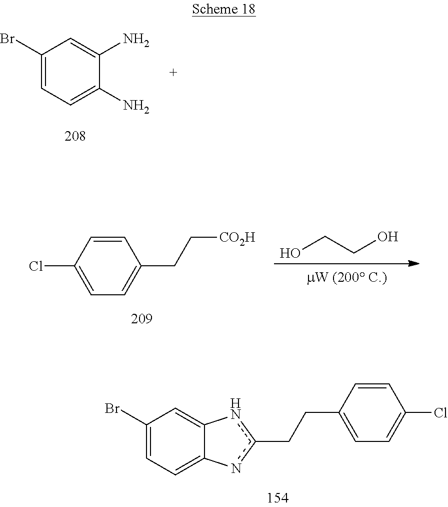

A mixture of 208 (100 mg, 0.53 mmol) and 209 (100 mg, 0.54 mmol) in ethylene glycol (0.5 mL) was heated by microwave at 200° C. for 30 min. The mixture was diluted with EtOAc (50 mL) and washed with H₂O (20 mL), saturated aqueous NaHCO₃ (20 mL) and brine (20 mL). The organic phase was dried over anhydrous Na₂SO₄ and concentrated. The crude product was purified by automated flash chromatography to give compound 154 (16 mg, 9%).

### 6-bromo-2-(4-chlorophenethyl)-1H-benzo[d]imidazole (154)

¹H NMR (400 MHz, MeOD) δ 7.63 (d, J=1.7 Hz, 1H), 7.39 (d, J=8.5 Hz, 1H), 7.30 (dd, J=8.5, 1.8 Hz, 1H), 7.23 (d, J=8.4 Hz, 2H), 7.15 (d, J=8.4 Hz, 2H), 3.18-3.08 (m, 4H). ¹³C NMR (101 MHz, MeOD) δ 155.65, 139.03, 131.80, 129.70, 129.56, 128.17, 128.16, 124.96, 117.08, 115.22, 114.67, 33.09, 30.20. Mass calculated for (C₁₅H₁₂BrClN₂+H)⁺ 335.0, found 334.9.

### Example 43: Synthesis of Compounds 155 and 156

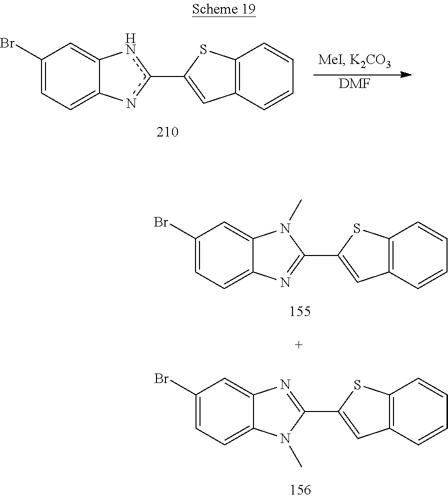

A mixture of 210 (40 mg, 0.12 mmol), MeI (15 μL, 0.24 mmol) and K₂CO₃ (80 mg, 0.58 mmol) in DMF (1.5 mL) was stirred at rt overnight. The mixture was diluted with EtOAc (50 mL) and washed with H₂O (20 mL) and brine (20 mL). The organic phase was dried over anhydrous Na₂SO₄ and concentrated. The crude product was purified by automated flash chromatography to give the desired product.

### 2-(Benzo[b]thiophen-2-yl)-6-bromo-1-methyl-1H-benzo[d]imidazole (155)

(13 mg, 32%)¹H NMR (600 MHz, DMSO) δ 8.22 (s, 1H), 8.08-8.05 (m, 1H), 8.03-7.99 (m, 2H), 7.66 (d, J=8.5 Hz, 1H), 7.50-7.47 (m, 2H), 7.41 (dd, J=8.5, 1.7 Hz, 1H), 4.14 (s, 3H). ¹³C NMR (151 MHz, DMSO) δ 148.35, 141.73, 140.46, 140.05, 138.62, 132.74, 126.48, 125.85, 125.67, 125.46, 125.31, 122.79, 121.14, 115.87, 114.13, 32.68. Mass calculated for (C₁₆H₁₁BrN₂S+H)⁺ 345.0, found 345.0.

### 2-(Benzo[b]thiophen-2-yl)-5-bromo-1-methyl-1H-benzo[d]imidazole (156)

(18 mg, 44%) ¹H NMR (600 MHz, DMSO) δ 8.22 (s, 1H), 8.08-8.05 (m, 1H), 8.03-7.99 (m, 1H), 7.92 (s, 1H), 7.70 (d, J=8.6 Hz, 1H), 7.50-7.46 (m, 3H), 4.15 (s, 3H). ¹³C NMR (151 MHz, DMSO) δ 148.65, 144.01, 140.42, 140.09, 136.56, 132.66, 126.52, 126.09, 125.81, 125.47, 125.33, 122.80, 121.82, 115.11, 113.01, 32.67. Mass calculated for (C₁₆H₁₁BrN₂S+H)⁺ 345.0, found 345.0.

### Example 44: Synthesis of Compound 160

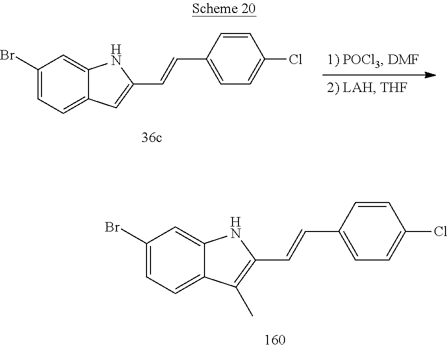 [SMILES: Clc1ccc(/C=C/c2cc3ccc(Br)cc3[nH]2)cc1 ; Cc1c(/C=C/c2ccc(Cl)cc2)[nH]c2cc(Br)ccc12]

To cooled DMF (3 mL) at 0° C. under Ar was added POCl₃ (1.4 mmol) and the mixture was allowed to warm to rt followed by the addition of 36c (1.0 mmol) in DMF (1 mL). The mixture was heated at 35° C. for 2h and then diluted with EtOAc (50 mL). The mixture was washed with H₂O (20 mL) and brine (20 mL). The organic phase was dried over anhydrous Na₂SO₄ and concentrated. The residue was purified by automated flash chromatography to give the correspond aldehyde. This intermediate was dissolved in THF (1 mL) and cooled to 0° C. under Ar followed by addition of LAH (3.0 mmol). The resulting mixture was stirred at rt overnight and then quenched with H₂O (1 mL). The mixture was diluted with EtOAc (50 mL) and washed with H₂O (20 mL) and brine (20 mL). The organic phase was dried over anhydrous Na₂SO₄ and concentrated. The crude product was purified by automated flash chromatography to give compound 160 (11 mg, 28% for 2 steps).

### (E)-6-bromo-2-(4-chlorostyryl)-3-methyl-1H-indole (160)

¹H NMR (400 MHz, DMSO) δ 11.28 (s, 1H), 7.65 (d, J=8.5 Hz, 2H), 7.47-7.42 (m, 4H), 7.38 (d, J=16.5 Hz, 1H), 7.15-7.05 (m, 2H), 2.37 (s, 3H). ¹³C NMR (101 MHz, DMSO) 138.08, 136.54, 133.99, 132.14, 129.25, 128.41, 128.37, 125.79, 121.96, 120.78, 118.53, 115.66, 113.51, 112.09, 8.87. Mass calculated for (C₁₇H₁₃BrClN—H)⁻ 346.0, found 345.9.

### Example 45: Synthesis of Compounds 140, 142 and 144

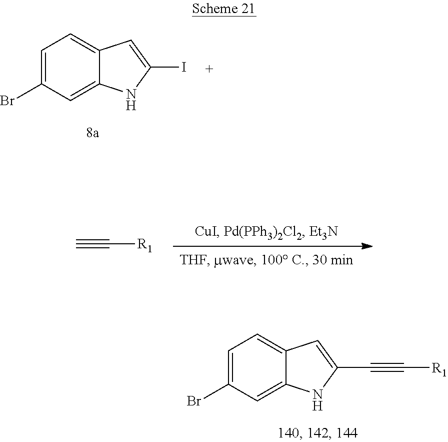

To a stirred solution of compound 8a (1 mmol) and the corresponding acetylene derivative (1 mmol) in anhydrous THF (4 ml) under argon was added CuI (0.15 mmol) and Pd(PPh₃)₂Cl₂ (0.10 mmol). The mixture was purged with argon for 10 min and then Et₃N (10 mmol) was added. The reaction mixture was heated at 100° C. by microwave for 30 min. The mixture was diluted with EtOAc and washed with H₂O, brine, dried over anhydrous Na₂SO₄ and concentrated. The crude product was purified by automated flash chromatography to give the desired product.

### 6-Bromo-2-((6-methoxypyridin-3-yl)ethynyl)-1H-indole (144)

Yield=10%, off-white solid. ¹H NMR (400 MHz, MeOD) δ 8.36 (dd, J=2.3, 0.8 Hz, 1H), 7.82 (dd, J=8.6, 2.3 Hz, 1H), 7.55-7.49 (m, 1H), 7.46 (d, J=8.5 Hz, 1H), 7.17 (dd, J=8.5, 1.8 Hz, 1H), 6.86 (dd, J=8.7, 0.8 Hz, 1H), 6.75 (d, J=1.0 Hz, 1H), 3.97 (s, 3H). ¹³C NMR (101 MHz, MeOD) δ 163.73, 149.45, 140.98, 126.64, 122.75, 121.38, 116.05, 113.38, 112.69, 110.47, 107.32, 88.20, 82.79, 52.89. Mass calculated for (C₁₆H₁₁BrN₂O+H)⁺ 327.0, found 327.0.

### 6-Bromo-2-(pyridin-2-ylethynyl)-1H-indole (140)

Yield=5%, yellow solid. ¹H NMR (400 MHz, MeOD) δ 8.59 (d, J=5.0 Hz, 1H), 7.91 (td, J=7.8, 1.8 Hz, 1H), 7.68 (dt, J=7.8, 1.1 Hz, 1H), 7.58-7.53 (m, 1H), 7.50 (d, J=8.5 Hz, 1H), 7.45 (ddd, J=7.7, 4.9, 1.2 Hz, 1H), 7.19 (dd, J=8.5, 1.7 Hz, 1H), 6.89 (d, J=1.0 Hz, 1H). ¹³C NMR (101 MHz, MeOD) δ 149.37, 142.27, 137.68, 137.28, 127.16, 126.47, 123.38, 123.03, 121.71, 118.27, 116.73, 113.60, 109.00, 90.28, 82.08. Mass calculated for (C₁₅H₉BrN₂+H)⁺ 297.0, found 296.9.

### 6-Bromo-2-((5-chloropyrimidin-2-yl)ethynyl)-1H-indole (142)

¹H NMR (400 MHz, DMSO) δ 12.21 (s, 1H), 9.01 (s, 2H), 7.67-7.48 (m, 2H), 7.23 (dd, J=8.5, 1.8 Hz, 1H), 7.10 (d, J=1.0 Hz, 1H). ¹³C NMR (101 MHz, DMSO) δ 156.93, 150.12, 138.18, 130.33, 126.44, 123.75, 123.17, 117.54, 117.39, 114.54, 111.29, 91.30, 81.45. Mass calculated for (C₁₄H₇BrClN₃—H)⁻ 330.0, found 329.8.

### Example 46: Synthesis of Compound 141

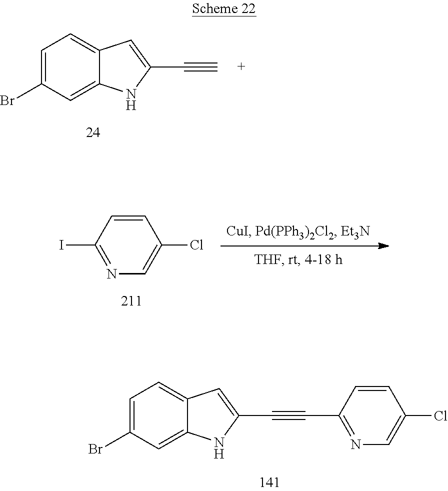 [SMILES: C#Cc1cc2ccc(Br)cc2[nH]1 ; Clc1ccc(I)nc1 ; Clc1ccc(C#Cc2cc3ccc(Br)cc3[nH]2)nc1]

A solution of compounds 24 (1 mmol) and 211 (1.2 mmol) in THF (10 ml) was purged with argon for 5 min followed by addition of CuI (0.15 mmol) and Pd(PPh₃)₂Cl₂ (0.1 mmol). After purging for an additional 5 min with argon, Et₃N (10 mmol) was added and the mixture was stirred at rt for 4-18 h. Upon completion of the reaction, as indicated by TLC or LC-MS, the mixture was diluted with EtOAc and filtered through celite. The filtrate was washed with H₂O, brine, dried over anhydrous Na₂SO₄ and concentrated. The residue was purified by automated flash chromatography to give compound 141 as a brown solid (53%).

### 6-bromo-2-((5-chloropyridin-2-yl)ethynyl)-1H-indole (141)

¹H NMR (400 MHz, DMSO) δ 12.05 (s, 1H), 8.70 (dd, J=2.6, 0.8 Hz, 1H), 8.04 (dd, J=8.4, 2.6 Hz, 1H), 7.72 (dd, J=8.4, 0.8 Hz, 1H), 7.60-7.53 (m, 2H), 7.21 (dd, J=8.4, 1.8 Hz, 1H), 6.98 (dd, J=2.0, 0.9 Hz, 1H). ¹³C NMR (101 MHz, DMSO) δ 149.52, 140.70, 137.92, 137.30, 131.44, 128.66, 126.55, 123.54, 122.93, 118.39, 116.86, 114.38, 109.80, 91.45, 83.08. Mass calculated for (C₁₅H₈BrClN₂—H)⁻ 329.0, found 328.9.

### Example 47: Synthesis of Compounds 143 and 145

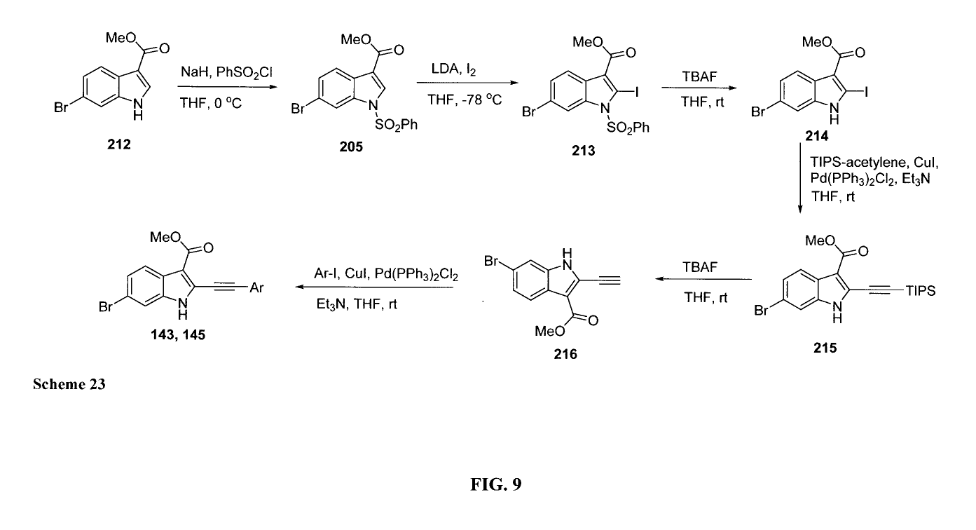

Compounds 143 and 145 were prepared according the general synthesis pathway shown in Scheme 23 (FIG. 9).

### Synthesis of methyl 6-bromo-1-(phenylsulfonyl)-1H-indole-3-carboxylate (205)

To a stirred solution of methyl carboxylate 212 (2.97 g, 11.7 mmol) in THF (50 ml) at 0° C. was added NaH (60% in oil, 560 mg, 14.0 mmol) gradually. After stirring at room temperature for 10 minutes, benzenesulphonyl chloride (1.80 ml, 14.1 mmol) was added and the mixture was further stirred at rt for 3h. The reaction was quenched with H₂O and extracted with EtOAc (2×50 ml). The combined organic phases were washed with brine, dried over anhydrous Na₂SO₄, filtered and concentrated in vacuo. The crude product was purified by automated flash chromatography to give compound 205 as a tan solid (4.24g, 92%). ¹H NMR (400 MHz, CDCl₃) δ 8.61 (d, J=1.7 Hz, 1H), 7.98-7.94 (m, 2H), 7.92 (d, J=8.6 Hz, 1H), 7.67-7.61 (m, 1H), 7.51 (t, J=7.9 Hz, 2H), 7.44 (dd, J=8.7, 1.7 Hz, 1H), 3.95 (s, 3H). Mass calculated for (C₁₆H₁₂BrNO₄S+H)⁺ 394.0, found 394.0.

### Synthesis of methyl 6-bromo-2-iodo-1-(phenylsulfonyl)-1H-indole-3-carboxylate (213)

To a stirred solution of 205 (4.24 g, 2.5 mmol) in anhydrous THF (100 mL) at −78° C. was added a solution of LDA (2.0 M in THF, 8.0 ml, 16 mmol) dropwise. The mixture was stirred at −78° C. for 10 min and then warmed to 0° C. for 30 min. The solution was re-cooled to −78° C. and then a solution of I₂ (4.11 g, 16.2 mmol) in THF (30 mL) was added. The reaction mixture was stirred at 0° C. for 15 minutes and then allowed to warm to rt for 2h. The reaction was quenched with saturated aqueous NH₄Cl solution and extracted with EtOAc (2×50 ml). The combined organic phases were washed with brine, dried over anhydrous Na₂SO₄, filtered and concentrated in vacuo. The crude product was purified by automated flash chromatography to give compound 213 as a tan solid (2.74g, 50%). ¹H NMR (400 MHz, CDCl₃) δ 8.61 (d, J=1.7 Hz, 1H), 7.96 (dd, 2H), 7.92 (d, J=8.6 Hz, 1H), 7.67-7.61 (m, 1H), 7.51 (t, J=7.9 Hz, 2H), 7.44 (dd, J=8.7, 1.7 Hz, 1H), 3.95 (s, 3H).

### Synthesis of methyl 6-bromo-2-iodo-1H-indole-3-carboxylate (214)

To a stirred solution of 213 (2.74 g, 5.3 mmol) in THF (66 ml) was added TBAF (10.6 mL, 1 M in THF, 10.6 mmol) and the mixture was stirred at rt for 18h. The reaction mixture was diluted with EtOAc and washed with H₂O, brine, dried over anhydrous Na₂SO₄ and concentrated. The crude product was purified by automated flash chromatography to give compound 214 as a white solid (1.60 g, 80%). ¹H NMR (400 MHz, MeOD) δ 7.92 (d, J=8.7 Hz, 1H), 7.53 (d, J=1.8 Hz, 1H), 7.26 (dd, J=8.6, 1.8 Hz, 1H), 3.91 (s, 3H). Mass calculated for (C₁₀H₇BrINO₂—H)⁻ 377.9, found 377.9.

### Synthesis of methyl 6-bromo-2-((triisopropylsilyl)ethynyl)-1H-indole-3-carboxylate (215)

A solution of compound 214 (1.00 g, 2.6 mmol) and triisopropylsilyl acetylene (0.7 mL, 3.2 mmol) in THF (10 ml) was purged with argon for 5 min followed by addition of CuI (77 mg, 0.4 mmol) and Pd(PPh₃)₂Cl₂ (186 mg, 0.26 mmol). After purging for an additional 5 min with argon, Et₃N (3.7 mL, 27.0 mmol) was added and the mixture was stirred at rt for 16 h. The mixture was diluted with EtOAc and filtered through a pad of celite. The filtrate was washed with H₂O, brine, dried over anhydrous Na₂SO₄ and concentrated. The residue was purified by automated flash chromatography to give compound 215 as a white solid (0.85 g, 74%). ¹H NMR (400 MHz, CDCl₃) δ 8.51 (s, 1H), 8.08 (d, J=8.6 Hz, 1H), 7.47 (d, J=1.7 Hz, 1H), 7.36 (dd, J=8.6, 1.7 Hz, 1H), 3.92 (s, 3H), 1.19-1.15 (m, 21H).

### Synthesis of methyl 6-bromo-2-ethynyl-1H-indole-3-carboxylate (216)

To a stirred solution of 215 (1.0 mmol) in THF (10 ml) was added TBAF (2.0 ml, 1 M, 2.0 mmol) and the mixture was stirred at rt for 18h. The reaction mixture was diluted with EtOAc and washed with H₂O, brine, dried over anhydrous Na₂SO₄ and concentrated. The crude product was purified by automated flash chromatography to give compound 216 as a white solid (83%). ¹H NMR (400 MHz, Methanol) δ 7.96 (d, J=8.6 Hz, 1H), 7.55 (d, J=1.7 Hz, 1H), 7.32 (dd, J=8.7, 1.7 Hz, 1H), 4.18 (s, 1H), 3.92 (s, 3H). Mass calculated for (C₁₂H₈BrNO₂—H)⁻ 276.0, found 276.0.

### General Method for the Synthesis of Compounds 143 and 145

A solution of compound 216 (1 mmol) and the corresponding iodide (1.2 mmol) in THF (10 ml) was purged with argon for 5 min followed by addition of CuI (0.15 mmol) and Pd(PPh₃)₂Cl₂ (0.1 mmol). After purging for an additional 5 min with argon, Et₃N (10 mmol) was added and the mixture was stirred at rt for 4-18 h. Upon completion of the reaction, as indicated by TLC or LC-MS, the mixture was diluted with EtOAc and filtered through a pad of celite. The filtrate was washed with H₂O, brine, dried over anhydrous Na₂SO₄ and concentrated. The residue was purified by automated flash chromatography to give the desired product.

### Methyl 6-bromo-2-((5-chloro-1H-indol-2-yl)ethynyl)-1H-indole-3-carboxylate (145)

Yield=42%, yellow solid. ¹H NMR (400 MHz, DMSO) δ 12.79 (s, 1H), 12.03 (d, 1H), 7.96 (d, J=8.6 Hz, 1H), 7.66 (d, J=2.1 Hz, 1H), 7.61 (d, J=1.8 Hz, 1H), 7.41 (d, J=8.6 Hz, 1H), 7.39 (dd, J=8.7, 1.8 Hz, 1H), 7.22 (dd, J=8.7, 2.1 Hz, 1H), 6.95 (dd, J=2.0, 0.9 Hz, 1H), 3.89 (s, 3H). ¹³C NMR (101 MHz, DMSO) δ 163.51, 136.72, 135.28, 128.14, 125.10, 124.65, 124.53, 123.76, 123.56, 122.79, 19.65, 118.62, 116.96, 114.43, 113.12, 109.93, 109.00, 89.36, 83.89, 51.20. Mass calculated for (C₂₀H₁₂BrClN₂O₂—H)⁻ 425.0, found 424.9.

### Methyl 6-bromo-2-((5-chloropyrimidin-2-yl)ethynyl)-1H-indole-3-carboxylate (143)

Yield=15%, yellow solid. ¹H NMR (400 MHz, DMSO-d₆) δ 13.07 (s, 1H), 9.06 (s, 2H), 7.99 (d, J=8.6 Hz, 1H), 7.65 (d, J=1.7 Hz, 1H), 7.51-7.35 (m, 1H), 3.89 (s, 3H). ¹³C NMR (101 MHz, DMSO) δ 163.73, 157.06, 149.80, 137.45, 130.94, 125.92, 124.85, 123.53, 122.54, 118.14, 115.19, 112.25, 94.39, 79.22, 51.80. Mass calculated for (C₁₆H₉BrClN₃O₂—H)⁻ 388.0, found 387.8.

### Example 48: Synthesis of Compounds 157, 158 and 163

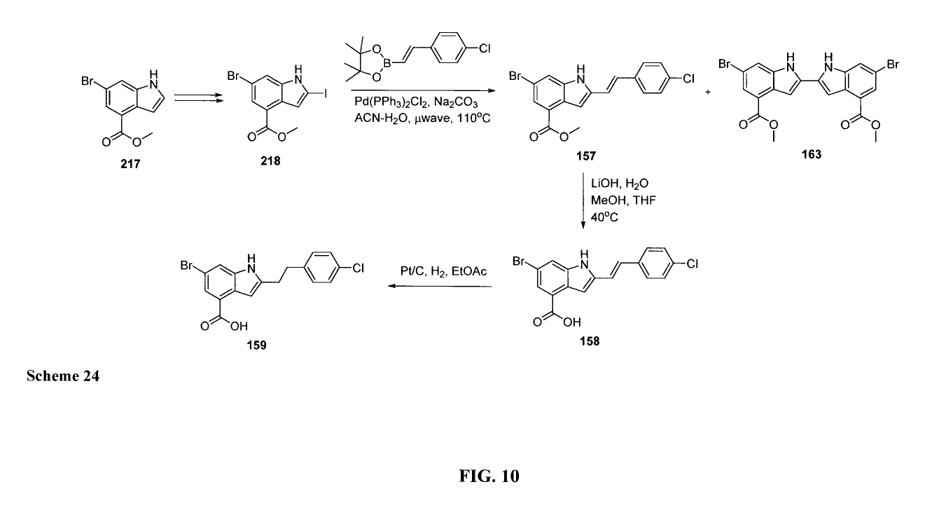

Compounds 157, 158 and 163 were prepared according the general synthesis pathway shown in Scheme 24 (FIG. 10).

### Synthesis of Compounds 157 and 163

Intermediate 218 was prepared from commercially available 217 following the procedure described to prepare intermediate 214. 214 was coupled to the corresponding Pinacol boronate ester following the procedure to make compound 161 to give 157 and bi-product 163.

### (E)-Methyl 6-bromo-2-(4-chlorostyryl)-1H-indole-4-carboxylate (157)

Yield=68%. ¹H NMR (600 MHz, DMSO) δ 11.93 (s, 1H), 7.79-7.76 (m, 2H), 7.64 (d, J=8.5 Hz, 2H), 7.48 (d, J=8.4 Hz, 2H), 7.35 (dd, J=46.9, 16.5 Hz, 2H), 7.14 (d, J=1.3 Hz, 1H), 3.93 (s, 3H). ¹³C NMR (151 MHz, DMSO) δ 165.89, 139.90, 139.02, 135.46, 132.36, 128.89, 128.47, 128.14, 126.85, 124.58, 121.38, 119.63, 117.98, 113.22, 103.77, 52.04.

### Dimethyl 6,6′-dibromo-1H,1′H-[2,2′-biindole]-4,4′-dicarboxylate (163)

¹H NMR (400 MHz, DMSO) δ 12.38 (s, 2H), 7.84 (dd, J=1.7, 0.8 Hz, 2H), 7.82 (d, J=1.7 Hz, 2H), 7.62 (d, J=1.3 Hz, 2H), 3.97 (s, 6H). Mass calculated for (C₂₀H₁₄Br₂N₂O₄—H)⁻ 504.93, found 504.8.

### Synthesis of Compound 158

To a stirred solution of 157 (0.1 mmol) in THF (1 mL) and MeOH (1 mL) was added an aqueous solution of LiOH (0.5 mmol in 1 mL H₂O). The mixture was stirred at 40° C. overnight and then acidified to pH of 1 with HCl (1M aqueous solution). The mixture was extracted with EtOAc and the organic phase was dried over anhydrous Na₂SO₄ and concentrated. The residue was purified by automated flash chromatography to give compound 158 (90%).

### (E)-6-Bromo-2-(4-chlorostyryl)-1H-indole-4-carboxylic acid (158)

¹H NMR (400 MHz, DMSO) δ 13.03 (s, 1H), 11.89 (s, 1H), 7.77 (s, 2H), 7.66 (d, J=8.6 Hz, 2H), 7.51 (d, J=8.5 Hz, 2H), 7.36 (dd, J=36.8, 16.5 Hz, 2H), 7.18 (d, J=1.7 Hz, 1H). ¹³C NMR (101 MHz, DMSO) δ 166.62, 140.23, 139.29, 139.08, 136.09, 132.61, 130.65, 129.33, 128.75, 128.50, 127.98, 127.75, 127.18, 126.53, 121.84, 120.35, 116.26, 114.12, 104.47, 43.07. Mass calculated for (C₁₇H₁₁BrClNO₂—H)⁻ 375.96, found 376.0.

### Example 49: Synthesis of Compound 159

Compound 159 was prepared using a similar protocol to that described for the synthesis of compound 36 (Example 2).

### 6-bromo-2-(4-chlorophenethyl)-1H-indole-4-carboxylic acid (159)

Yield=65%. ¹H NMR (400 MHz, DMSO) δ 12.84 (s, 1H), 11.47 (s, 1H), 7.69 (s, 2H), 7.33 (d, J=8.4 Hz, 2H), 7.27 (d, J=8.4 Hz, 2H), 6.71 (s, 1H), 3.08-2.97 (m, 4H), ¹³C NMR (101 MHz, MeOD) δ 168.64, 142.60, 139.88, 138.20, 131.49, 129.62, 128.02, 127.75, 124.73, 121.58, 117.37, 111.86, 100.55, 34.41, 29.75. Mass calculated for (C₁₇H₁₃BrClNO₂—H)⁻ 377.98, found 378.0.

### Example 50: Synthesis of Compounds 165 and 166

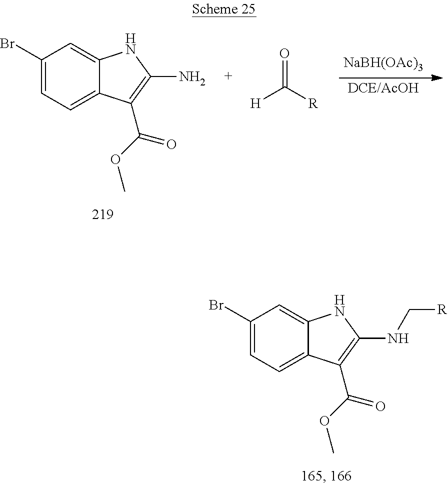

The 2-aminoindole intermediate 219 was prepared according to literature procedures (see WO 2011/056739). A mixture of 219 (1.0 mmol), corresponding aldehyde (2.0 mmol), NaBH(OAc)₃ (4.5 mmol), acetic acid (4.0 mmol) in DCE (5 mL) was stirred at ambient temperature for 1-2 days, slowly quenched with a saturated aqueous solution of NaHCO₃ (10 mL), diluted with water (15 mL) and extracted with EtOAc (3×25 mL). The combined organics was dried over MgSO₄, filtered and concentrated in vacuo. The crude product was purified by automated flash chromatography to give the desired product.

### Methyl 6-bromo-2-(4-chlorobenzylamino)-1H-indole-3-carboxylate (165)

¹H NMR (400 MHz, DMSO) δ 11.29 (s, 1H), 7.74 (t, J=6.9 Hz, 1H), 7.48 (d, J=8.3 Hz, 1H), 7.45-7.40 (m, 2H), 7.38 (d, J=8.5 Hz, 2H), 7.22 (d, J=2.0 Hz, 1H), 7.12 (dd, J=8.3, 1.9 Hz, 1H), 4.62 (d, J=6.9 Hz, 2H), 3.78 (s, 3H). ¹³C NMR (101 MHz, DMSO) δ 166.29, 153.99, 138.53, 134.31, 132.12, 129.18, 128.93, 126.42, 123.85, 119.74, 112.82, 111.49, 83.74, 50.55, 45.60. Mass calculated for (C₁₇H₁₄BrClN₂O₂+H)⁺ 393.0, found 393.3.

### Methyl 6-bromo-2-(imidazo[1,2-a]pyridin-2-ylmethylamino)-1H-indole-3-carboxylate (166)

¹H NMR (400 MHz, DMSO) δ 11.46 (s, 1H), 8.52 (dt, J=6.8, 1.2 Hz, 1H), 7.83 (s, 1H), 7.68 (s, 1H), 7.59-7.51 (m, 1H), 7.48 (d, J=8.3 Hz, 1H), 7.27 (d, J=2.0 Hz, 1H), 7.25-7.21 (m, 1H), 7.13 (dd, J=8.3, 1.8 Hz, 1H), 6.88 (td, J=6.8, 1.2 Hz, 1H), 4.72 (d, J=6.3 Hz, 2H), 3.78 (s, 3H). ¹³C NMR (101 MHz, DMSO) δ 166.37, 154.28, 144.90, 144.06, 134.41, 127.45, 126.41, 125.33, 123.80, 119.69, 116.83, 112.90, 112.45, 111.43, 110.38, 83.69, 50.54, 41.63. Mass calculated for (C₁₈H₁₅BrN₄O₂+H)⁺ 398.0, found 399.5.

### Example 51: Pyruvate Kinase Inhibition and Inhibition of Growth of Staphylococcus Aureus by Compounds 95-179

The antimicrobial activity against S. aureus ATCC 29213 and either the IC₅₀ or EC₅₀ for inhibition of MRSA PK for compounds 95-179 was tested according to the procedures provided under “General Methodologies” above. The results are presented below in TABLE 8. Compounds marked with an asterisk showed non-classical inhibition curves, but were still inhibitory. EC₅₀ values are provided for these compounds.

TABLE 8
Antimicrobial Activity and PK Inhibitory Activity for Compounds 95-169
and 171-179

| | | MIC |
|---|---|---|
| Compound | IC₅₀ (nM)^{†} | (μg/mL)^{‡} |
| 95 | A | 7.1, 8 |
| 96 | C | >64 |
| 97 | *¹ | >64 |
| 98 | A | >64 |
| 99 | B | 32 |
| 100 | D | >64 |
| 101 | D | >64 |
| 102 | C | 64 |
| 103 | D | >64 |
| 104 | D | >64 |
| 105 | E | >64 |
| 106 | D | >64 |
| 107 | B | >64 |
| 108 | C | >64 |
| 109 | D | >64 |
| 110 | A | >64 |
| 111 | D | n/d |
| 112 | E | n/d |
| 113 | D | n/d |
| 114 | D | n/d |
| 115 | D | n/d |
| 116 | D | n/d |
| 117 | D | n/d |
| 118 | B | 16 |
| 119 | E | >64 |
| 120 | B | >64 |
| 121 | D | >64 |
| 122 | C | 16, 1, 2 |
| 123 | C | 26, 64 |
| 124 | A | 4, 2 |
| 125 | D | >64 |
| 126 | E | 64 |
| 127 | E | 64 |
| 128 | 17 | >64 |
| 129 | A | 16 |
| 130 | A | >64 |
| 131 | *² | >64 |
| 132 | A | 16 |
| 133 | A | >64 |
| 134 | A | 64 |
| 135 | *³ | >64 |
| 136 | C | >64 |
| 137 | *⁴ | >64 |
| 138 | A | >64 |
| 139 | B | >64 |
| 140 | D | n/d |
| 141 | C | >64 (2) |
| 142 | B | >64 (2) |
| 143 | A | n/d |
| 144 | D | n/d |
| 145 | D | n/d |
| 146 | D | 32 |
| 147 | *⁵ | >64 |
| 148 | D | 32 |
| 149 | D | >64 |
| 150 | D | 8 |
| 151 | D | n/d |
| 152 | D | 64 |
| 153 | C | 32 |
| 154 | D | 16, 32 |
| 155 | C | n/d |
| 156 | D | n/d |
| 157 | D | >64 |
| 158 | D | 32 |
| 159 | C | 64, >64 |
| 160 | A | >64 |
| 161 | A | >64 |
| 162 | A | 4 |
| 163 | B | >64 |
| 164 | A | 1 |
| 165 | D | 4 |
| 166 | D | >64 |
| 167 | C | 2, 1 |
| 168 | C | 1, 2 |
| 169 | E | >64 |
| 171 | D | 1 |
| 172 | D | |
| 173 | D | >64 |
| 174 | D | 32 |
| 175 | C | 2 |
| 176 | D | |
| 177 | D | 2, 1 |
| 178 | A | 4, 8, 2, 1, 2 |
| 179 | A | 64, >64 |

^{†}A. <50 nm; B. 50-100 nm; C. 100-1000 nm; D. >1000 nm; E. not active at the concentrations tested (up to 100 μM)
^{‡}Tested at least in duplicate; where different values were obtained from different tests, both values are provided.
¹EC₅₀ = 8.6 nM;
²EC₅₀ = 15.0 nM;
³EC₅₀ = 31.7 nM;
⁴EC₅₀ = 13.2 nM;
⁵EC₅₀ = 244 nM

### Example 52: Antimicrobial Activity Against Gram Negative Bacteria and Other Gram Positive Bacteria

The antimicrobial activity of selected compounds against the gram negative bacteria Klebsiella pneumoniae, Acinetobacter baumannii, E. coli, Pseudomonas aeruginosa and Salmonella typhimurium, as well as additional gram positive bacteria, including drug-resistant strains, was tested according to the procedures provided under “General Methodologies” above. The results are presented below in TABLE 9.

TABLE 9
Antimicrobial Activity against Gram Negative and Gram Postive
Bacterial Strains

| | MIC (μg/mL) | | | | | | | | |
|---|---|---|---|---|---|---|---|---|---|
| | Gram negative | | | | | Gram positive | | | |
| | bacteria | | | | | bacteria | | | |
| Compound | A | B | C | D | E | F | G | H | I |
| 167 | 1 | >64 | 4, 8 | >64 | >64 | 1 | 1-0.5 | 1.5 (2) | 2 |
| 178 | 1 | >64 | >64 | >64 | >64 | 1 | 0.5 | 1 | 1 |
| 168 | — | — | — | — | — | 1 | 0.5 | 2 | — |
| 124 | 2 | >64 | >64 | >64 | >64 | 8 | 2 | 2 | — |
| 123 | 32 | 64 | 64 | >64 | >64 | 32 | 32 | 16 | — |
| Methicillin | 4 | >64 | — | — | — | 64 | 32 | 2 | 1 |
| Vancomycin | 4 | 64 | — | — | — | 64 | 1 | 1 | — |
| Ciprofloxacin | — | 0.125 | 0.0078 | 0.125 | <0.031 | — | — | — | 0.5 |

A. K. pneumoniae (C238);
B. A. baumannii (ATCC 19606);
C. E. coli (DAS 1-IMP);
D. P. aeruginosa (PA01);
E. S. typhimurium (SL 1344);
F. VRE#2 (2010A);
G. MRSA MW2 (USA400);
H. S. aureus (ATCC29213);
I. S. pyogenes (ATCC700294)

### Example 53: Generation of Resistance in MRSA

Method:

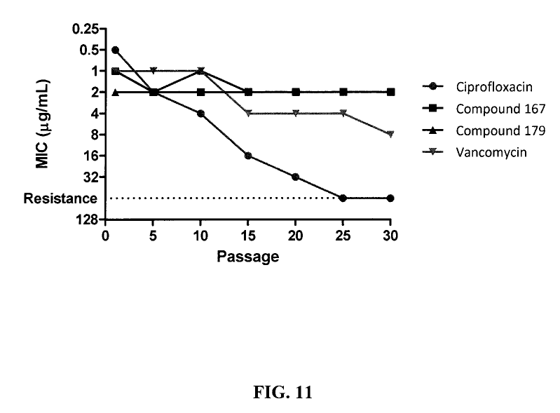

Changes in the susceptibilities of methicillin-resistant Staphylococcus aureus (MRSA), MW USA400, to compounds 167 and 178. vancomycin and ciprofloxacin (control) were monitored during 30 serial passages in MHCAB broth containing the highest sublethal concentration of each compound. MRSA MW USA400 was grown in 96-well assay plates for 24 hrs in the presence of several concentrations of compound (64 to 0.031 μg/ml). Bacterial cultures were then recovered from wells that contained compound concentration at 0.5× the MIC and at least 15% growth, when compared to untreated controls. For the subsequent 30 passages, 5×10⁵ CFU/ml of bacteria were inoculated in MHCAB which contained 0.5×MIC concentration of the preceding passage. Colonies were isolated on MH agar for passage 5, 10, 15, 20, 25 and 30 before the MIC was determined. Results are shown in FIG. 11 and are displayed as the MIC for the compounds for passage 1, 5, 10, 15, 20, 25 and 30.

Results:

Pyruvate kinase (PK) is a highly connected, essential hub protein which is critical for bacterial survival and thus it should be difficult for bacteria to develop resistance to compounds directed to this target. In order to assess the potential for bacteria to generate resistant mutants to representative compounds of general formula I, clinically relevant MRSA MW2 (USA400) was passaged for up to 30 consecutive generations in the presence of sub-lethal concentrations of test compounds. As shown in FIG. 11, after 30 subcultures in the presence of either compound 167 or 178 the MIC remained stable and no mutants were detected. For positive control ciprofloxacin, however, mutants were able to grow in the presence of 32 μg/mL ciprofloxacin after 25 passages, indicating the emergence of ciprofloxacin-resistant mutants.

### Example 54: In Vivo Tolerability and Bioavailability

The MTD and pharmacokinetic profiles of compounds 123 and 178 administered IV or PO were determined in female CD-1 mice (6-8 weeks of age). Compounds were dissolved in 3% DMSO/6% Solutol® HS 15/10 mM PB (pH7.4) for IV administration and in 3% DMSO/6% Solutol® HS 15/water for oral administration. Study groupings are shown in Tables 10 and 11.

TABLE 10
Tolerability Study Grouping

| | | | | | Dose | | |
|---|---|---|---|---|---|---|---|
| | Group | # of | Dose | Admin. | Volume | Dose | Time-point |
| Gp # | Name | Animals | (mg/kg) | Route | (mL/kg) | Schedule | (hr) |
| 1 | 178 | 3 | 3, 10, | IV | 10 | QD × 1 per dose | 24 after highest |
| | | | 30 | | | level | dose |
| | | | | | | 1 dose level/day | |
| 2 | 123 | 3 | 3, 10, | IV | 10 | QD × 1 per dose | 24 after highest |
| | | | 30 | | | level | dose |
| | | | | | | 1 dose level/day | |

TABLE 11
Pharmacokinetic Study Grouping

| | | | | | Dose | | |
|---|---|---|---|---|---|---|---|
| Gp | Group | # of | Dose | Admin. | Volume | Time-point | |
| # | Name | Animals | (mg/kg) | Route | (mL/kg) | (min) | |
| 4 | 178 | 21 | 3 | IV | 10 | 5, 15, 30 | min |
| | | (n = 3/ | | | | 1, 3, 6, 24 | hr |
| | | timepoint) | | | | | |
| 5 | 178 | 18 | 10 | PO | 10 | 15, 30 | min |
| | | (n = 3/ | | | | 1, 3, 6, 24 | hr |
| | | timepoint) | | | | | |
| 6 | 123 | 21 | 3 | IV | 10 | 5, 15, 30 | min |
| | | (n = 3/ | | | | 1, 3, 6, 24 | hr |
| | | timepoint) | | | | | |
| 7 | 123 | 18 | 10 | PO | 10 | 15, 30 | min |
| | | (n = 3/ | | | | 1, 3, 6, 24 | hr |
| | | timepoint) | | | | | |

For tolerability studies, one animal from each group was administered the lowest dose first and then observed for 60 min. If the animal tolerated the compound, then the remaining animals in the group received their respective administrations, and each was observed for an appropriate period of time to ensure they tolerated the compound. On the following day, the next higher dose was administered using the same procedure outlined above. Animals were euthanized 24 hours after the last injection, a cardiac puncture blood sample was collected and processed for plasma. Thigh muscle was collected and a necropsy performed.

For the pharmacokinetic study, animals were injected with their respective compound at the dose indicated in 11. At their respective time point, animals were deeply anesthetized with isoflurane and then asphyxiated with CO₂. Cardiac puncture blood was collected immediately following death, and the blood processed to generate plasma.

For compound 178, the above example established that IV doses of 30 mg/kg and PO doses of 10 mg/kg were well tolerated. For IV administration of compound 178 at 3 mg/kg, Cₘₐₓ was 18996 ng/mL and T_{1/2} was 136 min. For PO administration, bioavailability (% F) was 21%.

### Example 55: In Vivo Antimicrobial Activity

The antimicrobial activity of compound 178 against S. aureus ATCC 29123 was tested in vivo using the neutropenic mouse Staphylococcus aureus thigh infection model. Briefly, animals (female CD-1 mice, 5 weeks of age) were made neutropenic prior to S. aureus thigh infection by pre-treating with cyclophosphamide (150 mg/kg, IP, −4 and −1 days pre-inoculation). On the inoculation day (day 0), mice were infected with S. aureus as indicated in Table 12 at time zero (t=0). Animals were individually monitored for adverse reactions for 30 min post-infection.

Compound 178 was prepared for IV administration by dissolving in 3% DMSO/6% Solutol® HS 15/10 mM PB (pH7.4) and for oral administration by dissolving in 3% DMSO/6% Solutol® HS 15/water. Vancomycin was administered as a solution in PBS. The test compounds were administered as outlined in Table 12 at 2 and 8 hours post-infection and animals were individually monitored for adverse reactions for 30 min after each injection. All animals were then monitored hourly from 20 hours post infection to the endpoint (t=24 hr post infection). At the indicated timepoint, animals were sacrificed and the injected thigh collected.

Quantitative enumeration of bacterial load was determined by plating serial dilutions from homogenized thigh muscles. Homogenized muscle was in a total of 2 mL volume, from which a 1 in 10 dilution was prepared (100 μL into 900 μL saline). From this a series of dilutions were prepared and plated on Mueller Hinton agar plates. Plates were incubated overnight at 37° C. in 100% atmospheric air. At the end of this time colony counts were determined and the final CFU per mL calculated.

TABLE 12
Study Grouping for Thigh Infection Model

| | | Compound | | | | Bacteria | | | Post- |
|---|---|---|---|---|---|---|---|---|---|
| | | | Dose | | Dose | | Dose | | Infection |
| Group #/ | | Dose | Volume | Admin | Time | # of | Volume | Admin | Endpoint |
| Name | n | (mg/kg) | (mL/kg) | Route | Point (hr) | Cells | (μL/animal) | Route | (hr) |
| 1. Control | 4 | n/a | n/a | n/a | n/a | 1e4 | 25 | IM | 24 |
| 2. | 4 | 10 | 10 | IP | 2, 8 | 1e4 | 25 | IM | 24 |
| Vancomycin | | | | | | | | | |
| 3. 178 | 6 | 3 | 10 | IV | 2, 8 | 1e4 | 25 | IM | 24 |
| 4. 178 | 6 | 10 | 10 | IV | 2, 8 | 1e4 | 25 | IM | 24 |
| 5. 178 | 4 | 10 | 10 | PO | 2, 8 | 1e4 | 25 | IM | 24 |

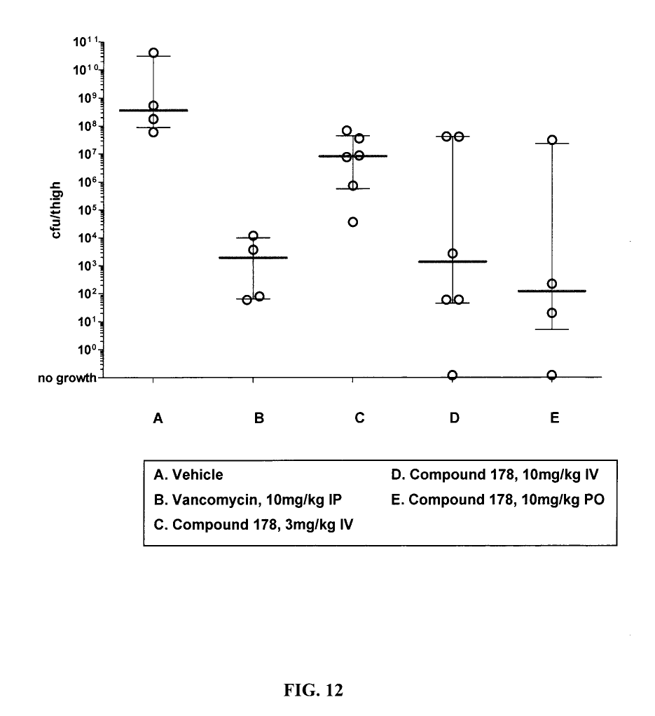

The results are presented in FIG. 12. The colony counts for the untreated control ranged from 6.08×10⁷ to 4.08×10¹⁰ CFU/thigh with a median of 3.6×10⁸. The counts for the vancomycin control showed a reduction in median CFU/thigh (1.91×10³ CFU/thigh). All of the compound 178 treated groups showed at least a 1 log reduction in median counts for CFU/thigh. In the IV treated group this reduction was dose dependent with group 3 (178 3 mg/kg) showing a median of 8.27×10⁶ CFU/thigh and group 4 (178 10 mg/kg) showing a median of 1.38×10³ CFU/thigh. The greatest reduction in median counts per thigh was observed in the orally treated group (group 5; 178 PO 10 mg/kg) which showed a 6 log reduction in median counts to 1.2×10² CFU/thigh. It should also be noted that one mouse in group 5 for which difficulties with oral gavage were encountered was only dosed once, but was still able to reduce the infection to 2.2×10² CFU/thigh.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method of treating a subject known to have or suspected of having a bacterial infection, the method comprising administering to the subject an effective amount of a compound of general formula I: 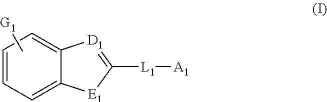
or a salt thereof, wherein:
L₁ is —CH₂CH₂—, —CHCH—, —CC—, —C(O)NH—, —NHC(O)—, —C(O)—, —N═CH—, —CH₂R₅—, —NHCH₂—, 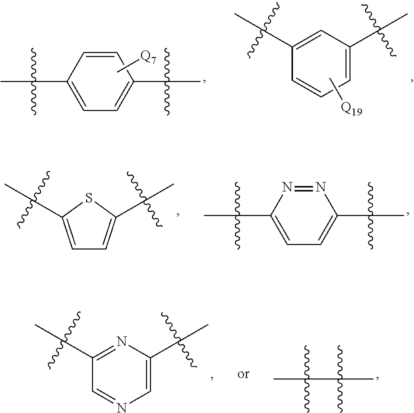
wherein R₅ is CH₂CH₂, NHCH₂, NH, SCH₂, O, or S, and wherein each Q₇ and Q₁₉ are independently H, NO₂, or OMe;
A₁ is 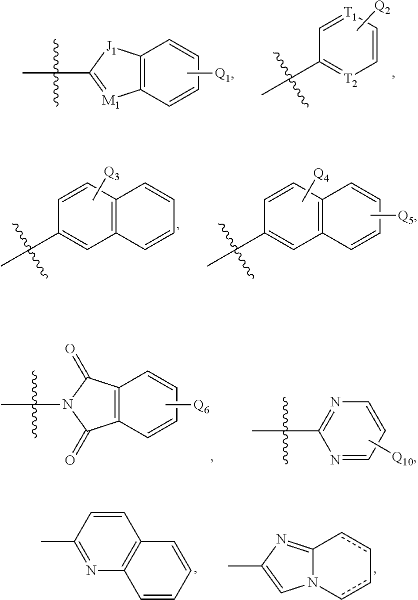
wherein [symbol] represents a single or double bond, or a 5-membered heteroaryl optionally substituted with Q₁₁ and containing 1 or 2 heteroatoms each selected from N, O and S;
each G₁ is independently H, Br, F, Cl, I, OR₁, SR₁, SO₂R₁, C(O)R₁, C(O)OR₁, unsubstituted phenyl, substituted phenyl, unsubstituted alkyl, substituted alkyl, unsubstituted allyl, or substituted allyl, wherein the alkyl or allyl is 1-6 carbons in length, wherein the substitutions to the phenyl, alkyl, or allyl are optionally Br, F, Cl, I, OH, OMe, or N₃, and wherein R₁ is H or Me;
D₁ is S, O, N—H, N—CH₃, N—CH₂—OCH₃, N—CH₂—CH₃, N—CH₂—COOH, N—CH₂—CH₂OH, CH₂, CH—CH₃, CH—CH₂—OCH₃, CH—CH₂—CH₃, CH—CH₂—COOH, CH—CH₂—CH₂OH, N—R₂, or CH—R₂, wherein R₂ is 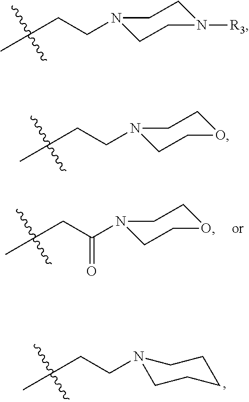
wherein R₃ is H, unsubstituted alkyl, or substituted alkyl, wherein the alkyl is 1-6 carbons in length, and the alkyl is optionally substituted with Br, F, Cl, I, OH, OMe, or N₃;
E₁ is N, C—H, C—CH₃, C—C(O)OR₄, C—C(O)R₆₃, C—Cl, C—Br, C—F, or C—COR₄, wherein R₄ is H or Me, and R₆₃ is CH₃, CH₂CH₃, CH₂CH₂CH₃, CH(CH₃)₂ or CF₃, and wherein if D₁ is CH₂, CH—CH₃, CH—CH₂—OCH₃, CH—CH₂—CH₃, CH—CH₂—COOH, CH—CH₂—CH₂OH, or CH—R₂, E₁ is N;
each Q₁ is independently H, Br, F, Cl, I, 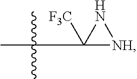 [SMILES: *C1(C(F)(F)F)NN1]
OR₆, SR₆, SO₂R₆, C(O)R₆, C(O)OR₆, N₃, unsubstituted phenyl, substituted phenyl, unsubstituted alkyl, substituted alkyl, unsubstituted allyl, or substituted allyl, wherein the alkyl or allyl is 1-6 carbons in length, wherein R₆ is H or Me, and wherein the substituted phenyl, alkyl, or allyl is optionally substituted with Q₈;
each Q₂ is independently H, Br, F, Cl, I, 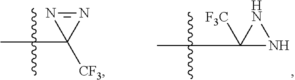 [SMILES: *C1(C(F)(F)F)N=N1 ; *C1(C(F)(F)F)NN1]
N₃, OR₇, SR₇, SO₂R₇, C(O)R₇, NO₂, 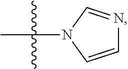 [SMILES: *n1ccnc1]
unsubstituted phenyl, substituted phenyl, unsubstituted alkyl, substituted alkyl, unsubstituted allyl, or substituted allyl, wherein the alkyl or allyl is 1-6 carbons in length, wherein R₇ is H or Me, and wherein the substituted phenyl, alkyl, or allyl is optionally substituted with Q₉;
each Q₃ is independently H, Br, F, Cl, I, or ORB, wherein R₈ is H or Me;
each Q₄ is independently H, Br, F, Cl, I, or OR₉, wherein R₉ is H or Me;
each Q₅ is independently H, Br, F, Cl, I, or OR₁₀, wherein R₁₀ is H or Me;
each Q₆ is independently H, Br, F, Cl, I, or OR₁₁, wherein R₁₁ is H or Me;
each Q₈ is independently Br, F, Cl, I, Me, or OR₁₂, wherein Rig is H or Me;
each Q₉ is independently Br, F, Cl, I, Me, or OR₁₃, wherein R₁₃ is H or Me;
each Q₁₀ is independently H, Br, F, Cl or I;
each Q₁₁ is independently H, Me, unsubstituted phenyl or substituted phenyl, wherein the substituted phenyl is optionally substituted with Q₈;
J₁ is S, O, N—H, N—CH₃, N—CH₂—OCH₃, N—CH₂—COOH, N—CH₂—CH₂OH, CH—CH₃, N—R₁₄, or CH—R₁₄, wherein R₁₄ is 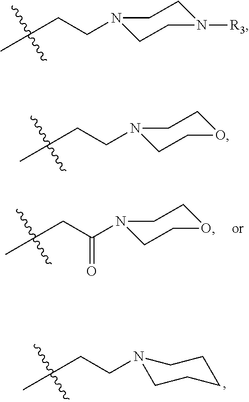
wherein R₃ is H, unsubstituted alkyl, or substituted alkyl, wherein the alkyl is 1-6 carbons in length, and the alkyl is optionally substituted with Br, F, Cl, I, OH, OMe, or N₃;
M₁ is N, C—H, C—CH₃, C—C(O)OR₄, C—C(O)R₆₃, or C—CH(CH₃)₂, wherein R₄ is H or Me, and R₆₃ is CH₃, CH₂CH₃, CH₂CH₂CH₃, CH(CH₃)₂ or CF₃;
T₁ is N or C—H; and
T₂ is N or C—H,
and wherein
(A) when A₁ is 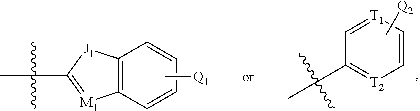
and T₁ and T₂ are each C—H, then at least one of Q₁ or Q₂ is Br, F, Cl or I; and
(B) the compound is not one of the following: 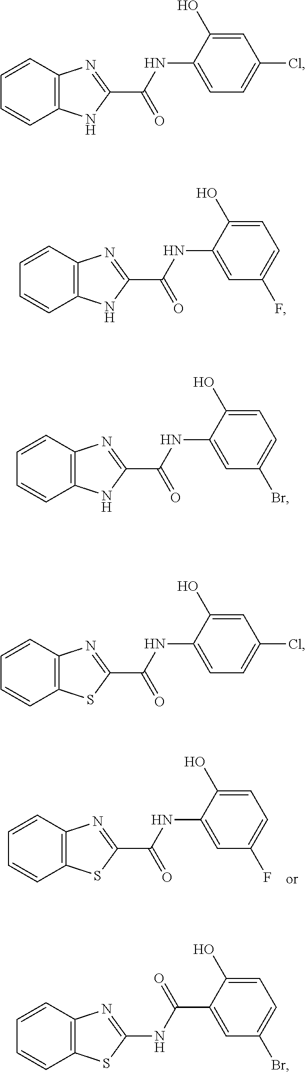
wherein the compound, or salt thereof, has anti-bacterial activity.

2. The method according to claim 1, wherein:
R₅ is NHCH₂, NH, SCH₂, or S;
A₁ is 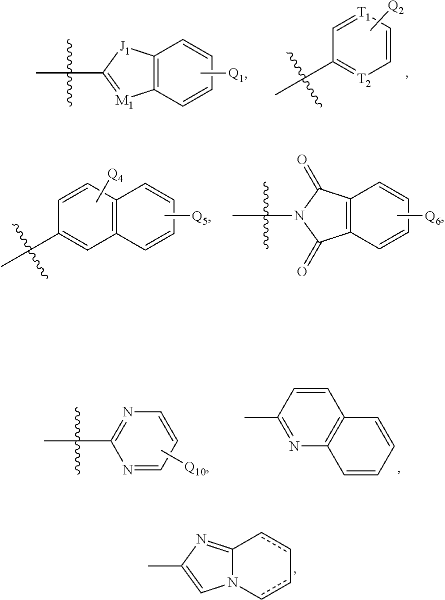
wherein [symbol] represents a single or double bond, or a 5-membered heteroaryl optionally substituted with Q₁₁ and containing 1 or 2 heteroatoms each selected from N, O and S;
each G₁ is independently H, Br, F, Cl, OR₁, C(O)R₁, C(O)OR₁, unsubstituted phenyl, substituted phenyl, unsubstituted alkyl, substituted alkyl, wherein the alkyl is 1-3 carbons in length, wherein the substitutions to the phenyl or alkyl are optionally Br, F, Cl, I, OH, OMe, or N₃, and wherein R₁ is H or Me;
D₁ is S, N—H, N—CH₃, N—CH₂—OCH₃, N—CH₂—CH₃, N—CH₂—COOH, N—CH₂—CH₂OH, N—R₂;
E₁ is N, C—H, C—CH₃, C—C(O)OR₄, C—C(O)R₆₃, C—Cl, or C—OR₄;
each Q₁ is independently; H, Br, F, Cl, 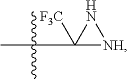 [SMILES: *C1(C(F)(F)F)NN1]
OR₆, C(O)R₆, C(O)OR₆, N₃, unsubstituted phenyl, substituted phenyl, unsubstituted alkyl, or substituted alkyl, wherein the alkyl is 1-3 carbons in length, wherein R₆ is H or Me, and wherein the substituted phenyl or substituted alkyl is optionally substituted with Q₈;
each Q₂ is independently H, Br, F, Cl, 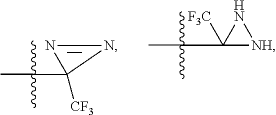 [SMILES: *C1(C(F)(F)F)NN1 ; *C1(C(F)(F)F)N=N1]
N₃, OR₇, C(O)R₇, NO₂, 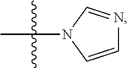 [SMILES: *n1ccnc1]
unsubstituted phenyl, substituted phenyl, unsubstituted alkyl, or substituted alkyl, wherein the alkyl is 1-3 carbons in length, wherein R₇ is H or Me, and wherein the substituted phenyl or substituted alkyl is optionally substituted with Q₉;
each Q₄ is independently H, Br, F, Cl, or OR₉;
each Q₅ is independently H, Br, F, Cl, or OR₁₀;
each Q₆ is independently H, Br, F, Cl, or OR₁₁;
each Q₈ is independently Br, F, Cl, Me, or OR₁₂;
each Q₉ is independently Br, F, Cl, Me, or OR₁₃;
each Q₁₀ is independently H, Br, F or Cl;
J₁ is S, O, N—H, N—CH₃, N—CH₂—OCH₃, N—CH₂—COOH, N—CH₂—CH₂OH, or N—R₁₄, wherein R₁₄ is 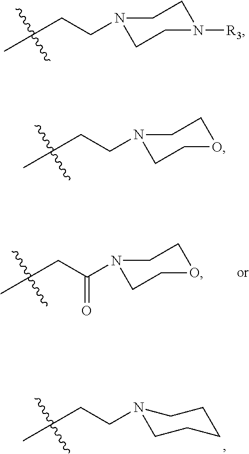
wherein R₃ is H or Me, and M₁ is N, C—H, C—CH₃, C—C(O)OR₄, or C—C(O)R₆₃.

3. The method according to claim 1, wherein:
D₁ is S, O, N—H, N—CH₃, N—CH₂—OCH₃, N—CH₂—CH₃, N—CH₂—COOH, N—CH₂—CH₂OH, or N—R₂;
and either:
A₁ is 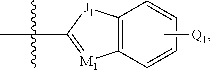
and E₁ is C—CH₃, C—C(O)OR₄, C—C(O)R₆₃, or C—OR₄, wherein R₄ is H or Me, and R₆₃ is CH₃, CH₂CH₃, CH₂CH₂CH₃, CH(CH₃)₂ or CF₃,
or
A₁ is 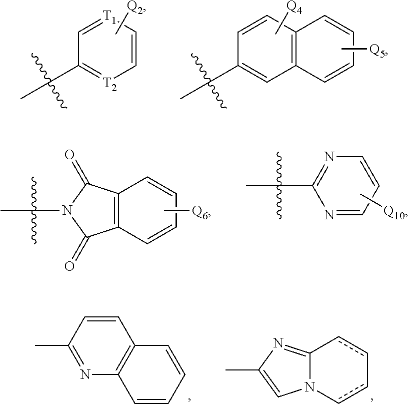
wherein [symbol] represents a single or double bond, or a 5-membered heteroaryl optionally substituted with Q₁₁ and containing 1 or 2 heteroatoms each selected from N, O and S, and E₁ is N, C—H, C—CH₃, C—C(O)OR₄, C—C(O)R₆₃, C—Cl, or C—OR₄.

4. The method according to claim 1, wherein the compound of general formula I is a compound of general formula II or general formula III:
wherein: 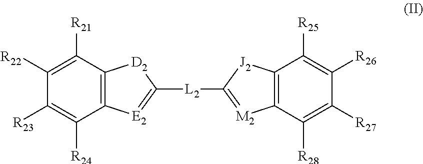
L₂ is —CH₂CH₂—, —CHCH—, —CC—, —C(O)NH—, —NHC(O)—, —C(O)—, —N═CH—, —CH₂R₃₅—, —NHCH₂—, 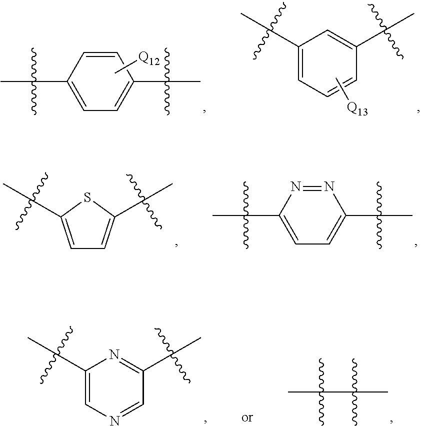
wherein R₃₅ is CH₂CH₂, NHCH₂, NH, SCH₂, S or O, and wherein each Q₁₂ and Q₁₃ are independently H, NO₂, or OMe;
D₂ is S, O, N—H, N—CH₃, N—CH₂—OCH₃, N—CH₂—CH₃, N—CH₂—COOH, N—CH₂—CH₂OH, or N—R₂₉, wherein R₂₉ is 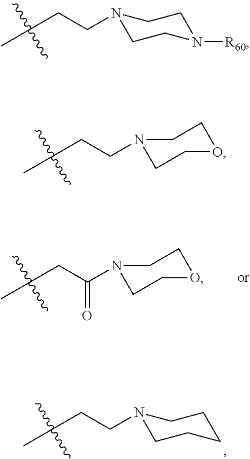
wherein R₆₀ is H, unsubstituted alkyl, or substituted alkyl, wherein the alkyl is 1-6 carbons in length, and the alkyl is optionally substituted with Br, F, Cl, I, OH, OMe, or N₃;
E₂ is C—CH₃, C—C(O)R₅₇, or C—C(O)OR₃₆, wherein R₃₆ is H or Me, and R₅₇ is CH₃, CH₂CH₃, CH₂CH₂CH₃, CH(CH₃)₂ or CF₃;
J₂ is S, O, N—H, N—CH₃, N—CH₂—OCH₃, N—CH₂—COOH, N—CH₂—CH₂OH, or N—R₃₈, wherein R₃₈ is 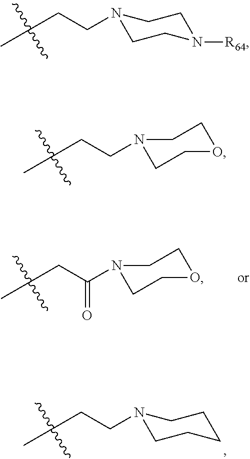
wherein R₆₄ is H, unsubstituted alkyl, or substituted alkyl, wherein the alkyl is 1-6 carbons in length, and the alkyl is optionally substituted with Br, F, Cl, I, OH, OMe, or N₃;
M2 is N, C—H, C—CH₃, C—C(O)R₅₇, or C—C(O)OR₃₆, wherein R₃₆ is H or Me, and R₅₇ is CH₃, CH₂CH₃, CH₂CH₂CH₃, CH(CH₃)₂ or CF₃;
each of R₂₁, R₂₂, R₂₃, R₂₄, R₂₅, R₂₆, R₂₇ and R₂₈ is independently H, Br, F, Cl, I, 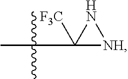 [SMILES: *C1(C(F)(F)F)NN1]
OR₂₉, C(O)R₂₉, C(O)OR₂₉, N₃, unsubstituted phenyl, substituted phenyl, unsubstituted alkyl, or substituted alkyl, wherein the alkyl is 1-6 carbons in length, wherein R₂₉ is H or Me, and wherein the substituted phenyl or substituted alkyl is optionally substituted with Q₁₄, and
each Q₁₄ is independently Br, F, Cl, I, Me, or OR₃₇, wherein R₃₇ is H or Me,
and wherein at least one of R₂₁, R₂₂, R₂₃, R₂₄, R₂₅, R₂₆, R₂₇ and R₂₈ is Br, F, Cl or I; 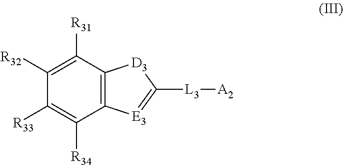
wherein:
L₃ is —CH₂CH₂—, —CHCH—, —CC—, —C(O)NH—, —NHC(O)—, —C(O)—, —N═CH—, —CH₂R₃₉—, —NHCH₂—, 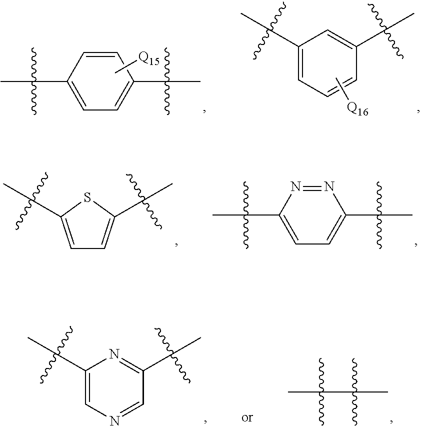
wherein R₃₉ is CH₂CH₂, NHCH₂, NH, SCH₂, S or O, and wherein each Q₁₅ and Q₁₆ are independently H, NO₂, or OMe;
A2 is 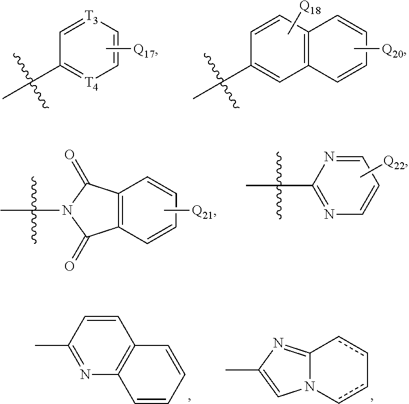
wherein [symbol] represents a single or double bond, or a 5-membered heteroaryl optionally substituted with Q₂₃ and containing 1 or 2 heteroatoms each selected from N, O and S;
D₃ is S, O, N—H, N—CH₃, N—CH₂—OCH₃, N—CH₂—CH₃, N—CH₂—COOH, N—CH₂—CH₂OH, or N—R₆₁, wherein R₆₁ is 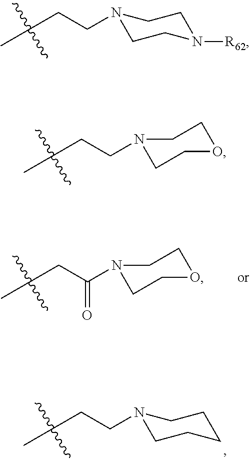
wherein R₆₂ is H, unsubstituted alkyl, or substituted alkyl, wherein the alkyl is 1-6 carbons in length, and the alkyl is optionally substituted with Br, F, Cl, I, OH, OMe, or N₃;
E₃ is N, C—H, C—Cl, C—CH₃, C—C(O)R₅₉, or C—C(O)OR₄₀, wherein R₄₀ is H or Me, and R₅₉ is CH₃, CH₂CH₃, CH₂CH₂CH₃, CH(CH₃)₂ or CF₃;
each of R₃₁, R₃₂, R₃₃ and R₃₄ is independently H, Br, F, Cl, I, 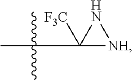 [SMILES: *C1(C(F)(F)F)NN1]
OR₄₁, C(O)R₄₁, C(O)OR₄₁, N₃, unsubstituted phenyl, substituted phenyl, unsubstituted alkyl, or substituted alkyl, wherein the alkyl is 1-6 carbons in length, wherein R₄₁ is H or Me, and wherein the substituted phenyl or substituted alkyl is optionally substituted with Q₂₄;
each Q₁₇ is independently H, Br, F, Cl, I, 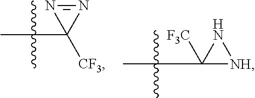 [SMILES: *C1(C(F)(F)F)N=N1 ; *C1(C(F)(F)F)NN1]
N₃, OR₄₂, C(O)R₄₂, NO₂, 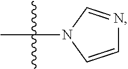 [SMILES: *n1ccnc1]
unsubstituted phenyl, substituted phenyl, unsubstituted alkyl, or substituted alkyl, wherein the alkyl is 1-3 carbons in length, wherein R₄₂ is H or Me, and wherein the substituted phenyl or substituted alkyl is optionally substituted with Q₂₄;
each Q₁₈ is independently H, Br, F, Cl, I, or OR₄₃, wherein R₄₃ is H or Me;
each Q₂₀ is independently H, Br, F, Cl, I, or OR₄₄, wherein R₄₄ is H or Me;
each Q₂₁ is independently H, Br, F, Cl, I, or OR₄₅, wherein R₄₅ is H or Me;
each Q₂₂ is independently H, Br, F, Cl or I;
each Q₂₃ is independently H, Me, unsubstituted phenyl, or substituted phenyl, wherein the substituted phenyl is optionally substituted with Q₂₄;
each Q₂₄ is independently Br, F, Cl, I, Me, or OR₄₆, wherein R₄₆ is H or Me;
T₃ is N or C—H; and
T₄ is N or C—H,
and wherein:
(A) when A₂ is 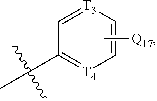
and T₃ and T₄ are each C—H, then at least one of R₃₁, R₃₂, R₃₃, R₃₄ or Q₁₇ is Br, F, Cl or I; and
(B) the compound is not one of the following:

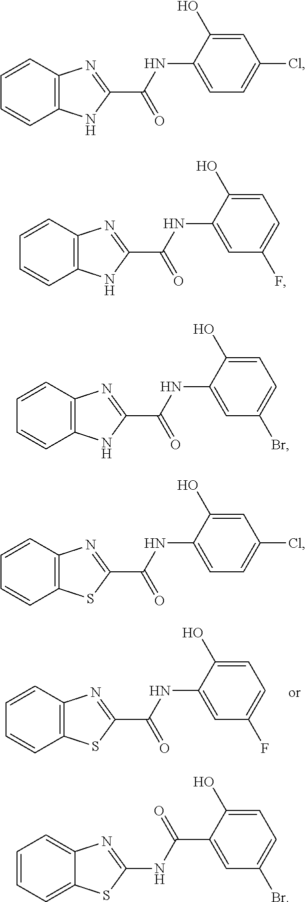

5. The method according to claim 4, wherein in general formula II:
R₃₅ is NHCH₂, NH, SCH₂, or S;
D₂ is S, N—H, N—CH₃, N—CH₂—OCH₃, N—CH₂—CH₃, N—CH₂—COOH, N—CH₂—CH₂OH, or N—R₂₉;
E₂ is C—CH₃, C—C(O)CH(CH₃)₂, C—C(O)CH₃, C—C(O)CF₃, or C—C(O)OR₃₆, wherein R₃₆ is H or Me;
each of R₂₁, R₂₂, R₂₃, R₂₄, R₂₅, R₂₆, R₂₇ and R₂₈ is independently H, Br, F, Cl, 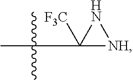 [SMILES: *C1(C(F)(F)F)NN1]
OR₂₉, C(O)R₂₉, C(O)OR₂₉, N₃, unsubstituted phenyl, substituted phenyl, unsubstituted alkyl, or substituted alkyl, wherein the alkyl is 1-3 carbons in length, wherein R₂₉ is H or Me, and wherein the substituted phenyl or substituted alkyl is optionally substituted with Q₁₄;
each Q₁₄ is independently Br, F, Cl, Me, or OR₃₇, and
M₂ is N, C—H, C—C(O)CH₃, C—C(O)CF₃, or C—C(O)OR₃₆.

6. The method according to claim 4, wherein in general formula III:
R₃₉ is NHCH₂, NH, SCH₂, or S;
D₃ is S, N—H, N—CH₃, N—CH₂—OCH₃, N—CH₂—CH₃, N—CH₂—COOH, N—CH₂—CH₂OH, or N—R₆₁;
E₃ is N, C—H, C—Cl, C—CH₃, C—C(O)CH(CH₃)₂, C—C(O)CH₃, C—C(O)CF₃, or C—C(O)OR₄₀;
each of R₃₁, R₃₂, R₃₃ and R₃₄ is independently H, Br, F, Cl, 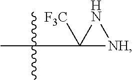 [SMILES: *C1(C(F)(F)F)NN1]
OR₄₁, C(O)R₄₁, C(O)OR₄₁, N₃, unsubstituted phenyl, substituted phenyl, unsubstituted alkyl, or substituted alkyl, wherein the alkyl is 1-3 carbons in length, wherein R₄₁ is H or Me, and wherein the substituted phenyl or substituted alkyl is optionally substituted with Q₂₄;
each Q₁₇ is independently H, Br, F, Cl, 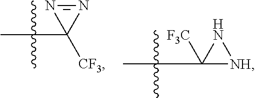 [SMILES: *C1(C(F)(F)F)N=N1 ; *C1(C(F)(F)F)NN1]
N₃, OR₄₂, C(O)R₄₂, NO₂, 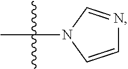 [SMILES: *n1ccnc1]
unsubstituted phenyl, substituted phenyl, unsubstituted alkyl, or substituted alkyl, wherein the alkyl is 1-3 carbons in length, wherein R₄₂ is H or Me, and wherein the substituted phenyl or substituted alkyl is optionally substituted with Q₂₄;
each Q₁₈ is independently H, Br, F, Cl, or OR₄₃;
each Q₂₀ is independently H, Br, F, Cl, or OR₄₄;
each Q₂₁ is independently H, Br, F, Cl, or OR₄₅;
each Q₂₂ is independently H, Br, F or Cl, and
each Q₂₄ is independently Br, F, Cl, Me, or OR₄₆.

7. The method according to claim 4, wherein the compound is a compound of general formula II.

8. The method according to claim 4, wherein the compound is a compound of general formula III.

9. The method according to claim 1, wherein the compound of general formula I is a compound of formula 7B or 7C: 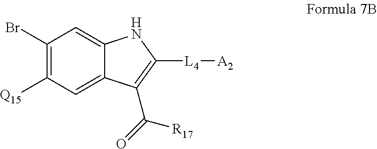
wherein:
Q₁₅ is H or Br;
R₁₇ is OH, CH₃, CH(CH₃)₂, CF₃, or OCH₃;
L4 is —CH₂CH₂—, —CHCH—, or 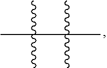 [SMILES: **]
and
A₂ is 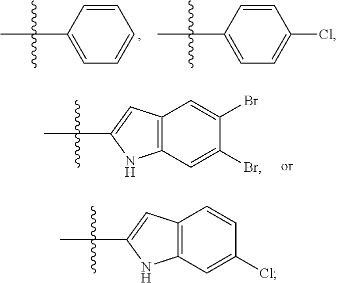
wherein: 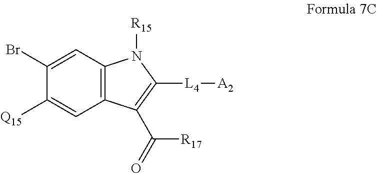
Q₁₅ is H or Br;
R₁₅ is H or CH₃;
R₁₇ is OH, CH₃, CH(CH₃)₂, CF₃, or OCH₃;
L4 is —CH₂CH₂—, —CHCH—, —C(O)NH—, —NHC(O)— or 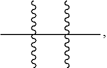 [SMILES: **]
and
A2 is 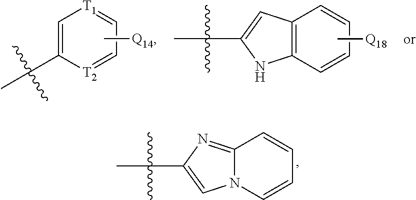
wherein each Q₁₄ is independently H, Cl, F, Br or OMe, each Q₁₅ is independently H, Cl, F, Br, OMe, substituted phenyl or unsubstituted phenyl, and T₁ and T₂ are each independently C—H or N.

10-13. (canceled)

14. The method according to claim 1, wherein the bacterial infection comprises an infection by a gram positive bacterium.

15-19. (canceled)

20. The method according to claim 1, wherein the bacterial infection comprises an infection by a gram negative bacterium.

21-25. (canceled)

26. The method according to claim 1, wherein the bacterial infection comprises an infection by a drug resistant bacterial strain.

27-34. (canceled)

35. The method according to claim 1, wherein the compound is administered parenterally, orally or topically.

36. The method according to claim 1, wherein the compound is administered in combination with another antibiotic.

37. A method of inhibiting a pyruvate kinase (PK) from a bacterial strain comprising contacting the pyruvate kinase with an effective amount of a compound of general formula I, as defined in claim 1, or a salt thereof, wherein the compound or salt thereof has bacterial PK inhibitory activity.

38. The method according to claim 37, wherein the method is an in vitro method.

39. The method according to claim 37, wherein the method is an in vivo method.

40. The method according to claim 39, wherein the contacting is by administering the compound of general formula I to a subject known to have or suspected of having a bacterial infection.

41. The method according to claim 40, wherein the compound is administered parenterally, orally or topically.

42. The method according to claim 40, wherein the compound is administered in combination with another antibiotic.

43-111. (canceled)
